(19) United States
(12) Patent Application Publication
(10) Pub. No.: US 2024/0376116 A1
(43) Pub. Date: Nov. 14, 2024

(54) IBOGAINE AND NORIBOGAINE ANALOGS AND METHODS OF USE

(71) Applicant: Atai Therapeutics, Inc., New York, NY (US)

(72) Inventors: Tanweer A. KHAN, Bridgewater, NJ (US); Glenn SHORT, Scituate, MA (US); Robert B. PERNI, Marlborough, MA (US); Alan C. GIBBS, Marlborough, MA (US); Jeffrey O'MEARA, Chertsey (CA); Harpreet KAUR, Brampton (CA); Ahmed Magdy ALI, Milton (CA)

(21) Appl. No.: 18/646,264

(22) Filed: Apr. 25, 2024

Related U.S. Application Data
(60) Provisional application No. 63/592,078, filed on Oct. 20, 2023.
(60) Provisional application No. 63/499,117, filed on Apr. 28, 2023.

(51) Int. Cl. C07D 487/22; C07D 491/22; C07D 495/22

(52) U.S. Cl. CPC C07D 487/22; C07D 491/22; C07D 495/22

(57) ABSTRACT

Provided herein are compounds of Formula (I), (II), (III), or pharmaceutically acceptable salt, prodrug, or steroisomer thereof, and compositions thereof, wherein R₁, R₁′, R₂, R₂′, R₃, R₆, R₇, X₁, X₂, X₃, X₄, and Z are defined herein. The disclosed compounds are useful for treating various conditions, including alcoholism, substance abuse disorder, and opioid use disorder.

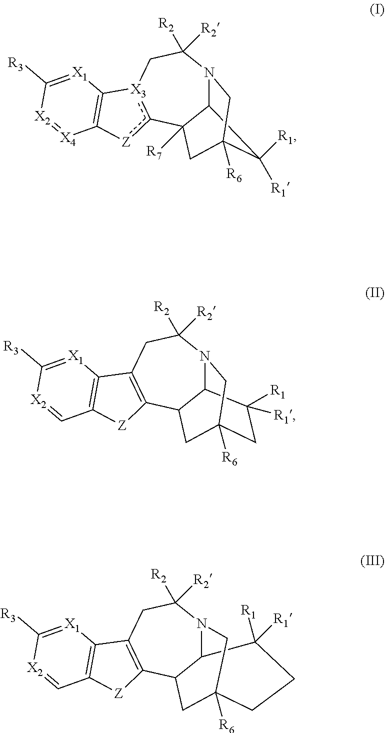

### CROSS REFERENCE TO RELATED APPLICATIONS

This application claims priority to U.S. Provisional Application No. U.S. 63/499,117, filed Apr. 28, 2023 and U.S. Provisional Application No. 63/592,078, filed Oct. 20, 2023, the disclosure of each of which is incorporated by reference in its entirety for all purposes.

### BACKGROUND

Ibogaine is a naturally occurring psychoactive compound with anticipated therapeutic uses for the treatment of substance use disorders. In particular, a recent animal research showed that ibogaine reduced self-administration of several drugs, including opiates, cocaine, and ethanol (Belgers et al., 2016). Ibogaine is metabolized in the body to its main metabolite, noribogaine, which is a non-hallucinogenic compound with an overlapping, but distinct profile of pharmacological effects.

The pharmacological basis for the therapeutic effects of ibogaine and noribogaine are unclear. Despite these benefits, the pharmacokinetic and biodistribution profile of ibogaine potentially limits its therapeutic utility.

There is a need for improved derivatives of ibogaine and noribogaine.

### BRIEF SUMMARY OF THE INVENTION

In embodiments, the present disclosure provides a compound of Formula (I):

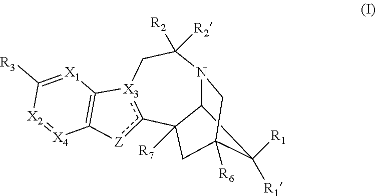

or a pharmaceutically acceptable salt, prodrug, or stereoisomer thereof, wherein:
X₁, X₂ and X₄ are independently C(R₃) or N;
X₃ is C or N;
R₁ and R₁′ are independently hydrogen, alkyl, —CF₃, —CH(Rₐ)(R_{b}), —CH₂ORₐ, —CH₂CH₂ORₐ, —CH₂NH₂, —CH₂NRₐRₐ, —CH₂SRₐ, —C(═O)ORₐ, —C(═O)NHRₐ, —ORₐ, or —C(═O)N(Rₐ)(R_{b}); R₂ and R₂′ are independently hydrogen, deuterium, —CH₂OH, —CH₂O-alkyl, —C(═O)OH, —C(═O)N(Rₐ)(R_{b}), or R₂ and R₂′ taken together with the atom to which they are attached form ═O, ═S, ═NH, or ═N-alkyl;
each R₃ is independently hydrogen, deuterium, halogen, alkyl, —ORₐ, —NO₂, —CN, —CF₃, cycloalkyl, aryl, heteroaryl, —OAc, —SRₐ, —NH₂, —NH(alkyl), —NH(alkenyl), —NH(alkynyl), —NH(aryl), —NH(heteroaryl), —N(cycloalkyl), —C(═O)R_{b}, —S(═O)R_{b}, —S(═O)(═NH)R_{b}, —S(═O)₂R_{b}, —NHS(═O)₂R_{b}, —OC(═O)R_{b}, SC(═O)R_{b}, —NHC(═O)R_{c}, or —NHC(═S)R_{c};
R₆ is hydrogen, alkyl, —CH₂ORₐ, —CH₂NH₂, —CH₂N(Rₐ)(R_{b}), —CH₂SRₐ, —C(═O)ORₐ, —C(═O)NHRₐ, or —C(═O)N(Rₐ)(R_{b});
R₇ is hydrogen, halogen, deuterium, alkyl, alkoxy, alkylene-OH, alkylene-O-alkyl, alkylene-NH₂, alkylene-NH(alkyl), alkylene-N(alkyl)₂, —C(═O)ORₐ, or —C(═O)NRₐ; each Rₐ is independently hydrogen, alkyl, deuterated alkyl, alkenyl, alkylene-aryl, alkylene-cycloalkyl, aryl, or heteroaryl;
each R_{b} is independently alkyl, aryl, heteroaryl, —OH, —O-alkyl, —NH₂, —NH(alkyl), or —N(alkyl)₂;
each R_{c} is independently alkyl, aryl, —O-alkyl, —S-alkyl, —S-aryl, —NH₂, —NH(alkyl), or —N(alkyl)₂; and
Z is O, S, or N(R₄), wherein R₄ is absent, hydrogen, alkyl, deuterated alkyl, heteroaryl, alkenyl, alkylene-aryl, alkylene-cycloalkyl, aryl, or —S(═O)₂Rₐ.

In embodiments, the present disclosure provides a compound of Formula (II):

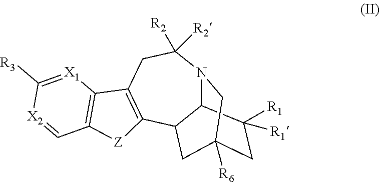

or a pharmaceutically acceptable salt, prodrug, or stereoisomer thereof, wherein:
X₁ and X₂ are independently —CR₃— or —N—, wherein at least one of X₁ and X₂ is —N—;
R₁ and R₁′ are independently hydrogen, alkyl, —CH₂ORₐ, —CH₂NH₂, —CH₂NRₐRₐ, —CH₂SRₐ, —C(═O)ORₐ, —CONHRₐ, or —CON(Rₐ)Rₐ;
R₂ and R₂′ are independently hydrogen, deuterium, —CH₂OH, —CH₂O-alkyl, —COOH, —CON(Rₐ)Rₐ, or R₂ and R₂′ taken together with the atom to which they are attached form ═O, ═S, ═NH, or ═N-alkyl;
each R₃ is independently hydrogen, deuterium, halogen, alkyl, —ORₐ, —NO₂, —CN, cycloalkyl, aryl, heteroaryl, —OAc, —SRₐ, —NH₂, —NH(alkyl), —NH(alkenyl), —NH(alkynyl), —NH(aryl), —NH(heteroaryl), —N(cycloalkyl), —C(═O)R_{b}, —S(═O)R_{b}, —S(═O)(═NH)R_{b}, —SO₂R_{b}, —NHSO₂R_{b}, —OC(═O)R_{b}, —SC(═O)R_{b}, —NHC(═O)R_{c} or —NHC(S)R_{c}; R₆ is hydrogen, alkyl, —CH₂ORₐ, —CH₂NH₂, —CH₂NRₐRₐ, —CH₂SRₐ, —C(═O)ORₐ, —CONHRₐ, or —CON(Rₐ)Rₐ;
each Rₐ is independently hydrogen, alkyl, deuterated alkyl, alkenyl, alkylene-aryl, alkylene-cycloalkyl, aryl, or heteroaryl;
each R_{b} is independently alkyl, aryl, heteroaryl, —OH, —O-alkyl, —NH₂, —NH(alkyl), or —N(alkyl)₂;
each R_{c} is independently alkyl, aryl, —O-alkyl, —S-alkyl, —S-aryl, —NH₂, —NH(alkyl), or —N(alkyl)₂; and
Z is O, S, or NR₄, wherein R₄ is hydrogen, alkyl, deuterated alkyl, heteroaryl, alkenyl, alkylene-aryl, alkylene-cycloalkyl, aryl, or —SO₂Rₐ.

In embodiments, the present disclosure provides a compound of Formula (II):

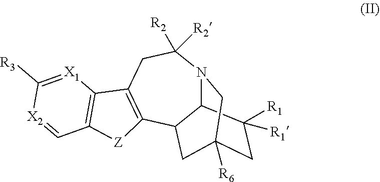

or a pharmaceutically acceptable salt, prodrug, or stereoisomer thereof, wherein:
X₁ and X₂ are independently —CR₃—, or —N—;
R₁ and R₁′ are independently hydrogen, alkyl, —CH₂ORₐ, —CH₂NH₂, —CH₂NRₐRₐ, —CH₂SRₐ, —C(═O)ORₐ, —CONHRₐ, or —C(═O)N(Rₐ)Rₐ;
R₂ and R₂′ are independently hydrogen, —CH₂OH, —CH₂O-alkyl, —COOH, —CON(Rₐ)Rₐ, or R₂ and R₂′ taken together with the atom to which they are attached form ═O, ═S, ═NH, or ═N-alkyl;
each R₃ is independently deuterium, halogen, alkyl, —ORₐ, —NO₂, —CN, cycloalkyl, aryl, heteroaryl, —OAc, —SRₐ, —NH₂, —NH(alkyl), —NH(alkenyl), —NH(alkynyl), —NH(aryl), —NH(heteroaryl), —N(cycloalkyl), —C(═O)R_{b}, —S(═O)R_{b}, —S(═O)(═NH)R_{b}, —SO₂R_{b}, —NHSO₂R_{b}, —SC(═O)R_{b}, —NHC(═O)R_{c} or —NHC(S)R_{c}; R₆ is hydrogen, alkyl, —CH₂ORₐ, —CH₂NH₂, —CH₂NRₐRₐ, —CH₂SRₐ, —C(═O)ORₐ, —CONHRₐ, or —CON(Rₐ)Rₐ;
each Rₐ is independently hydrogen, alkyl, deuterated alkyl, alkenyl, alkylene-aryl, alkylene-cycloalkyl, aryl, or heteroaryl;
each R_{b} is independently alkyl, aryl, heteroaryl, —OH, —O-alkyl, —NH₂, —NH(alkyl), or —N(alkyl)₂;
each R_{c} is independently alkyl, aryl, —O-alkyl, —S-alkyl, —S-aryl, —NH₂, —NH(alkyl), or —N(alkyl)₂;
Z is NR₄, wherein R₄ is hydrogen, alkyl, deuterated alkyl, heteroaryl, alkenyl, alkylene-aryl, alkylene-cycloalkyl, aryl, or —SO₂Rₐ; and
wherein when R₂ and R₂′ are hydrogen, R₃ is —OH, and X₁ and X₂ are CH, then R₁ is not —CH₂CH₃, —(CH₂)₂CN, —(CH₂)₂OCH₂C₆H₅, —CD₂CD₃, —CH₂CD₃, —(CH₂)₂OH, —(CH₂)₂OCH₂CH₃, or —(CH₂)₂CF₃;
wherein when R₁ is —CH₂CH₃ or —CH₂OH, and X₁ and X₂ are CH, then R₃ is not —OCH₂CH₃, —OC(CH₃)₃, or —CH═CH₂, or R₄ is not —(CH₂)₃N(CH₃)₂; and the compound is not

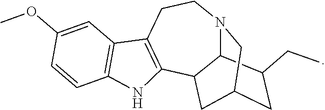 [SMILES: CCC1CC2CC3c4[nH]c5ccc(OC)cc5c4CCN(C2)C13]

In embodiments, the present disclosure provides a compound of Formula (III):

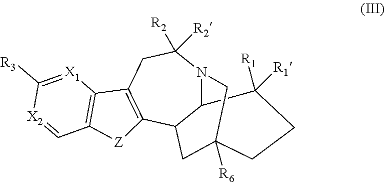

Or a pharmaceutically acceptable salt, prodrug, or stereoisomer thereof, wherein:
X₁ and X₂ are independently —CR₃—, or —N—;
R₁ and R₁′ are independently hydrogen, alkyl, —CH₂ORₐ, —CH₂CH₂ORₐ, —CH₂NH₂, —CH₂NRₐRₐ, —CH₂SRₐ, —C(═O)ORₐ, —CONHRₐ, or —CON(Rₐ)Rₐ;
R₂ and R₂′ are independently hydrogen, deuterium, —CH₂OH, —CH₂O-alkyl, —COOH, —CON(Rₐ)Rₐ, or R₂ and R₂′ taken together with the atom to which they are attached form ═O, ═S, ═NH, or ═N-alkyl;
Each R₃ is independently hydrogen, deuterium, halogen, alkyl, —ORₐ, —NO₂, —CN, cycloalkyl, aryl, heteroaryl, -Oac, —SRₐ, —NH₂, —NH(alkyl), —NH(alkenyl), —NH(alkynyl), —NH(aryl), —NH(heteroaryl), —N(cycloalkyl), —C(═O)R_{b}, —S(═O)R_{b}, —S(═O)(═NH)R_{b}, —SO₂R_{b}, —NHSO₂R_{b}, —OC(═O)R_{b}, SC(═O)R_{b}, —NHC(═O)R_{c}, or —NHC(S)R_{c};
R₆ is hydrogen, alkyl, —CH₂ORₐ, —CH₂NH₂, —CH₂NRₐRₐ, —CH₂SRₐ, —C(═O)ORₐ, —CONHRₐ, or —CON(Rₐ)Rₐ;
each Rₐ is independently hydrogen, alkyl, deuterated alkyl, alkenyl, alkylene-aryl, alkylene-cycloalkyl, aryl, or heteroaryl;
each R_{b} is alkyl, aryl, heteroaryl, —OH, —O-alkyl, —NH₂, —NH(alkyl), or —N(alkyl)₂;
each R_{c} is alkyl, aryl, —O-alkyl, —S-alkyl, —S-aryl, —NH₂, —NH(alkyl), or —N(alkyl)₂;
Z is N(R₄), wherein R₄ is hydrogen, alkyl, deuterated alkyl, heteroaryl, alkenyl, alkylene-aryl, alkylene-cycloalkyl, aryl, or —SO₂Rₐ; and
R₄ is hydrogen, alkyl, deuterated alkyl, heteroaryl, alkenyl, alkylene-aryl, alkylene-cycloalkyl, aryl, or —SO₂Rₐ.

### DETAILED DESCRIPTION OF THE INVENTION

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

### Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The terms “administer,” “administering” or “administration” as used herein refer to administering a compound or pharmaceutically acceptable salt of the compound or a composition or formulation comprising the compound or pharmaceutically acceptable salt of the compound to a patient.

The term “treating” as used herein with regard to a patient or subject, refers to improving at least one symptom of the patient's or subject's disorder. In embodiments, treating can be improving, or at least partially ameliorating a disorder or one or more symptoms of a disorder.

The term “therapeutically effective” applied to dose or amount refers to that quantity of a compound or pharmaceutical formulation that is sufficient to result in a desired clinical benefit after administration to a patient or subject in need thereof.

The phrase “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “salts” as used herein embraces pharmaceutically acceptable salts commonly used to form alkali metal salts of free acids and to form addition salts of free bases. For example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. The acids that may be used to prepare pharmaceutically acceptable acid addition salts of such basic compounds are those that form non-toxic acid addition salts, i.e., salts containing pharmaceutically acceptable anions, including but not limited to malate, oxalate, chloride, bromide, iodide, nitrate, acetate, tartrate, oleate, fumarate, formate, benzoate, glutamate, methanesulfonate, benzenesulfonate, and p-toluenesulfonate salts. Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e. g., lysine and arginine dicyclohexylamine and the like. Examples of metal salts include lithium, sodium, potassium, magnesium, calcium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like. Those skilled in the art will further recognize that acid addition salts may be prepared by reaction of the compounds with the appropriate inorganic or organic acid via any of a number of known methods.

When a range of values is listed, it is intended to encompass each value and sub-range within the range. For example, “C₁-C₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” or “alkyl group” refers to a fully saturated, straight or branched hydrocarbon chain having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, and the like. The alkylene chain is attached to the rest of the molecule through a single bond and to a radical group (e.g., those described herein) through a single bond. The points of attachment of the alkylene chain to the rest of the molecule and to the radical group can be through one carbon or any two carbons within the chain. Unless stated otherwise, an alkylene chain can be optionally substituted.

“Alkenyl” or “alkenyl group” refers to a straight or branched hydrocarbon chain having from two to twelve carbon atoms and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C2-C12 alkenyl, an alkenyl comprising up to 10 carbon atoms is a C2-C10 alkenyl, an alkenyl group comprising up to 6 carbon atoms is a C2-C6 alkenyl and an alkenyl comprising up to 5 carbon atoms is a C2-C5 alkenyl. A C2-C5 alkenyl includes C5 alkenyls, C4 alkenyls, C3 alkenyls, and C2 alkenyls. A C2-C6 alkenyl includes all moieties described above for C2-C5 alkenyls but also includes C6 alkenyls. A C2-C10 alkenyl includes all moieties described above for C2-C5 alkenyls and C2-C6 alkenyls, but also includes C7, C8, C9 and C10 alkenyls. Similarly, a C2-C12 alkenyl includes all the foregoing moieties, but also includes C11 and C12 alkenyls. Non-limiting examples of C2-C12 alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkynyl” or “alkynyl group” refers to a straight or branched hydrocarbon chain having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C2-C12 alkynyl, an alkynyl comprising up to 10 carbon atoms is a C2-C10 alkynyl, an alkynyl group comprising up to 6 carbon atoms is a C2-C6 alkynyl and an alkynyl comprising up to 5 carbon atoms is a C2-C5 alkynyl. A C2-C5 alkynyl includes C5 alkynyls, C4 alkynyls, C3 alkynyls, and C2 alkynyls. A C2-C6 alkynyl includes all moieties described above for C2-C5 alkynyls but also includes C6 alkynyls. A C2-C10 alkynyl includes all moieties described above for C2-C5 alkynyls and C2-C6 alkynyls, but also includes C7, C8, C9 and C10 alkynyls. Similarly, a C2-C12 alkynyl includes all the foregoing moieties, but also includes C11 and C12 alkynyls. Non-limiting examples of C2-C12 alkenyl include ethynyl, propynyl, butynyl, pentynyl and the like. Unless stated otherwise, an alkynyl group can be optionally substituted.

“Alkoxy” refers to a group of the formula —ORₐ where Rₐ is an alkyl, alkenyl or alknyl as defined above containing one to twelve carbon atoms. Unless stated otherwise, an alkoxy group can be optionally substituted.

“Aryl” refers to a hydrocarbon ring system comprising hydrogen, 6 to 18 carbon atoms and at least one aromatic ring, and which is attached to the rest of the molecule by a single bond. For purposes of this disclosure, the aryl can be a monocyclic, bicyclic, tricyclic or tetracyclic ring system, which can include fused or bridged ring systems. Aryls include, but are not limited to, aryls derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise, the “aryl” can be optionally substituted.

“Heteroaryl” refers to a 5- to 20-membered ring system comprising hydrogen atoms, one to nineteen carbon atoms, one to six heteroatoms selected from the group consisting of nitrogen, oxygen and sulfur, at least one aromatic ring, including compounds with aromatic resonance structures (e.g., 2-pyridone), and which is attached to the rest of the molecule by a single bond. For purposes of this disclosure, the heteroaryl can be a monocyclic, bicyclic, tricyclic or tetracyclic ring system, which can include fused or bridged ring systems; and the nitrogen, carbon or sulfur atoms in the heteroaryl can be optionally oxidized; the nitrogen atom can be optionally quaternized. Examples include, but are not limited to, azepinyl, acridinyl, benzimidazolyl, benzothiazolyl, benzindolyl, benzofuranyl, benzooxazolyl, benzothiadiazolyl, benzo[b][1,4]dioxepinyl, 1,4 benzodioxanyl, benzonaphthofuranyl, benzoxazolyl, benzodioxolyl, benzodioxinyl, benzopyranyl, benzopyranonyl, benzofuranonyl, benzothienyl (benzothiophenyl), benzotriazolyl, benzo[4,6]imidazo[1,2 a]pyridinyl, carbazolyl, cinnolinyl, dibenzofuranyl, dibenzothiophenyl, furanyl, furanonyl, isothiazolyl, imidazolyl, indazolyl, indolyl, isoindolyl, indolinyl, isoindolinyl, isoquinolyl, indolizinyl, isoxazolyl, naphthyridinyl, oxadiazolyl, 2 oxoazepinyl, oxazolyl, oxiranyl, 1-oxidopyridinyl, 1 oxidopyrimidinyl, 1-oxidopyrazinyl, 1-oxidopyridazinyl, 1 phenyl 1H pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise, a heteroaryl group can be optionally substituted.

“Cycloalkyl” refers to a stable non-aromatic monocyclic or polycyclic fully saturated hydrocarbon consisting solely of carbon and hydrogen atoms, which can include fused, bridged, or spirocyclic ring systems, having from three to twenty carbon atoms (e.g., having from three to ten carbon atoms) and which is attached to the rest of the molecule by a single bond. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, 7,7-dimethyl-bicyclo[2.2.1]heptanyl, and the like. Unless otherwise stated, a cycloalkyl group can be optionally substituted.

“Heterocyclyl,” “heterocyclic ring” or “heterocycle” refers to a stable saturated, unsaturated, or aromatic 3- to 20-membered ring which consists of two to nineteen carbon atoms and from one to six heteroatoms selected from the group consisting of nitrogen, oxygen and sulfur, and which is attached to the rest of the molecule by a single bond. Heterocyclyl or heterocyclic rings include heteroaryls, heterocyclylalkyls, heterocyclylalkenyls, and hetercyclylalkynyls. Unless stated otherwise specifically in the specification, the heterocyclyl can be a monocyclic, bicyclic, tricyclic or tetracyclic ring system, which can include fused, bridged, or spirocyclic ring systems; and the nitrogen, carbon or sulfur atoms in the heterocyclyl can be optionally oxidized; the nitrogen atom can be optionally quaternized; and the heterocyclyl can be partially or fully saturated. Examples of such heterocyclyl include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“Haloalkyl” refers to an alkyl, as defined above, that is substituted by one or more halo radicals, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise, a haloalkyl group can be optionally substituted.

The term “substituted” used herein means any of the groups described herein (e.g., alkyl, alkenyl, alkynyl, alkoxy, aryl, aralkyl, carbocyclyl, cycloalkyl, cycloalkenyl, cycloalkynyl, haloalkyl, heterocyclyl, and/or heteroaryl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkyl, haloalkyl, haloalkenyl, haloalkynyl, heterocyclyl, N-heterocyclyl, heterocyclylalkyl, heteroaryl, N-heteroaryl and/or heteroarylalkyl group. In some embodiments, “substituted” further means any alkyl, cycloalkyl or heterocyclylalkyl in which one or more hydrogen atoms is replaced by an isotope e.g., deuterium. In addition, each of the foregoing substituents can also be optionally substituted with one or more of the above substituents.

### Compounds

The present disclosure provides compounds that are analogs of ibogaine and noribogaine or modified core structures thereof, as well as pharmaceutical compositions thereof and uses thereof in treating various diseases and disorders.

In embodiments, the present disclosure provides a compound of Formula (I):

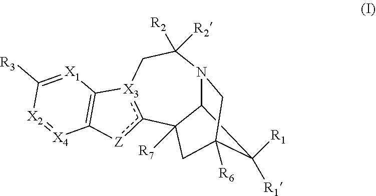

or a pharmaceutically acceptable salt, prodrug, or stereoisomer thereof, wherein:
X₁, X₂ and X₄ are independently C(R₃) or N;
X₃ is C or N;
R₁ and R₁′ are independently hydrogen, alkyl, —CF₃, —CH(Rₐ)(R_{b}), —CH₂ORₐ, —CH₂CH₂ORₐ, —CH₂NH₂, —CH₂NRₐRₐ, —CH₂SRₐ, —C(═O)ORₐ, —C(═O)NHRₐ, —ORₐ, or —C(═O)N(Rₐ)(R_{b});
R₂ and R₂′ are independently hydrogen, deuterium, —CH₂OH, —CH₂O-alkyl, —C(═O)OH, —C(═O)N(Rₐ)(R_{b}), or R₂ and R₂′ taken together with the atom to which they are attached form ═O, ═S, ═NH, or ═N-alkyl;
each R₃ is independently hydrogen, deuterium, halogen, alkyl, —ORₐ, —NO₂, —CN, —CF₃, cycloalkyl, aryl, heteroaryl, —OAc, —SRₐ, —NH₂, —NH(alkyl), —NH(alkenyl), —NH(alkynyl), —NH(aryl), —NH(heteroaryl), —N(cycloalkyl), —C(═O)R_{b}, —S(═O)R_{b}, —S(═O)(═NH)R_{b}, —S(═O)₂R_{b}, —NHS(═O)₂R_{b}, —OC(═O)R_{b}, SC(═O)R_{b}, —NHC(═O)R_{c}, or —NHC(═S)R_{c};
R₆ is hydrogen, alkyl, —CH₂ORₐ, —CH₂NH₂, —CH₂N(Rₐ)(R_{b}), —CH₂SRₐ, —C(═O)ORₐ, —C(═O)NHRₐ, or —C(═O)N(Rₐ)(R_{b});
R₇ is hydrogen, halogen, deuterium, alkyl, alkoxy, alkylene-OH, alkylene-O-alkyl, alkylene-NH₂, alkylene-NH(alkyl), alkylene-N(alkyl)₂, —C(═O)ORₐ, or —C(═O)NRₐ;
each Rₐ is independently hydrogen, alkyl, deuterated alkyl, alkenyl, alkylene-aryl, alkylene-cycloalkyl, aryl, or heteroaryl;
each R_{b} is independently alkyl, aryl, heteroaryl, —OH, —O-alkyl, —NH₂, —NH(alkyl), or —N(alkyl)₂;
each R_{c} is independently alkyl, aryl, —O-alkyl, —S-alkyl, —S-aryl, —NH₂, —NH(alkyl), or —N(alkyl)₂; and
Z is O, S, or N(R₄), wherein R₄ is absent, hydrogen, alkyl, deuterated alkyl, heteroaryl, alkenyl, alkylene-aryl, alkylene-cycloalkyl, aryl, or —S(═O)₂Rₐ.

In embodiments, the present disclosure provides a compound of Formula (I″),

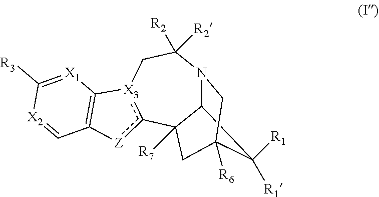

or a pharmaceutically acceptable salt, prodrug, or stereoisomer thereof, wherein:
X₁ and X₂ are independently C(R₃) or N;
X₃ is C or N;
R₁ and R₁′ are independently hydrogen, alkyl, —CF₃, —CH(Rₐ)(R_{b}), —CH₂ORₐ, —CH₂CH₂ORₐ, —CH₂NH₂, —CH₂NRₐRₐ, —CH₂SRₐ, —C(═O)ORₐ, —C(═O)NHRₐ, —ORₐ, or —C(═O)N(Rₐ)(R_{b});
R₂ and R₂′ are independently hydrogen, deuterium, —CH₂OH, —CH₂O-alkyl, —C(═O)OH, —C(═O)N(Rₐ)(R_{b}), or R₂ and R₂′ taken together with the atom to which they are attached form ═O, ═S, ═NH, or ═N-alkyl;
Each R₃ is independently hydrogen, deuterium, halogen, alkyl, —ORₐ, —NO₂, —CN, —CF₃, cycloalkyl, aryl, heteroaryl, -Oac, —SRₐ, —NH₂, —NH(alkyl), —NH(alkenyl), —NH(alkynyl), —NH(aryl), —NH(heteroaryl), —N(cycloalkyl), —C(═O)R_{b}, —S(═O)R_{b}, —S(═O)(═NH)R_{b}, —S(═O)₂R_{b}, —NHS(═O)₂R_{b}, —OC(═O)R_{b}, —SC(═O)R_{b}, —NHC(═O)R_{c} or —NHC(═S)R_{c};
R₆ is hydrogen, alkyl, —CH₂ORₐ, —CH₂NH₂, —CH₂N(Rₐ)(R_{b}), —CH₂SRₐ, —C(═O)ORₐ, —C(═O)NHRₐ, or —C(═O)N(Rₐ)(R_{b});
R₇ is hydrogen, halogen, deuterium, alkyl, alkoxy, —C(═O)ORₐ, or —C(═O)NRₐ;
each Rₐ is independently hydrogen, alkyl, deuterated alkyl, alkenyl, alkylene-aryl, alkylene-cycloalkyl, aryl, or heteroaryl;
each R_{b} is independently alkyl, aryl, heteroaryl, —OH, —O-alkyl, —NH₂, —NH(alkyl), or —N(alkyl)₂;
each R_{c} is independently alkyl, aryl, —O-alkyl, —S-alkyl, —S-aryl, —NH₂, —NH(alkyl), or —N(alkyl)₂; and
Z is O, S, or N(R₄), wherein R₄ is absent, hydrogen, alkyl, deuterated alkyl, heteroaryl, alkenyl, alkylene-aryl, alkylene-cycloalkyl, aryl, or —S(═O)₂Rₐ.

In embodiments, X₃ is C.

In embodiments, X₃ is N.

In embodiments, R₇ is hydrogen.

In embodiments, R₇ is O-alkyl. In embodiments, R₇ is OCH₃.

In embodiments, R₇ is halogen. In embodiments, R₇ is deuterium. In embodiments, R₇ is alkyl. In embodiments, R₇ is alkoxy. In embodiments, R₇ is —C(═O)ORₐ. In embodiments, R₇ is —C(═O)NRₐ. In embodiments, R₇ is alkylene-OH. In embodiments, R₇ is alkylene-O-alkyl. In embodiments, R₇ is alkylene-NH₂. In embodiments, R₇ is alkylene-NH(alkyl). In embodiments, R₇ is alkylene-N(alkyl)₂.

In embodiments, the present disclosure provides a compound of Formula (I′),

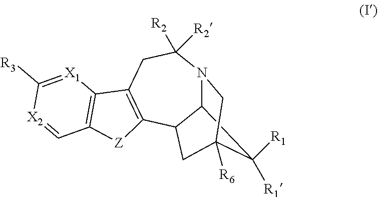

or a pharmaceutically acceptable salt, prodrug, or stereoisomer thereof, wherein:
X₁ and X₂ are independently —CR₃—, or —N—;
R₁ and R₁′ are each independently hydrogen, alkyl, —CH₂ORₐ, —CH₂CH₂ORₐ, —CH₂NH₂, —CH₂NRₐRₐ, —CH₂SRₐ, —C(═O)ORₐ, —CONHRₐ, or —CON(Rₐ)Rₐ;
R₂ and R₂′ are each independently hydrogen, deuterium, —CH₂OH, —CH₂O-alkyl, —COOH, —CON(Rₐ)Rₐ, or R₂ and R₂′ taken together with the atom to which they are attached form ═O, ═S, ═NH, or ═N-alkyl;
each R₃ is independently hydrogen, deuterium, halogen, alkyl, —ORₐ, —NO₂, —CN, cycloalkyl, aryl, heteroaryl, —OAc, —SRₐ, —NH₂, —NH(alkyl), —NH(alkenyl), —NH(alkynyl), —NH(aryl), —NH(heteroaryl), —N(cycloalkyl), —C(═O)R_{b}, —S(═O)R_{b}, —S(═O)(═NH)R_{b}, —SO₂R_{b}, —NHSO₂R_{b}, —OC(═O)R_{b}, SC(═O)R_{b}, —NHC(═O)R_{c} or —NHC(S)R_{c};
R₆ is hydrogen, alkyl, —CH₂ORₐ, —CH₂NH₂, —CH₂NRₐRₐ, —CH₂SRₐ, —C(═O)ORₐ, —CONHRₐ, or —CON(Rₐ)Rₐ;
each Rₐ is independently hydrogen, alkyl, deuterated alkyl, alkenyl, alkylene-aryl, alkylene-cycloalkyl, aryl, or heteroaryl;
each R_{b} is independently alkyl, aryl, heteroaryl, —OH, —O-alkyl, —NH₂, —NH(alkyl), or —N(alkyl)₂;
each R_{c} is independently alkyl, aryl, —O-alkyl, —S-alkyl, —S-aryl, —NH₂, —NH(alkyl), or —N(alkyl)₂; and
Z is O, S, or NR₄, wherein R₄ is hydrogen, alkyl, deuterated alkyl, heteroaryl, alkenyl, alkylene-aryl, alkylene-cycloalkyl, aryl, or SO₂Rₐ.

In embodiments, X₁ and X₂ are CR₃.

In embodiments, X₁ and X₂ are N.

In embodiments, X₁ is CR₃ and X₂ is N.

In embodiments, X₁ is N and X₂ is CR₃.

In embodiments, X₂ is COH. In embodiments, X₂ is COCH₃.

In embodiments, the compound of Formula (I), (I′), or (I″) is a compound of Formula (I-a) or a pharmaceutically acceptable salt, prodrug, or stereoisomer thereof.

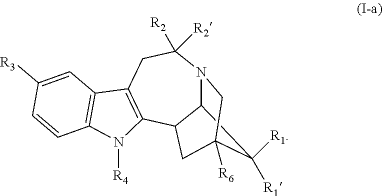

In embodiments, the compound of Formula (I), (I′), or (I″) is a compound of Formula (I-b) or a pharmaceutically acceptable salt, prodrug, or stereoisomer thereof:

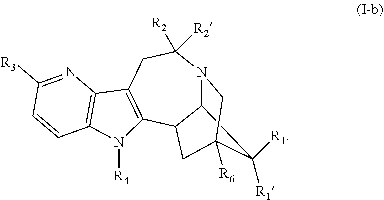

In embodiments of the compounds of Formula (I), (I′), (I″), (I-a), or (I-b), the compound is selected from:

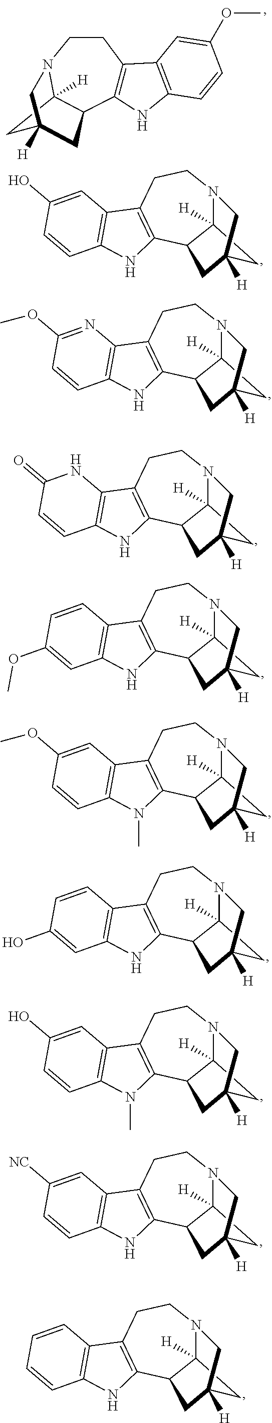

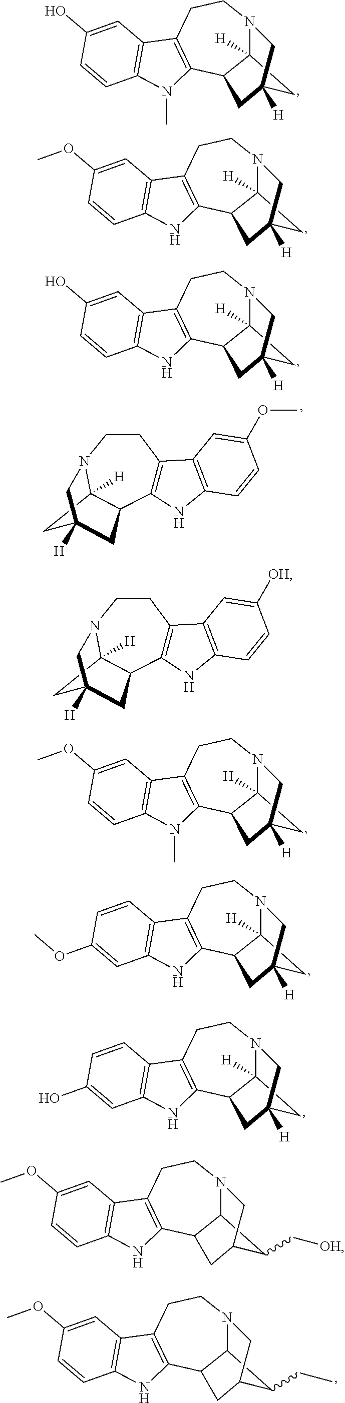

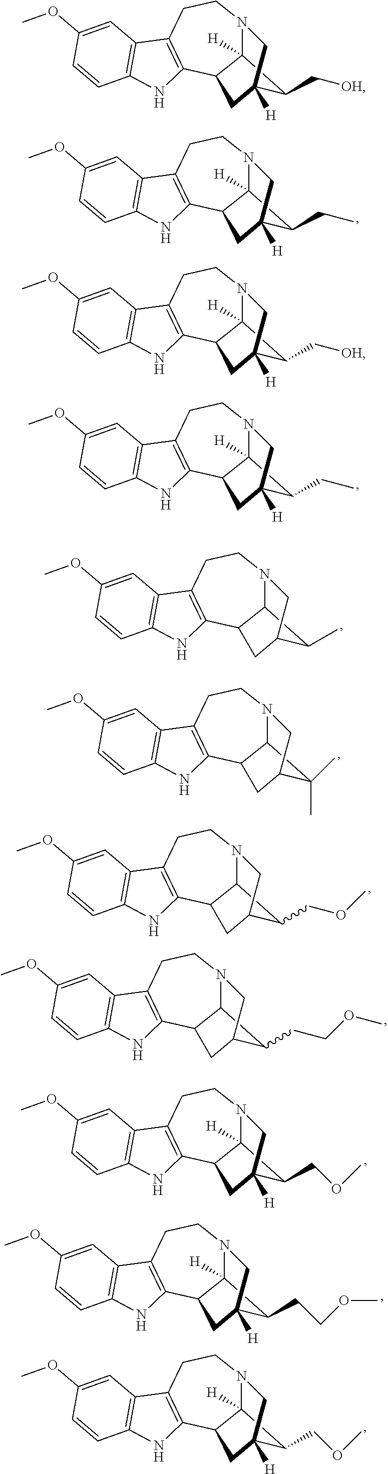

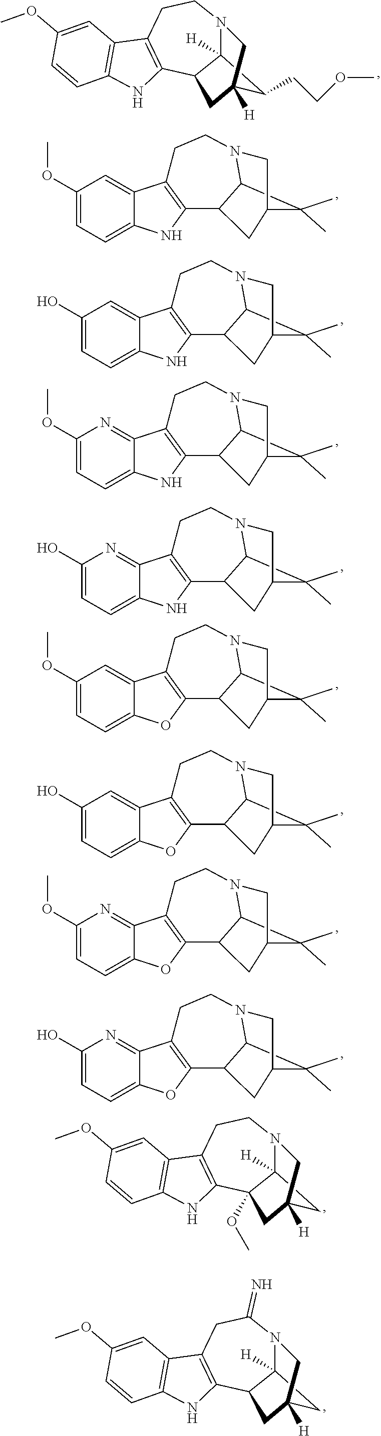

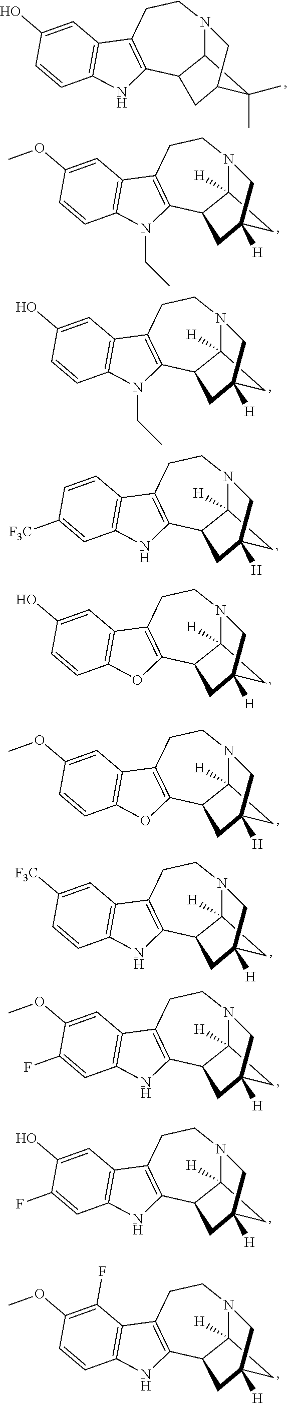

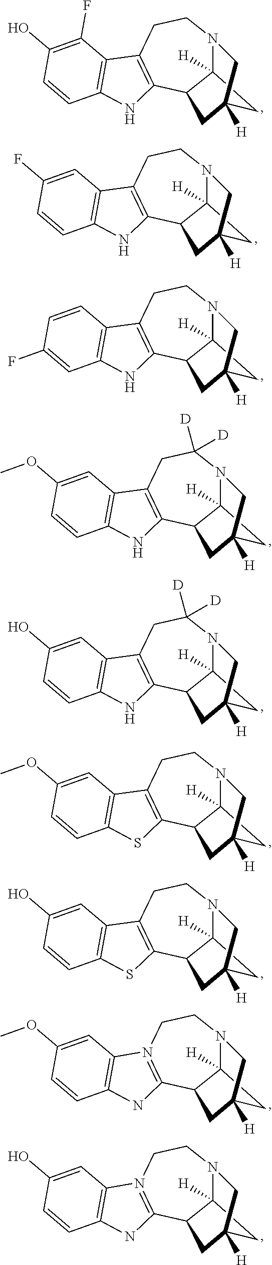

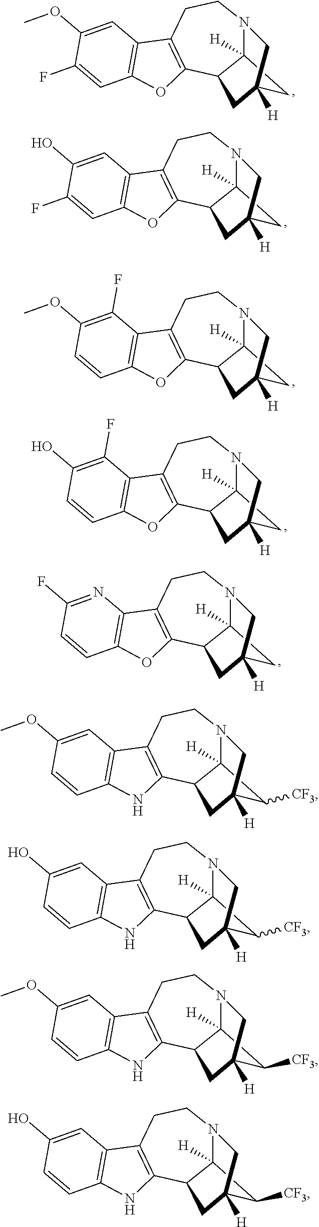

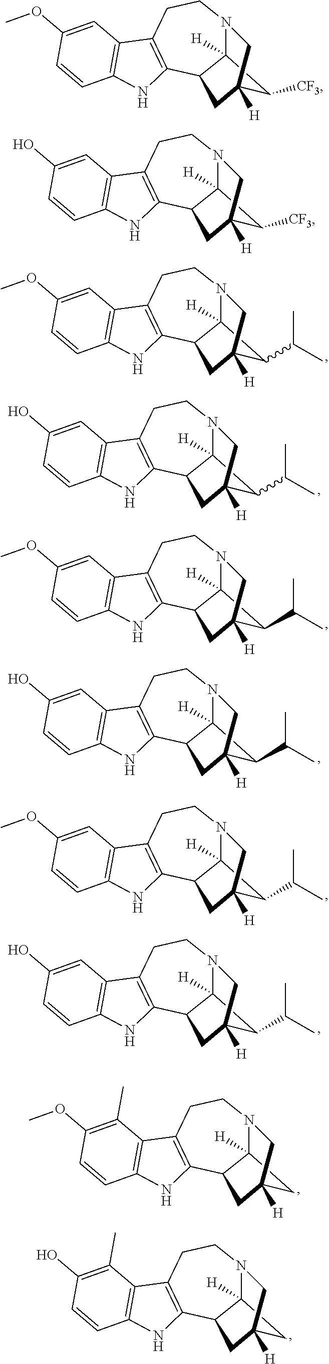

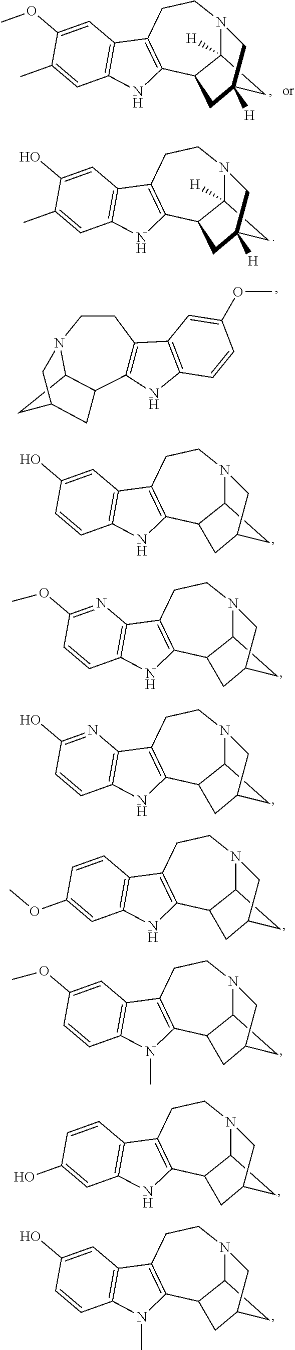

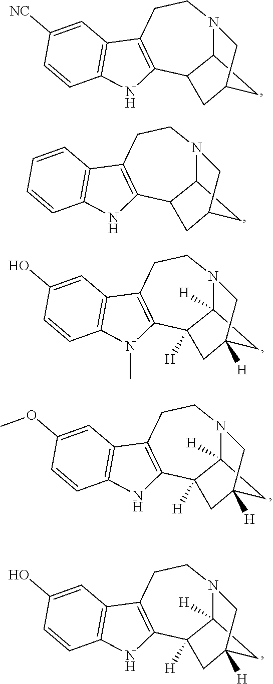

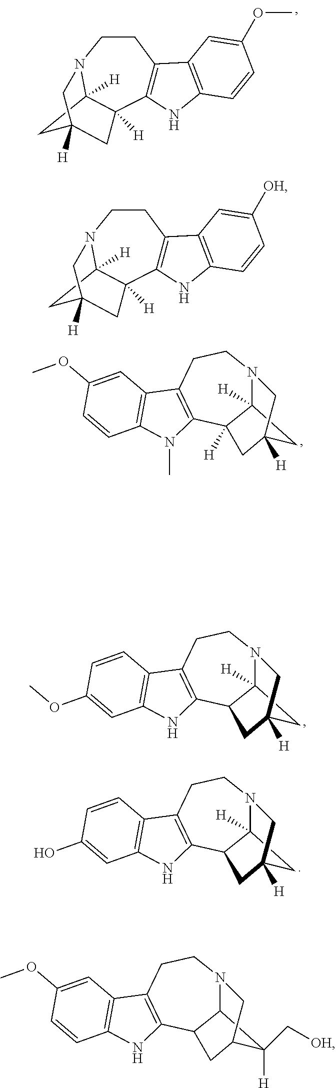

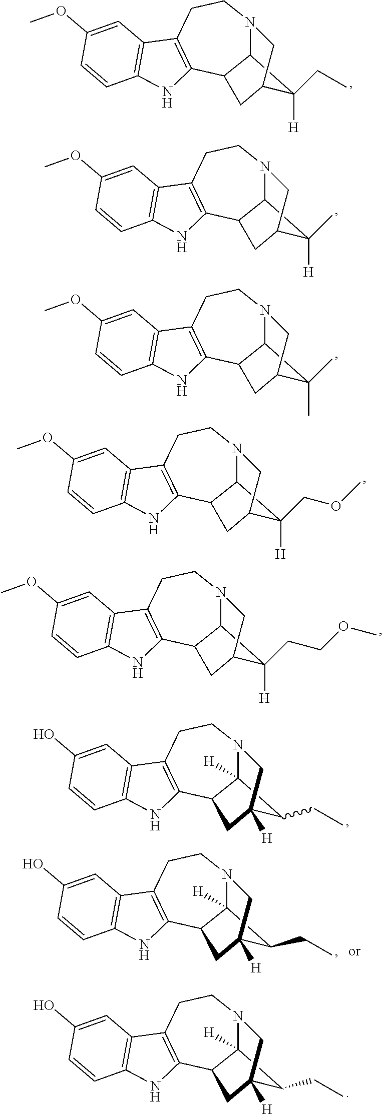

In embodiments, the present disclosure provides a compound of Formula (II):

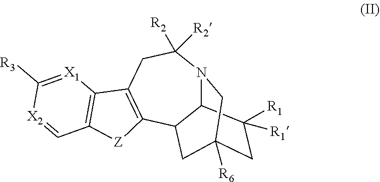

or a pharmaceutically acceptable salt, prodrug, or stereoisomer thereof, wherein:
X₁ and X₂ are independently CR₃ or N, wherein at least one of X₁ and X₂ is N;
R₁ and R₁′ are independently hydrogen, alkyl, —CH₂ORₐ, —CH₂NH₂, —CH₂NRₐRₐ, —CH₂SRₐ, —C(═O)ORₐ, —CONHRₐ, or —CON(Rₐ)Rₐ;
R₂ and R₂′ are independently hydrogen, deuterium, —CH₂OH, —CH₂O-alkyl, —COOH, —CON(Rₐ)Rₐ, or R₂ and R₂′ taken together with the atom to which they are attached form ═O, ═S, ═NH, or ═N-alkyl;
each R₃ is independently hydrogen, deuterium, halogen, alkyl, —ORₐ, —NO₂, —CN, cycloalkyl, aryl, heteroaryl, —OAc, —SRₐ, —NH₂, —NH(alkyl), —NH(alkenyl), —NH(alkynyl), —NH(aryl), —NH(heteroaryl), —N(cycloalkyl), —C(═O)R_{b}, —S(═O)R_{b}, —S(═O)(═NH)(R_{b}), —SO₂R_{b}, —NHSO₂R_{b}, —OC(═O)R_{b}, —SC(═O)R_{b}, —NHC(═O)R_{c}, or —NHC(S)R_{c};
R₆ is hydrogen, alkyl, —CH₂ORₐ, —CH₂NH₂, —CH₂NRₐRₐ, —CH₂SRₐ, —C(═O)ORₐ, —CONHRₐ, or —CON(Rₐ)Rₐ;
each Rₐ is independently hydrogen, alkyl, deuterated alkyl, alkenyl, alkylene-aryl, alkylene-cycloalkyl, aryl, or heteroaryl;
each R_{b} is independently alkyl, aryl, heteroaryl, —OH, —O-alkyl, —NH₂, —NH(alkyl), or —N(alkyl)₂;
each R_{c} is independently alkyl, aryl, —O-alkyl, —S-alkyl, —S-aryl, —NH₂, —NH(alkyl), or —N(alkyl)₂; and
Z is O, S, or NR₄, wherein R₄ is hydrogen, alkyl, deuterated alkyl, heteroaryl, alkenyl, alkylene-aryl, alkylene-cycloalkyl, aryl, or SO₂Rₐ.

In embodiments, X₁ and X₂ are N.

In embodiments, X₁ is CR₃, and X₂ is N.

In embodiments, X₁ is N, and X₂ is CR₃.

In embodiments, the compound of Formula (II) is a compound of the Formula (II′) or a pharmaceutically acceptable salt, prodrug, or stereoisomer thereof:

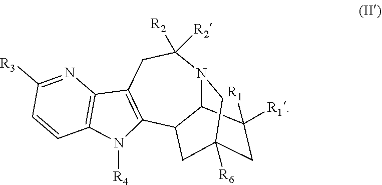

In embodiments, the compound of Formula (II) is a compound of the Formula (II″) or a pharmaceutically acceptable salt, prodrug, or stereoisomer thereof:

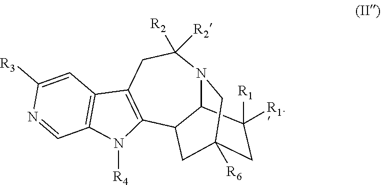

In embodiments of the compounds of Formula (II), (II′), or (II″), R₁ and R₁′ are independently hydrogen, —CH₂CH₃, —CH₂OH, or —CH₂ORₐ.

In embodiments, R₁ and R₁′ are hydrogen.

In embodiments, R₁ is alkyl. In embodiments, R₁ is —C₁-C₆ alkyl (for example, C₁, C₂, C₃, C₄, C₆, or C₅).

In embodiments, R₁ is —CH₂ORₐ.

In embodiments, R₁ is —CH₂NRₐRₐ. In embodiments, R₁ is —CH₂NH₂. In embodiments, R₁ is —CH₂SRₐ. In embodiments, R₁ is —C(═O)ORₐ. In embodiments, R₁ is —CONHRₐ. In embodiments, R₁ is —CON(Rₐ)Rₐ.

In embodiments, R₁ is —CH₂CH₃. In embodiments, R₁ is —CH₂OH.

In embodiments of the compounds of Formula (II), (II′), or (II″), the compound is selected from:

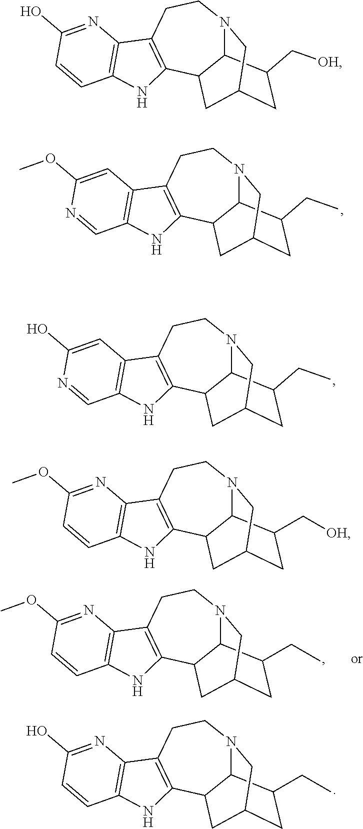

In embodiments, the present disclosure provides a compound of Formula (II):

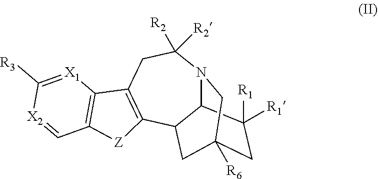

or a pharmaceutically acceptable salt, prodrug, or stereoisomer thereof, wherein:
X₁ and X₂ are independently CR₃, or N;
R₁ and R₁′ are independently hydrogen, alkyl, —CH₂ORₐ, —CH₂NH₂, —CH₂NRₐRₐ, —CH₂SRₐ, —C(═O)ORₐ, —CONHRₐ, or —CON(Rₐ)Rₐ;
R₂ and R₂′ are independently hydrogen, —CH₂OH, —CH₂O-alkyl, —COOH, —CON(Rₐ)Rₐ, or R₂ and R₂′ taken together with the atom to which they are attached form ═O, ═S, ═NH, or ═N-alkyl;
each R₃ is independently deuterium, halogen, alkyl, —ORₐ, —NO₂, —CN, cycloalkyl, aryl, heteroaryl, —OAc, —SRₐ, —NH₂, —NH(alkyl), —NH(alkenyl), —NH(alkynyl), —NH(aryl), —NH(heteroaryl), —N(cycloalkyl), —C(═O)R_{b}, —S(═O)R_{b}, —S(═O)(═NH)(R_{b}), —SO₂R_{b}, —NHSO₂R_{b}, —SC(═O)R_{b}, —NHC(═O)R_{c} or —NHC(S)R_{c};
R₆ is hydrogen, alkyl, —CH₂ORₐ, —CH₂NH₂, —CH₂NRₐRₐ, —CH₂SRₐ, —C(═O)ORₐ, —CONHRₐ, or —CON(Rₐ)Rₐ;
each Rₐ is independently hydrogen, alkyl, deuterated alkyl, alkenyl, alkylene-aryl, alkylene-cycloalkyl, aryl, or heteroaryl;
each R_{b} is independently alkyl, aryl, heteroaryl, —OH, —O-alkyl, —NH₂, —NH(alkyl), or —N(alkyl)₂;
each R_{c} is independently alkyl, aryl, —O-alkyl, —S-alkyl, —S-aryl, —NH₂, —NH(alkyl), or —N(alkyl)₂;
Z is NR₄, wherein R₄ is hydrogen, alkyl, deuterated alkyl, heteroaryl, alkenyl, alkylene-aryl, alkylene-cycloalkyl, aryl, or —SO₂Rₐ; and
when R₂ and R₂′ are hydrogen, R₃ is —OH, and X₁ and X₂ are CH, then R₁ or R₁′ is not —CH₂CH₃, —(CH₂)₂CN, —(CH₂)₂OCH₂C₆H 5, —CD₂CD₃, —CH₂CD₃, —(CH₂)₂OH, —(CH₂)₂OCH₂CH₃, or —(CH₂)₂CF₃;
when R₁ or R₁′ is —CH₂CH₃ or —CH₂OH, and X₁ and X₂ are CH, then R₃ is not —OCH₂CH₃, —OC(CH₃)₃, or —CH═CH₂, or R₄ is not —(CH₂)₃N(CH₃)₂;
and the compound is not

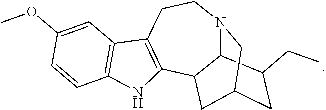 [SMILES: CCC1CC2CC3c4[nH]c5ccc(OC)cc5c4CCN(C2)C13]

In embodiments, the compound of Formula (II) is a compound of the Formula (II-a), or a pharmaceutically acceptable salt, prodrug, or stereoisomer thereof.

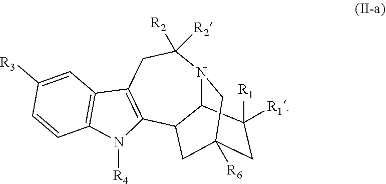

In embodiments of the compounds of Formula (II) or (II-a), R₃ is heteroaryl.

In embodiments, R₃ is a 5-6 membered heteroaryl ring.

In embodiments, R₃ is

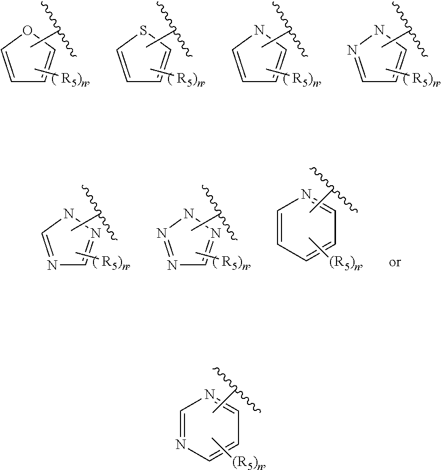

wherein n is 0, 1, or 2; and R₅ is hydrogen, halogen, —OH, —O-alkyl, or —C₁-C₃ alkyl.

In embodiments, R₃ is

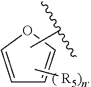

In embodiments, R₃ is

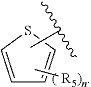

In embodiments, R₃ is

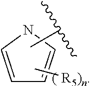

In embodiments,

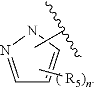

In embodiments,

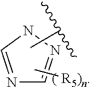

In embodiments,

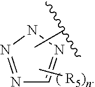

In embodiments,

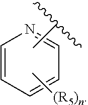

In embodiments,

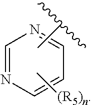

In embodiments, n is 0. In embodiments, n is 1. In embodiments, n is 2.

In embodiments of the compounds of Formula (II) or (II-a), R₅ is hydrogen, halogen, —OH, —O-alkyl, or —C₁-C₃ alkyl. In embodiments, R₅ is hydrogen. In embodiments, R₅ is halogen. In embodiments, R₅ is F, Cl, Br, or I. In embodiments, R₅ is F. In embodiments, R₅ is Cl. In embodiments, R₅ is Br. In embodiments, R₅ is I. In embodiments, R₅ is —OH. In embodiments, R₅ is —O-alkyl. In embodiments, R₅ is —C₁-C₃ alkyl. In embodiments, R₅ is —OCH₃. In embodiments, R₅ is O—CH₂CH₃. In embodiments, R₅ is —CH₃. In embodiments, R₅ is —CH₂CH₃. In embodiments, R₅ is —CH₂CH₂CH₃.

In embodiments of the compounds of Formula (II) or (II-a), R₃ is

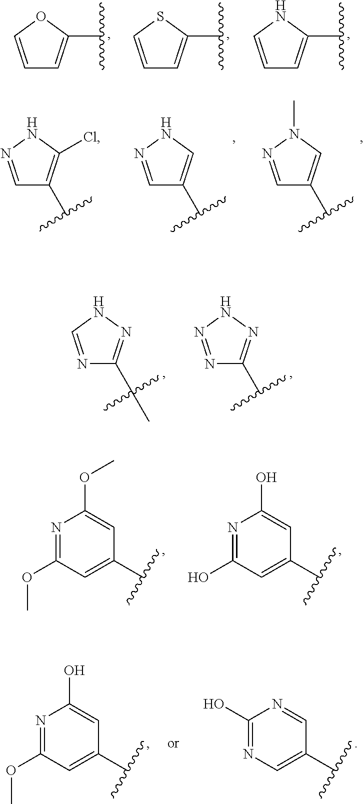

In embodiments, R₃ is

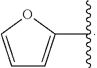 [SMILES: *c1ccco1]

In embodiments, R₃ is

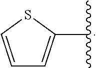 [SMILES: *c1cccs1]

In embodiments, R₃ is

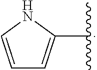 [SMILES: *c1ccc[nH]1]

In embodiments, R₃ is

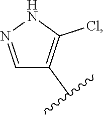 [SMILES: *c1cn[nH]c1Cl]

In embodiments, R₃ is

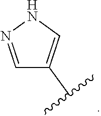 [SMILES: *c1cn[nH]c1]

In embodiments, R₃ is

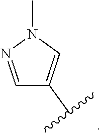 [SMILES: *c1cnn(C)c1]

In embodiments, R₃ is

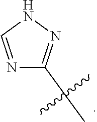 [SMILES: *c1nc[nH]n1]

In embodiments, R₃ is

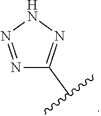 [SMILES: *c1nn[nH]n1]

In embodiments, R₃ is

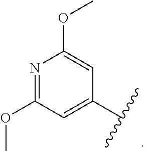 [SMILES: *c1cc(OC)nc(OC)c1]

In embodiments, R₃ is

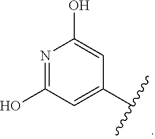 [SMILES: *c1cc(O)nc(O)c1]

In embodiments, R₃ is

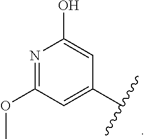 [SMILES: *c1cc(O)nc(OC)c1]

In embodiments, R₃ is

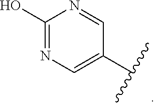 [SMILES: *c1cnc(O)nc1]

In embodiments of the compounds of Formula (II) or (II-a), R₃ is —CN.

In embodiments of the compounds of Formula (II) or (II-a), R₃ is S(═O)R_{b}, —SO₂R_{b}, or —S— alkyl.

In embodiments of the compounds of Formula (II) or (II-a), R₃ is —SCH₃.

In embodiments of the compounds of Formula (II) or (II-a), R₃ is —SO₂CH₃.

In embodiments of the compounds of Formula (II) or (II-a), R₃ is —S(═O)(═NH)(CH₃).

In embodiments of the compounds of Formula (II) or (II-a), R₃ is —NH₂.

In embodiments of the compounds of Formula (II) or (II-a), R₃ is —COR_{b}. In embodiments, R_{b} is O-alkyl or —N(alkyl)₂. In embodiments, R_{b} is —OCH₃ or —N(CH₃)₂.

In embodiments of the compounds of Formula (II) or (II-a), R₃ is —ORₐ.

In embodiments of the compounds of Formula (II) or (II-a), R₃ is —OCH(CH₃)OCH₃.

In embodiments of the compounds of Formula (II) or (II-a), R₃ is deuterium.

In embodiments of the compounds of Formula (II) or (II-a), R₃ is —OH.

In embodiments of the compounds of Formula (II) or (II-a), R₃ is —OCH₃.

In embodiments of the compounds of Formula (II) or (II-a), R₄ is hydrogen. In embodiments, R₄ is alkyl. In embodiments, R₄ is —CH₃.

In embodiments of the compounds of Formula (II) or (II-a), R₆ is hydrogen.

In embodiments of the compounds of Formula (II) or (II-a), the compound is selected from:

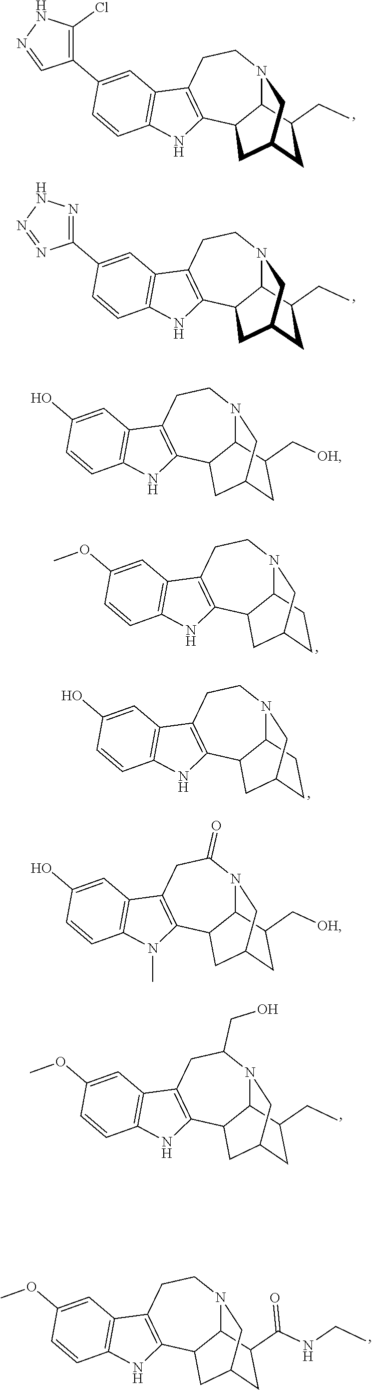

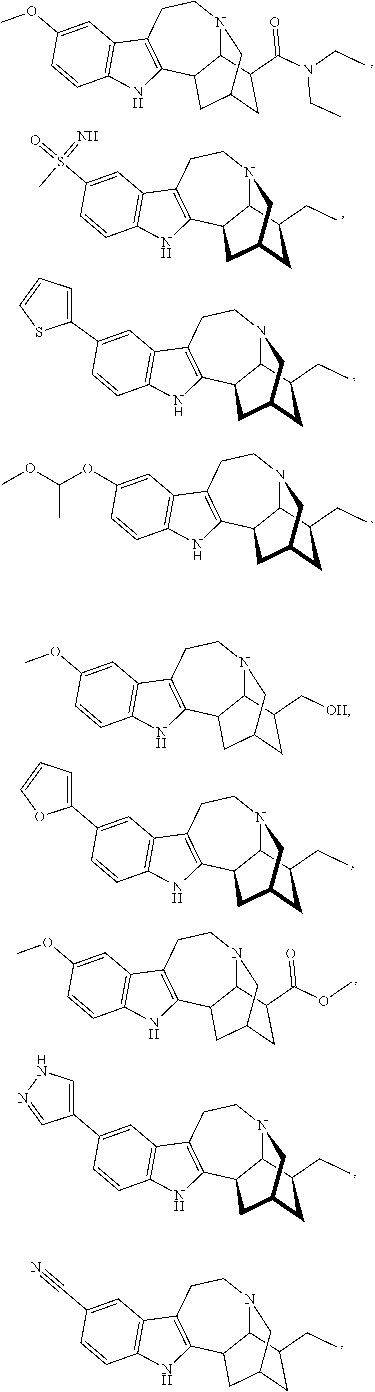

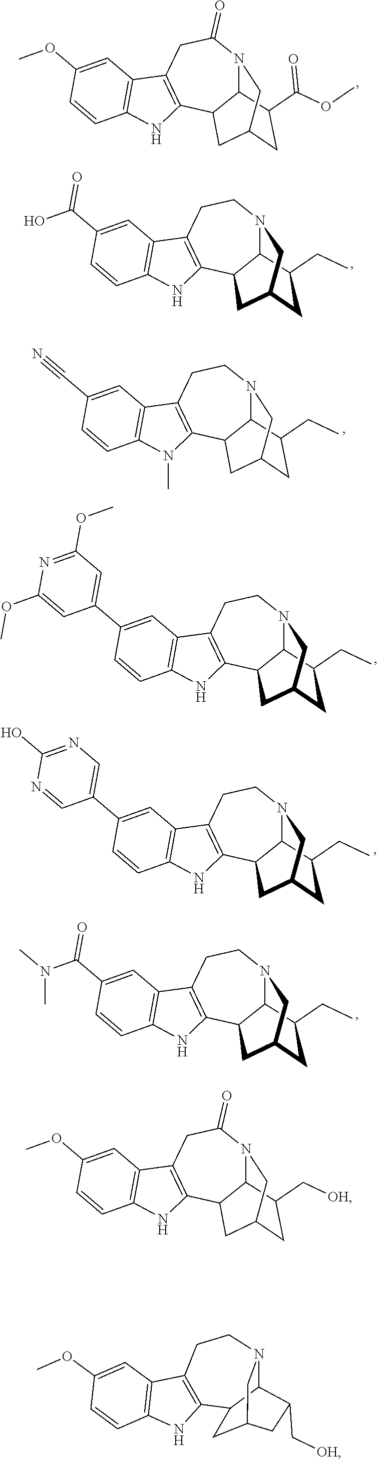

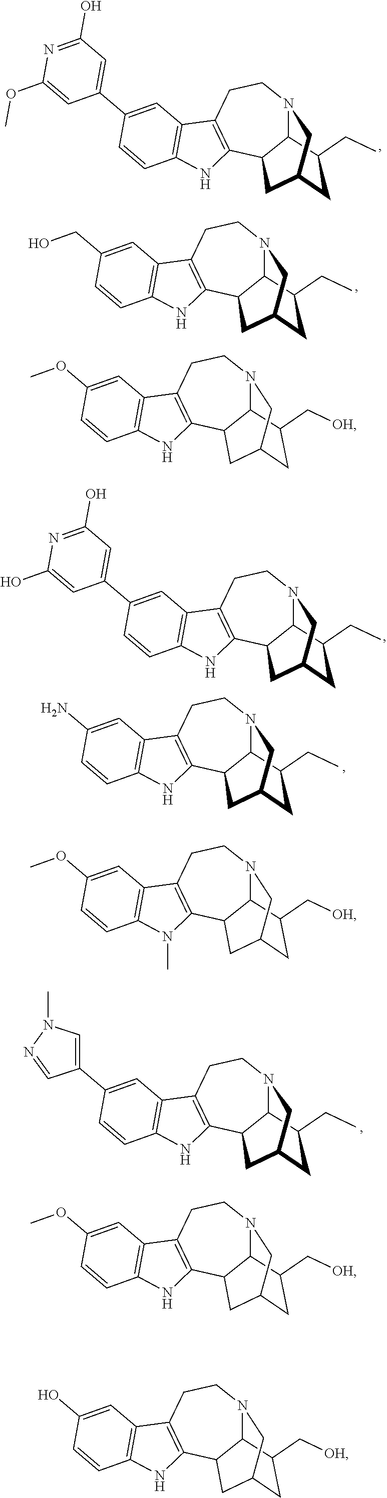

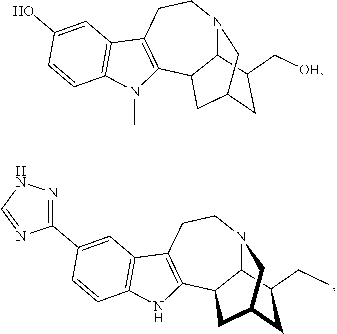

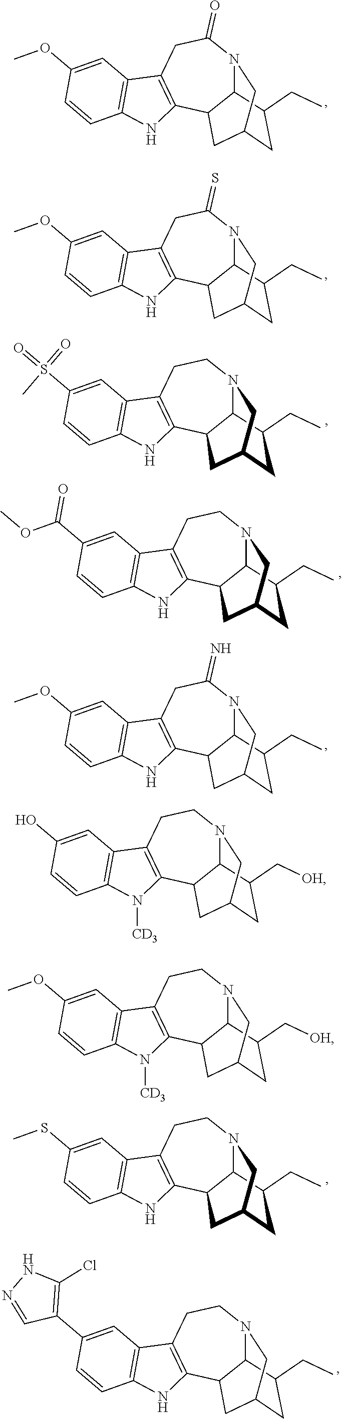

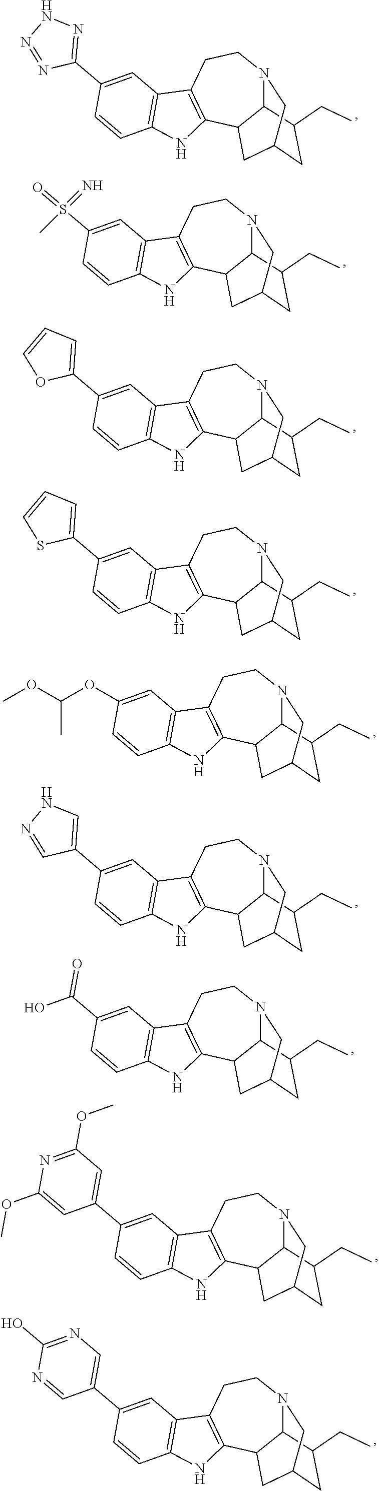

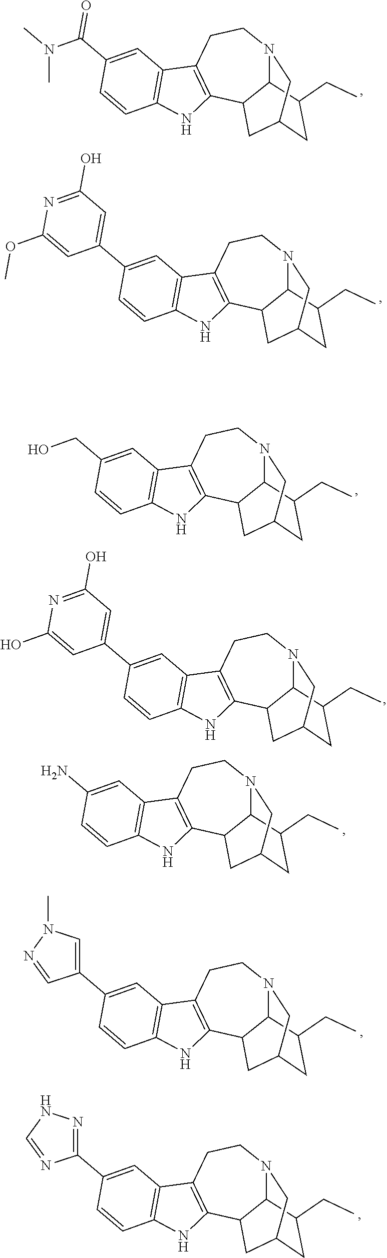

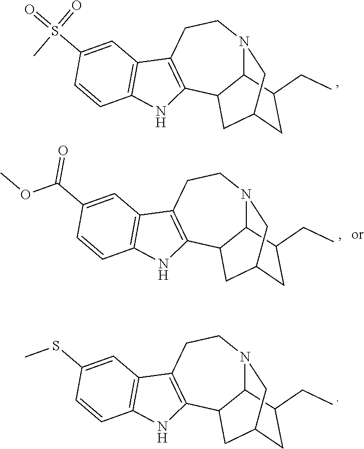

In embodiments, the present disclosure provides a compound of Formula (III):

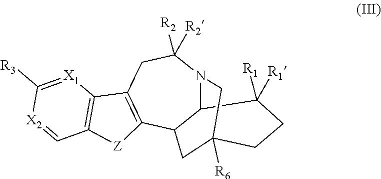

or a pharmaceutically acceptable salt, prodrug, or stereoisomer thereof, wherein:
X₁ and X₂ are independently CR₃, or N;
R₁ and R₁′ are independently hydrogen, alkyl, —CH₂ORₐ, —CH₂CH₂ORₐ, —CH₂NH₂, —CH₂NRₐRₐ, —CH₂SRₐ, —C(═O)ORₐ, —CONHRₐ, or —CON(Rₐ)Rₐ;
R₂ and R₂′ are independently hydrogen, deuterium, —CH₂OH, —CH₂O-alkyl, —COOH, —CON(Rₐ)Rₐ, or R₂ and R₂′ taken together with the atom to which they are attached form ═O, ═S, ═NH, or ═N-alkyl;
each R₃ is independently hydrogen, deuterium, halogen, alkyl, —ORₐ, —NO₂, —CN, cycloalkyl, aryl, heteroaryl, —OAc, —SRₐ, —NH₂, —NH(alkyl), —NH(alkenyl), —NH(alkynyl), —NH(aryl), —NH(heteroaryl), —N(cycloalkyl), —C(═O)R_{b}, —S(═O)R_{b}, —SO₂R_{b}, —NHSO₂R_{b}, —OC(═O)R_{b}, SC(═O)R_{b}, —NHC(═O)R_{c} or —NHC(S)R_{c};
R₆ is hydrogen, alkyl, —CH₂ORₐ, —CH₂NH₂, —CH₂NRₐRₐ, —CH₂SRₐ, —C(═O)ORₐ, —CONHRₐ, or —CON(Rₐ)Rₐ;
each Rₐ is independently hydrogen, alkyl, deuterated alkyl, alkenyl, alkylene-aryl, alkylene-cycloalkyl, aryl, or heteroaryl;
each R_{b} is independently alkyl, aryl, heteroaryl, —OH, —O-alkyl, —NH₂, —NH(alkyl), or —N(alkyl)₂;
each R_{c} is independently alkyl, aryl, —O-alkyl, —S-alkyl, —S-aryl, —NH₂, —NH(alkyl), or —N(alkyl)₂; and
Z is N(R₄), wherein R₄ is hydrogen, alkyl, deuterated alkyl, heteroaryl, alkenyl, alkylene-aryl, alkylene-cycloalkyl, aryl, or SO₂Rₐ.

In embodiments of the compounds of Formula (III), X₁ and X₂ are CR₃.

In embodiments, X₁ and X₂ are N.

In embodiments, X₁ is CR₃, and X₂ is N.

In embodiments, X₁ is N, and X₂ is CR₃.

In embodiments, the compound of Formula (III) is a compound of the Formula (III′), or a pharmaceutically acceptable salt, prodrug, or stereoisomer thereof.

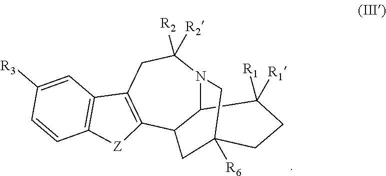

In embodiments, the compound of Formula (III) is a compound of the Formula (III″), or a pharmaceutically acceptable salt, prodrug, or stereoisomer thereof.

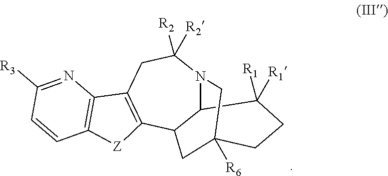

In embodiments of Formula (I), (I′), (I″), (I-a), (I-b), (II), (II′), (II″), (II-a), (III), (III′), or (III″), R₁ and R₁′ are each independently hydrogen, -alkyl, —CH₂ORₐ, or —CH₂SRₐ.

In embodiments of Formula (I), (I′), (I″), (I-a), (I-b), (III), (III′), or (III″), R₁ and R₁′ are each independently hydrogen, alkyl, —CF₃, —CH(Rₐ)(R_{b}), —CH₂ORₐ, —CH₂CH₂ORₐ, —CH₂NH₂, —CH₂NRₐRₐ, —CH₂SRₐ, —C(═O)ORₐ, —C(═O)NHRₐ, —ORₐ, or —C(═O)N(Rₐ)(R_{b}), wherein each Rₐ is independently hydrogen, alkyl, deuterated alkyl, alkenyl, alkylene-aryl, alkylene-cycloalkyl, aryl, or heteroaryl; and each R_{b} is independently alkyl, aryl, heteroaryl, —OH, —O-alkyl, —NH₂, —NH(alkyl), or —N(alkyl)₂.

In embodiments of Formula (I), (I′), (I″), (I-a), (I-b), (II), (II′), (II″), (II-a), (III), (III′), or (III″), R₁ is hydrogen.

In embodiments, R₁ is -alkyl. In embodiments, R₁ is —C₁-C₆ alkyl (for example, C₁, C₂, C₃, C₄, C₆, or C₅). In embodiments, R₁ is —CH₃. In embodiments, R₁ is —CH₂CH₃.

In embodiments, R₁ is —CH₂ORₐ. In embodiments, R₁ is —CH₂OH. In embodiments, R₁ is —CH₂OCH₃. In embodiments, R₁ is —CH₂CH₂OCH₃. In embodiments, R₁ is —CH₂OCH₂CH₃.

In embodiments, R₁ is —CH₂NH₂. In embodiments, R₁ is —CH₂NRₐRₐ. In embodiments, R₁ is —CH₂NH₂. In embodiments, R₁ is —CH₂NH-alkyl. In embodiments, R₁ is —CH₂NH—C₁-C₆ alkyl. In embodiments, R₁ is —CH₂NHCH₃.

In embodiments, R₁ is —CH₂SRₐ. In embodiments, R₁ is —CH₂SH. In embodiments, R₁ is —CH₂S—C₁-C₆ alkyl.

In embodiments, R₁ is —C(═O)ORₐ. In embodiments, R₁ is —C(═O)OH. In embodiments, R₁ is —C(═O)O-alkyl. In embodiments, R₁ is —C(═O)O—C₁-C₆ alkyl. In embodiments, R₁ is —C(═O)OCH₃. In embodiments, R₁ is —C(═O)OCH₂CH₃. In embodiments, R₁ is —C(═O)NHRₐ. In embodiments, R₁ is —C(═O)NH₂. In embodiments, R₁ is —C(═O)NH—C₁-C₆ alkyl. In embodiments, R₁ is —C(═O)NHCH₃. In embodiments, R₁ is —C(═O)N(Rₐ)(R_{b}). In embodiments, R₁ is —C(═O)N(CH₃)₂. In embodiments, R₁ is —C(═O)N(CH₂CH₃)₂.

In embodiments, R₁ is —CF₃.

In embodiments, R₁ is —CH(CH₃)₂.

In embodiments, R₁′ is hydrogen. In embodiments, R₁′ is -alkyl. In embodiments, R₁′ is —C₁-C₆ alkyl (for example, C₁, C₂, C₃, C₄, C₅, or C₆). In embodiments, R₁′ is CH₃. In embodiments, R₁′ is CH₂CH₃. In embodiments, R₁′ is —CH₂ORₐ. In embodiments, R₁′ is —CH₂OH. In embodiments, R₁′ is —CH₂OCH₃. In embodiments, R₁′ is —CH₂CH₂OCH₃. In embodiments, R₁′ is —CH₂OCH₂CH₃. In embodiments, R₁′ is —CH₂NH₂. In embodiments, R₁′ is —CH₂NRₐRₐ. In embodiments, R₁′ is —CH₂NH₂. In embodiments, R₁′ is —CH₂NH-alkyl. In embodiments, R₁′ is —CH₂NH—C₁-C₅ alkyl. In embodiments, R₁′ is —CH₂NHCH₃. In embodiments, R₁′ is —CH₂SRₐ. In embodiments, R₁′ is —CH₂SH. In embodiments, R₁′ is —CH₂S—C₁-C₅ alkyl. In embodiments, R₁′ is —C(═O)ORₐ. In embodiments, R₁′ is —C(═O)OH. In embodiments, R₁′ is —C(═O)O-alkyl. In embodiments, R₁′ is —C(═O)O—C₁-C₆ alkyl. In embodiments, R₁′ is —C(═O)OCH₃. In embodiments, R₁′ is —C(═O)OCH₂CH₃. In embodiments, R₁′ is —C(═O)NHRₐ. In embodiments, R₁′ is —C(═O)NH₂. In embodiments, R₁′ is —C(═O)NH—C₁-C₆ alkyl. In embodiments, R₁′ is —C(═O)NHCH₃. In embodiments, R₁′ is —C(═O)N(Rₐ)(R_{b}). In embodiments, R₁′ is —C(═O)N(CH₃)₂. In embodiments, R₁′ is —C(═O)N(CH₂CH₃)₂. In embodiments, R₁′ is —CF₃. In embodiments, R₁′ is —CH(CH₃)₂.

In embodiments of Formula (I), (I′), (I″), (I-a), (I-b), (II), (II′), (II″), (II-a), (III), (III′), or (III″), R₂ and R₂′ are each independently hydrogen, deuterium, —CH₂OH, —CH₂O-alkyl, —COOH, or —CON(Rₐ)Rₐ, wherein Rₐ is described above in Formula (I), (I′), or (I″). In embodiments of Formula (II-a), R₂ and R₂′ are each independently hydrogen, —CH₂OH, —CH₂O-alkyl, —COOH, or —CON(Rₐ)Rₐ, wherein Rₐ is described above in Formula (I), (I′), or (I″).

In embodiments Formula (I), (I′), (I″), (I-a), (I-b), (II), (II′), (II″), (II-a), (III), (III′), or (III″), R₂ and R₂′ taken together with the atom to which they are attached form ═O, ═S, ═NH, or ═N-alkyl.

In embodiments, R₂ is hydrogen. In embodiments, R₂ is deuterium. In embodiments, R₂ is —CH₂OH. In embodiments, R₂ is —CH₂O-alkyl. In embodiments, R₂ is —CH₂O—C₁-C₆ alkyl (for example, C₁, C₂, C₃, C₄, C₅, or C₆). In embodiments, R₂ is —COOH. In embodiments, R₂ is —CON(Rₐ)Rₐ. In embodiments, R₂ is —C(═O)NH₂. In embodiments, R₂ is —CON(CH₃)CH₃. In embodiments, R₂ is —CON(CH₂CH₃)CH₂CH₃. In embodiments, R₂ is —CONHCH₃. In embodiments, R₂ is —CONHCH₂CH₃.

In embodiments, R₂′ is hydrogen. In embodiments, R₂′ is deuterium. In embodiments, R₂′ is —CH₂OH. In embodiments, R₂′ is —CH₂O-alkyl. In embodiments, R₂′ is —CH₂O—C₁-C₅ alkyl (for example, C₁, C₂, C₃, C₄, or C₅). In embodiments, R₂′ is —COOH. In embodiments, R₂′ is —CON(Rₐ)Rₐ. In embodiments, R₂′ is —CONH₂. In embodiments, R₂′ is —CON(CH₃)CH₃. In embodiments, R₂′ is —CON(CH₂CH₃)CH₂CH₃. In embodiments, R₂′ is —CONHCH₃. In embodiments, R₂′ is —CONHCH₂CH₃.

In embodiments, R₂ and R₂′ are taken together with the atom to which they are attached form ═O. In embodiments, R₂ and R₂′ are taken together with the atom to which they are attached form ═S. In embodiments, R₂ and R₂′ are taken together with the atom to which they are attached form ═NH. In embodiments, R₂ and R₂′ are taken together with the atom to which they are attached form ═N-alkyl.

In embodiments of Formula (I), (I′), (I″), (I-a), (I-b), (II), (II′), (II″), (III), (III′), or (III″), R₃ is hydrogen, deuterium, halogen, alkyl, or —ORₐ, wherein Rₐ is described above in Formula (I). In embodiments of Formula (I), each R₃ is independently hydrogen, deuterium, halogen, alkyl, —ORₐ, —NO₂, —CN, —CF₃, cycloalkyl, aryl, heteroaryl, —OAc, —SRₐ, —NH₂, —NH(alkyl), —NH(alkenyl), —NH(alkynyl), —NH(aryl), —NH(heteroaryl), —N(cycloalkyl), —C(═O)R_{b}, —S(═O)R_{b}, —SO₂R_{b}, —NHSO₂R_{b}, —OC(═O)R_{b}, SC(═O)R_{b}, —NHC(═O)R_{c} or —NHC(S)R_{c}, wherein Rₐ, R_{b}, and R_{c} are described above in Formula (I).

In embodiments of Formula (II-a), R₃ is independently deuterium, halogen, alkyl, —ORₐ, —NO₂, —CN, cycloalkyl, aryl, heteroaryl, —OAc, —SRₐ, or —NH₂, wherein Rₐ is described above in Formula (II-a).

In embodiments, R₃ is hydrogen. In embodiments, R₃ is deuterium. In embodiments, R₃ is halogen. In embodiments, R₃ is —F. In embodiments, R₃ is —Cl. In embodiments, R₃ is —Br. In embodiments, R₃ is —I. In embodiments, R₃ is alkyl. In embodiments, R₃ is —ORₐ. In embodiments, R₃ is —OH. In embodiments, R₃ is —OCH₃. In embodiments, R₃ is —OCH₂CH₃.In embodiments, R₃ is —NO₂. In embodiments, R₃ is —CN. In embodiments, R₃ is —CF₃.In embodiments, R₃ is cycloalkyl. In embodiments, R₃ is aryl. In embodiments, R₃ is heteroaryl. In embodiments, R₃ is -OAc. In embodiments, R₃ is —SRₐ. In embodiments, R₃ is —NH₂. In embodiments, R₃ is —NH(alkyl). In embodiments, R₃ is —NH(alkenyl). In embodiments, R₃ is —NH(alkynyl). In embodiments, R₃ is —NH(aryl). In embodiments, R₃ is —NH(heteroaryl). In embodiments, R₃ is —N(cycloalkyl). In embodiments, R₃ is C(═O)R_{b}. In embodiments, R₃ is —S(═O)R_{b}, wherein R_{b} is described above in Formula (I). In embodiments, R₃ is —S(═N)(═O)(R_{b}). In embodiments, R₃ is —SO₂R_{b}. In embodiments, R₃ is —NHSO₂R_{b}. In embodiments, R₃ is —OC(═O)R_{b}. In embodiments, R₃ is —SC(═O)R_{b}. In embodiments, R₃ is —NHC(═O)R_{c}, wherein R_{c} is described above in Formula (I). In embodiments, R₃ is —NHC(S)R_{c}.

In embodiments of Formula (I), (I′), (I″), (I-a), (I-b), (II), (II′), (II″), (III), (III′), or (III″), X₁, X₂ and X₄ are independently C(R₃) or N. In embodiments, X₁, X₂ or X₄ is N. In embodiments, X₁ and X₂ are N. In embodiments, X₁ and X₄ are N. In embodiments, X₂ and X₄ are N. In embodiments, X₁, X₂ and X₄ are N. In embodiments, X₁, X₂ or X₄ is C(R₃), wherein R₃ is described above in Formula (I). In embodiments, X₁ and X₂ are CH. In embodiments, X₁ and X₄ are CH. In embodiments, X₂ and X₄ are CH. In embodiments, X₁, X₂ and X₄ are CH. In embodiments, X₁ is N, and X₂ is CH. In embodiments, X₁ is CF, and X₂ is CH. In embodiments, X₁ is CH, and X₂ is CF. In embodiments, X₁ is CCH₃, and X₂ is CH. In embodiments, X₁ is CH, and X₂ is CCH₃. In embodiments, X₁ is CH, and X₂ is COCH₃. In embodiments, X₁ is CH, and X₂ is COH. In embodiments, X₁ is COH, and X₂ is COH.

In embodiments of Formula (I), (I′), (I″), (I-a), (I-b), (II), (II′), (II″), (II-a), (III), (III′), or (III″), Rₐ is hydrogen, alkyl, or deuterated alkyl. In embodiments, it is independently hydrogen, alkyl, deuterated alkyl, alkenyl, alkylene-aryl, alkylene-cycloalkyl, aryl, or heteroaryl. In embodiments, itis hydrogen. In embodiments, itis alkyl. In embodiments, Rₐ is —C₁-C₆ alkyl (for example, C₁, C₂, C₃, C₄, C₅, or C₆). In embodiments, Rₐ is deuterated alkyl. In embodiments, Rₐ is —C₁-C₆ deuterated alkyl. In embodiments, Rₐ is alkenyl. In embodiments, Rₐ is —C₂-C₈ alkenyl (for example, C₂, C₃, C₄, C₅, C₆, C₇, C₈). In embodiments, Rₐ is alkylene-aryl. In embodiments, Rₐ is alkylene-cycloalkyl. In embodiments, Rₐ is aryl. In embodiments, Rₐ is heteroaryl.

In embodiments of Formula (I), (I′), (I″), (I-a), (I-b), (II), (II′), (II″), (II-a), (III), (III′), or (III″), R_{b} is alkyl, aryl, heteroaryl, —OH, —O-alkyl, or —NH₂. In embodiments, R_{b} is alkyl, aryl, heteroaryl, —OH, —O-alkyl, —NH₂, —NH(alkyl), or —N(alkyl)₂. In embodiments, R_{b} is alkyl. In embodiments, R_{b} is —C₁-C₆ alkyl (for example, C₁, C₂, C₃, C₄, C₅, or C₆). In embodiments, R_{b} is aryl. In embodiments, R_{b} is heteroaryl. In embodiments, R_{b} is —OH. In embodiments, R_{b} is —O— alkyl. In embodiments, R_{b} is —NH₂. In embodiments, R_{b} is —NH(alkyl). In embodiments, R_{b} is —N(alkyl)₂.

In embodiments of Formula (I), (I′), (I″), (I-a), (I-b), (II), (II′), (II″), (II-a), (III), (III′), or (III″), R_{c} is alkyl, aryl, —O-alkyl, —S-alkyl, —S-aryl, or —NH₂. In embodiments, R_{c} is alkyl, aryl, —O— alkyl, —S-alkyl, —S-aryl, —NH₂, —NH(alkyl), or —N(alkyl)₂. In embodiments, R_{c} is alkyl. In embodiments, R_{c} is —C₁-C₆ alkyl (for example, C₁, C₂, C₃, C₄, C₅, or C₆). In embodiments, R_{c} is aryl. In embodiments, R_{c} is —O-alkyl. In embodiments, R_{c} is —S-alkyl. In embodiments, R_{c} is —S-aryl. In embodiments, R_{c} is —NH₂.

In embodiments of Formula (I), Z is O, S, or N(R₄), wherein R₄ is absent, hydrogen, alkyl, deuterated alkyl, heteroaryl, alkenyl, alkylene-aryl, alkylene-cycloalkyl, aryl, or SO₂Rₐ. In embodiments of Formula (I), (I′), (I″), (I-a), (I-b), (II), (II′), (II″), (II-a), (III), (III′), or (III″), Z is O.

In embodiments of Formula (I), (I′), (I″), (I-a), (I-b), (II), (II′), (II″), (II-a), (III), (III′), or (III″), Z is S.

In embodiments of Formula (I), (I′), (I″), (I-a), (I-b), (II), (II′), (II″), (II-a), (III), (III′), or (III″), Z is NR₄, wherein R₄ is absent, hydrogen, alkyl, deuterated alkyl, heteroaryl, alkenyl, alkylene-aryl, alkylene-cycloalkyl, aryl, or —SO₂Rₐ. In embodiments of Formula (I), (I′), (I″), (I-a), (I-b), (II), (II′), (II″), (II-a), (III), (III′), or (III″), R₄ is hydrogen, alkyl, deuterated alkyl, or heteroaryl. In embodiments of Formula (I) or (I″) R₄ is absent, hydrogen, alkyl, deuterated alkyl, heteroaryl, alkenyl, alkylene-aryl, alkylene-cycloalkyl, aryl, or —SO₂Rₐ.

In embodiments, R₄ is hydrogen. In embodiments, R₄ is absent.

In embodiments, R₄ is alkyl. In embodiments, R₄ is —C₁-C₆ alkyl. In embodiments, R₄ is —C₁-C₆ CH₃. In embodiments, R₄ is CH₃. In embodiments, R₄ is deuterated alkyl. In embodiments, R₄ is heteroaryl. In embodiments, R₄ is alkenyl. In embodiments, R₄ is alkylene-aryl. In embodiments, R₄ is alkylene-cycloalkyl. In embodiments, R₄ is aryl. In embodiments, R₄ is —SO₂Rₐ wherein Rₐ is described above in Formula (I).

In embodiments of Formula (I), R₆ is hydrogen, alkyl, —CH₂ORₐ, —CH₂NH₂, —CH₂N(Rₐ)(R_{b}), —CH₂SRₐ, —C(═O)ORₐ, —CONHRₐ, or —CON(Rₐ)(R_{b}), wherein Rₐ and R_{b} are described above in Formula (I). In embodiments of Formula (I), (I′), (I″), (I-a), (I-b), (II), (II′), (II″), (II-a), (III), (III′), or (III″), R₆ is hydrogen. In embodiments, R₆ is -alkyl. In embodiments, R₆ is —C₁-C₆ alkyl (for example, C₁, C₂, C₃, C₄, C₆, or C₅). In embodiments, R₆ is —CH₃. In embodiments, R₆ is —CH₂CH₃. In embodiments, R₆ is —CH₂ORₐ, wherein Rₐ is described above in Formula (I). In embodiments, R₆ is —CH₂OH. In embodiments, R₆ is —CH₂OCH₃. In embodiments, R₆ is —CH₂OCH₂CH₃. In embodiments, R₆ is —CH₂NH₂. In embodiments, R₆ is —CH₂NRₐRₐ. In embodiments, R₆ is —CH₂NH₂. In embodiments, R₆ is —CH₂NH-alkyl. In embodiments, R₆ is —CH₂NH—C₁-C₆ alkyl. In embodiments, R₆ is —CH₂NHCH₃. In embodiments, R₆ is —CH₂SRₐ. In embodiments, R₆ is —CH₂SH. In embodiments, R₆ is —CH₂S—C₁-C₆ alkyl. In embodiments, R₆ is —C(═O)ORₐ. In embodiments, R₆ is —C(═O)OH. In embodiments, R₆ is —C(═O)O-alkyl. In embodiments, R₆ is —C(═O)O—C₁-C₆ alkyl. In embodiments, R₆ is —C(═O)OCH₃. In embodiments, R₆ is —C(═O)OCH₂CH₃. In embodiments, R₆ is —CONHRₐ. In embodiments, R₆ is —CONH₂. In embodiments, R₆ is —CONH—C₁-C₆ alkyl. In embodiments, R₆ is —CONHCH₃. In embodiments, R₆ is —CON(Rₐ)Rₐ. In embodiments, R₆ is —CON(CH₃)CH₃. In embodiments, R₆ is —CON(CH₂CH₃) CH₂CH₃.

In embodiment provided herein is one or more compounds in Tables 1 and 2.

In embodiment provided herein is a pharmaceutically acceptable salt or a stereoisomer of one or more compounds in Tables 1 and 2.

TABLE 1
Compounds

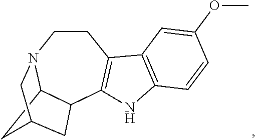 [SMILES: COc1ccc2[nH]c3c(c2c1)CCN1CC2CC3C1C2]

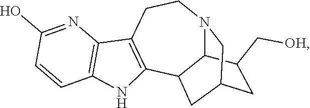 [SMILES: OCC1CC2CC3c4[nH]c5ccc(O)nc5c4CCN(C2)C13]

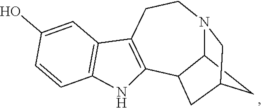 [SMILES: Oc1ccc2[nH]c3c(c2c1)CCN1CC2CC3C1C2]

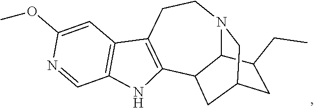 [SMILES: CCC1CC2CC3c4[nH]c5cnc(OC)cc5c4CCN(C2)C13]

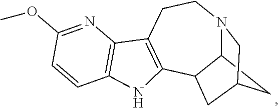 [SMILES: COc1ccc2[nH]c3c(c2n1)CCN1CC2CC3C1C2]

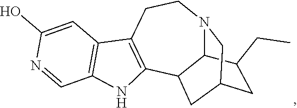 [SMILES: CCC1CC2CC3c4[nH]c5cnc(O)cc5c4CCN(C2)C13]

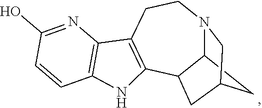 [SMILES: Oc1ccc2[nH]c3c(c2n1)CCN1CC2CC3C1C2]

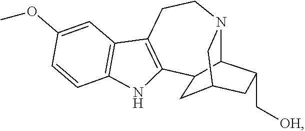 [SMILES: COc1ccc2[nH]c3c(c2c1)CCN1CC2CC(CO)C1C3C2]

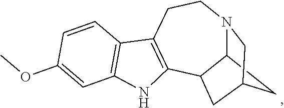 [SMILES: COc1ccc2c3c([nH]c2c1)C1CC2CC1N(CC3)C2]

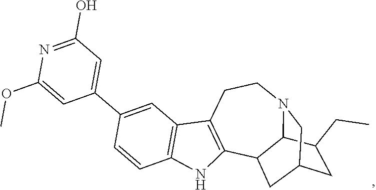 [SMILES: CCC1CC2CC3c4[nH]c5ccc(-c6cc(O)nc(OC)c6)cc5c4CCN(C2)C13]

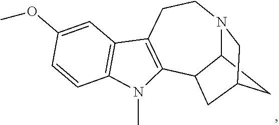 [SMILES: COc1ccc2c(c1)c1c(n2C)C2CC3CC2N(CC1)C3]

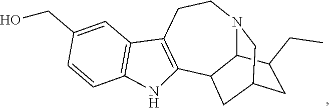 [SMILES: CCC1CC2CC3c4[nH]c5ccc(CO)cc5c4CCN(C2)C13]

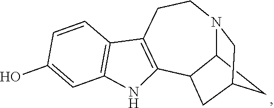 [SMILES: Oc1ccc2c3c([nH]c2c1)C1CC2CC1N(CC3)C2]

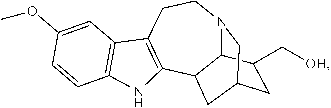 [SMILES: COc1ccc2[nH]c3c(c2c1)CCN1CC2CC(CO)C1C3C2]

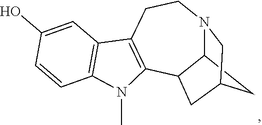 [SMILES: Cn1c2c(c3cc(O)ccc31)CCN1CC3CC2C1C3]

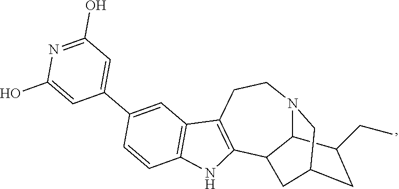 [SMILES: CCC1CC2CC3c4[nH]c5ccc(-c6cc(O)nc(O)c6)cc5c4CCN(C2)C13]

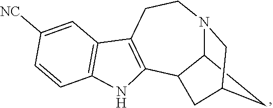 [SMILES: N#Cc1ccc2[nH]c3c(c2c1)CCN1CC2CC3C1C2]

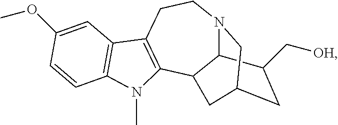 [SMILES: COc1ccc2c(c1)c1c(n2C)C2CC3CC(CO)C2N(CC1)C3]

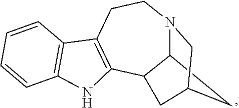 [SMILES: c1ccc2c3c([nH]c2c1)C1CC2CC1N(CC3)C2]

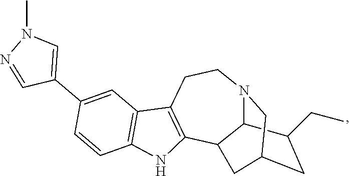 [SMILES: CCC1CC2CC3c4[nH]c5ccc(-c6cnn(C)c6)cc5c4CCN(C2)C13]

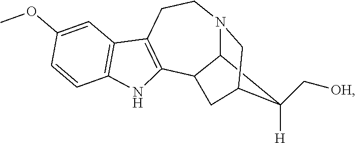 [SMILES: COc1ccc2[nH]c3c(c2c1)CCN1CC2CC3C1C2CO]

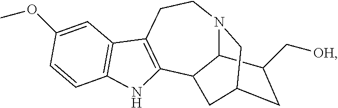 [SMILES: COc1ccc2[nH]c3c(c2c1)CCN1CC2CC(CO)C1C3C2]

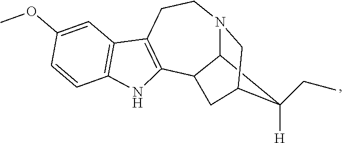 [SMILES: CCC1C2CC3c4[nH]c5ccc(OC)cc5c4CCN(C2)C31]

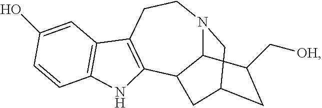 [SMILES: OCC1CC2CC3c4[nH]c5ccc(O)cc5c4CCN(C2)C13]

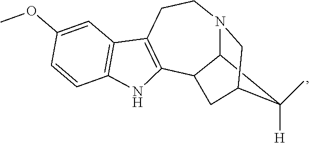

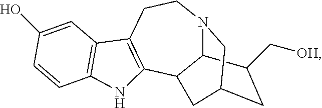 [SMILES: OCC1CC2CC3c4[nH]c5ccc(O)cc5c4CCN(C2)C13]

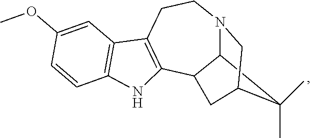

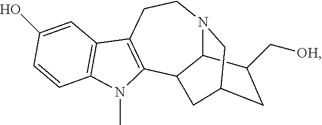 [SMILES: Cn1c2c(c3cc(O)ccc31)CCN1CC3CC(CO)C1C2C3]

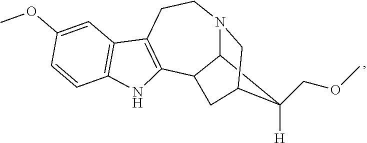

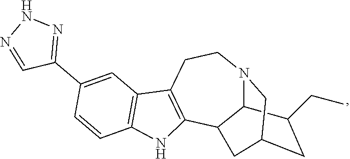 [SMILES: CCC1CC2CC3c4[nH]c5ccc(-c6cn[nH]n6)cc5c4CCN(C2)C13]

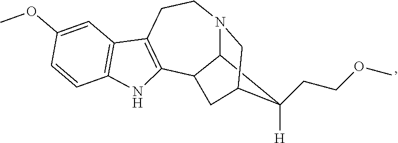

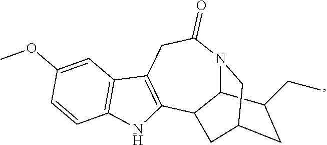 [SMILES: CCC1CC2CC3c4[nH]c5ccc(OC)cc5c4CC(=O)N(C2)C13]

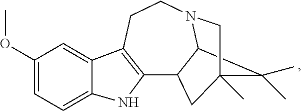 [SMILES: COc1ccc2[nH]c3c(c2c1)CCN1CC2(C)CC3C1C2(C)C]

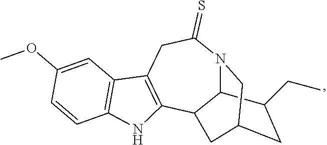 [SMILES: CCC1CC2CC3c4[nH]c5ccc(OC)cc5c4CC(=S)N(C2)C13]

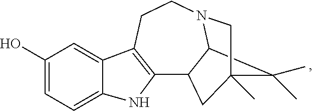 [SMILES: CC12CC3c4[nH]c5ccc(O)cc5c4CCN(C1)C3C2(C)C]

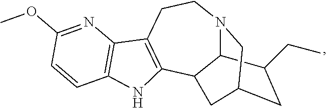 [SMILES: CCC1CC2CC3c4[nH]c5ccc(OC)nc5c4CCN(C2)C13]

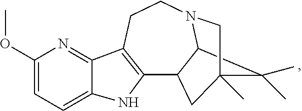 [SMILES: COc1ccc2[nH]c3c(c2n1)CCN1CC2(C)CC3C1C2(C)C]

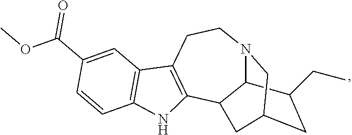 [SMILES: CCC1CC2CC3c4[nH]c5ccc(C(=O)OC)cc5c4CCN(C2)C13]

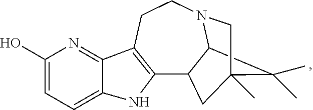 [SMILES: CC12CC3c4[nH]c5ccc(O)nc5c4CCN(C1)C3C2(C)C]

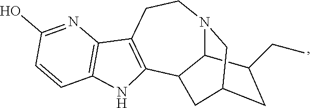 [SMILES: CCC1CC2CC3c4[nH]c5ccc(O)nc5c4CCN(C2)C13]

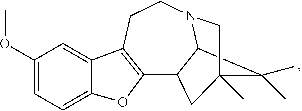 [SMILES: COc1ccc2oc3c(c2c1)CCN1CC2(C)CC3C1C2(C)C]

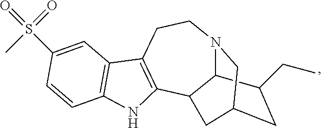 [SMILES: CCC1CC2CC3c4[nH]c5ccc(S(C)(=O)=O)cc5c4CCN(C2)C13]

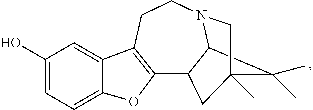 [SMILES: CC12CC3c4oc5ccc(O)cc5c4CCN(C1)C3C2(C)C]

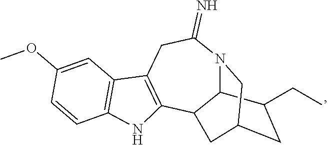 [SMILES: CCC1CC2CC3c4[nH]c5ccc(OC)cc5c4CC(=N)N(C2)C13]

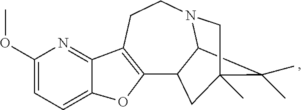 [SMILES: COc1ccc2oc3c(c2n1)CCN1CC2(C)CC3C1C2(C)C]

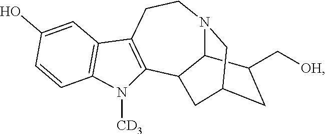 [SMILES: [2H]C([2H])([2H])n1c2c(c3cc(O)ccc31)CCN1CC3CC(CO)C1C2C3]

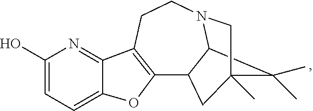 [SMILES: CC12CC3c4oc5ccc(O)nc5c4CCN(C1)C3C2(C)C]

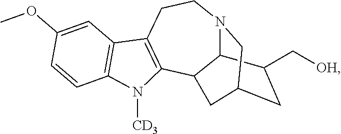 [SMILES: [2H]C([2H])([2H])n1c2c(c3cc(OC)ccc31)CCN1CC3CC(CO)C1C2C3]

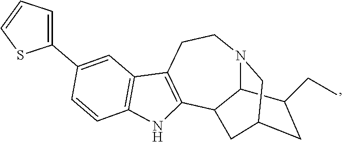 [SMILES: CCC1CC2CC3c4[nH]c5ccc(-c6cccs6)cc5c4CCN(C2)C13]

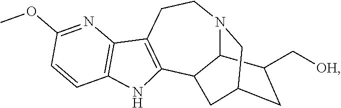 [SMILES: COc1ccc2[nH]c3c(c2n1)CCN1CC2CC(CO)C1C3C2]

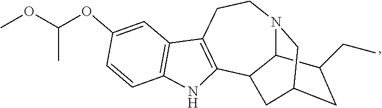 [SMILES: CCC1CC2CC3c4[nH]c5ccc(OC(C)OC)cc5c4CCN(C2)C13]

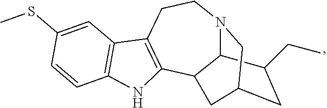 [SMILES: CCC1CC2CC3c4[nH]c5ccc(SC)cc5c4CCN(C2)C13]

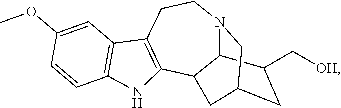 [SMILES: COc1ccc2[nH]c3c(c2c1)CCN1CC2CC(CO)C1C3C2]

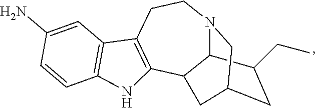 [SMILES: CCC1CC2CC3c4[nH]c5ccc(N)cc5c4CCN(C2)C13]

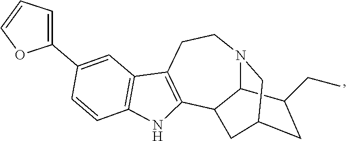 [SMILES: CCC1CC2CC3c4[nH]c5ccc(-c6ccco6)cc5c4CCN(C2)C13]

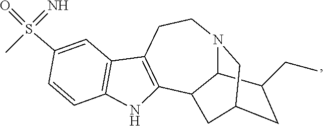 [SMILES: CCC1CC2CC3c4[nH]c5ccc(S(C)(=N)=O)cc5c4CCN(C2)C13]

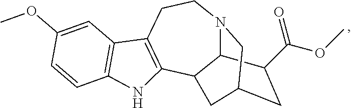

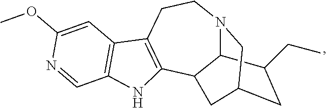 [SMILES: CCC1CC2CC3c4[nH]c5cnc(OC)cc5c4CCN(C2)C13]

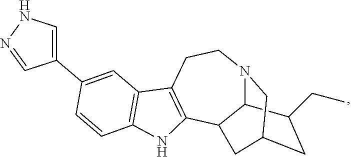 [SMILES: CCC1CC2CC3c4[nH]c5ccc(-c6cn[nH]c6)cc5c4CCN(C2)C13]

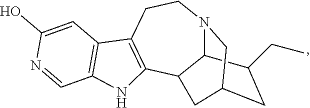 [SMILES: CCC1CC2CC3c4[nH]c5cnc(O)cc5c4CCN(C2)C13]

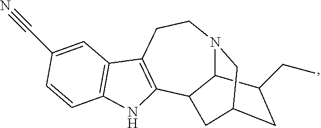 [SMILES: CCC1CC2CC3c4[nH]c5ccc(C#N)cc5c4CCN(C2)C13]

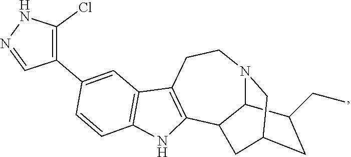 [SMILES: CCC1CC2CC3c4[nH]c5ccc(-c6cn[nH]c6Cl)cc5c4CCN(C2)C13]

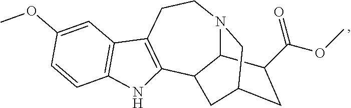

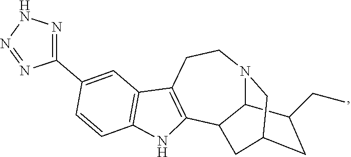 [SMILES: CCC1CC2CC3c4[nH]c5ccc(-c6nn[nH]n6)cc5c4CCN(C2)C13]

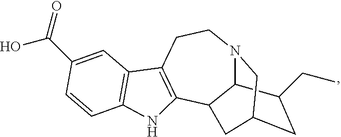 [SMILES: CCC1CC2CC3c4[nH]c5ccc(C(=O)O)cc5c4CCN(C2)C13]

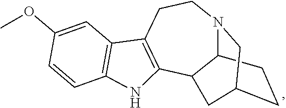 [SMILES: COc1ccc2[nH]c3c(c2c1)CCN1CC2CCC1C3C2]

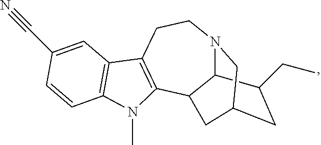 [SMILES: CCC1CC2CC3c4c(c5cc(C#N)ccc5n4C)CCN(C2)C13]

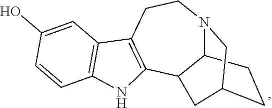 [SMILES: Oc1ccc2[nH]c3c(c2c1)CCN1CC2CCC1C3C2]

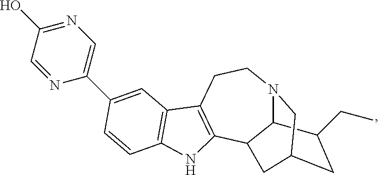 [SMILES: CCC1CC2CC3c4[nH]c5ccc(-c6cnc(O)cn6)cc5c4CCN(C2)C13]

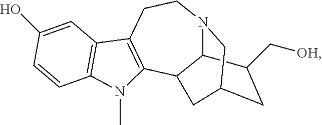 [SMILES: Cn1c2c(c3cc(O)ccc31)CCN1CC3CC(CO)C1C2C3]

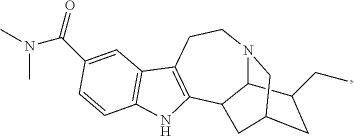 [SMILES: CCC1CC2CC3c4[nH]c5ccc(C(=O)N(C)C)cc5c4CCN(C2)C13]

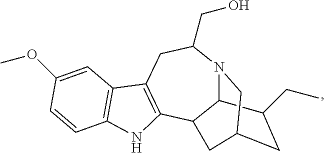 [SMILES: CCC1CC2CC3c4[nH]c5ccc(OC)cc5c4CC(CO)N(C2)C13]

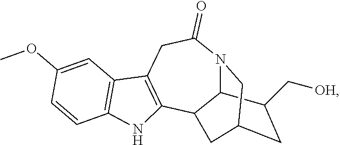 [SMILES: COc1ccc2[nH]c3c(c2c1)CC(=O)N1CC2CC(CO)C1C3C2]

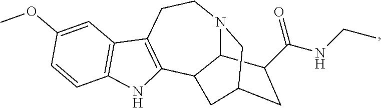 [SMILES: CCNC(=O)C1CC2CC3c4[nH]c5ccc(OC)cc5c4CCN(C2)C13]

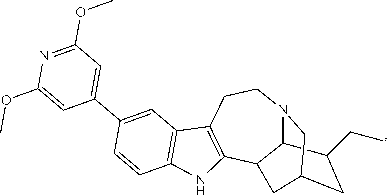 [SMILES: CCC1CC2CC3c4[nH]c5ccc(-c6cc(OC)nc(OC)c6)cc5c4CCN(C2)C13]

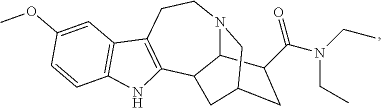 [SMILES: CCN(CC)C(=O)C1CC2CC3c4[nH]c5ccc(OC)cc5c4CCN(C2)C13]

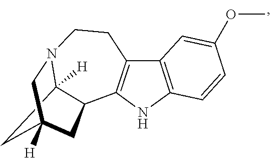

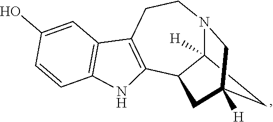

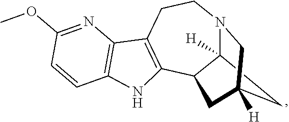

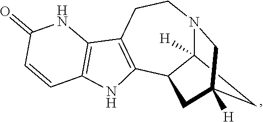

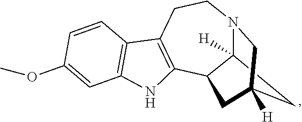

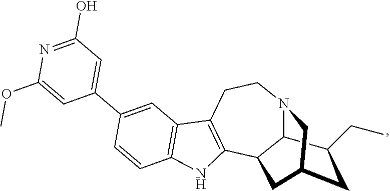

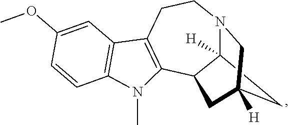

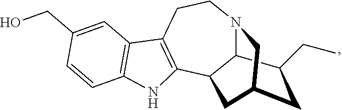

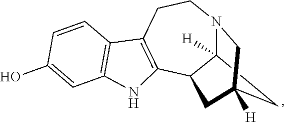

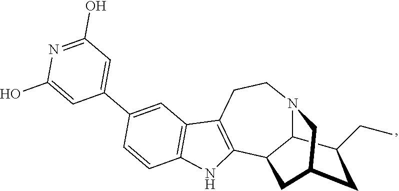

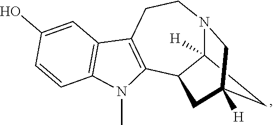

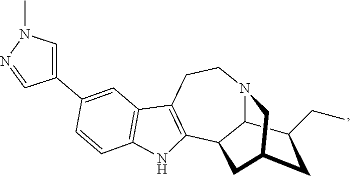

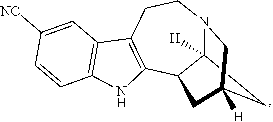

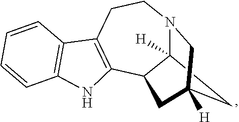

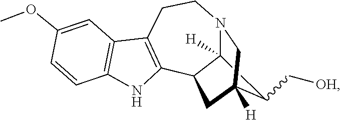

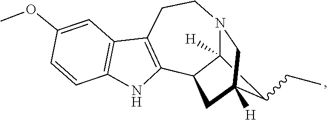

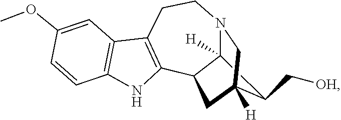

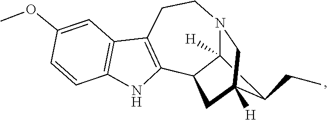

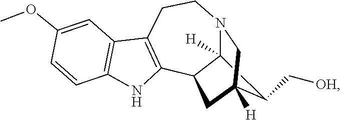

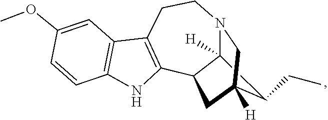

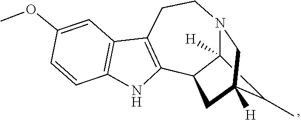

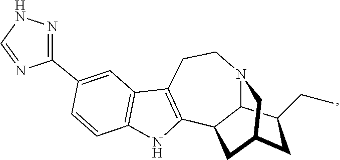

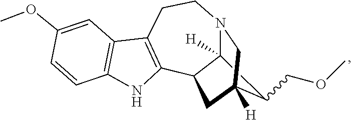

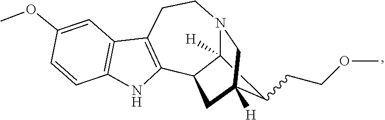

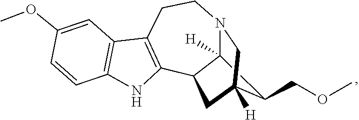

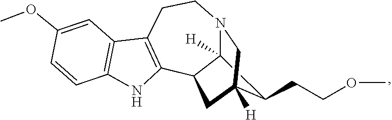

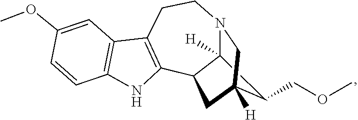

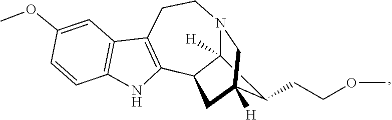

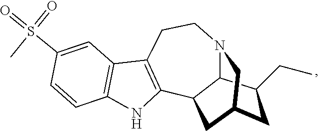

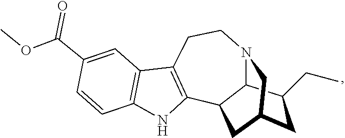

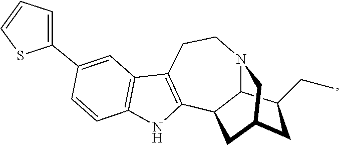

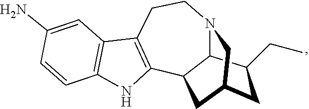

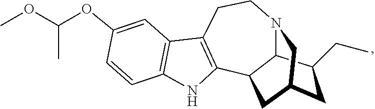

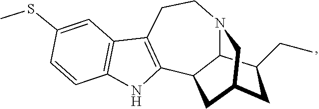

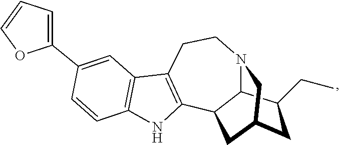

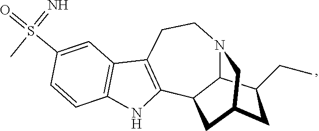

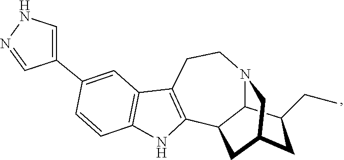

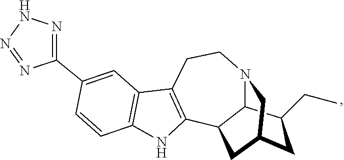

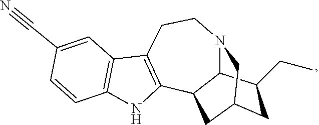 [SMILES: CC[C@H]1CC2C[C@H]3c4[nH]c5ccc(C#N)cc5c4CC[N@@](C2)C13]

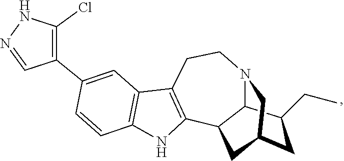 [SMILES: CC[C@H]1C[C@H]2C[C@H]3c4[nH]c5ccc(-c6cn[nH]c6Cl)cc5c4CC[N@@](C2)C13]

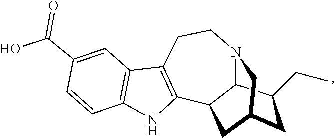

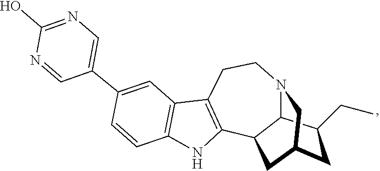

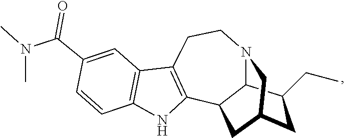

or

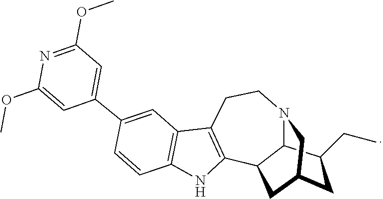

TABLE 2
Compounds

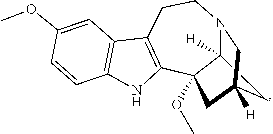

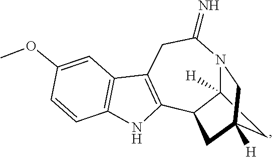

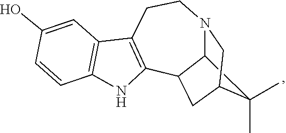 [SMILES: CC1(C)C2CC3c4[nH]c5ccc(O)cc5c4CCN(C2)C31]

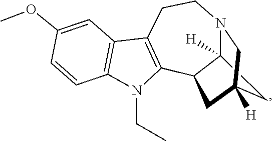

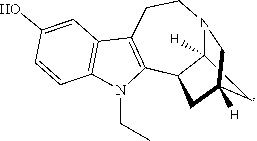

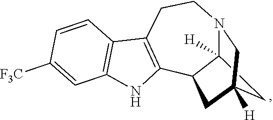

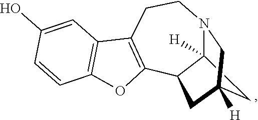

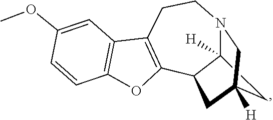

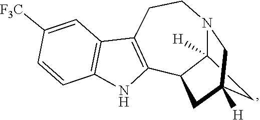

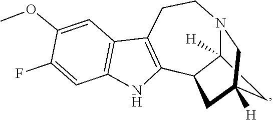

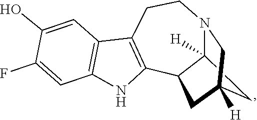

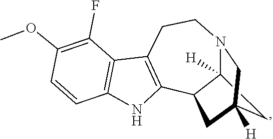

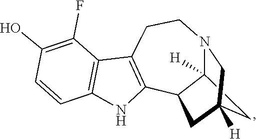

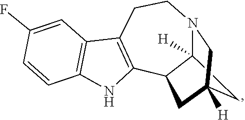

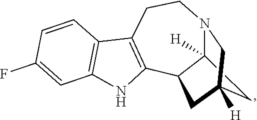

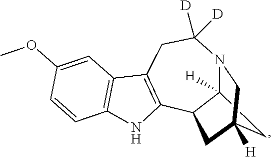

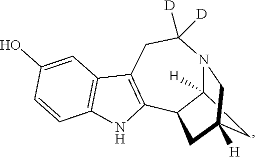

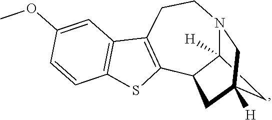

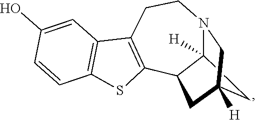

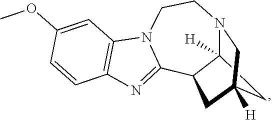

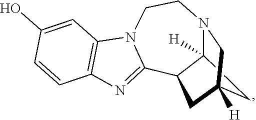

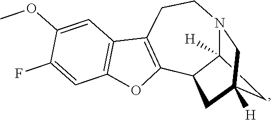

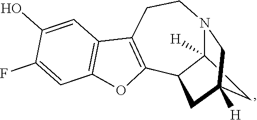

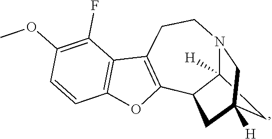

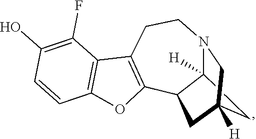

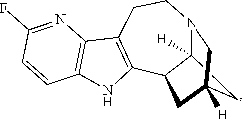

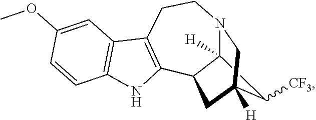

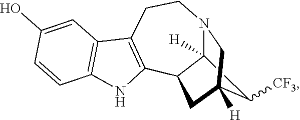

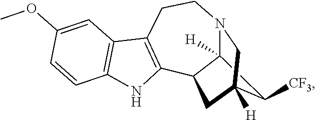

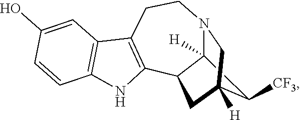

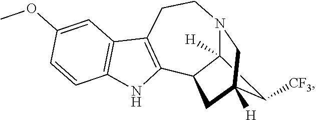

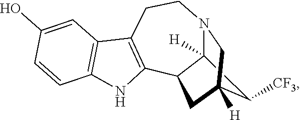

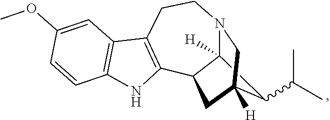

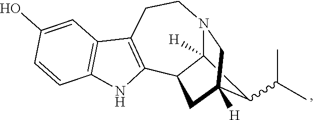

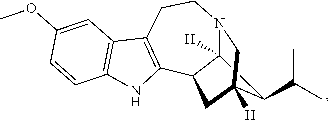

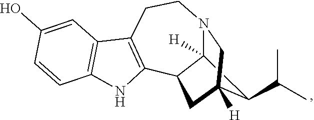

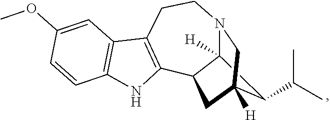

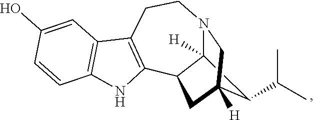

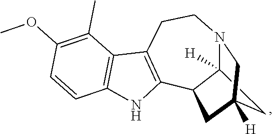

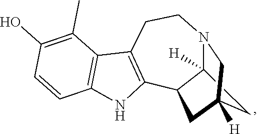

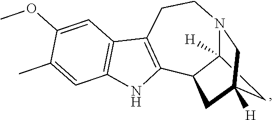

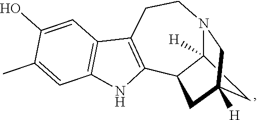

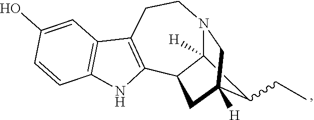

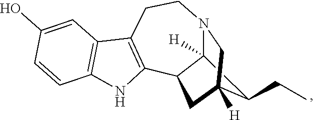

or

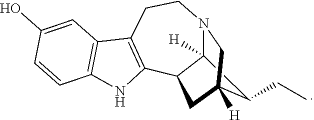

### Compositions

The present disclosure provides pharmaceutical compositions for treating various conditions or disorders in a subject in need thereof. In some embodiments, a pharmaceutical composition comprises one or more compounds of the present disclosure (e.g., a compound of Formula (I), (I′), (I″), (I-a), (I-b), (II), (II′), (II″), (II-a), (III), (III′), (III″), or Tables 1 and 2 or pharmaceutically acceptable salt, prodrug, or stereoisomer thereof. In embodiments, the pharmaceutical compositions comprise pharmaceutically acceptable excipients and adjuvants.

The pharmaceutically acceptable excipients and adjuvants are added to the composition or formulation for a variety of purposes. In embodiments, a pharmaceutical composition comprising one or more compounds disclosed herein, or a pharmaceutically acceptable salt thereof, further comprise a pharmaceutically acceptable carrier. In embodiments, a pharmaceutically acceptable carrier includes a pharmaceutically acceptable excipient, binder, and/or diluent. In embodiments, suitable pharmaceutically acceptable carriers include, but are not limited to, inert solid fillers or diluents and sterile aqueous or organic solutions. In embodiments, suitable pharmaceutically acceptable excipients include, but are not limited to, water, salt solutions, alcohol, polyethylene glycols, gelatin, lactose, amylase, magnesium stearate, talc, silicic acid, viscous paraffin, and the like.

For the purposes of this disclosure, the compounds of the present disclosure can be formulated for administration by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

Generally, the compounds of the present disclosure are administered in a therapeutically effective amount. The amount of the compound actually administered will typically be determined by a physician, in the light of the relevant circumstances, including the condition to be treated, the chosen route of administration, the actual compound-administered, the age, weight, and response of the individual patient, the severity of the patient's symptoms, and the like.

### Methods of Treatment

In embodiments, the present disclosure provides methods of treating a disease or disorder in a subject in need thereof, the methods comprising administering a therapeutically effective amount of a compound described herein (e.g., a compound of Formula (I), (I′), (I″), (I-a), (I-b), (II), (II′), (II″), (II-a), (III), (III′), (III″) or Tables 1 and 2), or pharmaceutically acceptable salt, prodrug, or stereoisomer thereof to the subject.

In embodiments, the present disclosure provides methods of treating alcoholism, substance abuse disorder, or opioid use disorder. In embodiments, the present disclosure provides methods of treating opioid use disorder. In embodiments, the present disclosure provides methods of treating the symptoms of detoxification and/or withdrawal that result from stopping or reducing the use of a medication or drug. In embodiments, the medication or drug is a substance with a high potential for dependency or abuse.

In embodiments, the present disclosure provides methods of treating a condition related to compulsive/repetitive behaviors, underlying neurocircuitries and neuroplastic effects (e.g., addictions such as gambling or sex, eating disorders, obsessive compulsive disorder (OCD), major depressive disorder (MDD), treatment-resistant depression (TRD), anxiety, post-traumatic stress disorder) (PTSD), attention-deficit/hyperactivity disorder (ADHD), autism spectrum disorder (ASD), and the like).

In embodiments, the present disclosure provides methods of treating one or more disorders or diseases selected from depression, major depression, chronic pain, acute pain, eating disorder, anxiety disorder, obsessive-compulsive disorder (OCD), stress disorder, post-traumatic stress disorder (PTSD), acute stress disorder, panic disorder, social anxiety disorder, generalized anxiety disorder, specific phobia, opioid use disorder (OUD), alcohol use disorder (AUD), polydrug use disorder, headache, migraine, traumatic brain injury (TBI), Parkinson's disease, substance use disorder (SUD), nicotine/tobacco use disorder, and opioid withdrawal symptoms, comprising administering a therapeutically effective amount of a compound disclosed herein (e.g., a compound of Formula (I), (I′), (I″), (I-a), (I-b), (II), (II′), (II″), (II-a), (III), (III′), (III″) or Tables 1 and 2), or pharmaceutically acceptable salt, prodrug, or stereoisomer thereof, or a pharmaceutical composition thereof to the subject.

In embodiments, the present disclosure provides methods of treating substance abuse disorder in a subject in need thereof, comprising administering a therapeutically effective amount of a compound disclosed herein (e.g., a compound of Formula (I), (I′), (I″), (I-a), (I-b), (II), (II′), (II″), (II-a), (III), (III′), (III″) or Tables 1 and 2), or pharmaceutically acceptable salt, prodrug, or stereoisomer thereof, or a pharmaceutical composition thereof to the subject.

In embodiments, the present disclosure provides methods of treating opioid use disorder in a subject in need thereof, comprising administering a therapeutically effective amount of a compound disclosed herein (e.g., a compound of Formula (I), (I′), (I″), (I-a), (I-b), (II), (II′), (II″), (II-a), (III), (III′), (III″) or Tables 1 and 2), or pharmaceutically acceptable salt, prodrug, or stereoisomer thereof, or a pharmaceutical composition thereof to the subject.

### Numbered Embodiments

In addition to the disclosure above, the Examples below, and the appended claims, the disclosure sets for the following numbered embodiments.
1. A compound of Formula (I′):

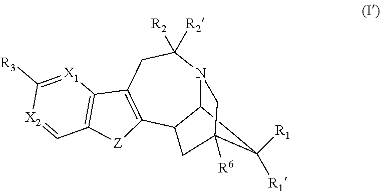

or a pharmaceutically acceptable salt, prodrug, or stereoisomer thereof, wherein:
X₁ and X₂ are independently —CR₃—, or —N—;
R₁ and R₁′ are independently hydrogen, alkyl, —CH₂ORₐ, —CH₂CH₂ORₐ —CH₂NH₂, —CH₂NRₐRₐ, —CH₂SRₐ, —C(═O)ORₐ, —CONHRₐ, or —CON(Rₐ)Rₐ;
R₂ and R₂′ are independently hydrogen, deuterium, —CH₂OH, —CH₂O-alkyl, —COOH, —CON(Rₐ)Rₐ, or R₂ and R₂′ taken together with the atom to which they are attached form ═O, ═S, ═NH, or ═N-alkyl;
each R₃ is independently hydrogen, deuterium, halogen, alkyl, —ORₐ, —NO₂, —CN, cycloalkyl, aryl, heteroaryl, —OAc, —SRₐ, —NH₂, —NH(alkyl), —NH(alkenyl), —NH(alkynyl), —NH(aryl), —NH(heteroaryl), —N(cycloalkyl), —C(═O)R_{b}, —S(═O)R_{b}, —S(═O)(═NH)R_{b}, —SO₂R_{b}, —NHSO₂R_{b}, —OC(═O)R_{b}, SC(═O)R_{b}, —NHC(═O)R_{c}, or —NHC(S)R_{c};
R₆ is hydrogen, alkyl, —CH₂ORₐ, —CH₂NH₂, —CH₂NRₐRₐ, —CH₂SRₐ, —C(═O)ORₐ, —CONHRₐ, or —CON(Rₐ)Rₐ;
each Rₐ is independently hydrogen, alkyl, deuterated alkyl, alkenyl, alkylene-aryl, alkylene-cycloalkyl, aryl, or heteroaryl;
each R_{b} is independently alkyl, aryl, heteroaryl, —OH, —O-alkyl, —NH₂, —NH(alkyl), or —N(alkyl)₂;
each R_{c} is independently alkyl, aryl, —O-alkyl, —S-alkyl, —S-aryl, —NH₂, —NH(alkyl), or —N(alkyl)₂; and
Z is O, S, or NR₄, wherein R₄ is hydrogen, alkyl, deuterated alkyl, heteroaryl, alkenyl, alkylene-aryl, alkylene-cycloalkyl, aryl, or —SO₂Rₐ.
1a. A compound of Formula (I″):

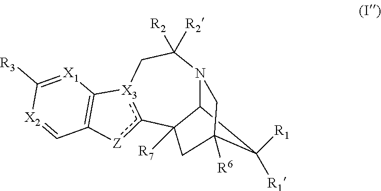

or a pharmaceutically acceptable salt, prodrug, or stereoisomer thereof, wherein:
X₁ and X₂ are independently C(R₃) or N;
X₃ is C or N;
R₁ and R₁′ are independently hydrogen, alkyl, —CF₃, —CH(Rₐ)(R_{b}), —CH₂ORₐ, —CH₂CH₂ORₐ, —CH₂NH₂, —CH₂NRₐRₐ, —CH₂SRₐ, —C(═O)ORₐ, —CONHRₐ, —ORₐ, or —CON(Rₐ)(R_{b});
R₂ and R₂′ are independently hydrogen, deuterium, —CH₂OH, —CH₂O-alkyl, —COOH, —CON(Rₐ)(R_{b}), or R₂ and R₂′ taken together with the atom to which they are attached form ═O, ═S, ═NH, or ═N-alkyl;
Each R₃ is independently hydrogen, deuterium, halogen, alkyl, —ORₐ, —NO₂, —CN, —CF₃, cycloalkyl, aryl, heteroaryl, -Oac, —SRₐ, —NH₂, —NH(alkyl), —NH(alkenyl), —NH(alkynyl), —NH(aryl), —NH(heteroaryl), —N(cycloalkyl), —C(═O)R_{b}, —S(═O)R_{b}, —S(═O)(═NH)R_{b}, —SO₂R_{b}, —NHSO₂R_{b}, —OC(═O)R_{b}, SC(═O)R_{b}, —NHC(═O)R_{c}, or —NHC(S)R_{c};
R₆ is hydrogen, alkyl, —CH₂ORₐ, —CH₂NH₂, —CH₂N(Rₐ)(R_{b}), —CH₂SRₐ, —C(═O)ORₐ, —CONHRₐ, or —CON(Rₐ)(R_{b});
R₇ is hydrogen, halogen, deuterium, alkyl, alkoxy, —C(═O)ORₐ, or —C(═O)NRₐ;
each Rₐ is independently hydrogen, alkyl, deuterated alkyl, alkenyl, alkylene-aryl, alkylene-cycloalkyl, aryl, or heteroaryl;
each R_{b} is independently alkyl, aryl, heteroaryl, —OH, —O-alkyl, —NH₂, —NH(alkyl), or —N(alkyl)₂;
each R_{c} is independently alkyl, aryl, —O-alkyl, —S-alkyl, —S-aryl, —NH₂, —NH(alkyl), or —N(alkyl)₂; and
Z is O, S, or N(R₄), wherein R₄ is absent, hydrogen, alkyl, deuterated alkyl, heteroaryl, alkenyl, alkylene-aryl, alkylene-cycloalkyl, aryl, or —SO₂Rₐ.
1b. A compound of Formula (T)

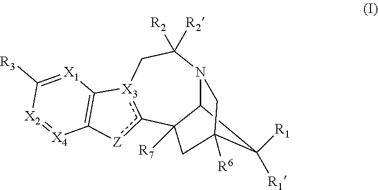

or a pharmaceutically acceptable salt, prodrug, or stereoisomer thereof, wherein:
X₁, X₂ and X₄ are independently C(R₃) or N;
X₃ is C or N;
R₁ and R₁′ are independently hydrogen, alkyl, —CF₃, —CH(Rₐ)(R_{b}), —CH₂ORₐ, —CH₂CH₂ORₐ, —CH₂NH₂, —CH₂NRₐRₐ, —CH₂SRₐ, —C(═O)ORₐ, —C(═O)NHRₐ, —ORₐ, or —C(═O)N(Rₐ)(R_{b});
R₂ and R₂′ are independently hydrogen, deuterium, —CH₂OH, —CH₂O-alkyl, —C(═O)OH, —C(═O)N(Rₐ)(R_{b}), or R₂ and R₂′ taken together with the atom to which they are attached form ═O, ═S, ═NH, or ═N-alkyl;
each R₃ is independently hydrogen, deuterium, halogen, alkyl, —ORₐ, —NO₂, —CN, —CF₃, cycloalkyl, aryl, heteroaryl, —OAc, —SRₐ, —NH₂, —NH(alkyl), —NH(alkenyl), —NH(alkynyl), —NH(aryl), —NH(heteroaryl), —N(cycloalkyl), —C(═O)R_{b}, —S(═O)R_{b}, —S(═O)₂R_{b}, —S(═O)(═NH)R_{b}, —NHS(═O)₂R_{b}, —OC(═O)R_{b}, SC(═O)R_{b}, —NHC(═O)R_{c}, or —NHC(═S)R_{c};
R₆ is hydrogen, alkyl, —CH₂ORₐ, —CH₂NH₂, —CH₂N(Rₐ)(R_{b}), —CH₂SRₐ, —C(═O)ORₐ, —C(═O)NHRₐ, or —C(═O)N(Rₐ)(R_{b});
R₇ is hydrogen, halogen, deuterium, alkyl, alkoxy, alkylene-OH, alkylene-O-alkyl, alkylene-NH₂, alkylene-NH(alkyl), alkylene-N(alkyl)₂, —C(═O)ORₐ, or —C(═O)NRₐ;
each Rₐ is independently hydrogen, alkyl, deuterated alkyl, alkenyl, alkylene-aryl, alkylene-cycloalkyl, aryl, or heteroaryl;
each R_{b} is independently alkyl, aryl, heteroaryl, —OH, —O-alkyl, —NH₂, —NH(alkyl), or —N(alkyl)₂;
each R_{c} is independently alkyl, aryl, —O-alkyl, —S-alkyl, —S-aryl, —NH₂, —NH(alkyl), or —N(alkyl)₂; and
Z is O, S, or N(R₄), wherein R₄ is absent, hydrogen, alkyl, deuterated alkyl, heteroaryl, alkenyl, alkylene-aryl, alkylene-cycloalkyl, aryl, or —SO₂Rₐ.
2. The compound of embodiment 1, wherein X₂ is —CORₐ.
3. The compound of embodiment 2, wherein X₂ is —OH or —COCH₃.
4. The compound of embodiment 1, having the Formula (I-a), or a pharmaceutically acceptable salt, prodrug, or stereoisomer thereof:

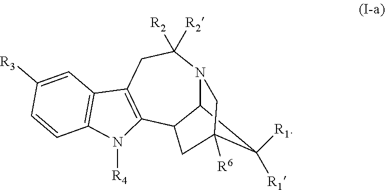

5. The compound of embodiment 1, having the Formula (I-b), or a pharmaceutically acceptable salt, prodrug, or stereoisomer thereof:

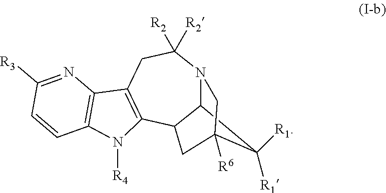

6. The compound of any one of embodiments 1-5, wherein R₁ and R₁′ are independently hydrogen, -alkyl, —CH₂ORₐ, —CH₂CH₂ORₐ, —CH₂SRₐ, —CH₂NH₂, —CH₂NRₐRₐ, —COORₐ, —CONHRₐ, or —CON(Rₐ)Rₐ.
7. The compound of any one of embodiments 1-6, wherein R₁ and R₁′ are hydrogen.
8. The compound of any one of embodiments 1-6, wherein R₁ and R₁′ are CH₃.
9. The compound of any one of embodiments 1-6, wherein R₁ is —CH₂CH₃, —CH₂OH, —CH₂OCH₃ or —CH₂CH₂OCH₃, and R₁′ is hydrogen.
10. The compound of any one of embodiments 1-9, wherein R₂ and R₂′ are hydrogen.
11. The compound of any one of embodiments 1-9, wherein R₂ or R₂′ is —COOH.
12. The compound of any one of embodiments 1-9, wherein R₂ or R₂′ is —CON(Rₐ)Rₐ.
13. The compound of any one of embodiments 1-9, wherein R₂ and R₂′ taken together with the atom to which they are attached form ═O.
14. The compound of any one of embodiments 1-9, wherein R₂ and R₂′ taken together with the atom to which they are attached form ═S.
15. The compound of any one of embodiments 1-9, wherein R₂ and R₂′ taken together with the atom to which they are attached form ═NH.
16. The compound of any one of embodiments 1-15, wherein R₃ is hydrogen.
17. The compound of any one of embodiments 1-15, wherein R₃ is —OH.
18. The compound of any one of embodiments 1-15, wherein R₃ is —OCH₃.
19. The compound of any one of embodiments 1-15, wherein R₃ is heteroaryl.
20. The compound of any one of embodiments 1-19, wherein R₄ is hydrogen.
21. The compound of any one of embodiments 1-19, wherein R₄ is alkyl.
22. The compound of any one of embodiments 1-19, wherein R₄ is —CH₃.
23. The compound of any one of embodiments 1-22, wherein R₆ is hydrogen or —CH₃.
24. The compound of any one of embodiments 1-23, wherein Z is NR₄.
25. The compound of embodiment 1, or a pharmaceutically acceptable salt, prodrug, or stereoisomer thereof, having the formula:

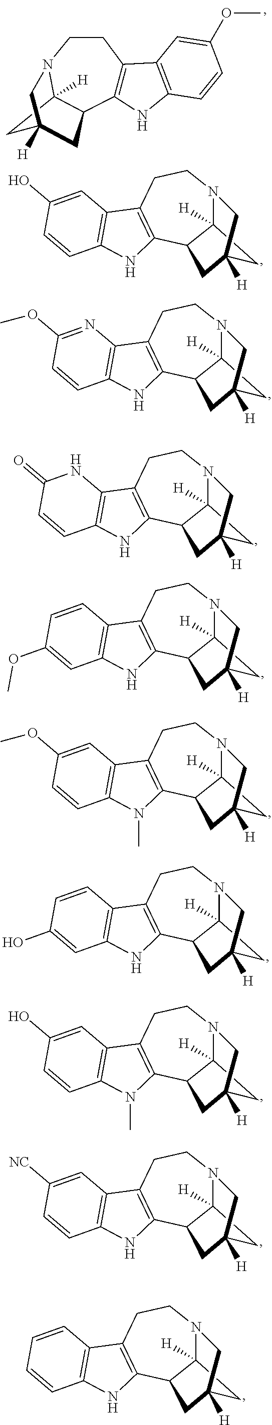

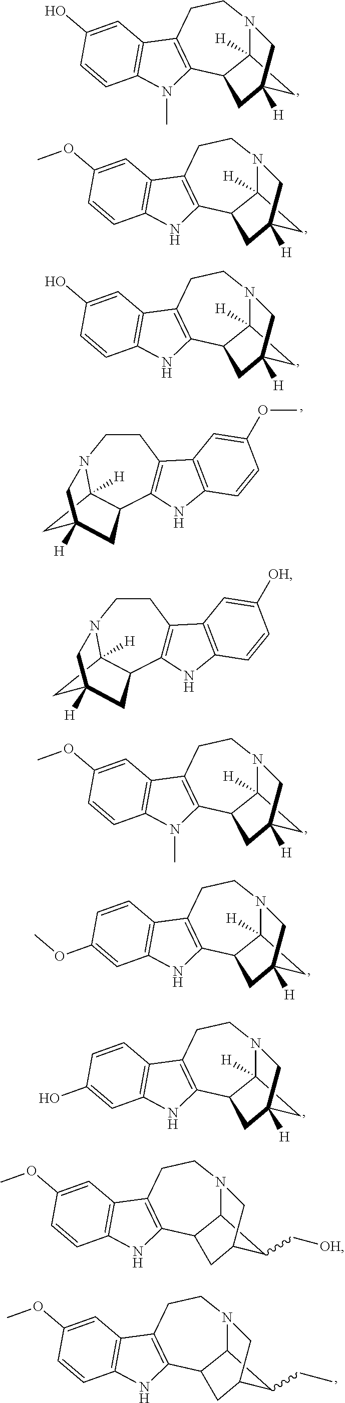

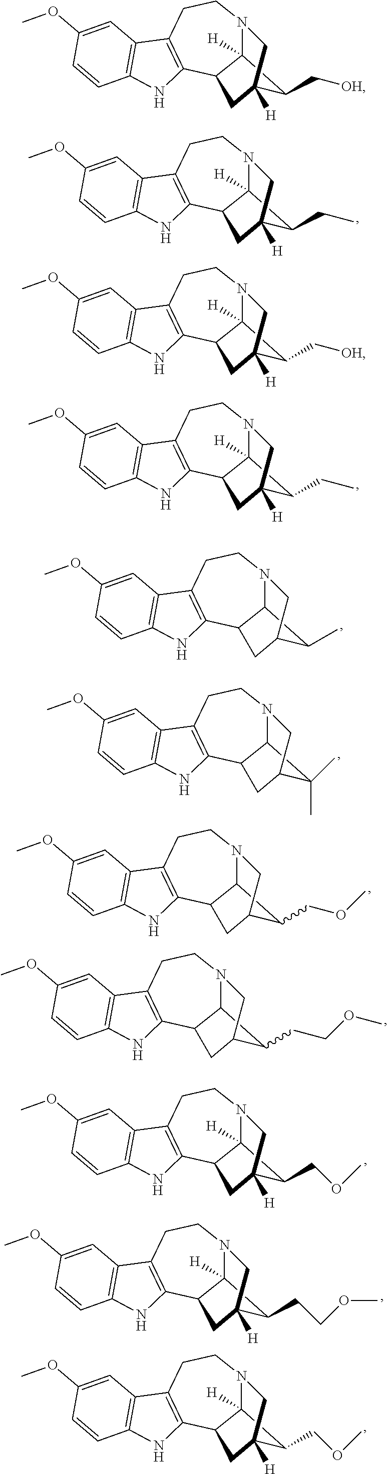

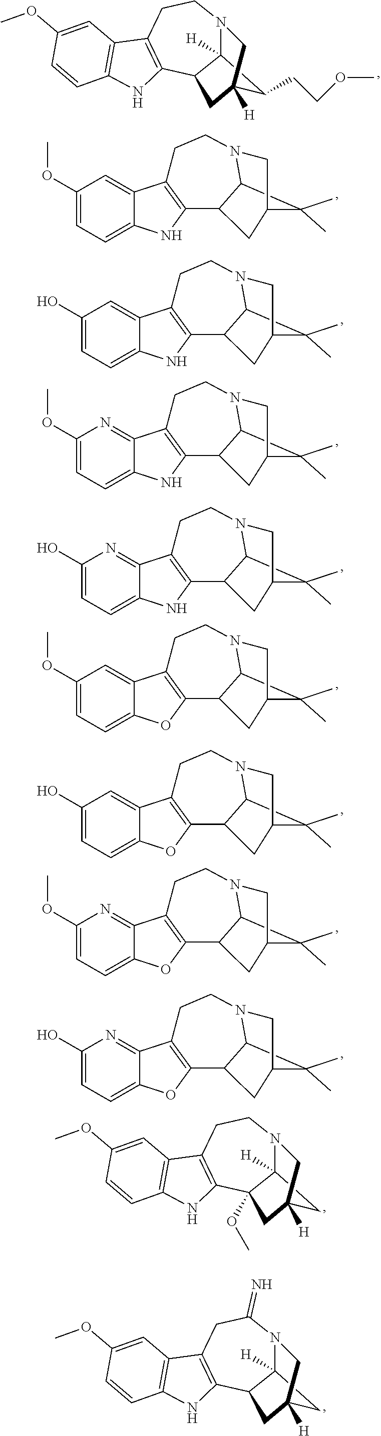

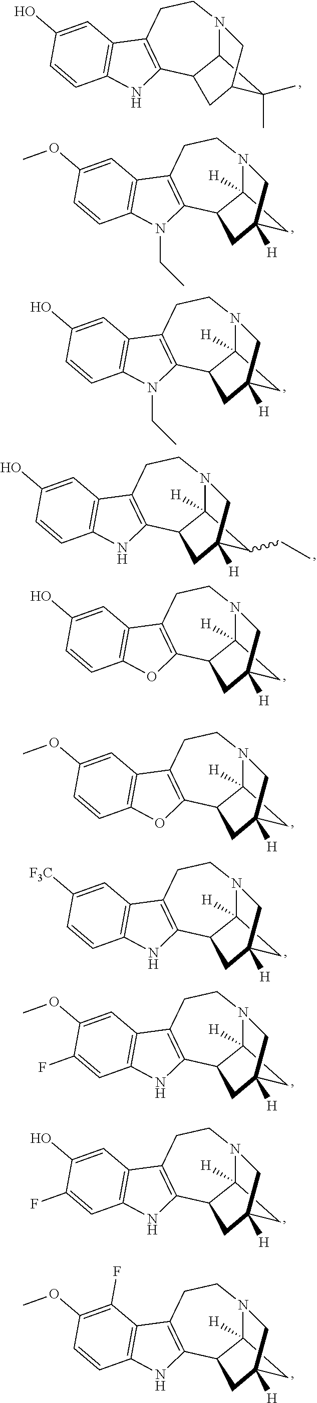

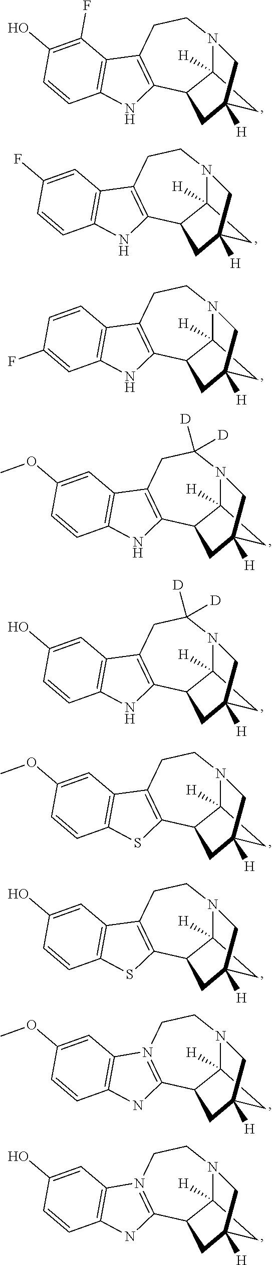

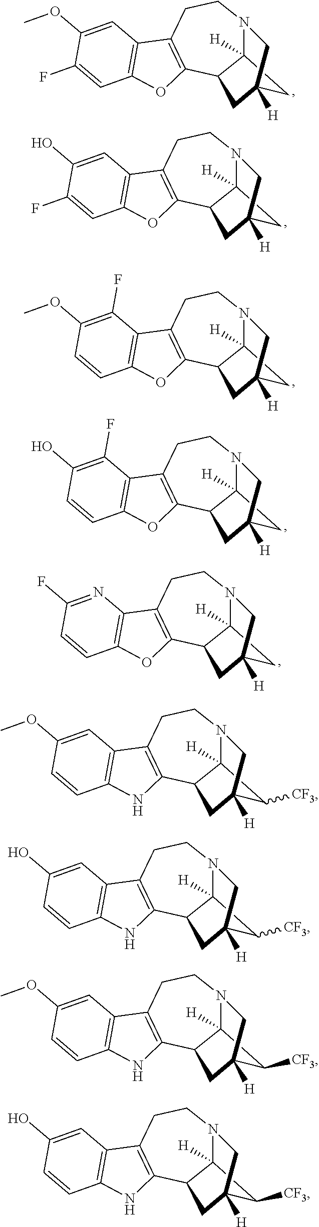

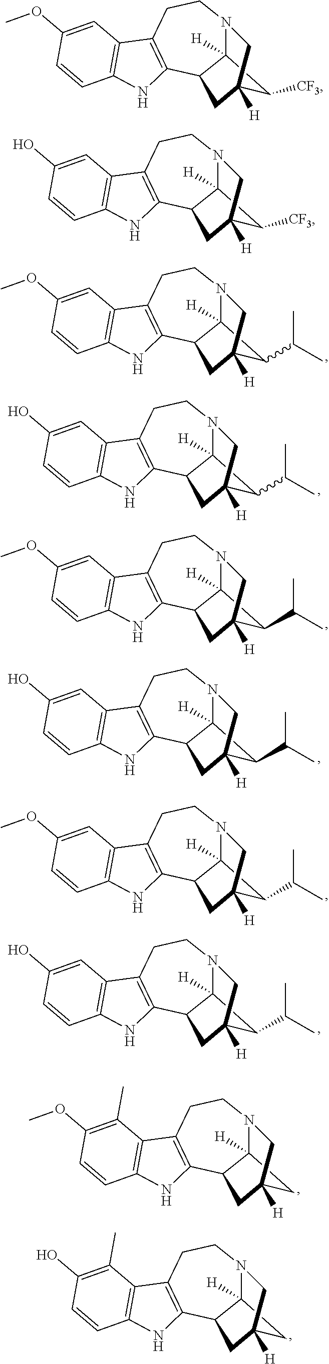

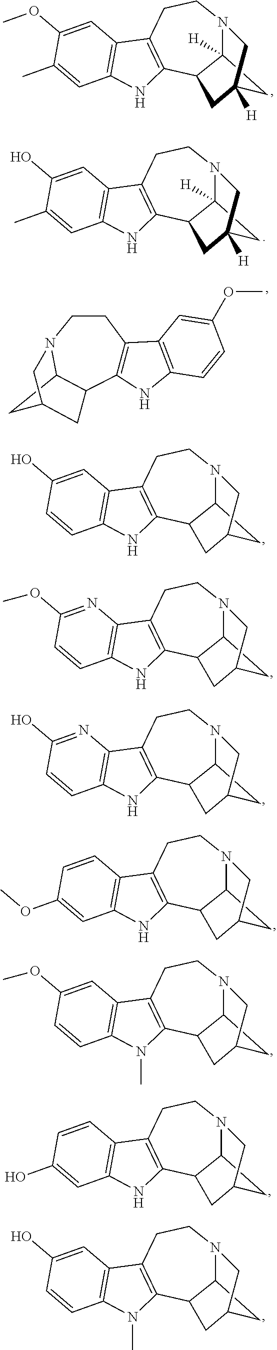

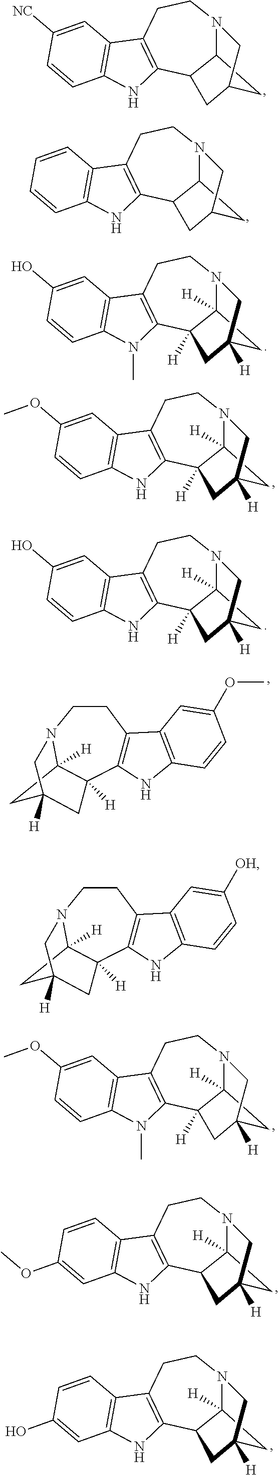

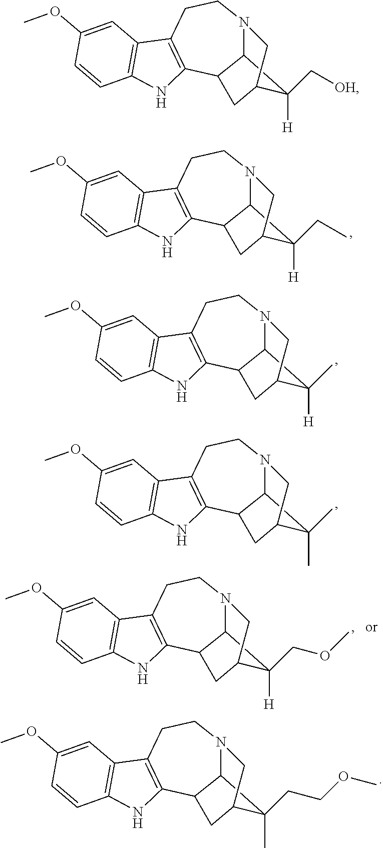

26. A compound of Formula (II):

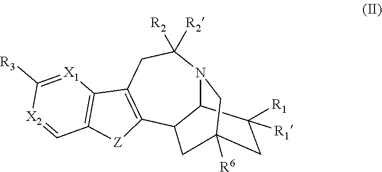

or a pharmaceutically acceptable salt, prodrug, or stereoisomer thereof, wherein:
X₁ and X₂ are independently —CR₃— or —N—, wherein at least one of X₁ and X₂ is —N—;
R₁ and R₁′ are independently hydrogen, alkyl, —CH₂ORₐ, —CH₂NH₂, —CH₂NRₐRₐ, —CH₂SRₐ, —C(═O)ORₐ, —CONHRₐ, or —CON(Rₐ)Rₐ;
R₂ and R₂′ are independently hydrogen, deuterium, —CH₂OH, —CH₂O-alkyl, —COOH, —CON(Rₐ)Rₐ, or R₂ and R₂′ taken together with the atom to which they are attached form ═O, ═S, ═NH, or ═N-alkyl;
each R₃ is independently hydrogen, deuterium, halogen, alkyl, —ORₐ, —NO₂, —CN, cycloalkyl, aryl, heteroaryl, —OAc, —SRₐ, —NH₂, —NH(alkyl), —NH(alkenyl), —NH(alkynyl), —NH(aryl), —NH(heteroaryl), —N(cycloalkyl), —C(═O)R_{b}, —S(═O)R_{b}, —SO₂R_{b}, —NHSO₂R_{b}, —OC(═O)R_{b}, —SC(═O)R_{b}, —NHC(═O)R_{c} or —NHC(S)R_{c};
R₆ is hydrogen, alkyl, —CH₂ORₐ, —CH₂NH₂, —CH₂NRₐRₐ, —CH₂SRₐ, —C(═O)ORₐ, —CONHRₐ, or —CON(Rₐ)Rₐ;
each Rₐ is independently hydrogen, alkyl, deuterated alkyl, alkenyl, alkylene-aryl, alkylene-cycloalkyl, aryl, or heteroaryl;
R_{b} is alkyl, aryl, heteroaryl, —OH, —O-alkyl, —NH₂, —NH(alkyl), or —N(alkyl)₂;
R_{c} is alkyl, aryl, —O-alkyl, —S-alkyl, —S-aryl, —NH₂, —NH(alkyl), or —N(alkyl)₂; and
Z is O, S, or NR₄, wherein R₄ is hydrogen, alkyl, deuterated alkyl, heteroaryl, alkenyl, alkylene-aryl, alkylene-cycloalkyl, aryl, or —SO₂Rₐ.
27. The compound of embodiment 26, having the Formula (II′), or a pharmaceutically acceptable salt, prodrug, or stereoisomer thereof:

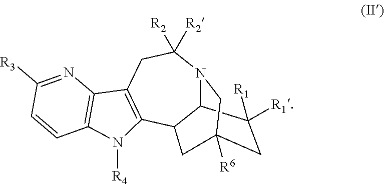

28. The compound of embodiment 26, having the Formula (II″), or a pharmaceutically acceptable salt, prodrug, or stereoisomer thereof:

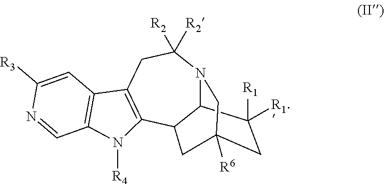

29. The compound of any one of embodiments 26-28, wherein R₁ and R₁′ are independently hydrogen, —CH₂CH₃, —CH₂OH, —CH₂ORₐ, —CH₂SH, —CH₂SRₐ, —CH₂NH₂, —CH₂NRₐRₐ, —COORₐ, —CONHRₐ, or —CON(Rₐ)Rₐ.
30. The compound of any one of embodiments 26-28, wherein R₁ or R₁′ is —CH₂CH₃.
31. The compound of any one of embodiments 26-28, wherein R₁ or R₁′ is —CH₂OH.
32. The compound of any one of embodiments 26-31, wherein R₂ and R₂′ are hydrogen.
33. The compound of any one of embodiments 26-32, wherein R₃ is —OH.
34. The compound of any one of embodiments 26-32, wherein R₃ is —OCH₃.
35. The compound of any one of embodiments 26-34, wherein R₄ is hydrogen.
36. The compound of any one of embodiments 26-35, wherein R₆ is hydrogen.
37. The compound of embodiment 26, or a pharmaceutically acceptable salt, prodrug, or stereoisomer thereof, having the formula:

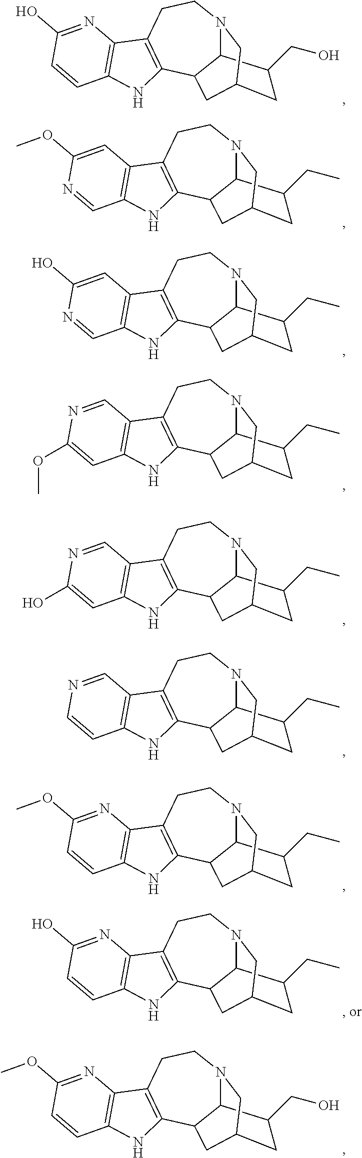

38. A compound of Formula (II):

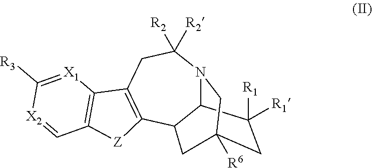

or a pharmaceutically acceptable salt, prodrug, or stereoisomer thereof, wherein:
X₁ and X₂ are independently —CR₃—, or —N—;
R₁ and R₁′ are independently hydrogen, alkyl, —CH₂ORₐ, —CH₂NH₂, —CH₂NRₐRₐ, —CH₂SRₐ,
—C(═O)ORₐ, —CONHRₐ, or —CON(Rₐ)Rₐ; R₂ and R₂′ are independently hydrogen, —CH₂OH, —CH₂O-alkyl, —COOH, —CON(Rₐ)Rₐ, or R₂ and R₂′ taken together with the atom to which they are attached form ═O, ═S, ═NH, or ═N-alkyl;
each R₃ is independently deuterium, halogen, alkyl, —ORₐ, —NO₂, —CN, cycloalkyl, aryl, heteroaryl, —OAc, —SRₐ, —NH₂, —NH(alkyl), —NH(alkenyl), —NH(alkynyl), —NH(aryl), —NH(heteroaryl), —N(cycloalkyl), —C(═O)R_{b}, —S(═O)R_{b}, —SO₂R_{b}, —NHSO₂R_{b}, —SC(═O)R_{b}, —NHC(═O)R_{c} or —NHC(S)R_{c};
R₆ is hydrogen, alkyl, —CH₂ORₐ, —CH₂NH₂, —CH₂NRₐRₐ, —CH₂SRₐ, —C(═O)ORₐ, —CONHRₐ, or —CON(Rₐ)Rₐ;
each Rₐ is independently hydrogen, alkyl, deuterated alkyl, alkenyl, alkylene-aryl, alkylene-cycloalkyl, aryl, or heteroaryl;
R_{b} is alkyl, aryl, heteroaryl, —OH, —O-alkyl, —NH₂, —NH(alkyl), or —N(alkyl)₂;
R_{c} is alkyl, aryl, —O-alkyl, —S-alkyl, —S-aryl, —NH₂, —NH(alkyl), or —N(alkyl)₂;
Z is NR₄, wherein R₄ is hydrogen, alkyl, deuterated alkyl, heteroaryl, alkenyl, alkylene-aryl, alkylene-cycloalkyl, aryl, or —SO₂Rₐ; and
when R₂ and R₂′ are hydrogen, R₃ is —OH, and X₁ and X₂ are CH, then R₁ is not —CH₂CH₃, —(CH₂)₂CN, —(CH₂)₂OCH₂C₆H5, —CD₂CD₃, —CH₂CD₃, —(CH₂)₂OH, —(CH₂)₂OCH₂CH₃, or —(CH₂)₂CF₃;
when R₁ or R₁′ is —CH₂CH₃ or —CH₂OH, and X₁ and X₂ are CH, then R₃ is not —OCH₂CH₃, —OC(CH₃)₃, or —CH═CH₂, or R₄ is not —(CH₂)₃N(CH₃)₂; and the compound is not

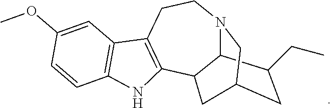 [SMILES: CCC1CC2CC3c4[nH]c5ccc(OC)cc5c4CCN(C2)C13]

39. The compound of embodiment 38, having the Formula (II-a), or a pharmaceutically acceptable salt, prodrug, or stereoisomer thereof:

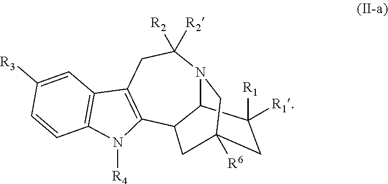

40. The compound of embodiment 38 or 39, wherein R₁ and R₁′ are independently hydrogen, -alkyl, —CH₂ORₐ, —CH₂CH₂ORₐ, —CH₂SRₐ, —CH₂NRₐRₐ, —C(═O)ORₐ, —CONHRₐ, or —CON(Rₐ)Rₐ.
41. The compound of any one of embodiments 38-40, wherein R₂ and R₂′ are hydrogen.
42. The compound of any one of embodiments 38-40, wherein R₂ and R₂′ taken together with the atom to which they are attached form ═O.
43. The compound of any one of embodiments 38-40, wherein R₂ and R₂′ taken together with the atom to which they are attached form ═S.
44. The compound of any one of embodiments 38-40, wherein R₂ and R₂′ taken together with the atom to which they are attached form ═NH.
45. The compound of any one of embodiments 38-44, wherein R₃ is heteroaryl.
46. The compound of embodiment 45, wherein R₃ is a 5-6 membered heteroaryl ring.
47. The compound of embodiment 45 or 46, wherein R₃ is

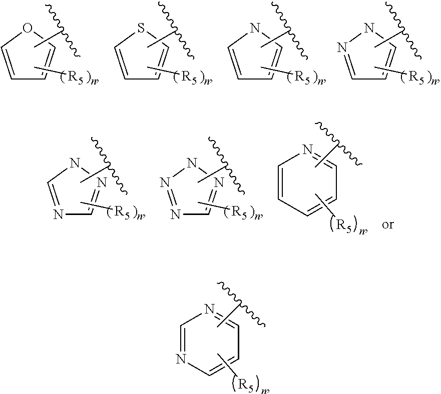

wherein n is 0, 1, or 2; and R₅ is hydrogen, halogen, OH, O-alkyl, or C₁-C₃ alkyl.
48. The compound of embodiment 47, wherein R₅ is hydrogen, Cl, OH, O-alkyl, or C₁-C₃ alkyl.
49. The compound of any one of embodiments 45-48, wherein R₃ is

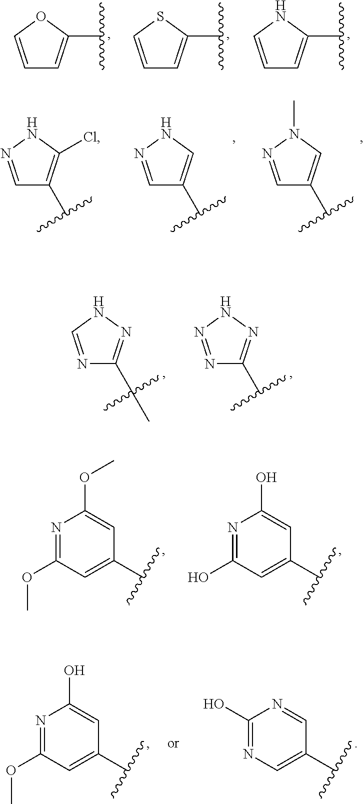

50. The compound of any one of embodiments 38-44, wherein R₃ is —CN.
51. The compound of any one of embodiments 38-44, wherein R₃ is —S(═O)R_{b}, —SO₂R_{b}, or —S— alkyl.
52. The compound of any one of embodiments 38-44, wherein R₃ is —SCH₃.
53. The compound of any one of embodiments 38-44, wherein R₃ is —SO₂CH₃.
54. The compound of any one of embodiments 38-44, wherein R₃ is —S(═O)(═NH)(CH₃).
55. The compound of any one of embodiments 38-44, wherein R₃ is —NH₂.
56. The compound of any one of embodiments 38-44, wherein R₃ is —COR_{b}.
57. The compound of embodiment 56, wherein R_{b} is O-alkyl or —N(alkyl)₂.
58. The compound of embodiment 56, wherein R_{b} is —OCH₃ or —N(CH₃)₂.
59. The compound of any one of embodiments 38-44, wherein R₃ is —ORₐ.
60. The compound of embodiment 59, wherein R₃ is —OCH(CH₃)OCH₃.
61. The compound of any one of embodiments 38-44, wherein R₃ is deuterium.
62. The compound of any one of embodiments 38-44, wherein R₃ is —OH.
63. The compound of any one of embodiments 38-44, wherein R₃ is —OCH₃.
64. The compound of any one of embodiments 38-63, wherein R₄ is hydrogen.
65. The compound of any one of embodiments 38-63, wherein R₄ is alkyl.
66. The compound of any one of embodiments 38-63, wherein R₄ is —CH₃.
67. The compound of any one of embodiments 38-66, wherein R₆ is hydrogen.
68. The compound of embodiment 38, or a pharmaceutically acceptable salt, prodrug, or stereoisomer thereof, having the formula:

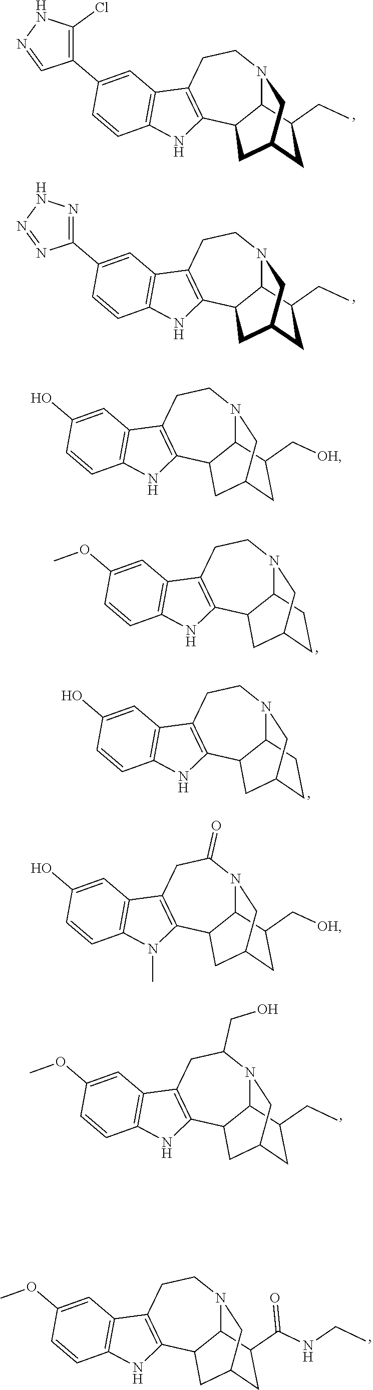

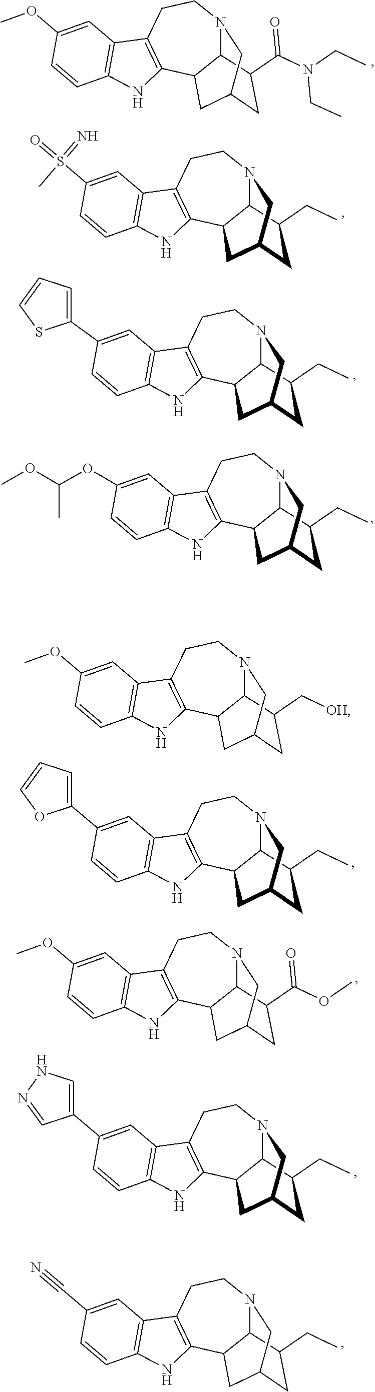

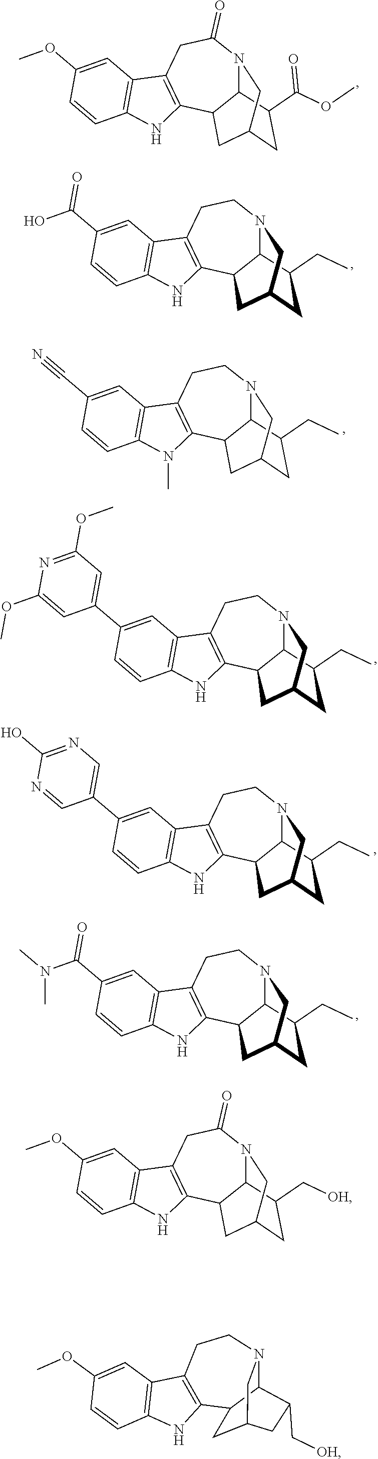

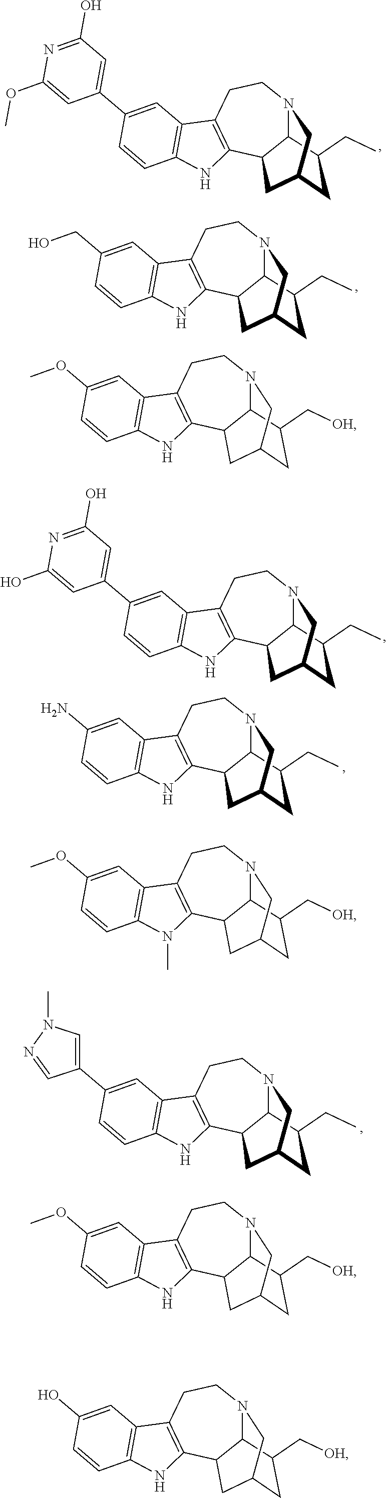

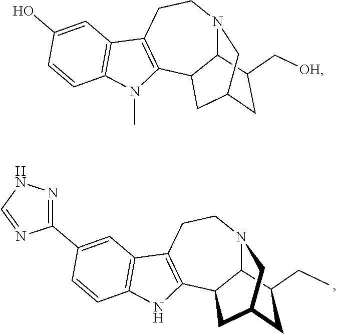

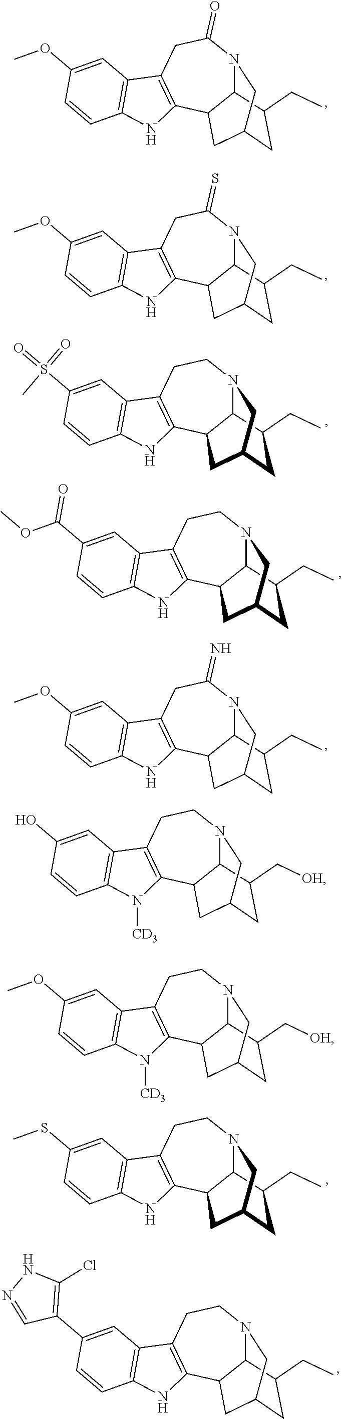

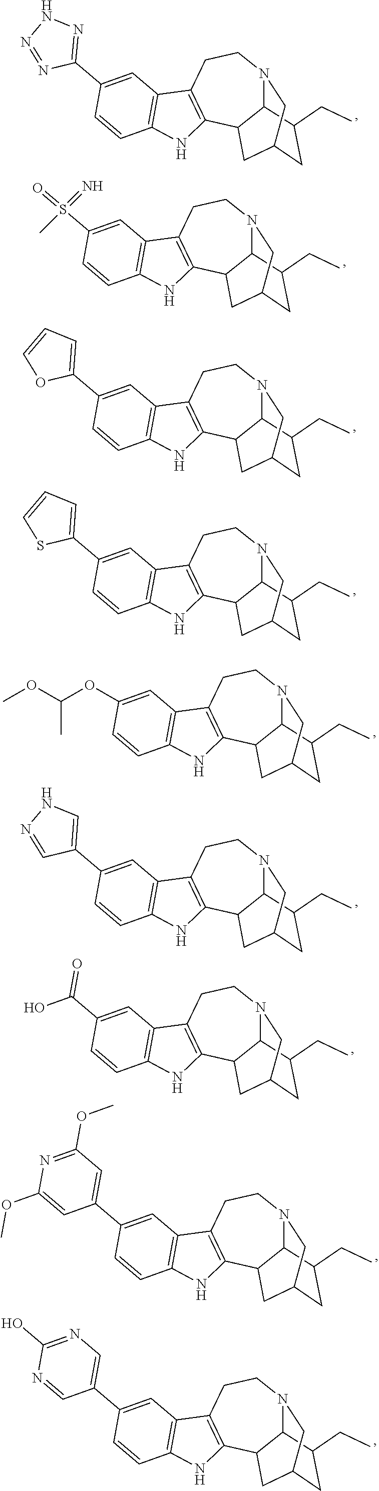

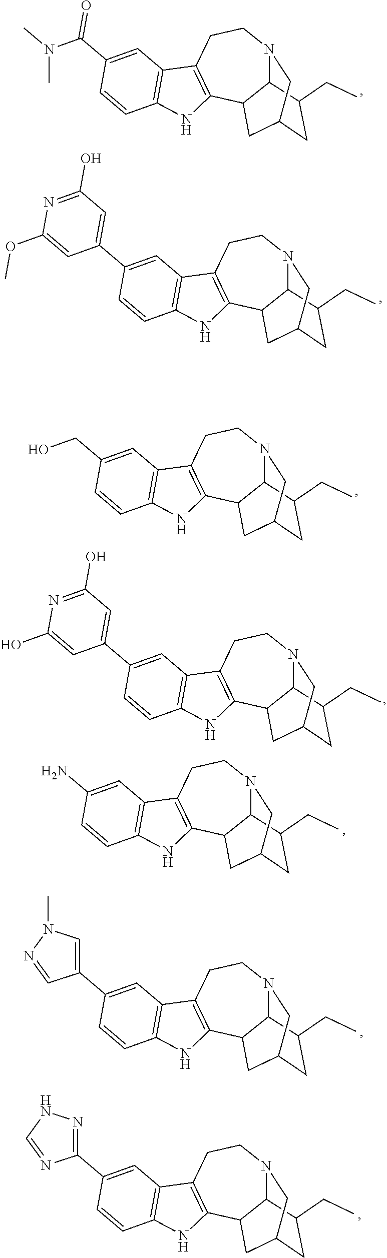

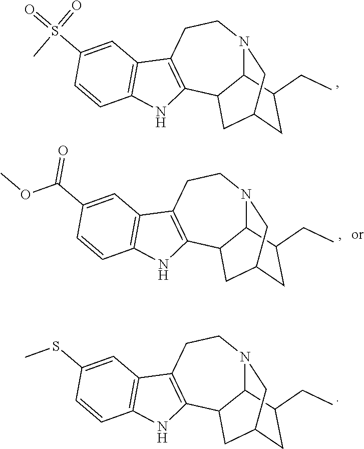

69. A compound of Formula (III):

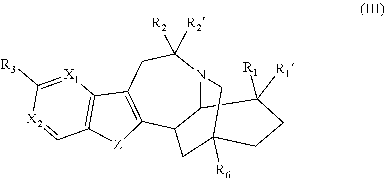

or a pharmaceutically acceptable salt, prodrug, or stereoisomer thereof, wherein:
X₁ and X₂ are independently —CR₃—, or —N—;
R₁ is hydrogen, alkyl, —CH₂ORₐ, —CH₂CH₂ORₐ, —CH₂NH₂, —CH₂NRₐRₐ, —CH₂SRₐ, —C(═O)ORₐ, —CONHRₐ, or —CON(Rₐ)Rₐ;
R₂ and R₂′ are independently hydrogen, deuterium, —CH₂OH, —CH₂O-alkyl, —COOH, —CON(Rₐ)Rₐ, or R₂ and R₂′ taken together with the atom to which they are attached form ═O, ═S, ═NH, or ═N-alkyl;
each R₃ is independently hydrogen, deuterium, halogen, alkyl, —ORₐ, —NO₂, —CN, cycloalkyl, aryl, heteroaryl, —OAc, —SRₐ, —NH₂, —NH(alkyl), —NH(alkenyl), —NH(alkynyl), —NH(aryl), —NH(heteroaryl), —N(cycloalkyl), —C(═O)R_{b}, —S(═O)R_{b}, —SO₂R_{b}, —NHSO₂R_{b}, —OC(═O)R_{b}, SC(═O)R_{b}, —NHC(═O)R_{c} or —NHC(S)R_{c};
R₆ is hydrogen, alkyl, —CH₂ORₐ, —CH₂NH₂, —CH₂NRₐRₐ, —CH₂SRₐ, —C(═O)ORₐ, —CONHRₐ, or —CON(Rₐ)Rₐ;
each Rₐ is independently hydrogen, alkyl, deuterated alkyl, alkenyl, alkylene-aryl, alkylene-cycloalkyl, aryl, or heteroaryl;
each R_{b} is independently alkyl, aryl, heteroaryl, —OH, —O-alkyl, —NH₂, —NH(alkyl), or —N(alkyl)₂;
each R_{c} is independently alkyl, aryl, —O-alkyl, —S-alkyl, —S-aryl, —NH₂, —NH(alkyl), or —N(alkyl)₂;
Z is N(R₄), wherein R₄ is hydrogen, alkyl, deuterated alkyl, heteroaryl, alkenyl, alkylene-aryl, alkylene-cycloalkyl, aryl, or —SO₂Rₐ; and
R₄ is hydrogen, alkyl, deuterated alkyl, heteroaryl, alkenyl, alkylene-aryl, alkylene-cycloalkyl, aryl, or —SO₂Rₐ.
70. The compound of embodiment 69, having the Formula (III′), or a pharmaceutically acceptable salt, prodrug, or stereoisomer thereof:

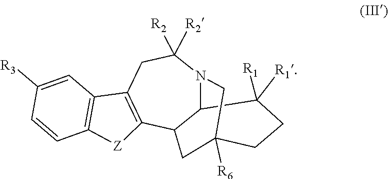

71. The compound of embodiment 69, having the Formula (III″), or a pharmaceutically acceptable salt, prodrug, or stereoisomer thereof:

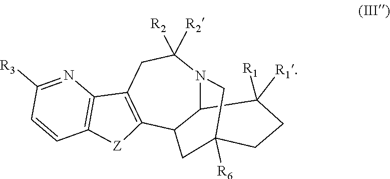

72. The compound of any one of embodiments 69-71, wherein R₁ and R₁′ are independently hydrogen, -alkyl, —CH₂ORₐ, —CH₂CH₂ORₐ, —CH₂SRₐ, —CH₂NH₂, —CH₂NRₐRₐ, —COORₐ, —CONHRₐ, or —CON(Rₐ)Rₐ.
73. The compound of any one of embodiments 69-72, wherein R₁ and R₁′ are hydrogen.
74. The compound of any one of embodiments 69-73, wherein R₂ and R₂′ are hydrogen.
75. The compound of any one of embodiments 69-73, wherein R₂ or R₂′ is —COOH.
76. The compound of any one of embodiments 69-73, wherein R₂ or R₂′ is —CON(Rₐ)Rₐ.
77. The compound of any one of embodiments 69-73, wherein R₂ and R₂′ taken together with the atom to which they are attached form ═O.
78. The compound of any one of embodiments 69-73, wherein R₂ and R₂′ taken together with the atom to which they are attached form ═S.
79. The compound of any one of embodiments 69-73, wherein R₂ and R₂′ taken together with the atom to which they are attached form ═NH.
80. The compound of any one of embodiments 69-79, wherein R₃ is hydrogen.
81. The compound of any one of embodiments 69-79, wherein R₃ is —OH.
82. The compound of any one of embodiments 69-79, wherein R₃ is —OCH₃.
83. The compound of any one of embodiments 69-79, wherein R₃ is heteroaryl.
84. The compound of any one of embodiments 69-83, wherein R₄ is hydrogen.
85. The compound of any one of embodiments 69-83, wherein R₄ is alkyl.
86. The compound of any one of embodiments 69-83, wherein R₄ is —CH₃.
87. The compound of any one of embodiments 69-86, wherein R₆ is hydrogen.
88. The compound of any one of embodiments 69-87, wherein Z is NR₄.
89. A pharmaceutical composition comprising any one of compounds of embodiments 1-88 or pharmaceutically acceptable salt, prodrug, or stereoisomer thereof.
90. A pharmaceutical composition comprising any one of compounds of embodiments 1-88 or pharmaceutically acceptable salt, prodrug, or stereoisomer thereof and a pharmaceutically acceptable excipient.
91. A method of treating a disease or disorder in a subject in need thereof comprising administering a therapeutically effective amount of any one of compounds of embodiments 1-80 or the pharmaceutical composition of embodiment 89 or 90 to the subject.
92. The method of embodiment 91, wherein the diseases or disorder is alcoholism, substance abuse disorder, or opioid use disorder.

### EXAMPLES

The disclosure now being generally described, it will be more readily understood by reference to the following examples which are included merely for purposes of illustration of certain aspects and embodiments of the present disclosure and are not intended to limit the disclosure.

The compounds of the present disclosure can be synthesized using the methods as hereinafter described below, together with synthetic methods known in the art of synthetic organic chemistry or variations thereon as appreciated by those skilled in the art.

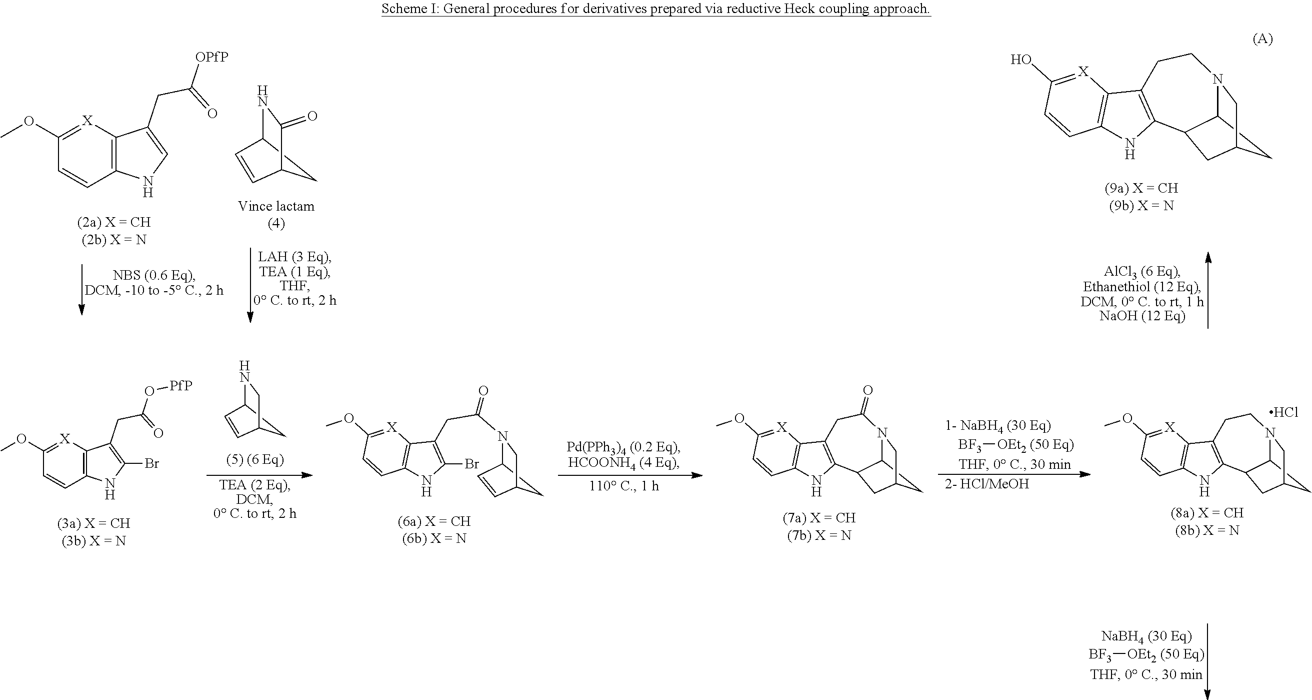

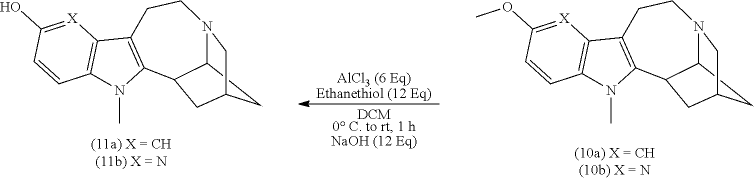

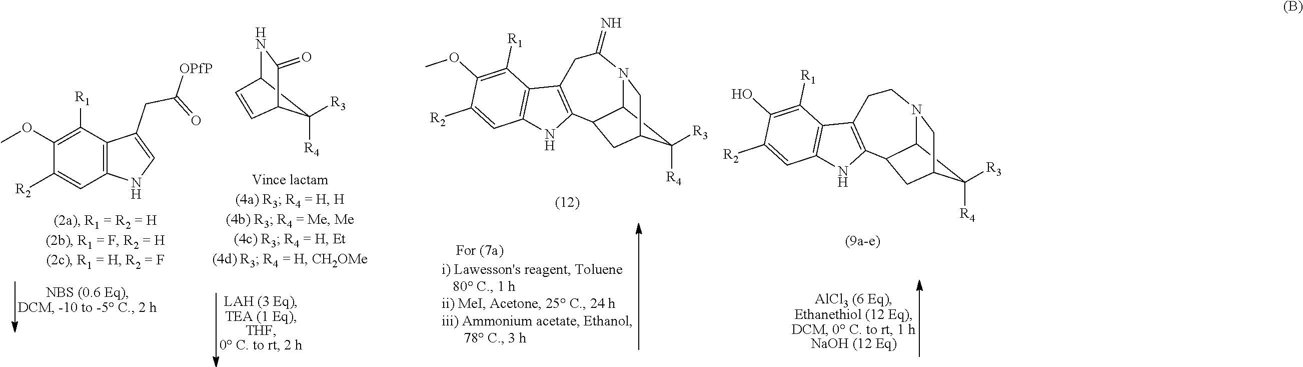

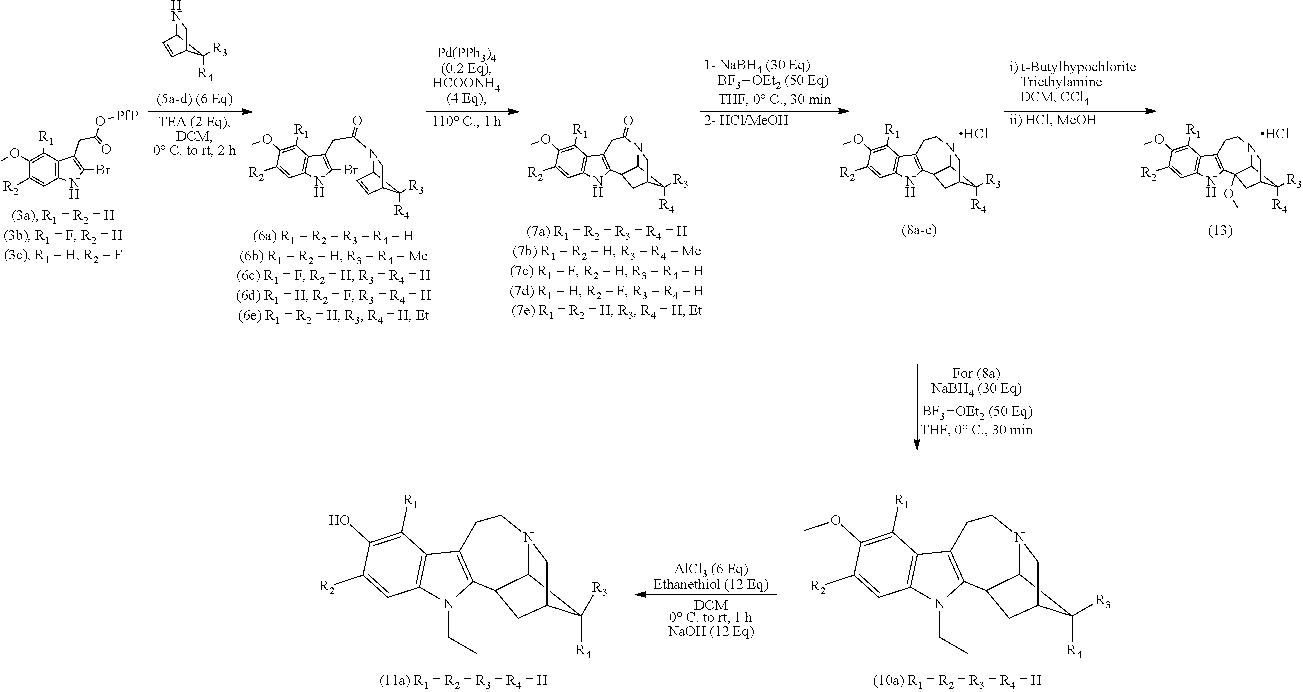

### General Procedure (I′); Synthesis of Substituted Heteroaryl Acetic Acid Pentaflourophenyl Esters (2)

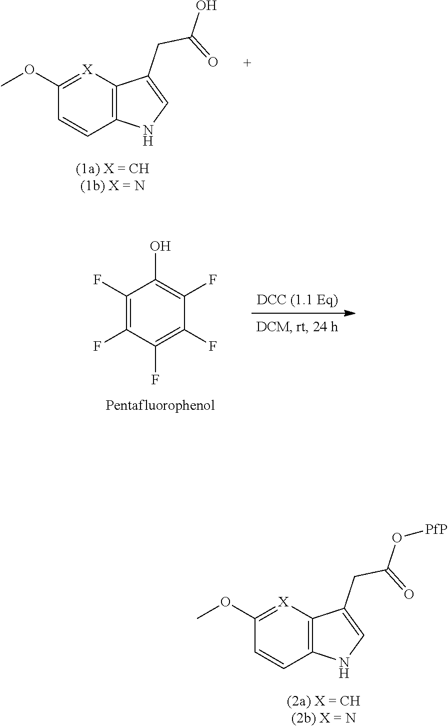

Under an inert atmosphere of argon gas, the substituted heteroaryl acetic acid (1 eq) and pentafluorophenol (1.1 eq) were dissolved in 1:1 of DCM/THF (0.24 M), then DCC (1.1 eq) was added. The reaction was stirred for 24 hours at rt. The reaction mixture was kept in the fridge to cool down and precipitate DCU as an impurity. The precipitated impurity was filtered by suction filtration and washed with cold DCM. The filtrate was concentrated under reduced pressure to afford the desired product.

### General Procedure (II); Synthesis of 2-Bromo Substituted Heteroaryl Acetic Acid Pentaflourophenyl Esters (3)

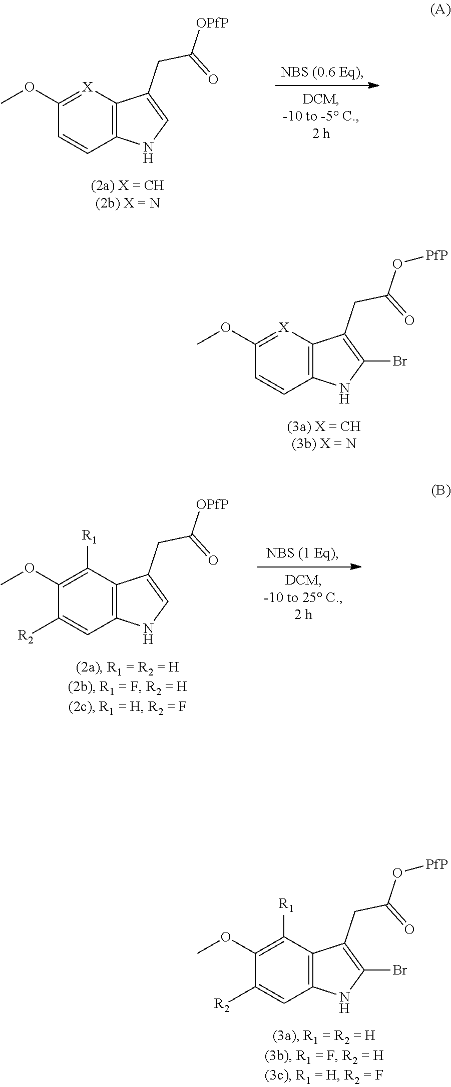

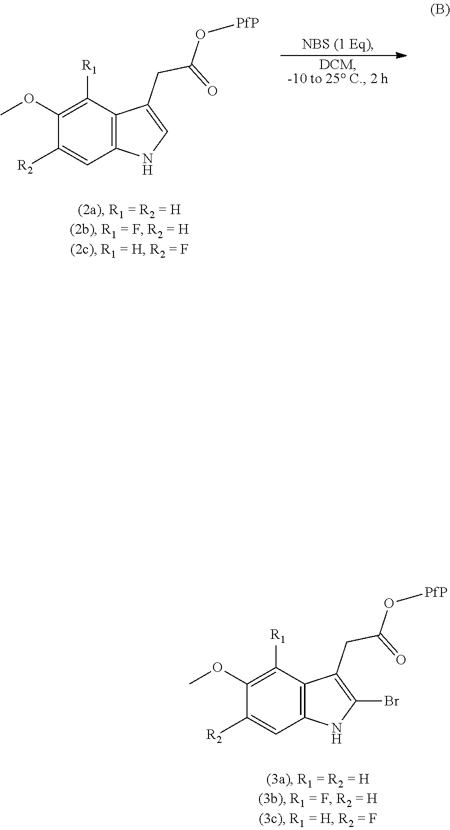

(A) Under an inert atmosphere of argon gas, perfluorophenyl (indol-3-yl)acetate analog (2) (1 eq) was dissolved in DCM (0.014 M) and cooled to −10° C. Subsequently, N-Bromo succinimide (0.6 eq) was added to the reaction mixture portion wise over 2 hours at −10° C. and then reaction was warmed to 0° C. The reaction was quenched with an aqueous solution of sodium thiosulfate. The reaction mixture was diluted with water, organic phase was separated, washed with brine and water, and dried over Na₂SO₄. The organic phase was dried under vacuum and the crude reaction mixture was purified by normal phase silica gel column chromatography, running a mobile phase of Ethyl acetate/Hexane. The product containing pure fractions was dried under reduced pressure to afford the desired product.

(B) Under an inert atmosphere of argon gas, pentafluorophenyl (indol-3-yl)acetate analog (2) (1 eq) was dissolved in DCM (0.014 M) and cooled to −10° C. Subsequently, N-bromosuccinimide (1 eq) was added to the reaction mixture portion-wise over 2 hours at −10° C. and then the reaction was warmed gradually to 0° C. then 25° C. The reaction was quenched with an aqueous solution of sodium thiosulfate. The reaction mixture was diluted with water, the organic phase was separated, washed with brine and water, and dried over Na₂SO₄. The organic phase was dried under vacuum and the crude reaction mixture was purified by normal phase silica gel column chromatography, running a mobile phase of Ethyl acetate/Hexane. The product containing pure fractions was dried under reduced pressure to afford the desired product.

### General Procedure (III); Synthesis of 2-Azabicyclo[2.2.1]Hept-5-Ene (5) from Vince Lactam (4)

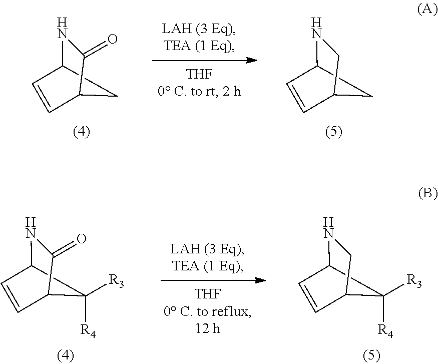

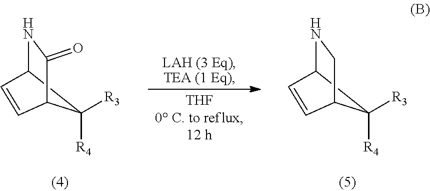

Under an inert atmosphere of argon gas, lithium aluminum hydride (3 eq) was suspended in ice bath cold THF (0.53 M). Subsequently, Vince lactam (4) (1 eq) was slowly added to the reaction mixture at 0° C. After warming to room temperature, the mixture was stirred for 1 hour and then heated to reflux for 11 hour. The reaction mixture was cooled to 0° C. and quenched with water. The precipitated solid was diluted with ether and the organic phase was dried with Na₂SO₄. Triethylamine (1 eq) was added to the ethereal solution and filtrated through a celite pad.

### General Procedure (IV); Synthesis of Reductive Heck Precursor (6)

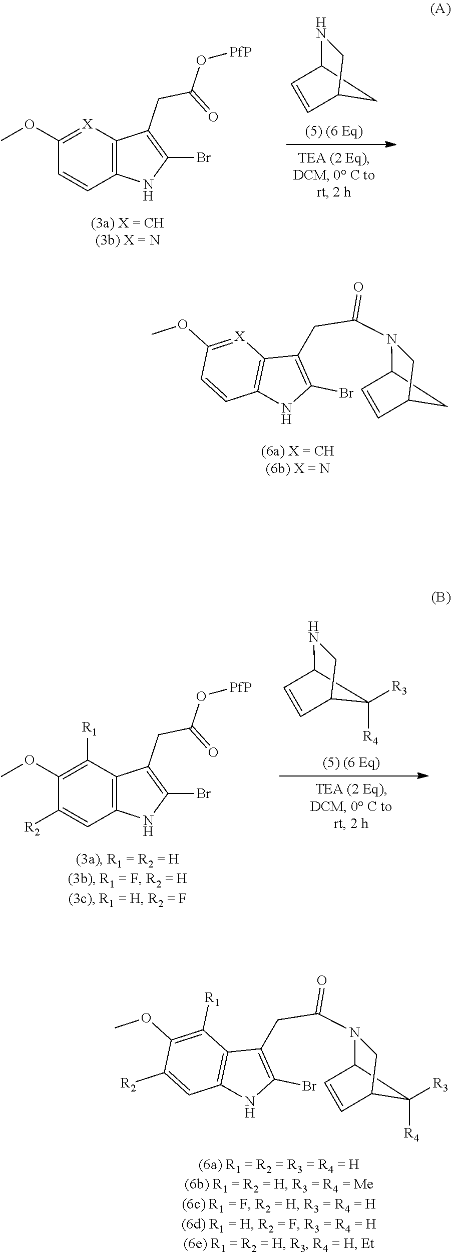

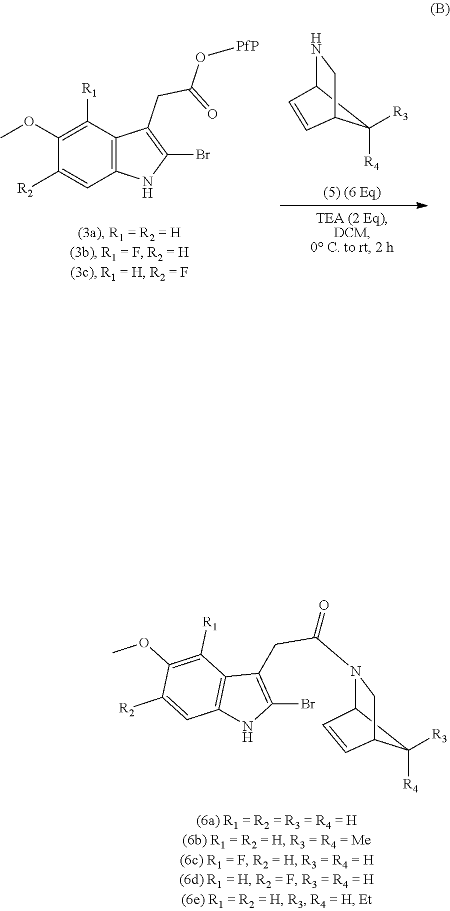

Under an inert atmosphere of argon gas, 5-azabicyclo[2.2.1]hept-2-ene ethereal solution (4 eq) was cooled to 0° C. and then triethylamine (4 eq) was added. Subsequently, a solution of 2-bromo substituted indol-3-yl acetic acid pentaflourophenyl derivative (3) (1 eq) in DCM (0.28 M) was added to the mixture. The reaction was warmed to rt and stirred for 1 hour. The reaction completion was confirmed by LCMS and TLC. The organic solvent was removed under reduced pressure at 36° C. and dried under high reduced pressure vacuum. The crude was purified by normal phase silica gel column chromatography, running a mobile phase of 20% ethyl acetate in hexane, and the product containing fractions were dried under reduced pressure to afford the desired product.

### General Procedure (V); Cyclization Via Reductive Heck Coupling Approach

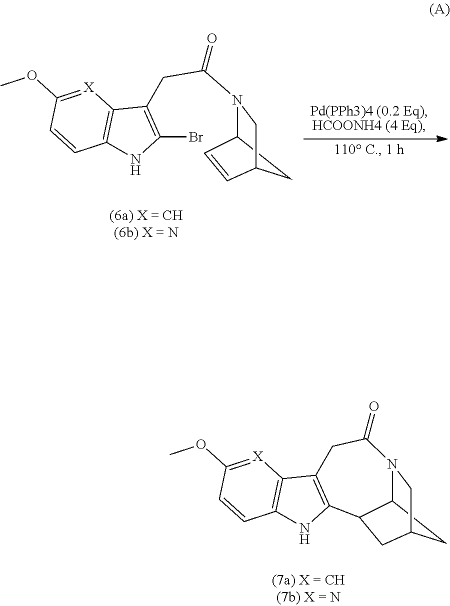

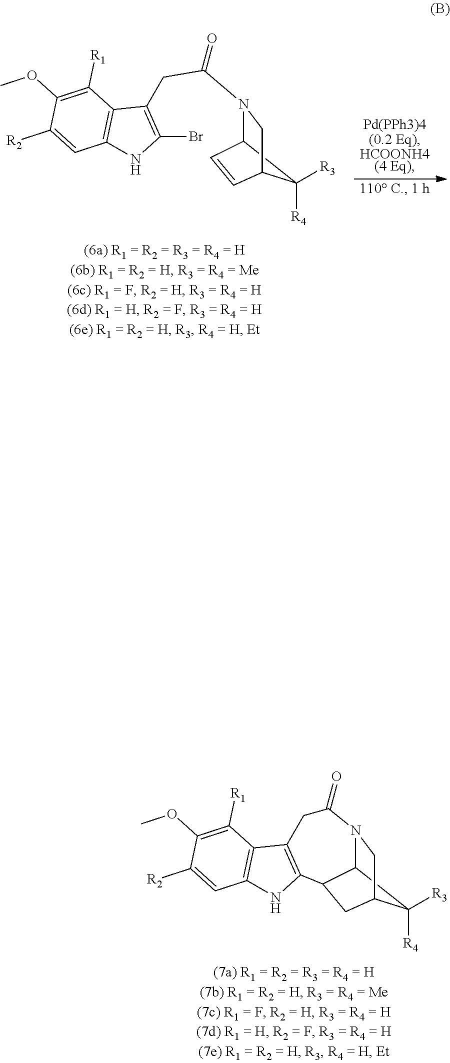

Under an inert atmosphere of argon gas, reductive Heck precursor (6) (1 eq), palladium (0) tetrakis(triphenylphosphine) (0.1 eq) and sodium formate (4 eq) were placed in a microwave vial. The vial was sealed, evacuated, and purged with argon gas multiple times. DMSO (10 mL) was added and purged with argon for 10 min. The mixture was then heated to 110° C. for 4 hours. The reaction was then diluted with water and extracted with DCM. The combined organics were dried over Na₂SO₄ and concentrated to afford the crude product. The crude reaction mixture was purified by normal phase silica gel column chromatography, running a mobile phase of 5% MeOH in 95% DCM (A) or 5% to 50% ACN in water (B), and the product-containing fractions were dried under reduced pressure to afford the desired product.

### General Procedure (VI); Amide Reduction of Reductive Heck Coupling Product

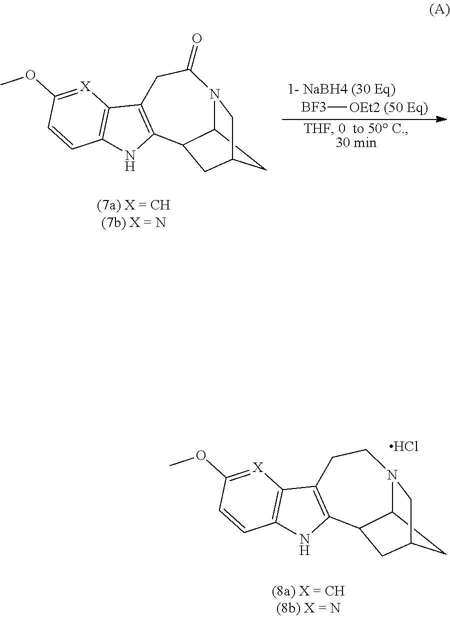

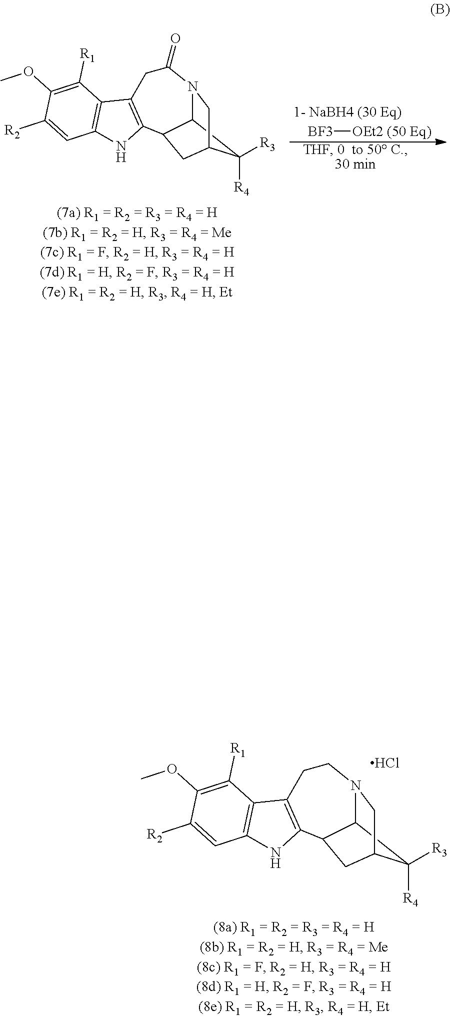

Under an inert atmosphere of argon gas, reductive Heck coupling product (7) (1 eq) was suspended in THF (0.046 M) and cooled to 0° C., then sodium borohydride (30 eq) was added followed by dropwise addition of boron trifluoride diethyl etherate (50 eq). The reaction was stirred for 30 minutes in an ice bath and then removed from the ice bath and stirred for 2 h at 50° C. After completion of the reaction, it was cooled to 0° C. and quenched with methanol. THF was removed under reduced pressure and methanol (50 ml) and water (2 ml) were added followed by the addition of (4 eq HCl, 6M) and stirred at rt for 12 h. Then methanol was removed under reduced pressure and diluted with more water. The mixture was basified to pH-8 with a saturated solution of Na₂CO₃ and extracted with DCM. The organic phase was washed with brine and dried over Na₂SO₄. The organic mixture was concentrated under reduced pressure. The crude reaction mixture was purified by normal phase silica gel column chromatography, running a mobile phase of 20% MeOH in DCM, and the product-containing fractions were dried under reduced pressure. The product was dissolved in MeOH and followed by the addition of HCl (1.5 eq, 6M). The mixture was dried and lyophilized to afford the desired product as HCl salt.

### General Procedure (VII); Synthesis of Ni-Methyl Ibogalogs (10)

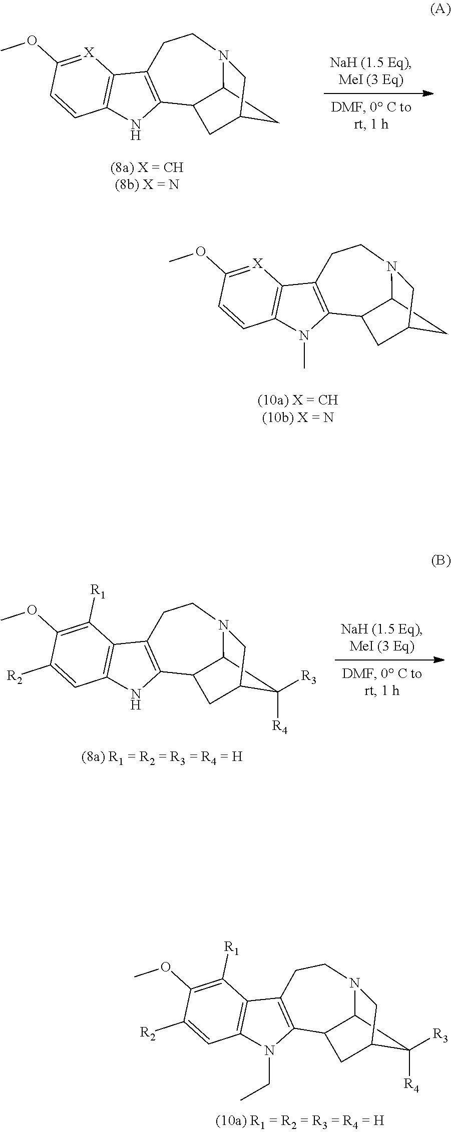

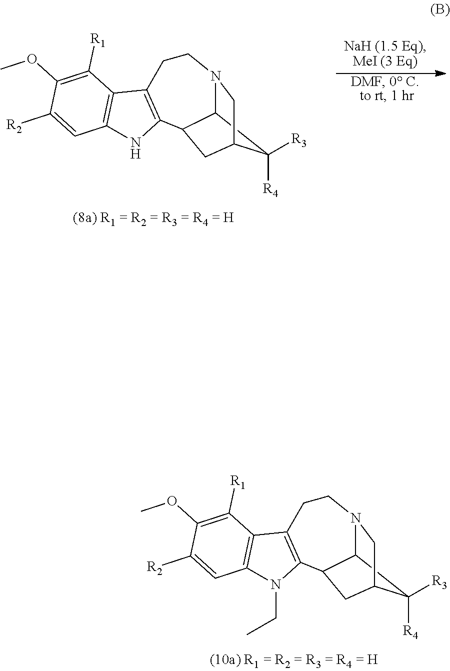

Under an inert atmosphere of argon gas, sodium hydride (60% in mineral oil) (1.5 eq) was dissolved in DMF (0.027 M) and cooled to 0° C. before ibogalogs derivative (8) (1 eq.) was added and stirred for 15 minutes. Subsequently, a solution of iodomethane (3 eq.) in DMF was added dropwise. The reaction was warmed and maintained at rt for 1 hour. The reaction mixture was cooled to 0° C. and quenched with (2 ml) of water. The reaction mixture was diluted with ethyl acetate (50 mL), and the organic phase was washed with brine, it was dried over Na₂SO₄, and the solvent was removed under reduced pressure. The crude reaction mixture was purified by normal phase silica gel column chromatography, running a mobile phase of 2% MeOH in DCM, and the product containing fractions was dried under reduced pressure to afford the desired product as HCl salt.

### General Procedure (VIII); Synthesis of Noribogalogs (9, 11)

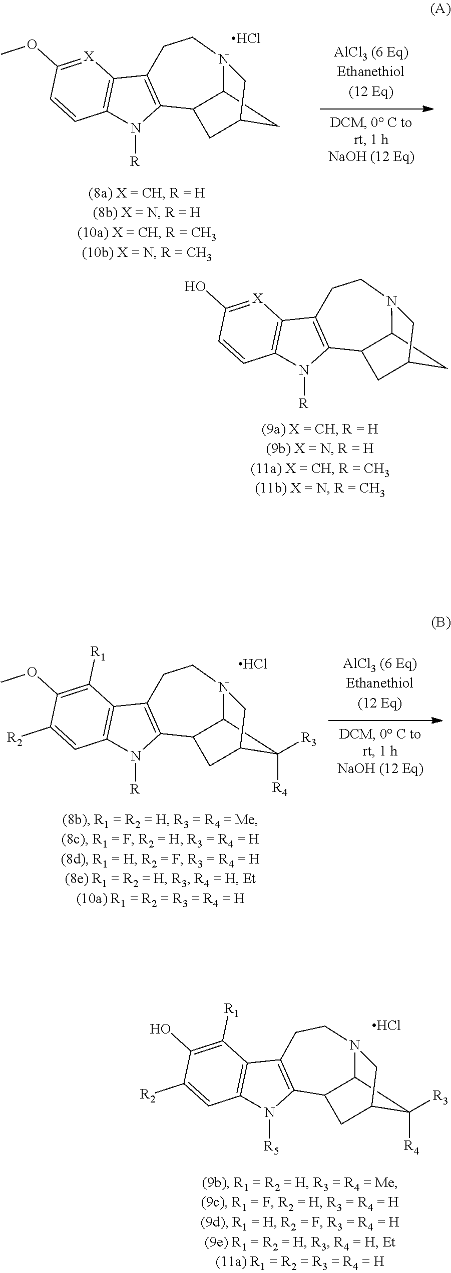

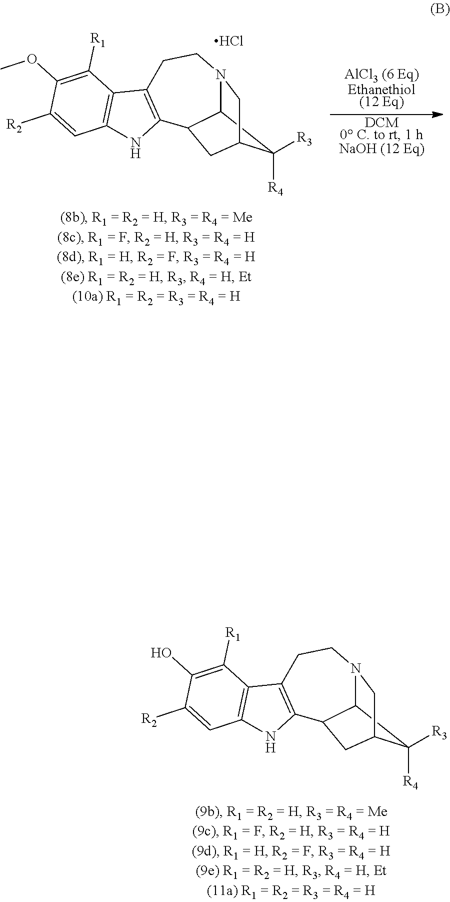

Under an inert atmosphere of argon gas, corresponding ibogalog derivative (8 or 10) (1 eq.) was dissolved in DCM (0.016 M) and cooled to 0° C. While stirring at 0° C., aluminum chloride (6 eq) and ethanethiol (12 eq) were added in one portion and caped. The mixture was moved to rt and stirred for 1 h at rt. Then it was cooled to 0° C. and quenched with dropwise addition of sodium hydroxide solution (18 eq). The pH should be between 7 to 8. Then 4 mL of methanol was added to facile stirring and stirred for 5 minutes. The solvents were removed by reduced pressure and placed under a high vacuum for 1 h. The crude reaction mixture was purified by normal phase silica gel column chromatography, running a mobile phase of 20% MeOH in 80% DCM, and the product-containing fractions were dried under reduced pressure to afford the desired product.

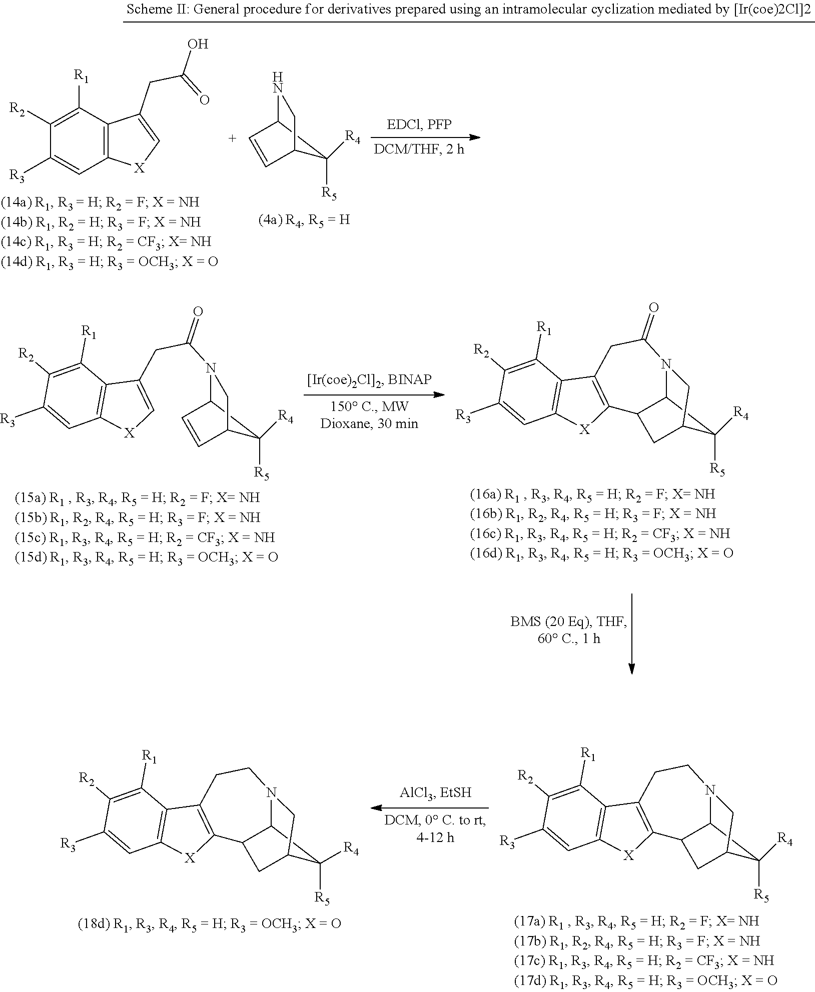

### General Procedure (IX); Synthesis of [Ir(Coe)2Cl]2 Mediated Intramolecular Cyclization Precursor (15)

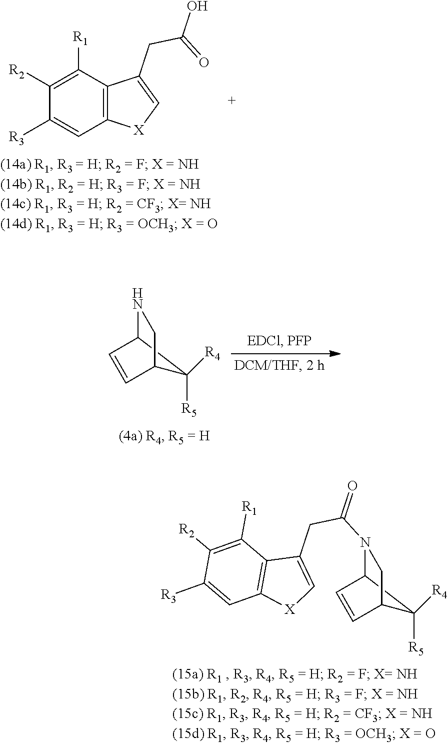

Under an inert atmosphere of argon gas, substituted heteroaryl acetic acid (14) (1 eq) and pentafluoro phenol (1.1 eq) were dissolved in 1:1 of DCM/THF (0.24 M), then EDCI (1.1 eq) was added. The reaction was stirred for 1-2 hrs at rt. After complete consumption of acid added 2-Azabicyclo[2.2.1]hept-5-ene (6 eq.) to the reaction mixture and continued to stir it for an additional 2 hrs. The volatiles were removed from the reaction mixture on the under reduced pressure, redissolved in DCM and washed with water, sodium bicarbonate solution, and brine. The organic layer was dried over sodium sulfate, filtered it and the filtrate was concentrated. The crude product was purified by normal phase silica gel column chromatography, running a mobile phase of 80-100% Ethyl acetate in Hexane, and the product containing fractions were dried under reduced pressure to afford the desired product (15).

### General Procedure (X); Iridium-Catalyzed Intramolecular Hydroheteroarylation of Bicycloalkene (15)

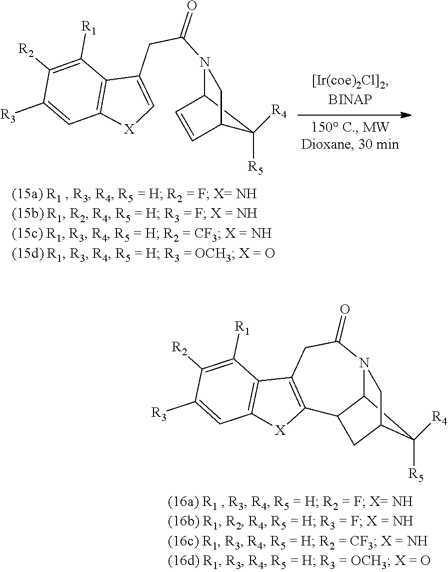

An oven-dried microwave vial was charged with 4 A^{o} molecular sieves (100 mg/2 mL), BINAP (0.3 eq.), the corresponding starting material (15) (1.0 eq.), and a magnetic stir bar. Dioxane (0.36 M) was added to the solids, while purging the argon gas into the reaction mixture added [Ir(coe)2Cl]2 (0.15 eq.) and continued purging the inert gas for an additional 3 minutes. Closed the reaction vial and heated to 150° C. for 30 minutes under the microwave. After bringing it to room temperature reaction mixture was diluted with DCM and passed through celite pad. Filtrate was concentrated and the residue was purified by flash column chromatography.

### General Procedure (XI); Amide Reduction of Intramolecular Cyclization Product (16)

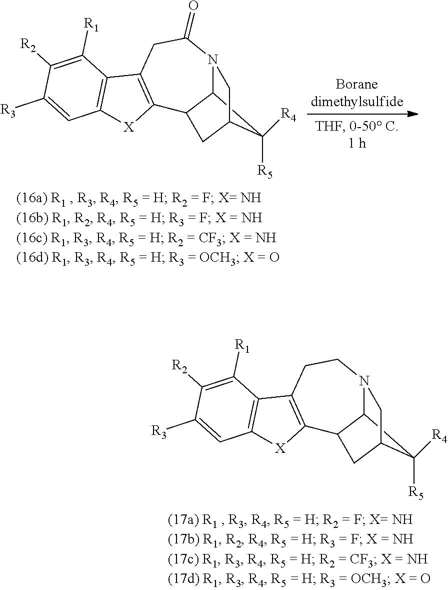

Under an inert atmosphere of argon gas, amide (16) (1 eq) was suspended in THF (0.046 M) and cooled to 0° C., then added borane dimethylsulfide. The reaction was stirred for 30 minutes in an ice bath, removed from the ice bath, and stirred for 1 h at 50° C. After completion of the reaction, it was cooled to 0° C. and quenched with methanol. THF was removed under reduced pressure and added methanol (50 ml), HCl (10 eq. 12M) and stirred at 60° C. for 2 hrs. Then the volatiles were removed under reduced pressure, and the crude reaction mixture was purified by reverse phase silica gel column chromatography and the product-containing fractions were dried under reduced pressure.

### Example 1. Synthesis of (2,3,4,5,6-pentafluorophenyl) 2-(5-methoxy-1H-indol-3-yl)acetate (2a)

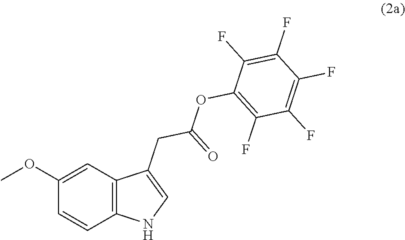 [SMILES: COc1ccc2[nH]cc(CC(=O)Oc3c(F)c(F)c(F)c(F)c3F)c2c1]

The title compound, (2,3,4,5,6-pentafluorophenyl) 2-(5-methoxy-1H-indol-3-yl)acetate (2a), was prepared according to the protocol described in general procedure (I) starting from 2-(5-methoxy-1H-indol-3-yl)acetic acid and Pentafluorophenol to give the desired compound (10 g, 93.97% yield). ¹H NMR (400 MHz, CDCl₃) δ 8.07 (s, 1H), 7.30 (dd, J=8.9, 1.2 Hz, 1H), 7.27-7.22 (m, 1H), 7.08 (d, J=2.4 Hz, 1H), 6.93 (ddd, J=8.8, 2.5, 1.2 Hz, 1H), 4.12 (s, 2H), 3.90 (s, 3H). ¹⁹F NMR (376 MHz, CDCl₃) δ −152.39-−152.81 (m, 2F), −158.01 (t, J=21.7 Hz, 1F), −162.2-−162.48 (m, 2F). ESI-MS: measured m/z 372.07 [M+H]⁺.

### Example 2. Synthesis of Perfluorophenyl 2-(4-fluoro-5-methoxy-1H-indol-3-yl)acetate (2b)

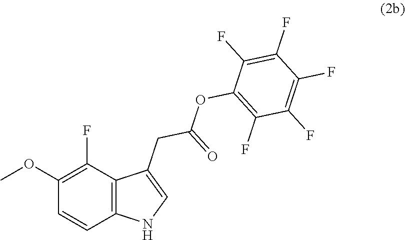 [SMILES: COc1ccc2[nH]cc(CC(=O)Oc3c(F)c(F)c(F)c(F)c3F)c2c1F]

The title compound, perfluorophenyl 2-(4-fluoro-5-methoxy-1H-indol-3-yl)acetate (2b), was prepared according to the protocol described in general procedure (I) starting from 2-(4-fluoro-5-methoxy-1H-indol-3-yl)acetic acid and pentafluorophenol to give the desired compound as a white solid (960 mg, 66% yield). ¹H NMR (400 MHz, CDCl₃) δ 8.09 (brs, 1H), 7.20 (d, J=2.5 Hz, 1H), 7.08 (d, J=8.8, 0.7 Hz, 1H), 7.03-6.95 (m, 1H), 4.27 (s, 2H), 3.96 (s, 3H). ESI-MS: measured m/z 390.13 [M+H]⁺.

### Example 3. Synthesis of Pentafluorophenyl 2-(6-fluoro-5-methoxy-1H-indol-3-yl)acetate (2c)

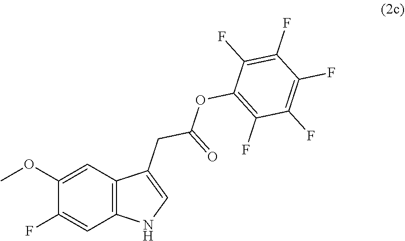 [SMILES: COc1cc2c(CC(=O)Oc3c(F)c(F)c(F)c(F)c3F)c[nH]c2cc1F]

The title compound, Perfluorophenyl 2-(6-fluoro-5-methoxy-1H-indol-3-yl)acetate (2c), was prepared according to the protocol described in general procedure (I) starting from 2-(6-fluoro-5-methoxy-1H-indol-3-yl)acetic acid to give the desired compound as a white solid (80 mg, 91% yield). ¹H NMR (400 MHz, CDCl₃) δ 8.16 (brs, 1H), 7.27-7.21 (m, 1H), 7.20-7.05 (m, 2H), 4.11 (s, 2H), 3.96 (s, 3H). ESI-MS: measured m/z 390.13 [M+H]⁺.

### Example 4: Synthesis of (2,3,4,5,6-pentafluorophenyl) 2-(2-bromo-5-methoxy-1H-indol-3-yl)acetate (3a)

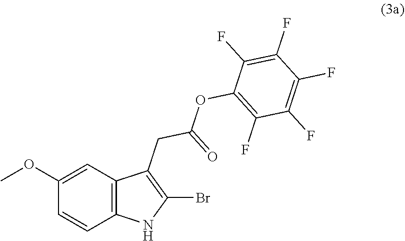 [SMILES: COc1ccc2[nH]c(Br)c(CC(=O)Oc3c(F)c(F)c(F)c(F)c3F)c2c1]

The title compound, (2,3,4,5,6-pentafluorophenyl) 2-(2-bromo-5-methoxy-1H-indol-3-yl)acetate (3a), was prepared according to the protocol described in general procedure (II) starting from (2,3,4,5,6-pentafluorophenyl) 2-(5-methoxy-1H-indol-3-yl)acetate (2a) to afford the desired compound (353 mg, 30% yield). ¹H NMR (400 MHz, MeOD) δ 7.23 (d, J=8.8 Hz, 1H), 7.03 (d, J=2.4 Hz, 1H), 6.81 (dd, J=8.8, 2.4 Hz, 1H), 4.12 (s, 2H), 3.83 (s, 3H). ¹⁹F NMR (376 MHz, CDCl3) δ −152.32-−152.54 (m, 2F), −157.89 (t, J=21.8 Hz, 1F), −162.10-−162.47 (m, 2F). ESI-MS: measured m/z 449.87 [M+1, ⁷⁹Br]⁺, 451.87.00 [M+H, ⁸¹Br]⁺.

### Example 5: Synthesis of Perfluorophenyl 2-(2-bromo-4-fluoro-5-methoxy-1H-indol-3-yl)acetate (3b)

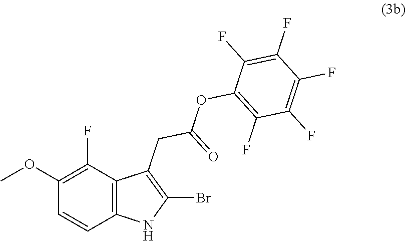 [SMILES: COc1ccc2[nH]c(Br)c(CC(=O)Oc3c(F)c(F)c(F)c(F)c3F)c2c1F]

The title compound, perfluorophenyl 2-(2-bromo-4-fluoro-5-methoxy-1H-indol-3-yl)acetate (3b), was prepared according to the protocol described in general procedure (II) starting from perfluorophenyl 2-(4-fluoro-5-methoxy-1H-indol-3-yl)acetate (2b) to afford the desired compound as a white solid (11 mg, 22% yield). ¹H NMR (400 MHz, CDCl₃) δ 8.18 (s, 1H), 7.04-6.91 (m, 2H), 4.20 (s, 2H), 3.94 (s, 3H). ESI-MS: m/z 469.87 [M+H]⁺.

### Example 6: Synthesis of Perfluorophenyl 2-(2-bromo-6-fluoro-5-methoxy-1H-indol-3-yl)acetate (3c)

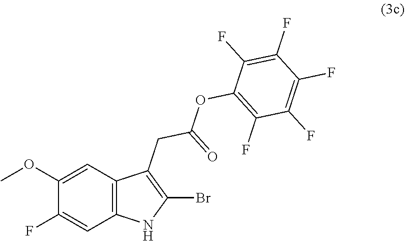 [SMILES: COc1cc2c(CC(=O)Oc3c(F)c(F)c(F)c(F)c3F)c(Br)[nH]c2cc1F]

The title compound, perfluorophenyl 2-(2-bromo-6-fluoro-5-methoxy-1H-indol-3-yl)acetate (3c), was prepared according to the protocol described in general procedure (II) starting from perfluorophenyl 2-(6-fluoro-5-methoxy-1H-indol-3-yl)acetate (2c) to afford the desired compound as a solid (920 mg, 71% yield). ¹H NMR (400 MHz, CDCl₃) δ 8.31-8.09 (m, 1H), 7.15-6.99 (m, 2H), 4.07 (s, 2H), 3.95 (s, 3H). ESI-MS: m/z 468.93 [M+H]⁺.

### Example 7: Synthesis of (1S,4R)-2-azabicyclo[2.2.1]hept-5-ene (5a) and ((1S)-5)

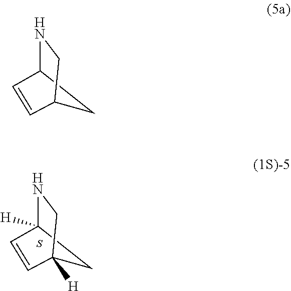

(5a) The title compound, (1S,4R)-2-azabicyclo[2.2.1]hept-2-ene (5a), was prepared according to the protocol described in general procedure (III) starting from (1S,4R)-2-azabicyclo[2.2.1]hept-5-en-3-one. ESI-MS: measured m/z 96.07 [M+H]⁺.

(1S-5) The title compound, (1S,4R)-2-azabicyclo[2.2.1]hept-2-ene (1S)-5, was prepared according to the protocol described in general procedure (III) starting from (1S,4R)-2-azabicyclo[2.2.1]hept-5-en-3-one (1S)-4. ESI-MS: measured m/z 96.07 [M+H]⁺.

### Example 8: Synthesis of (f)-7,7-dimethyl-2-azabicyclo[2.2.1]hept-5-en-3-one (4b)

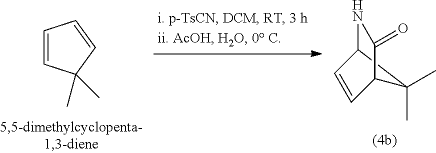

The Tosyl Cyanide (1.0 eq.) was added in 5,5-dimethylcyclopenta-1,3-diene (524 mg, 5.57 mmol, 1.0 eq.) at −20° C. and brought to rt over 40 min and continued for 3 hrs at rt. Then, the reaction mixture was cooled to 0° C. and cold acetic acid, glacial (5.57 eq.) was added rapidly with stirring. The mixture was quickly poured into ice-cold water, resulting in the formation of a white precipitate, which were filtered through celite pad. It was washed with cold water and dichloromethane. The filtrate was cooled below 20° C., neutralized with cold saturated sodium bicarbonate, and extracted with dichloromethane and washed with cold brine solution. The organics were dried over sodium sulfate and dried with an air stream at rt to give crude product (f)-7,7-dimethyl-3-azabicyclo[2.2.1]hept-5-en-2-one (4b) which was used in the following step without further purification. ESI-MS: measured m/z 138.07 [M+H]⁺.

### Example 9: Synthesis of (f)-7,7-dimethyl-2-azabicyclo[2.2.1]hept-5-ene (5b)

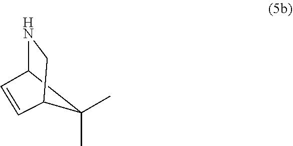

The title compound, (f)-7,7-dimethyl-2-azabicyclo[2.2.1]hept-5-ene (5b), was prepared according to the protocol described in general procedure (III) starting from (±)-7,7-dimethyl-2-azabicyclo[2.2.1]hept-5-en-3-one (4b) to afford the desired compound. ESI-MS: measured m/z 124.07 [M+H]⁺.

### Example 10: Synthesis of 7-(hydroxymethyl)-3-[(4-methoxyphenyl)methyl]-3-azabicyclo[2.2.1]hept-5-en-2-one (20)

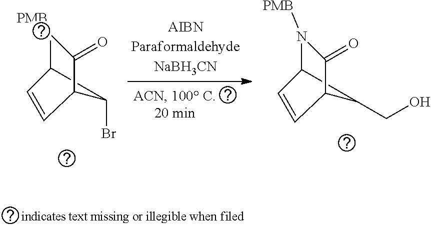

A magnetic stirring bar, 7-bromo-3-[(4-methoxyphenyl)methyl]-3-azabicyclo[2.2.1]hept-5-en-2-one (19) (600 mg, 1.95 mmol), NaBH₃CN (3.1 eq.), paraformaldehyde (12.4 eq.) and AIBN (6.5 eq.), were placed in a microwave vial. Purged three times with nitrogen, then CH₃CN (0.13 M) was added to the mixture. The mixture was heated at 100° C. for 20 min. under microwave irradiation. Methanol (3 mL) was added to the reaction mixture and stirring it for 10 min, the reaction mixture was filtered through celite pad, then concentrated. The residue was purified by flash chromatography on silica gel to give the corresponding alcohol (110 mg, 21.8%). ¹H NMR (400 MHz, CDCl₃) δ 7.13 (d, J=8.6 Hz, 2H), 6.87 (d, J=8.6 Hz, 2H), 6.64-6.55 (m, 2H), 4.35 (d, J=14.5 Hz, 1H), 4.11 (d, J=14.6 Hz, 1H), 4.02-3.96 (m, 1H), 3.82 (s, 3H), 3.75-3.60 (m, 2H), 3.29-3.23 (m, 1H), 2.70-2.62 1.88-1.70 (brs, 1H). ESI-MS: measured m/z 260.13 [M+H]⁺.

### Example 11: Synthesis of 7-(iodomethyl)-3-[(4-methoxyphenyl)methyl]-3-azabicyclo[2.2.1]hept-5-en-2-one (21)

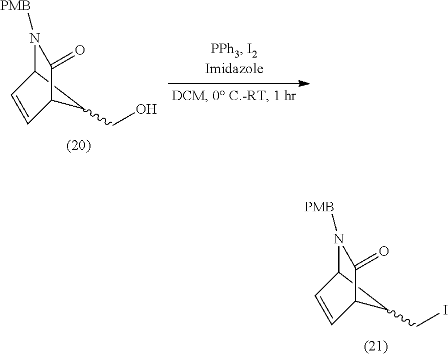

To a solution of Imidazole (1.5 eq.) Triphenylphosphine (1.5 eq.) in DCM (10 mL) cooled to 0° C. was added Iodine (1.5 eq.). After stirring for 30 min in the dark, 7-(hydroxymethyl)-3-[(4-methoxyphenyl)methyl]-3-azabicyclo[2.2.1]hept-5-en-2-one (21) (650 mg, 2.50 mmol) was added dissolved in minimal DCM. After 0.5 hour at 0° C. and 1 hr at rt, LC-MS showed the complete conversion of the starting material. The reaction was quenched by adding saturated Na₂S₂O₃ and saturated NaHCO₃ solution at 0° C. The biphasic mixture was extracted with EtOAc (2×100 mL), dried over Na₂SO₄ and concentrated. The residue was purified by flash column chromatography (eluting with 0-100% EtOAc in Hexane) and obtained colorless gel (615 mg, 66%). Isolated the desired compound (22) as colorless gel (34 mg, 68% yield. ¹H NMR (400 MHz, CDCl₃) δ 7.14 (d, J=8.6 Hz, 2H), 6.87 (d, J=8.6 Hz, 2H), 6.61-6.52 (m, 2H), 4.35 (d, J=14.5 Hz, 1H), 4.14 (d, J=14.5 Hz, 1H), 3.97-3.93 (m, 1H), 3.81 (s, 3H), 3.30-3.22 (m, 2H), 3.20-3.13 (m, 1H), 2.87-2.80 (m, 1H). ESI-MS: measured m/z 370.0 [M+H]⁺.

### Example 12: Synthesis of 7-ethyl-3-[(4-methoxyphenyl)methyl]-3-azabicyclo[2.2.1]hept-5-en-2-one (22)

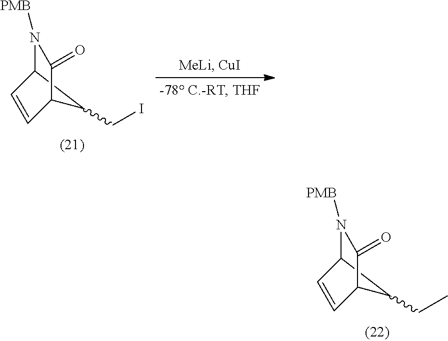

To oven dried 50 mL RBF added Copper(I) iodide (3.0 eq.) and dissolved in THF (0.03 M) cooled to −78° C. Added Methyllithium solution (6 eq. 1.6 M sol.) in a dropwise manner. Stirred it for 30 min at the same temperature then left it to 0° C. Added 7-(iodomethyl)-3-[(4-methoxyphenyl)methyl]-3-azabicyclo[2.2.1]hept-5-en-2-one (21) (600 mg, 1.63 mmol) dissolved in THF(10 mL) at 0° C. Continued to stir it for 45 min, LC-MS showed the complete conversion of starting material, reaction was quenched with Sat. NH₄Cl solution. Added 100 mL of EtOAc to the reaction mixture, separate the layers. Organic layer was dried on Na₂SO₄, filtered. Filtrate was concentrated, and the residue was purified on silica gel column (0-100% EtOAc in Hexane) and obtained the product (290 mg, 69%). Isolated the desired compound (22) as colorless gel (380 mg, 68% yield). ¹H NMR (400 MHz, CDCl₃) δ 7.15 (d, J=8.6 Hz, 2H), 6.87 (d, J=8.6 Hz, 2H), 6.65-6.53 (m, 2H), 4.21 (dd, J=153.4, 14.6 Hz, 2H), 3.82 (s, 3H), 3.81-3.78 (m, 1H), 3.24-3.18 (m, 1H), 2.38-2.27 (m, 1H), 1.60-1.43 (m, 2H), 0.82 (t, J=7.5 Hz, 3H). ESI-MS: measured m/z 258.07 [M+H]⁺.

### Example 13: Synthesis of 7-ethyl-5-azabicyclo[2.2.1]hept-2-ene (5c)

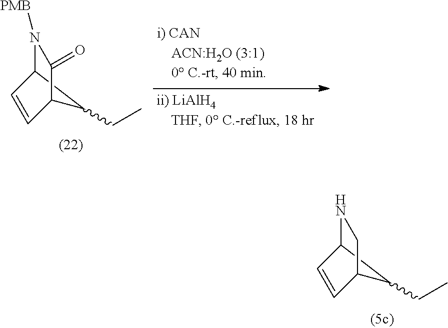

7-ethyl-3-[(4-methoxyphenyl)methyl]-3-azabicyclo[2.2.1]hept-5-en-2-one (22) (110 mg, 427.47 μmol) was dissolved in Acetonitrile (3 mL) Water (1 mL) and cooled to 0° C. Then Cerium ammonium nitrate (2.5 eq.). Reaction mixture left at room temperature, after 40 min reaction was completed based on LC-MS. Reaction mixture was diluted with 2 mL water and 10 mL of EtOAc, transferred to a separating funnel. Separated the layers and the organic layer was washed with brine. Organic layer was dried on Na₂SO₄, filtered, concentrated the filtrate. Crude reaction mixture was directly used in next step without further purification. Next, it was subjected to the general reduction procedure (III) to afford the desired compound, which directly used in the following step without further purification.

### Example 14: 1-((1S,4R)-2-azabicyclo[2.2.1]hept-5-en-2-yl)-2-(2-bromo-5-methoxy-1H-indol-3-yl)ethan-1-one ((S)-6a)

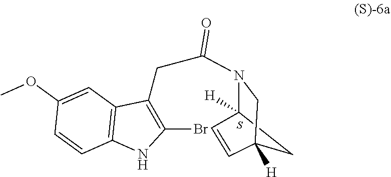

The title compound, 1-((1S,4R)-2-azabicyclo[2.2.1]hept-5-en-2-yl)-2-(2-bromo-5-methoxy-1H-indol-3-yl)ethan-1-one ((S)-6a), was prepared according to the protocol described in general procedure (IV) starting from (1S,4R)-2-azabicyclo[2.2.1]hept-2-ene (5a) to afford the desired product (150 mg, 98% yield). ¹H NMR (400 MHz, CDCl₃) δ 8.40-8.23 (m, 1H), 7.23-7.08 (m, 2H), 6.81 (dt, J=8.8, 2.4 Hz, 1H), 6.42-6.36 (m, 0.45H), 6.31-6.26 (m, 0.47H), 6.25-6.20 (m, 0.54H), 5.85-5.80 (m, 0.54H), 5.20 (s, 0.43H), 4.76 (s, 0.55H), 3.85 (s, 1.5H), 3.83 (s, 1.5H), 3.80-3.74 (m, 0.5H), 3.64-3.59 (m, 0.5H), 3.54-3.40 (m, 1H), 3.29-3.17 (m, 1H), 2.87-3.78 (m, 1H), 2.07 (s, 1H), 1.67-1.57 (m, 1H), 1.54-1.49 (m, 1H). ESI-MS: measured m/z 361.00 [M+1, ⁷⁹Br]⁺, 363.00 [M+H, ⁸¹Br]⁺.

### Example 15: 2-(2-bromo-5-methoxy-1H-indol-3-yl)-1-(-7,7-dimethyl-2-azabicyclo[2.2.1]hept-5-en-2-yl)ethan-1-one (6b)

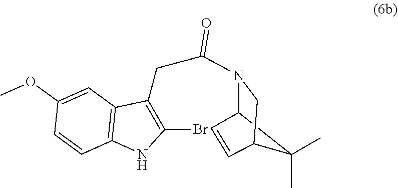

The title compound, 2-(2-bromo-5-methoxy-1H-indol-3-yl)-1-(7,7-dimethyl-2-azabicyclo[2.2.1]hept-5-en-2-yl)ethan-1-one (6b), was prepared according to the protocol described in general procedure (IV) starting from 7,7-dimethyl-2-azabicyclo[2.2.1]hept-5-ene (5b) to afford the desired product (720 mg, 50% yield). ¹H NMR (400 MHz, CDCl₃) δ 8.15 (d, J=11.4 Hz, 1H), 7.20 (d, J=2.5 Hz, 0.5H), 7.17-7.11 (m, 1H), 7.13 (d, J=3.5 Hz, 0.5H), 6.82 (dt, J=8.8, 2.2 Hz, 1H), 6.24-6.16 (m, 1H), 5.83-5.78 (m, 0.5H), 4.21-4.16 (m, 0.6H), 3.85 (d, J=3.7 Hz, 3H), 3.75 (s, 1H), 3.61 (d, J=5.2 Hz, 1H), 3.60-3.56 (m, 0.5H), 3.52 (dd, J=10.8, 3.2 Hz, 0.5H), 2.73 (d, J=2.5 Hz, 0.5H), 2.71 (s, 0.5H), 2.60-2.56 (m, 0.5H), 2.54-2.49 (m, 0.5H), 1.00 (s, 3H), 0.92 (d, J=8.9 Hz, 3H). ESI-MS: measured m/z 389.13 [M+H, ⁷⁹Br]⁺, 391.07 [M+H, ⁸¹Br]⁺. Purity by HPLC: 88% at 254 nm.

### Example 16: 1-((1S,4R)-2-azabicyclo[2.2.1]hept-5-en-2-yl)-2-(2-bromo-4-fluoro-5-methoxy-1H-indol-3-yl)ethan-1-one ((S)-6c)

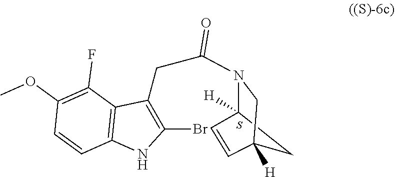

The title compound, 1-((1S,4R)-2-azabicyclo[2.2.1]hept-5-en-2-yl)-2-(2-bromo-4-fluoro-5-methoxy-1H-indol-3-yl)ethan-1-one ((S)-6c), was prepared according to the protocol described in general procedure (IV) starting from (1S,4R)-2-azabicyclo[2.2.1]hept-2-ene ((1S)-5a) to afford the desired product (265 mg, 82% yield). ¹H NMR (400 MHz, CDCl₃) δ 9.22 (d, J=8.6 Hz, 1H), 6.70-6.58 (m, 2H), 6.49-6.45 (m, 0.5H), 6.43-6.36 (m, 1.5H), 5.29-5.26 (m, 1H), 4.89-4.85 (m, 1H), 3.90-3.83 (m, 4H), 3.74-3.69 (m, 1H), 3.65-3.57 (m, 0.5H), 3.55-3.47 (m, 0.5H), 3.39-3.34 (m, 0.5H), 3.31-3.26 (m, 0.5H), 3.00-2.92 (m, 1H), 1.83-1.74 (m, 1H). ESI-MS: m/z 379.01 [M+1]⁺.

### Example 17: 1-((1S,4R)-2-azabicyclo[2.2.1]hept-5-en-2-yl)-2-(2-bromo-6-fluoro-5-methoxy-1H-indol-3-yl)ethan-1-one ((S)-6d)

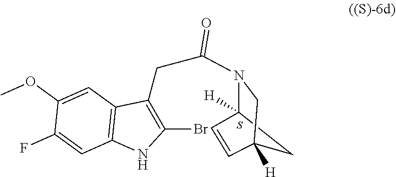

The title compound, 1-((1S,4R)-2-azabicyclo[2.2.1]hept-5-en-2-yl)-2-(2-bromo-6-fluoro-5-methoxy-1H-indol-3-yl)ethan-1-one ((S)-6d), was prepared according to the protocol described in general procedure (IV) starting from (1S,4R)-2-azabicyclo[2.2.1]hept-2-ene ((1S)-5a) to afford the desired compound as a white solid (300 mg, 40% yield). ¹H NMR (400 MHz, CDCl₃) δ 8.39-8.26 (m, 1H), 7.27-7.23 (m, 1H), 7.01-6.93 (m, 1H), 6.41-6.21 (m, 1.5H), 5.88-5.80 (m, 0.5H), 5.25-5.14 (m, 0.5H), 4.70-4.71 (m, 0.5H), 3.93 (m, 2.5H), 3.90 (s, 1.5H), 3.79-3.73 (m, 1H), 3.62-3.57 (m, 1H), 3.56-3.40 (m, 1H), 3.32-3.18 (m, 1H), 2.90-2.77 (m, 1H), 1.73-1.63 (m, 1H). ESI-MS: m/z 379.00 [M+1]⁺.

### Example 18: 2-(2-bromo-5-methoxy-1H-indol-3-yl)-1-((1R,4S)-7-ethyl-2-azabicyclo[2.2.1]hept-5-en-2-yl)ethan-1-one ((S)-6e)

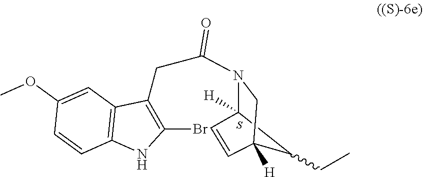

The title compound, 2-(2-bromo-5-methoxy-1H-indol-3-yl)-1-((1R,4S)-7-ethyl-2-azabicyclo[2.2.1]hept-5-en-2-yl)ethan-1-one ((S)-6e), was prepared according to the protocol described in general procedure (IV) starting from (1R,4S)-7-ethyl-2-azabicyclo[2.2.1]hept-5-ene (5c) to afford the corresponding amide and product was used in the following step without further purification. ESI-MS: measured m/z 391.07 [M+H, ⁷⁹Br]⁺, 393.00 [M+H, ⁸¹Br]⁺.

### Example 19: 1-((1S,4R)-2-azabicyclo[2.2.1]hept-5-en-2-yl)-2-(5-fluoro-1H-indol-3-yl)ethan-1-one ((S)-15a)

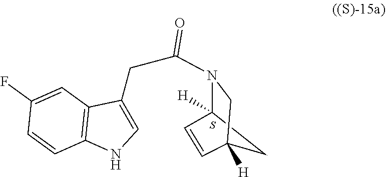

The title compound, 1-((1S,4R)-2-azabicyclo[2.2.1]hept-5-en-2-yl)-2-(5-fluoro-1H-indol-3-yl)ethan-1-one ((S)-15a), was prepared according to the protocol described in general procedure (IX) starting from (1S,4R)-2-azabicyclo[2.2.1]hept-2-ene (5a) to afford the desired compound as a white solid (220 mg, 97% yield). ¹H NMR (400 MHz, MeOD) δ 7.36-7.13 (m, 3H), 6.93-6.84 (m, 1H), 6.39-6.35 (m, 0.8H), 6.32-6.27 (m, 0.5H), 5.88-5.84 (m, 0.5H), 5.51-5.54 (m, 0.2H), 5.09 (s, 0.4H), 4.90 (s, 0.6H), 3.93-3.77 (m, 1H), 3.68-3.57 (m, 1.3H), 3.43-3.36 (m, 0.7H), 3.31-3.19 (m, 1H), 2.88-2.72 (m, 1H), 1.68-1.55 (m, 2H). ESI-MS: measured m/z 271.07 [M+H]⁺.

### Example 20: 1-((1S,4R)-2-azabicyclo[2.2.1]hept-5-en-2-yl)-2-(6-fluoro-1H-indol-3-yl)ethan-1-one ((S)-15b)

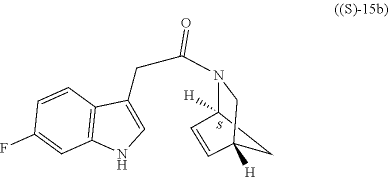

The title compound, 1-((1S,4R)-2-azabicyclo[2.2.1]hept-5-en-2-yl)-2-(6-fluoro-1H-indol-3-yl)ethan-1-one ((S)-15b), was prepared according to the protocol described in general procedure (IX) starting from (1S,4R)-2-azabicyclo[2.2.1]hept-2-ene (5a) to afford the desired compound as a white solid (80 mg, 35% yield). ¹H NMR (400 MHz, MeOD) δ 7.42 (dd, J=8.7, 5.2 Hz, 1H), 6.96 (dd, J=9.8, 2.4 Hz, 1H), 6.80 (ddd, J=9.7, 8.6, 2.3 Hz, 1H), 4.72 (s, 1H), 4.08 (dd, J=14.6, 2.3 Hz, 1H), 3.50 (d, J=14.6 Hz, 1H), 3.44-3.36 (m, 2H), 3.29-3.22 (m, 1H), 2.71 (s, 1H), 2.47-2.33 (m, 1H), 2.12-1.91 (m, 2H), 1.36-1.29 (m, 1H). ESI-MS: measured m/z 271.27 [M+H]⁺.

### Example 21: 1-((1S,4R)-2-azabicyclo[2.2.1]hept-5-en-2-yl)-2-(5-(trifluoromethyl)-1H-indol-3-yl)ethan-1-one ((S)-15c)

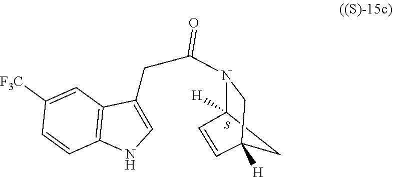

The title compound, 1-((1S,4R)-2-azabicyclo[2.2.1]hept-5-en-2-yl)-2-(5-(trifluoromethyl)-1H-indol-3-yl)ethan-1-one ((S)-15c), was prepared according to the protocol described in general procedure (IX) starting from (1S,4R)-2-azabicyclo[2.2.1]hept-2-ene (5a) to afford the desired compound as a white solid (140 mg, 70% yield). ¹H NMR (400 MHz, CDCl₃) δ 9.05-8.98 (m, 1H), 7.86 (s, 0.5H), 7.82 (s, 0.5H), 7.78 (s, 1H), 7.38-7.29 (m, 1H), 7.29-7.22 (m, 1H), 7.06-6.99 (m, 1H), 6.48-6.41 (m, 0.6H), 6.40-6.32 (m, 1H), 6.17-6.10 (m, 0.5H), 5.28-5.22 (m, 0.6H), 4.78-4.72 (m, 0.5H), 3.93-3.76 (m, 1H), 3.74-3.58 (m, 2H), 3.53-3.45 (m, 0.5H), 3.36-3.25 (m, 1H), 2.96-2.90 (m, 0.5H), 2.90-2.83 (m, 0.5H), 1.79-1.75 (m, 1H), 1.75-1.66 (m, 2H), 1.66-1.59 (m, 0.5H). ¹⁹F NMR (376 MHz, CDCl₃) δ−60.1, −60.1. ESI-MS: measured m/z 321.13 [M+H]⁺.

### Example 22: 1-((1S,4R)-2-azabicyclo[2.2.1]hept-5-en-2-yl)-2-(5-methoxybenzofuran-3-yl)ethan-1-one ((S)-15d)

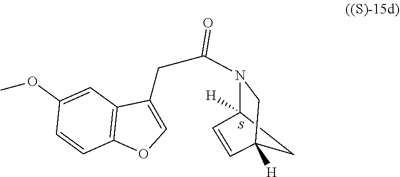

The title compound, 1-((1S,4R)-2-azabicyclo[2.2.1]hept-5-en-2-yl)-2-(5-methoxybenzofuran-3-yl)ethan-1-one ((S)-15d), was prepared according to the protocol described in general procedure (IX) starting from (1S,4R)-2-azabicyclo[2.2.1]hept-2-ene (5a) to afford the desired compound as a white solid (6 g, 91% yield). ¹H NMR (400 MHz, CDCl₃) δ 7.56 (m, 1H), 7.40-7.32 (m, 1H), 7.07 (m, 1H), 6.91 (m, 1H), 6.43 (m, 0.5H), 6.32 (m, 1H), 6.09 (m, 0.5H), 5.21 (m, 0.5H), 4.69 (m, 0.5H), 3.87 (s, 3H), 3.73 (m, 1H), 3.58-3.43 (m, 2H), 3.27-3.21 (m, 1H), 2.8-2.78 (m, 1H), 1.70-1.56 (m, 2H). ESI-MS: measured m/z 284.1 [M+H]⁺.

### Example 23: Synthesis of (2S,12R,12aS)-8-methoxy-2,3,6,11,12,12a-hexahydro-2,12-methanopyrrolo[1′,2′:1,2]azepino[4,5-b]indol-5(1H)-one ((S)-7a)

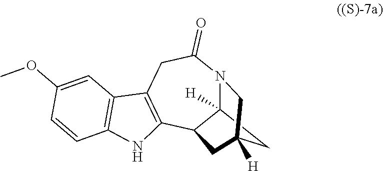

The title compound, (2S,12R,12aS)-8-methoxy-2,3,6,11,12,12a-hexahydro-2,12-methanopyrrolo[1′,2′:1,2]azepino[4,5-b]indol-5(1H)-one ((S)-7a), was prepared according to the protocol described in general procedure (V) starting from 1-((1S,4R)-2-azabicyclo[2.2.1]hept-5-en-2-yl)-2-(2-bromo-5-methoxy-1H-indol-3-yl)ethan-1-one ((S)-6a) to afford the desired product (25 mg, 31% yield). ¹H NMR (400 MHz, MeOD) δ 7.14 (d, J=8.7 Hz, 1H), 6.98 (d, J=2.4 Hz, 1H), 6.71 (dd, J=8.7, 2.4 Hz, 1H), 5.50 (s, 1H), 4.60 (s, 1H), 4.03-3.95 (m, 1H), 3.81 (s, 3H), 3.48 (d, J=14.6 Hz, 1H), 3.39-3.34 (m, 1H), 3.26-3.20 (m, 1H), 2.68-2.62 (m, 1H), 2.43-2.30 (m, 1H), 2.07-1.98 (m, 1H), 1.91-1.83 (m, 1H), 1.31-1.23 (m, 1H). ESI-MS: measured m/z 283.20 [M+H]⁺.

### Example 24: Synthesis of 8-methoxy-1,1-dimethyl-2,3,6,11,12,12a-hexahydro-2,12-methanopyrrolo[1′,2′:1,2]azepino[4,5-b]indol-5(1H)-one ((S)-7b)

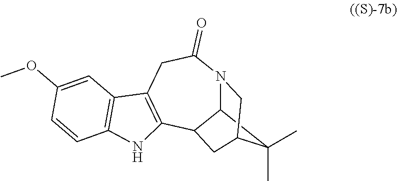

The title compound, 8-methoxy-1,1-dimethyl-2,3,6,11,12,12a-hexahydro-2,12-methanopyrrolo[1′,2′:1,2]azepino[4,5-b]indol-5(1H)-one ((S)-7b), was prepared according to the protocol described in general procedure (V) starting from 2-(2-bromo-5-methoxy-1H-indol-3-yl)-1-((1R,4S)-7,7-dimethyl-2-azabicyclo[2.2.1]hept-5-en-2-yl)ethan-1-one ((S)-6b) to afford the desired product (104.1 mg, 18% yield). ¹H NMR (400 MHz, MeOD) δ 7.15 (d, J=8.7 Hz, 1H), 6.98 (d, J=2.4 Hz, 1H), 6.72 (dd, J=8.7, 2.4 Hz, 1H), 4.57 (s, 1H), 4.12 (s, 1H), 4.00 (dd, J=14.4, 2.1 Hz, 1H), 3.83 (s, 3H), 3.75-3.61 (m, 2H), 3.48 (d, J=14.4 Hz, 1H), 3.15 (d, J=10.4 Hz, 1H), 2.60 (m, 1H), 2.07 (t, J=3.9 Hz, 1H), 1.29 (d, J=8.4 Hz, 6H). ESI-MS: measured m/z 311.33 [M+H]⁺. Purity by HPLC: 98% at 254 nm.

### Example 25: Synthesis of (2S,12R,12aS)-7-fluoro-8-methoxy-2,3,6,11,12,12a-hexahydro-2,12-methanopyrrolo[1′,2′:1,2]azepino[4,5-b]indol-5(1H)-one ((S)-7c)

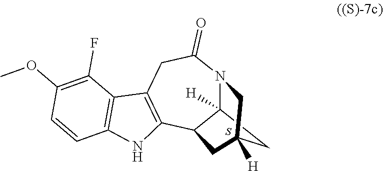

The title compound, (2S,12R,12aS)-7-fluoro-8-methoxy-2,3,6,11,12,12a-hexahydro-2,12-methanopyrrolo[1′,2′:1,2]azepino[4,5-b]indol-5(1H)-one ((S)-7c), was prepared according to the protocol described in general procedure (V) starting from 1-((1S,4R)-2-azabicyclo[2.2.1]hept-5-en-2-yl)-2-(2-bromo-4-fluoro-5-methoxy-1H-indol-3-yl)ethan-1-one ((S)-6c) to afford the desired product (104 mg, 51% yield). ¹H NMR (400 MHz, MeOD) δ 7.00-6.93 (m, 1H), 6.91-6.85 (m, 1H), 4.71 (s, 1H), 4.18 (dd, J=14.7, 2.1 Hz, 1H), 3.90-3.79 (m, 4H), 3.44-3.35 (m, 2H), 3.29 (dd, J=10.1, 2.1 Hz, 1H), 2.74-2.68 (m, 1H), 2.50-2.34 (m, 1H), 2.13-1.90 (m, 2H), 1.32 (ddd, J=12.8, 5.6, 2.2 Hz, 1H). ¹⁹F NMR (376 MHz, MeOD) δ −150.71 (d, J=7.9 Hz). ESI-MS: measured m/z 301.27 [M+H]⁺.

### Example 26: Synthesis of (2S,12R,12aS)-9-fluoro-8-methoxy-2,3,6,11,12,12a-hexahydro-2,12-methanopyrrolo[1′,2′:1,2]azepino[4,5-b]indol-5(1H)-one ((S)-7d)

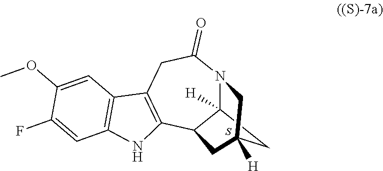

The title compound, (2S,12R,12aS)-9-fluoro-8-methoxy-2,3,6,11,12,12a-hexahydro-2,12-methanopyrrolo[1′,2′:1,2]azepino[4,5-b]indol-5(1H)-one ((S)-7d), was prepared according to the protocol described in general procedure (V) starting from 1-((1S,4R)-2-azabicyclo[2.2.1]hept-5-en-2-yl)-2-(2-bromo-6-fluoro-5-methoxy-1H-indol-3-yl)ethan-1-one ((S)-6d) to afford the desired compound as a white solid (100 mg, 51% yield). ¹H NMR (400 MHz, MeOD) δ 7.11 (d, J=8.2 Hz, 1H), 7.00 (d, J=11.6 Hz, 1H), 4.73-4.62 (m, 1H), 4.14-3.97 (m, 1H), 3.90 (s, 3H), 3.53-3.44 (m, 1H), 3.44-3.34 (m, 2H), 3.30-3.23 (m, 1H), 2.74-2.62 (m, 1H), 2.48-2.33 (m, 1H), 2.11-2.02 (m, 1H), 1.97-1.89 (m, 1H), 1.35-1.19 (m, 1H). ESI-MS: measured m/z 301.27 [M+H]⁺.

### Example 27: Synthesis of (2S,12R,12aS)-1-ethyl-8-methoxy-2,3,6,11,12,12a-hexahydro-2,12-methanopyrrolo[1′,2′:1,2]azepino[4,5-b]indol-5(1H)-one ((S)-7e)

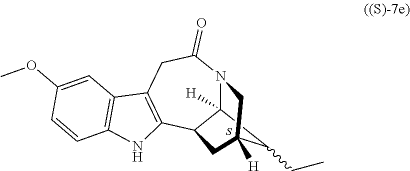

The title compound, (2S,12R,12aS)-1-ethyl-8-methoxy-2,3,6,11,12,12a-hexahydro-2,12-methanopyrrolo[1′,2′:1,2]azepino[4,5-b]indol-5(1H)-one ((S)-7e), was prepared according to the protocol described in general procedure (V) starting from 2-(2-bromo-5-methoxy-1H-indol-3-yl)-1-((1R,4S)-7-ethyl-2-azabicyclo[2.2.1]hept-5-en-2-yl)ethan-1-one ((S)-6e) to afford the desired compound as a white solid (29 mg, 66% yield). ¹H NMR (400 MHz, MeOD) δ 7.14 (d, J=8.7 Hz, 1H), 6.98 (d, J=2.4 Hz, 1H), 6.71 (dd, J=8.7, 2.4 Hz, 1H), 4.43-4.38 (m, 1H), 4.06-3.97 (m, 1H), 3.82 (s, 3H), 3.56-3.50 (m, 1H), 3.50-3.45 (m, 1H), 3.44-3.36 (m, 1H), 3.22-3.15 (m, 1H), 2.49-2.37 (m, 2H), 2.13 (t, J=7.4 Hz, 1H), 1.72-1.48 (m, 2H), 1.35-1.29 (m, 1H), 1.07 (t, J=7.5 Hz, 3H). ESI-MS: measured m/z 311.33 [M+H].

### Example 28: Synthesis of (2S,12R,12aS)-8-fluoro-2,3,6,11,12,12a-hexahydro-2,12-methanopyrrolo[1′,2′:1,2]azepino[4,5-b]indol-5(1H)-one ((S)-16a)

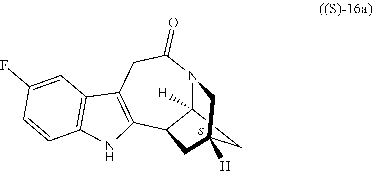

The title compound, (2S,12R,12aS)-8-fluoro-2,3,6,11,12,12a-hexahydro-2,12-methanopyrrolo[1′,2′:1,2]azepino[4,5-b]indol-5(1H)-one ((S)-16a), was prepared according to the protocol described in general procedure (X) starting from 1-((1S,4R)-2-azabicyclo[2.2.1]hept-5-en-2-yl)-2-(5-fluoro-1H-indol-3-yl)ethan-1-one ((S)-15a) to afford the desired product (98 mg, 44% yield). ¹H NMR (400 MHz, MeOD) δ 7.21 (dd, J=8.8, 4.4 Hz, 1H), 7.14 (dd, J=9.9, 2.5 Hz, 1H), 6.82 (td, J=9.1, 2.5 Hz, 1H), 4.72 (s, 1H), 4.07 (dd, J=14.5, 2.0 Hz, 1H), 3.49-3.37 (m, 3H), 3.27 (dd, J=10.1, 2.1 Hz, 1H), 2.71 (s, 1H), 2.49-2.37 (m, 1H), 2.09 (dd, J=10.3, 2.0 Hz, 1H), 1.94 (d, J=10.3 Hz, 1H), 1.32 (ddd, J=12.7, 5.6, 2.2 Hz, 1H). ESI-MS: measured m/z 271.27 [M+H]⁺.

### Example 29: Synthesis of (2S,12R,12aS)-9-fluoro-2,3,6,11,12,12a-hexahydro-2,12-methanopyrrolo[1′,2′:1,2]azepino[4,5-b]indol-5(1H)-one ((S)-16b)

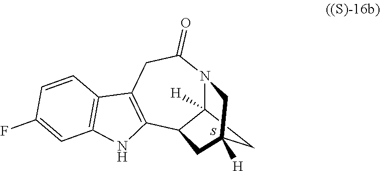

The title compound, (2S,12R,12aS)-9-fluoro-2,3,6,11,12,12a-hexahydro-2,12-methanopyrrolo[1′,2′:1,2]azepino[4,5-b]indol-5(1H)-one ((S)-16b), was prepared according to the protocol described in general procedure (X) starting from 1-((1S,4R)-2-azabicyclo[2.2.1]hept-5-en-2-yl)-2-(6-fluoro-1H-indol-3-yl)ethan-1-one ((S)-15b) to afford the desired product (80 mg, 35% yield). ¹H NMR (400 MHz, MeOD) δ 7.42 (dd, J=8.7, 5.2 Hz, 1H), 6.96 (dd, J=9.8, 2.4 Hz, 1H), 6.80 (ddd, J=9.7, 8.6, 2.3 Hz, 1H), 4.72 (s, 1H), 4.08 (dd, J=14.6, 2.3 Hz, 1H), 3.50 (d, J=14.6 Hz, 1H), 3.44-3.36 (m, 2H), 3.29-3.22 (m, 1H), 2.71 (s, 1H), 2.47-2.33 (m, 1H), 2.12-1.91 (m, 2H), 1.36-1.29 (m, 1H). ESI-MS: measured m/z 271.27 [M+H]⁺.

### Example 30: Synthesis of (2S,12R,12aS)-8-(trifluoromethyl)-2,3,6,11,12,12a-hexahydro-2,12-methanopyrrolo[1′,2′:1,2]azepino[4,5-b]indol-5(1H)-one ((S)-16c)

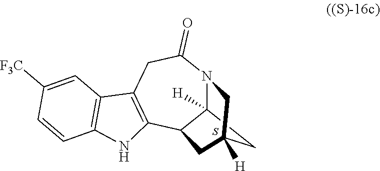

The title compound, (2S,12R,12aS)-8-(trifluoromethyl)-2,3,6,11,12,12a-hexahydro-2,12-methanopyrrolo[1′,2′:1,2]azepino[4,5-b]indol-5(1H)-one ((S)-16c), was prepared according to the protocol described in general procedure (X) starting from 1-((1S,4R)-2-azabicyclo[2.2.1]hept-5-en-2-yl)-2-(5-(trifluoromethyl)-1H-indol-3-yl)ethan-1-one ((S)-15c) to afford the desired product (35 mg, 26% yield). ¹H NMR (400 MHz, MeOD) δ 7.80 (s, 1H), 7.40 (d, J=8.5 Hz, 1H), 7.32 (dd, J=8.5, 1.7 Hz, 1H), 4.73-4.67 (m, 1H), 4.14-4.05 (m, 1H), 3.59-3.51 (m, 1H), 3.46-3.37 (m, 2H), 3.30-3.22 (m, 1H), 2.73-2.67 (m, 1H), 2.50-2.37 (m, 1H), 2.12-2.02 (m, 1H), 2.01-1.90 (m, 1H), 1.35-1.28 (m, 1H). ¹⁹F NMR (376 MHz, MeOD) δ−61.55. ESI-MS: measured m/z 321.27 [M+H]⁺.

### Example 31: Synthesis of (2S,12R,12aS)-8-methoxy-1,2,3,6,12,12a-hexahydro-5H-2,12-methanobenzofuro[2,3-d]pyrrolo[1,2-a]azepin-5-one ((S)-16d)

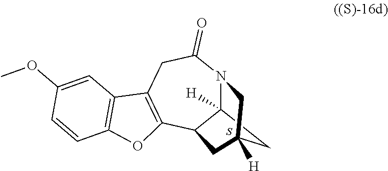

The title compound, (2S,12R,12aS)-8-methoxy-1,2,3,6,12,12a-hexahydro-5H-2,12-methanobenzofuro[2,3-d]pyrrolo[1,2-a]azepin-5-one ((S)-16d), was prepared according to the protocol described in general procedure (X) starting from 1-((1S,4R)-2-azabicyclo[2.2.1]hept-5-en-2-yl)-2-(5-methoxybenzofuran-3-yl)ethan-1-one ((S)-15d) to afford the desired product (360 mg, 30% yield). ¹H NMR (400 MHz, CDCl₃) δ 7.28 (m, 1H), 6.94 (m, 1H), 6.85 (m, 1H), 4.51 (m, 1H), 3.89 (m, 1H), 3.86-3.84 (m, 3H), 3.53-3.44 (m, 2H), 3.44-3.31 (m, 2H), 2.74 (m, 1H), 2.35 (m, 1H), 2.09 (m, 1H), 1.92-1.84 (m, 1H), 1.49 (m, 1H). ESI-MS: measured m/z 284.27 [M+H]⁺.

### Example 32: (2S,12R,12aS)-8-methoxy-2,3,4,5,6,11,12,12a-octahydro-1H-2,12-methanopyrrolo[1′,2′:1,2]azepino[4,5-b]indol-4-ium Chloride (8a)

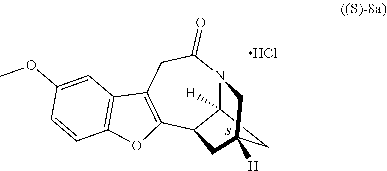

The title compound, (2S,12R,12aS)-8-methoxy-2,3,4,5,6,11,12,12a-octahydro-1H-2,12-methanopyrrolo[1′,2′:1,2]azepino[4,5-b]indol-4-ium Chloride (8a), was prepared according to the protocol described in general procedure (VI) starting from (2S,12R,12aS)-8-methoxy-2,3,6,11,12,12a-hexahydro-2,12-methanopyrrolo[1′,2′:1,2]azepino[4,5-b]indol-5(1H)-one (7a) and isolated as a withe solid (5.5 mg, 25% yield). ¹H NMR (400 MHz, MeOD) δ 7.18 (d, J=8.7 Hz, 1H), 6.97 (d, J=2.4 Hz, 1H), 6.75 (dd, J=8.7, 2.4 Hz, 1H), 4.23 (s, 1H), 3.83 (s, 3H), 3.71-3.46 (m, 4H), 3.43-3.36 (m, 1H) 3.30-3.19 (m, 2H), 2.89-2.84 (m, 1H), 2.55-2.45 (m, 1H), 2.16-2.04 (m, 2H), 1.58-1.50 (m, 1H). ¹³C NMR, DEPT-135 (101 MHz, MeOD) δ111.09 (CH), 111.06 (CH), 99.19 (CH), 65.70 (CH), 54.88 (CH₃), 53.50 (CH₂), 50.66 (CH₂) 38.62 (CH), 38.32 (CH₂), 36.63 (CH), 35.74 (CH₂), 17.26 (CH₂). ESI-MS: measured m/z 269.27 [M+H]⁺. Purity by HPLC: 99% at 254 nm.

### Example 33: 8-methoxy-1,1-dimethyl-1,2,3,5,6,11,12,12a-octahydro-2,12-methanopyrrolo[1′,2′:1,2]azepino[4,5-b]indole (8b)

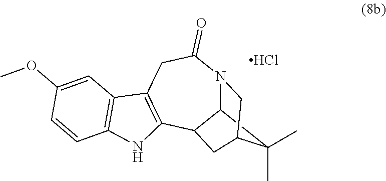

The title compound, 8-methoxy-1,1-dimethyl-1,2,3,5,6,11,12,12a-octahydro-2,12-methanopyrrolo[1′,2′:1,2]azepino[4,5-b]indole (8b), was prepared according to the protocol described in general procedure (VI) starting from 8-methoxy-1,1-dimethyl-2,3,6,11,12,12a-hexahydro-2,12-methanopyrrolo[1′,2′:1,2]azepino[4,5-b]indol-5(1H)-one (8b) and isolated the desired compound (6.4 mg, 65% yield). ¹H NMR (400 MHz, MeOD) δ 7.19 (d, J=8.7 Hz, 1H), 6.98 (d, J=2.4 Hz, 1H), 6.76 (dd, J=8.7, 2.4 Hz, 1H), 3.91 (ddd, J=12.3, 6.1, 2.6 Hz, 1H), 3.83 (s, 3H), 3.82-3.77 (m, 1H), 3.73-3.70 (m, 2H), 3.61-3.52 (m, 1H), 3.44 (d, J=11.6 Hz, 1H), 3.30-3.20 (m, 1H), 3.19-3.11 (m, 1H), 2.74-2.62 (m, 1H), 2.29 (t, J=4.2 Hz, 1H), 1.55 (dd, J=13.3, 6.1 Hz, 1H), 1.39 (s, 6H). ¹³C NMR, DEPT-135 (101 MHz, MeOD δ 111.23 (CH), 111.14 (CH), 99.24 (CH), 71.76 (CH), 54.91 (CH₃), 54.11 (CH₂), 52.77 (CH₂), 43.10 (CH), 36.45 (CH), 35.49 (CH₂), 19.83 (CH₃), 18.78 (CH₃), 17.34 (CH₂). ESI-MS: measured m/z 297.33 [M+H]⁺. Purity by HPLC: 98% at 254 nm.

### Example 34: (2S,12R,12aS)-7-fluoro-8-methoxy-1,2,3,5,6,11,12,12a-octahydro-2,12-methanopyrrolo[1′,2′:1,2]azepino[4,5-b]indole (8c)

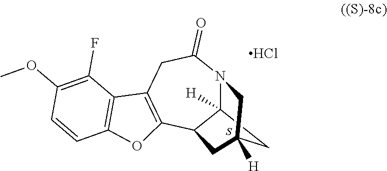

The title compound, (2S,12R,12aS)-7-fluoro-8-methoxy-1,2,3,5,6,11,12,12a-octahydro-2,12-methanopyrrolo[1′,2′:1,2]azepino[4,5-b]indole ((S)-8c), was prepared according to the protocol described in general procedure (VI) starting from (2S,12R,12aS)-7-fluoro-8-methoxy-2,3,6,11,12,12a-hexahydro-2,12-methanopyrrolo[1′,2′:1,2]azepino[4,5-b]indol-5(1H)-one ((S)-7c) and isolated the desired compound as a white solid (82 mg, 66% yield). ¹H NMR (400 MHz, MeOD) δ 8.56 (s, 1H), 7.00 (d, J=8.7 Hz, 1H), 6.91 (t, J=8.2 Hz, 1H), 4.26-4.15 (m, 1H), 3.87 (s, 3H), 3.69-3.60 (m, 2H), 3.60-3.46 (m, 2H), 3.46-3.39 (m, 2H), 3.39-3.34 (m, 1H), 2.90-2.79 (m, 1H), 2.56-2.40 (m, 1H), 2.15-1.96 (m, 2H), 1.63-1.43 (m, 1H). ¹³C NMR (101 MHz, MeOD) (Complicated by C-F coupling) δ 111.6 (CH), 106.0 and 105.9 (CH), 65.2 (CH), 58.0 (CH₃), 53.6 (CH₂), 50.6 (CH₂), 38.6 (CH), 38.3 (CH₂), 36.6 (CH), 35.4 (CH₂), 18.4 (CH₂). ¹⁹F NMR (376 MHz, MeOD) δ −151.27 (d, J=7.7 Hz). ESI-MS: measured m/z 287.27 [M+H]⁺. Purity by HPLC: 97.0% at 254 nm.

### Example 35: (2S,12R,12aS)-9-fluoro-8-methoxy-1,2,3,5,6,11,12,12a-octahydro-2,12-methanopyrrolo[1′,2′:1,2]azepino[4,5-b]indole ((S)-8d)

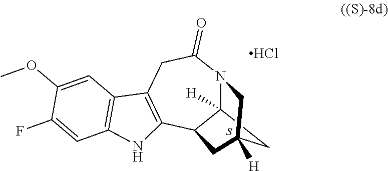

The title compound, (2S,12R,12aS)-9-fluoro-8-methoxy-1,2,3,5,6,11,12,12a-octahydro-2,12-methanopyrrolo[1′,2′:1,2]azepino[4,5-b]indole ((S)-8d), was prepared according to the protocol described in general procedure (VI) starting from (2S,12R,12aS)-9-fluoro-8-methoxy-2,3,6,11,12,12a-hexahydro-2,12-methanopyrrolo[1′,2′:1,2]azepino[4,5-b]indol-5(1H)-one ((S)-7d) and isolated the desired compound as a white solid (21 mg, 18% yield). ¹H NMR (400 MHz, MeOD) δ 8.51 (brs, 1H), 7.09 (d, J=8.1 Hz, 1H), 7.03 (d, J=11.5 Hz, 1H), 4.25-4.17 (m, 1H), 3.89 (s, 3H), 3.70-3.48 (m, 4H), 3.34-3.07 (m, 3H), 2.87-2.79 (m, 1H), 2.54-2.41 (m, 1H), 2.12-2.01 (m, 2H), 1.56-1.47 (m, 1H). ¹³C NMR, DEPT-135 (101 MHz, MeOD) (Complicated by C-F coupling) δ 101.3 and 101.3 (CH), 97.8 and 97.6 (CH), 65.5 (CH), 56.1 (CH), 53.4 (CH₂), 50.5 (CH₂), 38.5 (CH), 38.2 (CH₂), 36.6 (CH), 35.8 (CH₂), 17.2 (CH₂). ¹⁹F NMR (376 MHz, MeOD) δ −142.99 (dd, J=11.5, 8.2 Hz). ESI-MS: measured m/z 287.27 [M+H]⁺. Purity by HPLC: 98.7% at 254 nm.

### Example 36: (2S,12R,12aS)-1-ethyl-8-methoxy-1,2,3,5,6,11,12,12a-octahydro-2,12-methanopyrrolo[1′,2′:1,2]azepino[4,5-b]indole ((S)-8e)

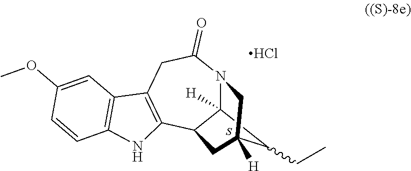

The title compound, (2S,12R,12aS)-1-ethyl-8-methoxy-1,2,3,5,6,11,12,12a-octahydro-2,12-methanopyrrolo[1′,2′:1,2]azepino[4,5-b]indole ((S)-8e), was prepared according to the protocol described in general procedure (VI) starting from (2S,12R,12aS)-1-ethyl-8-methoxy-2,3,6,11,12,12a-hexahydro-2,12-methanopyrrolo[1′,2′:1,2]azepino[4,5-b]indol-5(1H)-one ((S)-7e) and isolated the desired compound as a white solid (35 mg, 28% yield). ¹H NMR (400 MHz, MeOD) δ 8.55 (brs, 1H), 7.18 (d, J=8.7 Hz, 1H), 6.96 (d, J=2.4 Hz, 1H), 6.75 (dd, J=8.7, 2.4 Hz, 1H), 4.01-3.96 (m, 1H), 3.83 (s, 3H), 3.72-3.50 (m, 4H), 3.38-3.34 (m, 1H), 3.29-3.17 (m, 1H), 3.15-3.06 (m, 1H), 2.62-2.57 (m, 1H), 2.57-2.46 (m, 1H), 2.32 (t, J=7.5 Hz, 1H), 1.71-1.55 (m, 2H), 1.55-1.47 (m, 1H), 1.09 (t, J=7.3 Hz, 3H). ¹³C NMR, DEPT-135 (101 MHz, MeOD) δ 111.1 (CH), 111.0 (CH), 99.2 (CH), 67.0 (CH), 54.9 (CH₃), 53.4 (CH), 52.2 (CH₂), 51.6 (CH₂), 39.3 (CH), 38.8 (CH), 36.1 (CH₂), 18.2 (CH₂), 17.2 (CH₂), 11.7 (CH₃). ESI-MS: measured m/z 297.33 [M+H]⁺. Purity by HPLC: 99.2% at 254 nm.

### Example 37: (2S,12R,12aS)-8-fluoro-1,2,3,5,6,11,12,12a-octahydro-2,12-methanopyrrolo[1′,2′:1,2]azepino[4,5-b]indole (17a)

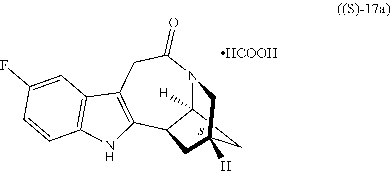

The title compound, (2S,12R,12aS)-8-fluoro-1,2,3,5,6,11,12,12a-octahydro-2,12-methanopyrrolo[1′,2′:1,2]azepino[4,5-b]indole ((S)-17a), was prepared according to the protocol described in general procedure (XI) starting from (2S,12R,12aS)-8-fluoro-2,3,6,11,12,12a-hexahydro-2,12-methanopyrrolo[1′,2′:1,2]azepino[4,5-b]indol-5(1H)-one ((S)-16a) and isolated the desired compound as a white solid (10 mg, 11% yield). ¹H NMR (400 MHz, MeOD) δ 8.46 (s, 1H), 7.25 (dd, J=8.8, 4.4 Hz, 1H), 7.14 (dd, J=9.8, 2.5 Hz, 1H), 6.89-6.81 (m, 1H), 4.22 (s, 1H), 3.70-3.35 (m, 5H), 3.31-3.19 (m, 1H), 3.16-3.05 (m, 1H), 2.88-2.82 (m, 1H), 2.56-2.43 (m, 1H), 2.09 (s, 2H), 1.55 (dd, J=12.8, 5.8 Hz, 1H).

¹³C NMR, DEPT-135 (101 MHz, MeOD) (spectrum complicated by F-C coupling) δ 111.23 and 111.13 (CH), 109.15 and 108.89 (CH), 101.89 and 101.65 (CH), 65.50 (CH), 53.46 (CH₂), 50.44 (CH₂), 38.54 (CH), 38.26 (CH₂), 36.60 (CH), 35.62 (CH₂), 17.18 (CH₂). ESI-MS: measured m/z 257.27 [M+H]⁺. Purity by HPLC: 97% at 254 nm.

### Example 38: (2S,12R,12aS)-9-fluoro-1,2,3,5,6,11,12,12a-octahydro-2,12-methanopyrrolo[1′,2′:1,2]azepino[4,5-b]indole (17b)

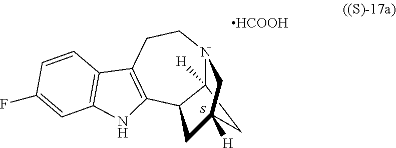

The title compound, (2S,12R,12aS)-9-fluoro-1,2,3,5,6,11,12,12a-octahydro-2,12-methanopyrrolo[1′,2′:1,2]azepino[4,5-b]indole ((S)-17b), was prepared according to the protocol described in general procedure (XI) starting from (2S,12R,12aS)-9-fluoro-2,3,6,11,12,12a-hexahydro-2,12-methanopyrrolo[1′,2′:1,2]azepino[4,5-b]indol-5(1H)-one ((S)-16b) and isolated the desired compound as a white solid (5.5 mg, 7.6% yield). ¹H NMR (400 MHz, MeOD) δ 8.56 (s, 1H), 7.42 (dd, J=8.7, 5.2 Hz, 1H), 6.99 (dd, J=9.8, 2.3 Hz, 1H), 6.85-6.77 (m, 1H), 4.24 (s, 1H), 3.72-3.47 (m, 4H), 3.43-3.35 (m, 1H), 3.29-3.11 (m, 2H), 2.90-2.84 (s, 1H), 2.56-2.44 (m, 1H), 2.14-2.03 (m, 2H), 1.55 (ddd, J=12.8, 5.7, 1.8 Hz, 1H).

¹³C NMR, DEPT-135 (101 MHz, MeOD) (spectrum complicated by F-C coupling) δ 117.98 and 117.86 (CH), 107.23 and 107.03 (CH), 96.13 and 96.27 (CH), 65.59 (CH), 53.54 (CH₂), 50.51 (CH₂), 38.36 (CH), 38.25 (CH₂), 36.61 (CH), 35.74 (CH₂), 17.18 (CH₂). ESI-MS: measured m/z 257.27 [M+H]⁺. Purity by HPLC: 98% at 254 nm.

### Example 39: (2S,12R,12aS)-8-(trifluoromethyl)-1,2,3,5,6,11,12,12a-octahydro-2,12-methanopyrrolo[1′,2′:1,2]azepino[4,5-b]indole (17c)

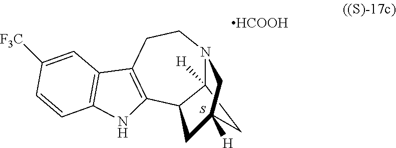

The title compound, (2S,12R,12aS)-8-(trifluoromethyl)-1,2,3,5,6,11,12,12a-octahydro-2,12-methanopyrrolo[1′,2′:1,2]azepino[4,5-b]indole ((S)-17c), was prepared according to the protocol described in general procedure (XI) starting from (2S,12R,12aS)-8-(trifluoromethyl)-2,3,6,11,12,12a-hexahydro-2,12-methanopyrrolo[1′,2′:1,2]azepino[4,5-b]indol-5(1H)-one ((S)-16c) and isolated the desired compound as a white solid (6.5 mg, 31% yield). ¹H NMR (400 MHz, MeOD) δ 8.56 (s, 1H), 7.78 (s, 1H), 7.41 (d, J=8.5 Hz, 1H), 7.33 (dd, J=8.5, 1.7 Hz, 1H), 4.05-4.01 (m, 1H), 3.61-3.48 (m, 3H), 3.42-3.36 (m, 1H), 3.32-3.25 (m, 1H), 3.24-3.19 (m, 1H), 3.11-3.03 (m, 1H), 2.81-2.75 (m, 1H), 2.51-2.42 (m, 1H), 2.04-1.94 (m, 2H), 1.55-1.48 (m, 1H). ¹³C NMR, DEPT-135 (101 MHz, MeOD) δ 117.2 (CH), 113.5 (CH), 110.6 (CH), 64.7 (CH), 52.9 (CH₂), 49.9 (CH₂), 40.1 (CH), 38.4 (CH₂), 37.1 (CH), 36.4 (CH₂), 17.7 (CH₂). ¹⁹F NMR (376 MHz, MeOD) δ −61.5. ESI-MS: measured m/z 307.33 [M+H]⁺. Purity by HPLC: 97.4% at 254 nm.

### Example 40: (2S,12R,12aS)-8-methoxy-2,3,5,6,12,12a-hexahydro-1H-2,12-methanobenzofuro[2,3-d]pyrrolo[1,2-a]azepine (17d)

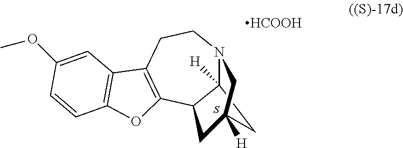

The title compound, (2S,12R,12aS)-8-methoxy-2,3,5,6,12,12a-hexahydro-1H-2,12-methanobenzofuro[2,3-d]pyrrolo[1,2-a]azepine ((S)-17d), was prepared according to the protocol described in general procedure (XI) starting from (2S,12R,12aS)-8-methoxy-1,2,3,6,12,12a-hexahydro-5H-2,12-methanobenzofuro[2,3-d]pyrrolo[1,2-a]azepin-5-one ((S)-16d) and isolated the desired compound as a white solid (10 mg, 55% yield). ¹H NMR (400 MHz, MeOD) δ 8.49 (s, 1H), 7.25 (d, J=8.9 Hz, 1H), 6.99 (d, J=2.5 Hz, 1H), 6.84 (dd, J=8.9, 2.6 Hz, 1H), 4.11 (m, 1H), 3.81 (s, 3H), 3.67-3.56 (m, 3H), 3.44 (m, 1H), 3.24-3.16 (m, 1H), 3.16-3.09 (m, 1H), 3.00-2.92 (m, 1H), 2.83-2.76 (m, 1H), 2.51-2.38 (m, 1H), 2.06-1.93 (m, 2H), 1.52 (m, 1H). ¹³C NMR, DEPT-135 (101 MHz, MeOD) δ 112.28 (CH), 110.78 (CH), 101.02 (CH), 64.32 (CH₃), 54.93 (CH), 52.58 (CH₂), 49.28 (CH₂), 38.90 (CH), 38.03 (CH₂), 36.85 (CH), 36.10 (CH₂), 15.97 (CH₂). ESI-MS: measured m/z 270.27 [M+H]⁺. Purity by HPLC: 99.0% at 254 nm.

### Example 41: (2S,12aR)-1,1-dimethyl-1,2,3,5,6,11,12,12a-octahydro-2,12-methanopyrrolo[1′,2′:1,2]azepino[4,5-b]indol-8-ol (9b)

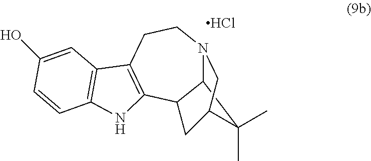 [SMILES: Cl ; CC1(C)C2CC3c4[nH]c5ccc(O)cc5c4CCN(C2)C31]

The title compound, (2S,12aR)-1,1-dimethyl-1,2,3,5,6,11,12,12a-octahydro-2,12-methanopyrrolo[1′,2′:1,2]azepino[4,5-b]indol-8-ol (9b), was prepared (4.1 mg, 8.4% yield) according to the protocol described in general procedure (VIII) starting from 8-methoxy-1,1-dimethyl-1,2,3,5,6,11,12,12a-octahydro-2,12-methanopyrrolo[1′,2′:1,2]azepino[4,5-b]indole (8b). ¹H NMR (400 MHz, MeOD) δ 8.47 (s, 1H), 7.11 (d, J=8.6 Hz, 1H), 6.83 (d, J=2.3 Hz, 1H), 6.67 (dd, J=8.6, 2.3 Hz, 1H), 3.91-3.84 (m, 1H), 3.78 (dt, J=11.5, 3.6 Hz, 1H), 3.72-3.64 (m, 2H), 3.60-3.50 (m, 1H), 3.42 (d, J=11.5 Hz, 1H), 3.28-3.18 (m, 1H), 3.10-3.00 (m, 1H), 2.74-2.61 (m, 1H), 2.27 (t, J=4.1 Hz, 1H), 1.55 (dd, J=13.3, 6.1 Hz, 1H), 1.40-1.36 (m, 6H). ¹³C NMR, DEPT-135 (101 MHz, MeOD) δ 111.01 (CH), 110.84 (CH), 101.30 (CH), 71.60 (CH), 53.88 (CH₂), 52.50 (CH₂), 43.15 (CH), 36.73 (CH), 35.62 (CH₂), 19.85 (CH₃), 18.69 (CH₃), 17.46 (CH₂). ESI-MS: measured m/z 283.27 [M+H]⁺. Purity by HPLC: 95% at 254 nm.

### Example 42: (2S,12R,12aS)-7-fluoro-1,2,3,5,6,11,12,12a-octahydro-2,12-methanopyrrolo[1′,2′:1,2]azepino[4,5-b]indol-8-ol ((S)-9c)

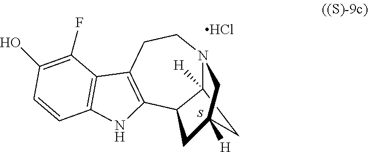

The title compound, (2S,12R,12aS)-7-fluoro-1,2,3,5,6,11,12,12a-octahydro-2,12-methanopyrrolo[1′,2′:1,2]azepino[4,5-b]indol-8-ol ((S)-9c), was prepared as a white solid (7.8 mg, 23% yield) according to the protocol described in general procedure (VIII) starting from (2S,12R,12aS)-7-fluoro-8-methoxy-1,2,3,5,6,11,12,12a-octahydro-2,12-methanopyrrolo [1′,2′:1,2]azepino[4,5-b]indole ((S)-8c).). ¹H NMR (400 MHz, MeOD) δ 8.52 (brs, 1H), 6.90 (d, J=8.6 Hz, 1H), 6.73 (t, J=8.4 Hz, 1H), 4.22-4.16 (m, 1H), 3.67-3.59 (m, 2H), 3.57-3.49 (m, 1H), 3.49-3.39 (m, 3H), 3.39-3.35 (m, 1H), 2.89-2.79 (m, 1H), 2.56-2.40 (m, 1H), 2.06 (m, 2H), 1.63-1.51 (m, 1H). ¹³C NMR (101 MHz, MeOD) (Complicated by C-F coupling) δ 113.0 (CH), 106.0 and 106.0 (CH), 65.3 (CH), 53.7 (CH₂), 50.7 (CH₂), 38.8 (CH), 38.3 (CH₂), 36.6 (CH), 35.4 (CH₂), 18.6 (CH₂). ¹⁹F NMR (376 MHz, MeOD) δ −155.23 (d, J=8.2 Hz). ESI-MS: measured m/z 273.27 [M+H]⁺. Purity by HPLC: 97.2% at 254 nm.

### Example 43: (2S,12R,12aS)-9-fluoro-1,2,3,5,6,11,12,12a-octahydro-2,12-methanopyrrolo[1′,2′:1,2]azepino[4,5-b]indol-8-ol ((S)-9d)

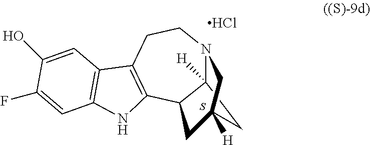

The title compound, (2S,12R,12aS)-9-fluoro-1,2,3,5,6,11,12,12a-octahydro-2,12-methanopyrrolo[1′,2′:1,2]azepino[4,5-b]indol-8-ol ((S)-9d), was prepared (10 mg, 45% yield) according to the protocol described in general procedure (VIII) starting from (2S,12R,12aS)-9-fluoro-8-methoxy-1,2,3,5,6,11,12,12a-octahydro-2,12-methanopyrrolo[1′,2′:1,2]azepino[4,5-b]indole ((S)-8d). ¹H NMR (400 MHz, MeOD) δ 8.55 (brs, 1H), 6.99 (d, J=11.3 Hz, 1H), 6.94 (d, J=8.4 Hz, 1H), 4.15-4.11 (m, 1H), 3.67-3.41 (m, 4H), 3.32-3.17 (m, 2H), 3.05-2.94 (m, 1H), 2.86-2.79 (m, 1H), 2.55-2.40 (m, 1H), 2.03 (dd, J=4.2, 1.9 Hz, 2H), 1.55-1.49 (m, 1H). ¹³C NMR (101 MHz, MeOD) (Complicated by C-F coupling) δ 103.7 and 103.7 (CH), 97.3 and 97.1 (CH), 65.6 (CH), 53.5 (CH₂), 50.5 (CH₂), 38.5 (CH), 38.3 (CH₂), 36.6 (CH), 35.7 (CH₂), 17.3 (CH₂). ¹⁹F NMR (376 MHz, MeOD) δ −144.42 (dd, J=11.2, 8.5 Hz). ESI-MS: measured m/z 273.27 [M+H]⁺. Purity by HPLC: 98.3% at 254 nm.

### Example 44: (2S,12R,12aS)-1-ethyl-1,2,3,5,6,11,12,12a-octahydro-2,12-methanopyrrolo[1′,2′:1,2]azepino[4,5-b]indol-8-ol ((S)-9e)

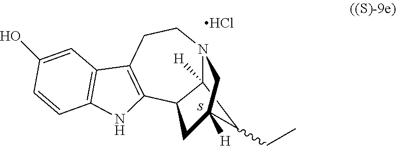

The title compound, (2S,12R,12aS)-1-ethyl-1,2,3,5,6,11,12,12a-octahydro-2,12-methanopyrrolo[1′,2′:1,2]azepino[4,5-b]indol-8-ol ((S)-9e), was prepared (13 mg, 56% yield) according to the protocol described in general procedure (VIII) starting from (2S,12R,12aS)-1-ethyl-8-methoxy-1,2,3,5,6,11,12,12a-octahydro-2,12-methanopyrrolo[1′,2′:1,2]azepino[4,5-b]indole ((S)-8e). ¹H NMR (400 MHz, MeOD) δ 8.58 (brs, 1H), 7.08 (d, J=8.6 Hz, 1H), 6.80 (d, J=2.3 Hz, 1H), 6.62 (dd, J=8.6, 2.3 Hz, 1H), 3.72-3.64 (m, 1H), 3.55-3.37 (m, 4H), 3.27-3.13 (m, 2H), 2.89-2.78 (m, 1H), 2.49-2.38 (m, 2H), 2.11 (t, J=7.5 Hz, 1H), 1.68-1.53 (m, 2H), 1.49-1.43 (m, 1H), 1.06 (t, J=7.4 Hz, 3H). ¹³C NMR, DEPT-135 (101 MHz, MeOD) δ 110.6 (CH), 110.3 (CH), 101.3 (CH), 66.2 (CH), 53.3 (CH), 51.1 (CH₂), 50.9 (CH₂), 40.6 (CH), 39.5 (CH), 37.3 (CH₂), 18.6 (CH₂), 18.4 (CH₂), 11.9 (CH₃). ESI-MS: measured m/z 283.27 [M+H]⁺. Purity by HPLC: 97.1% at 254 nm.

### Example 45: (2S,12R,12aS)-11-ethyl-8-methoxy-1,2,3,5,6,11,12,12a-octahydro-2,12-methanopyrrolo[1′,2′:1,2]azepino[4,5-b]indole ((S)-10a)

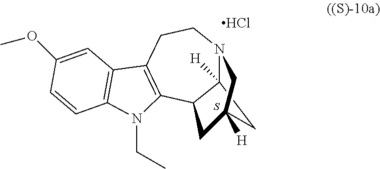

The title compound, (2S,12R,12aS)-11-ethyl-8-methoxy-1,2,3,5,6,11,12,12a-octahydro-2,12-methanopyrrolo[1′,2′:1,2]azepino[4,5-b]indole ((S)-10a), was prepared (49 mg, 21% yield) according to the protocol described in general procedure (VII); ¹H NMR (400 MHz, MeOD) δ 7.25 (d, J=8.9 Hz, 1H), 7.00 (d, J=2.4 Hz, 1H), 6.82 (dd, J=8.9, 2.4 Hz, 1H), 4.30 (s, 1H), 4.23-4.09 (m, 2H), 3.87-3.75 (m, 4H), 3.70-3.53 (m, 3H), 3.44-3.38 (m, 1H), 3.30-3.15 (m, 2H), 2.93-2.87 (m, 1H), 2.63-2.53 (m, 1H), 2.28-2.04 (m, 2H), 1.55 (ddd, J=12.7, 6.2, 2.2 Hz, 1H), 1.29 (t, J=7.2 Hz, 3H). ESI-MS: measured m/z 297.33 [M+H]⁺. Purity by HPLC: 97% at 254 nm.

### Example 46: (2S,12R,12aS)-11-ethyl-1,2,3,5,6,11,12,12a-octahydro-2,12-methanopyrrolo[1′,2′:1,2]azepino[4,5-b]indol-8-ol ((S)-11a)

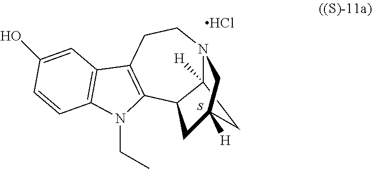

The title compound, (2S,12R,12aS)-11-ethyl-1,2,3,5,6,11,12,12a-octahydro-2,12-methanopyrrolo[1′,2′:1,2]azepino[4,5-b]indol-8-ol ((S)-11a), was prepared (49 mg, 21% yield) according to the protocol described in general procedure (VIII) starting from (2S,12R,12aS)-11-ethyl-8-methoxy-1,2,3,5,6,11,12,12a-octahydro-2,12-methanopyrrolo[1′,2′:1,2]azepino[4,5-b]indole; ¹H NMR (400 MHz, MeOD) δ 7.25 (d, J=8.9 Hz, 1H), 7.00 (d, J=2.4 Hz, 1H), 6.82 (dd, J=8.9, 2.4 Hz, 1H), 4.30 (s, 1H), 4.23-4.09 (m, 2H), 3.87-3.75 (m, 4H), 3.70-3.53 (m, 3H), 3.44-3.38 (m, 1H), 3.30-3.15 (m, 2H), 2.93-2.87 (m, 1H), 2.63-2.53 (m, 1H), 2.28-2.04 (m, 2H), 1.55 (ddd, J=12.7, 6.2, 2.2 Hz, 1H), 1.29 (t, J=7.2 Hz, 3H). ¹³C NMR (101 MHz, MeOD) δ 111.2 (CH), 109.4 (CH), 101.6 (CH), 65.5 (CH), 55.0 (CH₂), 51.3 (CH₂), 38.3 (CH₂), 37.2 (CH₂), 36.6 (CH), 36.4 (CH), 34.8 (CH₂), 17.5 (CH₂), 14.8 (CH₃). ESI-MS: measured m/z 297.33 [M+H]⁺. Purity by HPLC: 97% at 254 nm.

### Example 47: (2S,12R,12aS)-2,3,5,6,12,12a-hexahydro-1H-2,12-methanobenzofuro[2,3-d]pyrrolo[1,2-a]azepin-8-ol ((S)-18a)

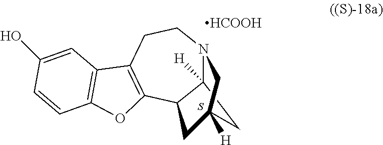

The title compound, (2S,12R,12aS)-2,3,5,6,12,12a-hexahydro-1H-2,12-methanobenzofuro[2,3-d]pyrrolo[1,2-a]azepin-8-ol ((S)-18a), was prepared according to the protocol described in general procedure (VIII) starting from (2S,12R,12aS)-8-methoxy-2,3,5,6,12,12a-hexahydro-1H-2,12-methanobenzofuro[2,3-d]pyrrolo[1,2-a]azepine ((S)-17a) and isolated the desired compound as a white solid (8 mg, 74% yield). ¹H NMR (400 MHz, MeOD) δ 7.21 (d, J=8.8 Hz, 1H), 6.86 (d, J=2.5 Hz, 1H), 6.75 (dd, J=8.8, 2.5 Hz, 1H), 4.17 (s, 1H), 3.71-3.62 (m, 3H), 3.50-3.43 (m, 1H), 3.30-3.25 (m, 1H), 3.23-3.12 (m, 1H), 2.99-2.90 (m, 1H), 2.88-2.81 (m, 1H), 2.54-2.43 (m, 1H), 2.04 (s, 2H), 1.56 (dd, J=12.7, 5.2 Hz, 1H). ¹³C NMR, DEPT-135 (101 MHz, MeOD) δ 112.36 (CH), 110.54 (CH), 102.99 (CH), 64.52 (CH), 52.75 (CH₂), 49.49 (CH₂), 38.81 (CH), 38.09 (CH₂), 36.84 (CH), 36.02 (CH₂), 16.04 (CH₂). ESI-MS: measured m/z 256.27 [M+H]⁺. Purity by HPLC: 99% at 254 nm.

### Example 48: Synthesis of (2S,12R,12aS)-8-methoxy-2,3,6,11,12,12a-hexahydro-2,12-methanopyrrolo[1′,2′:1,2]azepino[4,5-b]indol-5(1H)-imine (12)

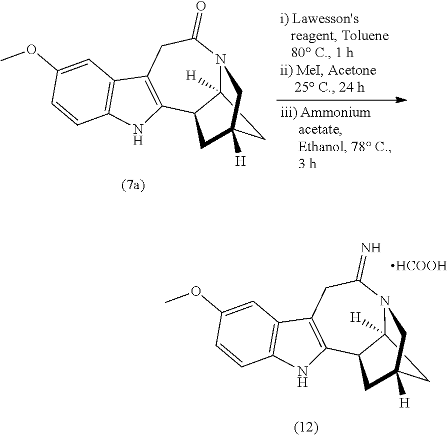

Under an inert atmosphere of argon gas, a solution of (2S,12R,12aS)-8-methoxy-2,3,6,11,12,12a-hexahydro-2,12-methanopyrrolo[1′,2′:1,2]azepino[4,5-b]indol-5(1H)-one (7a) (1 eq) in dry toluene (0.05 M) at 25° C. was added Lawesson's reagent (0.55 eq). The reaction was heated at 80° C. and stirred for 1 hour. The reaction completion was confirmed by LC-MS and TLC. The organic solvent was removed under reduced pressure and dried. The crude was purified by normal phase silica gel column chromatography, running a mobile phase of 20% Ethyl acetate in 80% Hexane, and the product containing fractions were dried under reduced pressure to afford the corresponding thione. Under an inert atmosphere thione (1.0 eq) in acetone (0.02 M) at 25° C. was added iodomethane (2.5 eq). The reaction was stirred for 24 hours. The reaction completion was confirmed by LCMS. The organic solvent was removed under reduced pressure and dried. The obtained crude was dissolved in ethanol (0.04 M) and added ammonium acetate (10 eq) at 25° C. The reaction was heated at 78° C. and stirred for 3 h. LCMS confirmed the formation of product. The organic solvent was removed under reduced pressure and dried. The crude was purified by reverse phase (C18) column chromatography, running a mobile phase of acetonitrile in 80% water, and 0.1% formic acid. The product containing fractions were dried under reduced pressure to afford the desired product (6 mg, 38% yield). ¹H NMR (400 MHz, MeOD) δ 8.56 (brs, 1H), 7.18 (d, J=8.8 Hz, 1H), 6.99 (d, J=2.4 Hz, 1H), 6.76 (dd, J=8.8, 2.4 Hz, 1H), 4.98 (s, 1H), 4.29-4.18 (m, 1H), 3.88-3.77 (m, 4H), 3.72-3.64 (m, 1H), 3.54-3.45 (m, 1H), 3.19-3.11 (m, 1H), 2.91-2.83 (m, 1H), 2.54-2.45 (m, 1H), 2.19-2.12 (m, 1H), 2.03-1.98 (m, 1H), 1.39-1.35 (m, 1H). ¹³C NMR, DEPT-135 (101 MHz, MeOD) δ 111.24 (CH), 111.21 (CH), 99.18 (CH), 61.96 (CH₃), 59.68 (CH₂), 54.85 (CH), 39.94 (CH), 38.14 (CH), 38.03 (CH₂), 33.90 (CH₂), 27.22 (CH₂). ESI-MS: measured m/z 282.27 [M+H]⁺. Purity by HPLC: 98.5% at 254 nm.

### Example 49: (2R,12S,12aS)-8,12-dimethoxy-1,2,3,5,6,11,12,12a-octahydro-2,12-methanopyrrolo[1′,2′:1,2]azepino[4,5-b]indole ((S)-13)

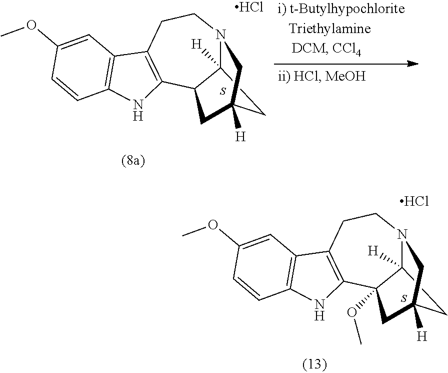

The hydrochloride salt of (2S,12R,12aS)-8-methoxy-1,2,3,5,6,11,12,12a-octahydro-2,12-methanopyrrolo[1′,2′:1,2]azepino[4,5-b]indole (8a). (60 mg, 196.84 μmol) was dissolved in DCM (5 mL) and cooled to 0° C. in an ice bath and Triethylamine (40 mg, 394 μmol, 55 μL) was added. Tert-Butyl hypochlorite (43 mg, 394 μmol) was diluted in carbon tetrachloride (2 mL) and added to the reaction mixture at 0° C. over 20 minutes. After the addition, the reaction was continued for 40 minutes. The reaction mixture was treated with sodium carbonate solution and diluted with methylene chloride and the organic phase was successively washed with water, dried on sodium sulfate. Filtered, the filtrate was concentrated and dissolved in 5 mL MeOH, treated with 2 M HCl in Ether (5 μL). Continued stirring it for overnight. Removed all the volatiles from the reaction mixture and the crude was purified by reverse phase (C18) column chromatography. The product containing fractions were dried under reduced pressure to afford the desired product (8.0 mg, 14% yield).

¹H NMR (400 MHz, MeOD) δ 8.53 (brs, 1H), 7.25 (d, J=8.8 Hz, 1H), 7.00 (d, J=2.4 Hz, 1H), 6.81 (dd, J=8.8, 2.4 Hz, 1H), 4.68 (s, 1H), 3.98-3.91 (m, 1H), 3.83 (s, 3H), 3.68-3.61 (m, 1H), 3.32-3.19 (m, 2H), 3.19-3.15 (m, 1H), 3.14 (s, 3H), 2.88-2.81 (m, 1H), 2.79-2.75 (m, 1H), 2.42-2.34 (m, 1H), 2.34-2.25 (m, 1H), 2.03-1.92 (m, 2H). ¹³C NMR, DEPT-135 (101 MHz, MeOD) δ 112.3 (CH), 111.5 (CH), 99.6 (CH), 64.1 (CH), 57.9 (CH₂), 54.9 (CH₃), 53.6 (CH₂), 51.0 (CH), 45.9 (CH₂) 35.3 (CH), 34.7 (CH₂), 19.0 (CH₂). ESI-MS: measured m/z 299.27 [M+H]⁺. Purity by HPLC: 95.0% at 254 nm.

### Example 50: 1-((1R,4S)-2-azabicyclo[2.2.1]hept-5-en-2-yl)-2-(2-bromo-5-methoxy-1H-indol-3-yl)ethan-1-one ((R)-6a)

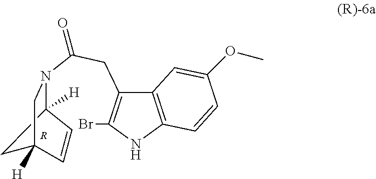

The title compound, 1-((1R,4S)-2-azabicyclo[2.2.1]hept-5-en-2-yl)-2-(2-bromo-5-methoxy-1H-indol-3-yl)ethan-1-one ((R)-6a), was prepared according to the protocol described in general procedure (IV) starting from (1R,4S)-2-azabicyclo[2.2.1]hept-2-ene ((1R)-5) to afford the desired product (210 mg, 87% yield). ¹H NMR (400 MHz, CDCl₃) δ 8.20-8.04 (m, 1H), 7.22-7.12 (m, 2H), 6.87-6.79 (m, 1H), 6.42-6.38 (m, 0.46H), 6.31-6.26 (m, 0.48H), 6.26-6.19 (m, 0.59H), 5.86-5.79 (m, 0.54H), 5.20 (s, 0.43H), 4.75 (s, 0.57H), 3.86 (s, 1.5H), 3.84 (s, 1.5H), 3.80-3.74 (m, 0.5H), 3.64-3.59 (m, 0.5H), 3.54-3.49 (m, 1H), 3.47-3.39 (m, 1H), 3.29-3.25 (m, 1H), 3.22-3.18 (m, 1H), 2.87-2.80 (m, 1H), 1.66-1.62 (m, 1H), 1.54-1.50 (m, 1H). ESI-MS: measured m/z 361.07 [M+H, ⁷⁹Br]⁺, 363.00 [M+H, ⁸¹Br]⁺.

### Example 51: Synthesis of (2R,12S,12aR)-8-methoxy-2,3,6,11,12,12a-hexahydro-2,12-methanopyrrolo[1′,2′:1,2]azepino[4,5-b]indol-5(1H)-one ((R)-7a)

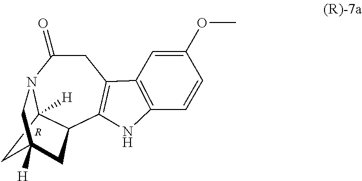

The title compound, (2R,12S,12aR)-8-methoxy-2,3,6,11,12,12a-hexahydro-2,12-methanopyrrolo[1′,2′:1,2]azepino[4,5-b]indol-5(1H)-one ((R)-7a), was prepared according to the protocol described in general procedure (V) starting from 1-((1R,4S)-2-azabicyclo[2.2.1]hept-5-en-2-yl)-2-(2-bromo-5-methoxy-1H-indol-3-yl)ethan-1-one ((R)-6a) to afford the desired product (91.0 mg, 45% yield). ¹H NMR (400 MHz, MeOD) δ 7.15 (d, J=8.7 Hz, 1H), 6.98 (d, J=2.4 Hz, 1H), 6.72 (dd, J=8.7, 2.4 Hz, 1H), 4.70 (s, 1H), 4.05 (dd, J=14.5, 2.1 Hz, 1H), 3.83 (s, 3H), 3.53-3.44 (m, 1H), 3.43-3.36 (m, 2H), 3.30-3.24 (m, 1H), 2.73-2.68 (m, 1H), 2.46-2.36 (m, 1H), 2.11-2.05 (m, 1H), 1.96-1.91 (m, 1H), 1.35-1.28 (m, 1H). ESI-MS: measured m/z 283.27 [M+H]⁺.

### Example 52: (2R,12S,12aR)-8-methoxy-2,3,4,5,6,11,12,12a-octahydro-1H-2,12-methanopyrrolo[1′,2′:1,2]azepino[4,5-b]indol-4-ium Chloride ((R)-8a)

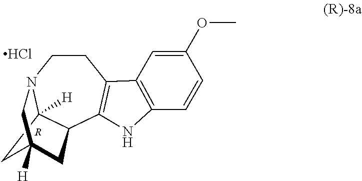

The title compound, (2R,12S,12aR)-8-methoxy-1,2,3,5,6,11,12,12a-octahydro-2,12-methanopyrrolo[1′,2′:1,2]azepino[4,5-b]indole ((R)-8a), was prepared according to the protocol described in in general procedure (VI) starting from (2R,12S,12aR)-8-methoxy-2,3,6,11,12,12a-hexahydro-2,12-methanopyrrolo[1′,2′:1,2]azepino[4,5-b]indol-5(1H)-one ((R)-7a) and isolated the desired compound (35 mg, 61% yield). ¹H NMR (400 MHz, MeOD) δ 7.17 (d, J=8.6 Hz, 1H), 6.96 (d, J=2.3 Hz, 1H), 6.75 (dd, J=8.7, 2.1 Hz, 1H), 4.27-4.20 (m, 1H), 3.82 (s, 3H), 3.68-3.47 (m, 4H), 3.45-3.37 (m, 1H), 3.25-3.11 (m, 2H), 2.91-2.83 (m, 1H), 2.55-2.45 (m, 1H), 2.14-2.07 (m, 2H), 1.59-1.52 (m, 1H). ESI-MS: measured m/z 269.27 [M+H]⁺. Purity by HPLC: 98% at 254 nm.

### Example 53: (2S,12R,12aS)-1,2,3,5,6,11,12,12a-octahydro-2,12-methanopyrrolo [1′,2′:1,2]azepino[4,5-b]indol-8-ol ((S)-9a)

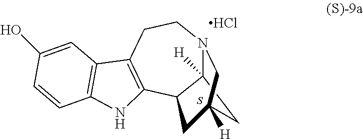

The title compound, (2S,12R,12aS)-1,2,3,5,6,11,12,12a-octahydro-2,12-methanopyrrolo[1′,2′:1,2]azepino[4,5-b]indol-8-ol ((S)-9a), was prepared (3 mg, 35% yield) according to the protocol described in general procedure (VIII) starting from (2S,12R,12aS)-8-methoxy-2,3,4,5,6,11,12,12a-octahydro-1H-2,12-methanopyrrolo[1′,2′:1,2]azepino[4,5-b]indol-4-ium Chloride ((S)-8a): (¹H NMR (400 MHz, MeOD) δ 7.11 (d, J=8.6 Hz, 1H), 6.83 (d, J=2.4 Hz, 1H), 6.65 (dd, J=8.6, 2.4 Hz, 1H), 4.14 (s, 1H), 3.67-3.44 (m, 4H), 3.28-3.16 (m, 2H), 3.02 (d, J=17.4 Hz, 1H), 2.84 (s, 1H), 2.51-2.44 (m, 1H), 2.07 (s, 2H), 1.55-1.49 (m, 1H). ¹³C NMR, DEPT-135 (101 MHz, MeOD) δ 110.82 (CH), 110.76 (CH), 101.29 (CH), 65.52 (CH), 53.30 (CH₂), 50.55 (CH₂), 39.06 (CH), 38.39 (CH₂), 36.75 (CH), 35.94 (CH₂), 17.46 (CH₂). ESI-MS: measured m/z 255.27 [M+H]⁺. Purity by HPLC: 98% at 254 nm.

### Example 54: (2R,12S,12aR)-1,2,3,5,6,11,12,12a-octahydro-2,12-methanopyrrolo[1′,2′:1,2]azepino[4,5-b]indol-8-ol ((R)-9a)

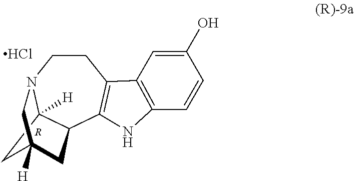

The title compound, (2R,12S,12aR)-1,2,3,5,6,11,12,12a-octahydro-2,12-methanopyrrolo[1′,2′:1,2]azepino[4,5-b]indol-8-ol ((R)-9a), was prepared (5.4 mg, 30% yield) according to the protocol described in general procedure (VIII) starting from (2R,12S,12aR)-8-methoxy-1,2,3,5,6,11,12,12a-octahydro-2,12-methanopyrrolo[1′,2′:1,2]azepino[4,5-b]indole ((R)-8a); ¹H NMR (400 MHz, MeOD) δ 7.10 (d, J=8.6 Hz, 1H), 6.82 (d, J=2.4 Hz, 1H), 6.65 (dd, J=8.6, 2.4 Hz, 1H), 4.07 (s, 1H), 3.62-3.38 (m, 4H), 3.29-3.14 (m, 2H), 3.01-2.92 (m, 1H), 2.82-2.76 (m, 1H), 2.49-2.38 (m, 1H), 2.08-1.97 (m, 2H) 1.52-1.44 (m, 1H). ESI-MS: measured m/z 255.20 [M+H]⁺. Purity by HPLC: 99% at 254 nm.

### Example 55: (2S,12R,12aS)-8-methoxy-11-methyl-1,2,3,5,6,11,12,12a-octahydro-2,12-methanopyrrolo[1′,2′:1,2]azepino[4,5-b]indole ((S)-10a)

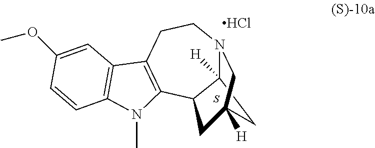

The title compound, (2S,12R,12aS)-8-methoxy-11-methyl-1,2,3,5,6,11,12,12a-octahydro-2,12-methanopyrrolo[1′,2′:1,2]azepino[4,5-b]indole ((S)-10a), was prepared (18 mg, 41% yield) according to the protocol described in general procedure (VII); ¹H NMR (400 MHz, MeOD) δ 7.23 (d, J=8.9 Hz, 1H), 6.99 (d, J=2.4 Hz, 1H), 6.83 (dd, J=8.9, 2.4 Hz, 1H), 4.34 (s, 1H), 3.85-3.79 (m, 4H), 3.76-3.67 (m, 2H), 3.66 (s, 3H), 3.64-3.55 (m, 2H), 3.24-3.18 (m, 2H), 2.90 (s, 1H), 2.62-2.52 (m, 1H), 2.23-2.06 (m, 2H), 1.53-1.47 (m, 1H). ¹³C NMR, DEPT-135 (101 MHz, MeOD) δ 111.29 (CH), 109.55 (CH), 99.41 (CH), 65.67 (CH), 55.95 (CH₂), 54.90 (CH), 52.03 (CH₂), 38.34 (CH₂), 36.64 (CH), 36.51 (CH), 34.41 (CH₂), 28.45 (CH), 17.62 (CH₂). ESI-MS: measured m/z 283.27 [M+H]⁺. Purity by HPLC: 97.66% at 254 nm.

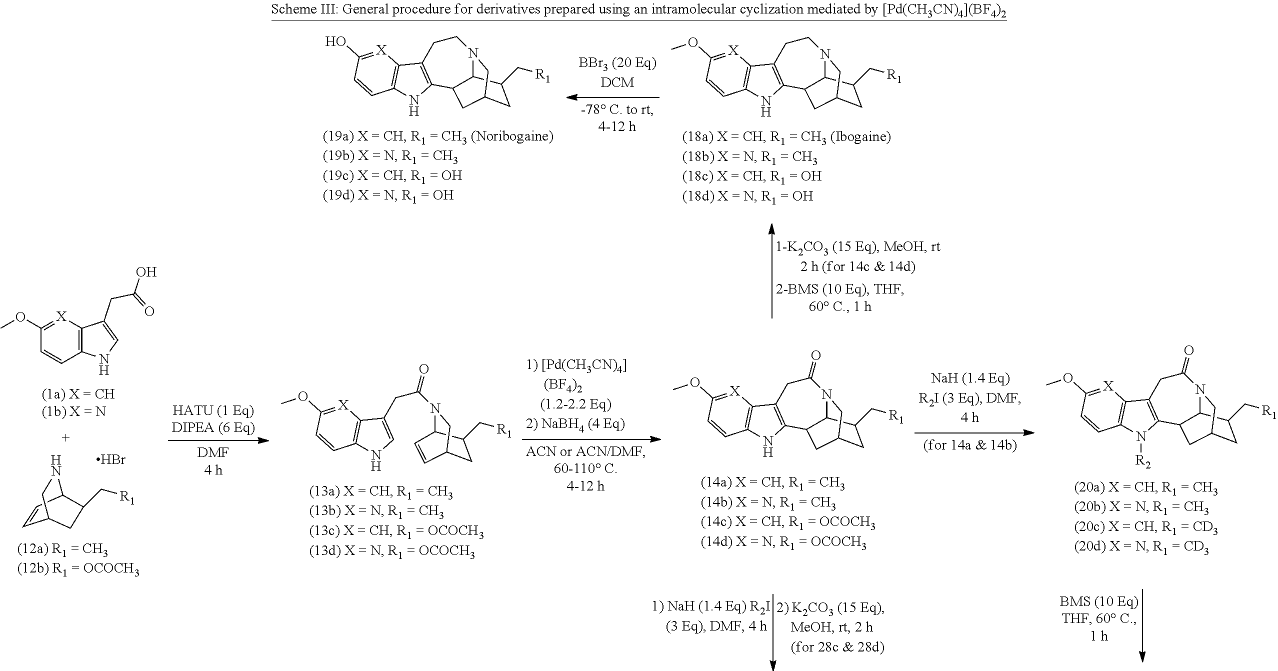

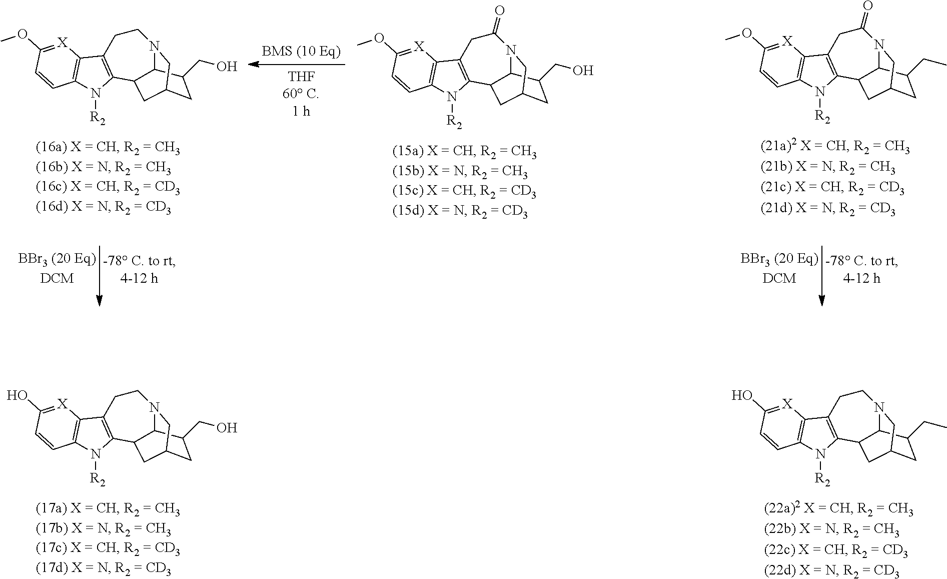

### General Procedure (XII); HATU Mediated Amide Formation Between Substituted Isoquinuclidine and Substituted Heteroaryl Acetic Acid (13)

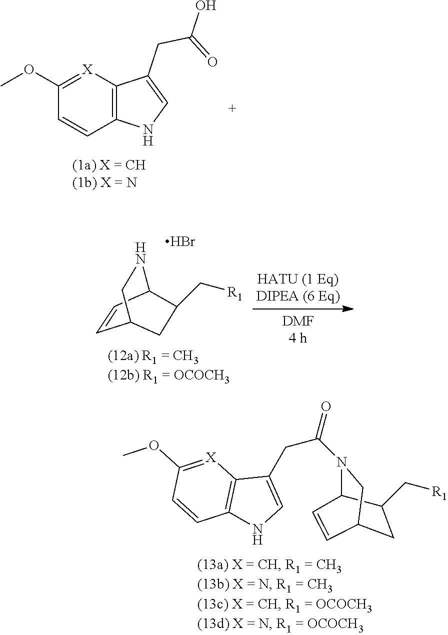

Under an inert atmosphere of argon gas, substituted heteroaryl acetic acid (1) (1 eq) and DIPEA (6 eq) were suspended in DMF (0.15 M) and cooled to 5-10° C. Subsequently, HATU (1 eq) was added. The reaction was stirred for 5 minutes and then the substituted isoquinuclidine (12) was added. The reaction was stirred for 4 h at rt. DCM was added and the organic phase washed with a sodium bicarbonate solution (5%, aqueous), water, brine, and dried with Na₂SO₄. The crude compound was used in the next step without purification or purified by normal phase silica gel column chromatography, running a mobile phase of DCM/Methanol.

### General Procedure (XIII); Intramolecular Cyclization Using [Pd(CH₃CN)₄](BF₄)₂ (14)

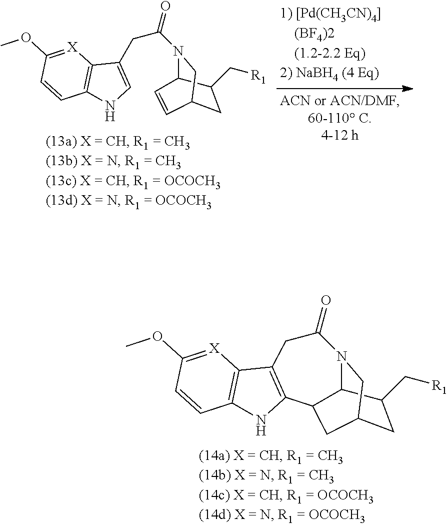

Under an inert atmosphere of argon gas, the appropriately substituted heteroaryl derivative (13) (X=CH, 1 eq) was placed in a microwave vial and suspended in acetonitrile (0.2 M, when X=CH) or in acetonitrile:DMF (4:1, 0.1 M, when X=N). Then tetrakis(acetonitrile)palladium(II) tetrafluoroborate (1.2 eq when X=CH or 2.2 eq when X=N) was added and the reaction was stirred for 4 h at 110° C. (when X=N) and 4 h at 60° C. and a further 20 h at rt (when X=CH). The suspension was cooled to −20° C., methanol (1 mL) was added followed by NaBH₄ (4 eq) in three portions. The reaction was warmed to rt over 1 h and stirred 2 h at rt. The reaction was filtered through celite and concentrated under reduced pressure. DCM was added and the organic phase washed with a sodium bicarbonate solution (5%, aqueous), water, brine, dried with Na₂SO₄ and concentrated. The crude compound was used in the next step without purification or purified by normal phase silica gel column chromatography, running a mobile phase of DCM/Methanol.

### General Procedure (XIV); Alkylation Using Iodomethane (R₂=CH₃) or Iodomethane-d3 (R₂=CD₃)

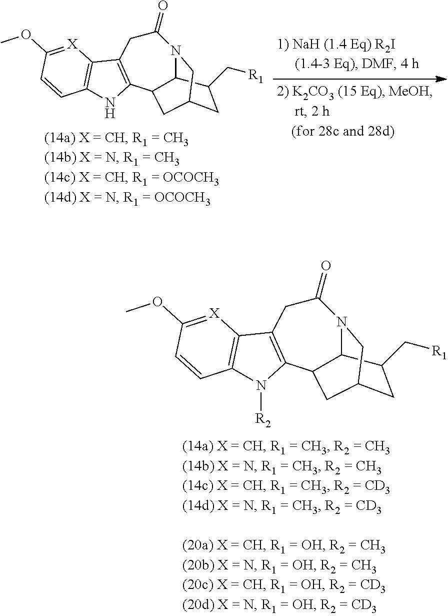

Under an inert atmosphere of argon gas, the appropriately substituted heteroaryl derivative (14) (1 eq) was suspended in THF:DMF (3:2, 0.3 M) cooled to 5° C. Then sodium hydride (60% dispersion in mineral oil, 1.4 eq) was added and stirred for 5 minutes. Then R₂-I (1.4-3 eq) was added and stirred for 3 h at rt. For 28c and 28d, methanol (5 mL) and K₂CO₃ (3 eq) were added to remove the acetate protecting group. After 1 h the reaction was concentrated under reduced pressure. DCM was added and the organic phase washed with water, brine, dried with Na₂SO₄ and concentrated. The crude compound was used in the next step without purification or purified by normal phase silica gel column chromatography, running a mobile phase of DCM/Methanol.

### General Procedure (XV); Borane Dimethyl Sulfide Complex (BMS) Mediated Reduction

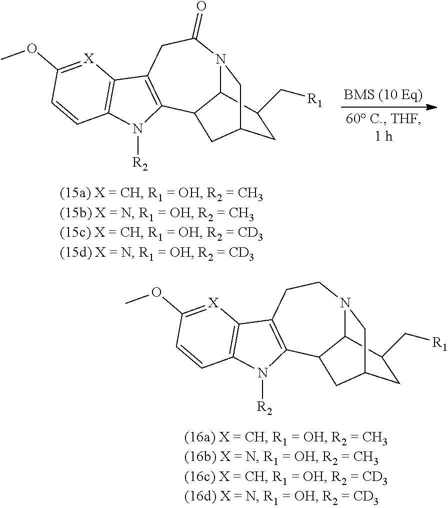

For compounds (14c-d), general procedure XIV was performed to remove the acetate protecting group, prior to reduction.

Under an inert atmosphere of argon gas, the appropriately substituted heteroaryl derivative (14, 15, or 20) (1 eq) was suspended in THF (0.015 M) and cooled to 0° C. Then BMS (10 eq) was added and the reaction was gradually warmed to 60° C. and stirred for 1 h. Reaction was cooled to 0° C. and methanol (1 mL) was added and the solution was concentrated under reduced pressure. 1M aqueous HCl was added (1 mL) then excess sodium bicarbonate solution (5%, aqueous). The aqueous phase was extracted with DCM. The organic phase washed with water, brine, dried with Na₂SO₄ and concentrated. The crude compound was used in the next step without purification or purified by normal phase silica gel column chromatography, running a mobile phase of DCM/Methanol. The purified compound was converted to the hydrochloride salt by treatment with 1N HCl in diethyl ether and was concentrated under reduced pressure or the purified compound was converted to the formate salt by treatment with formic acid in methanol and was concentrated under reduced pressure. Alternatively, the crude compound was purified by reverse phase (C18) column chromatography, running a mobile phase of water/methanol with 0.1% formic acid.

### General Procedure (XVI); BBr₃ Mediated Demethylation

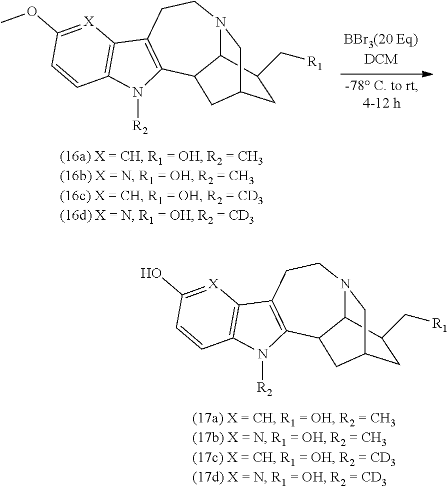

Under an inert atmosphere of argon gas, the appropriately substituted heteroaryl derivative (16, 18, or 21) (1 eq) was dissolved in DCM (0.4 M, X=CH) or DCM:1,2-DCE (3:1, 0.1 M, X=N) and cooled to −78° C. Then BBr₃ (20 eq) was added and the reaction was gradually warmed to rt and stirred for 2-24 h. Then cooled to 0° C. and water (3 mL) was added. Reaction was concentrated and purified by reverse phase (C18) column chromatography, running a mobile phase of water/methanol or water/acetonitrile with 0.1% formic acid.

### General Procedure (XVII); Acetate Removal

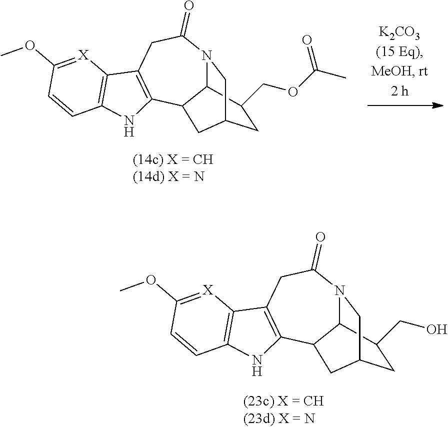

Under an inert atmosphere of argon gas, the appropriately substituted heteroaryl derivative (14c-d) (1 eq) was dissolved methanol (0.12 M) then K₂CO₃ (15 eq) was added. Reaction was stirred for 2 h, filtered and concentrated. DCM was added and the organic phase washed with water, brine, dried with Na₂SO₄ and concentrated. The crude compound was used in the next step without purification or purified by normal phase silica gel column chromatography, running a mobile phase of DCM/Methanol.

### Example 56: Synthesis of racemic (exo-benzyl 7-(hydroxymethyl)-2-azabicyclo[2.2.2]oct-5-ene-2-carboxylate (25)

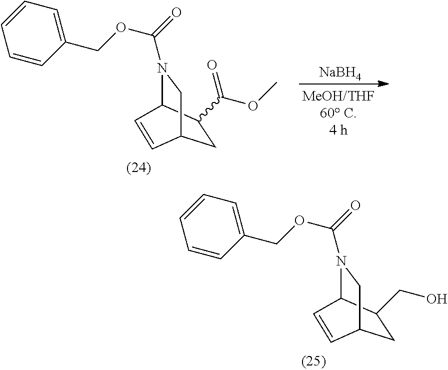 [SMILES: COC(=O)C1CC2C=CC1N(C(=O)OCc1ccccc1)C2 ; "NaBH4" ; O=C(OCc1ccccc1)N1CC2C=CC1C(CO)C2]

Compound (24) (2.25 g, 7.47 mmol) was dissolved in THF (30 mL) and sodium borohydride (3.3 g, 87.2 mmol) was added. The suspension was heated to 60° C. and methanol (5 mL) was added dropwise over 1 h and then stirred for 3 h at 60° C. Reaction was cooled to 0° C. and methanol (20 mL) was added and stirred for 1 hour at rt (H₂ evolved). Then aqueous 1N HCl (5 mL) was added followed by saturated sodium bicarbonate (50 mL) and brine (100 mL). The aqueous phase was extracted with DCM (2×100 ml) and the combined organics were washed with brine, dried with Na₂SO₄ and concentrated. Purified by normal phase silica gel column chromatography, running a mobile phase of Hexane:Ethyl acetate 8:2 to 2:8 to give exo-(25) as a colorless oil (0.72 g, 35% yield). ¹H NMR (400 MHz, CDCl₃) δ 7.48-7.27 (m, 5H), 6.46 (m, 2H), 5.28-5.01 (m, 2H), 4.89-4.60 (m, 1H), 3.69 (m, 1H), 3.28-3.11 (m, 2H), 3.03 (m, 1H), 2.76-2.62 (m, 1H), 1.93 (m, 1H), 1.55 (m, 1H), 0.93-0.75 (m, 1H). ESI-MS: measured m/z 274.0. Characterization data for exo-(25) agrees with the literature.

### Example 57: Synthesis of racemic (exo-2-azabicyclo[2.2.2]oct-7-en-6-yl)methyl acetate hydrobromide (12b)

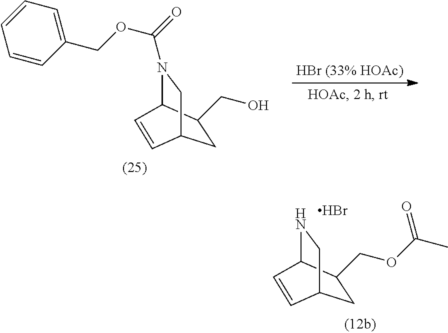 [SMILES: O=C(OCc1ccccc1)N1CC2C=CC1C(CO)C2 ; CC(=O)OCC1CC2C=CC1NC2 ; Br]

Exo-(25) (3.97 g, 14.63 mmol) was dissolved in acetic acid (25 mL) and hydrogen bromide 33% soln. in acetic acid (40.0 mL, 692.13 mmol) was added. The solution was stirred for 2 h and evaporated. To the oil was added hexanes (10 mL) and the hexane decanted. The oil was dried under high vacuum to give (12b) desired product (3.8 g, 93% yield). ¹H NMR (400 MHz, CDCl₃) δ 9.11 (br s, 1H), 8.56 (br s, 1H), 6.65 (m, 1H), 6.55 (m, 1H), 4.54-4.32 (m, 3H), 3.41 (s, 1H), 3.08-2.87 (m, 2H), 2.14 (s, 3H), 1.71 (t, J=12.5 Hz, 1H), 1.33 (m, 1H). ESI-MS: measured m/z 182.1.

### Example 58: Synthesis of racemic 1-(7-ethyl-2-azabicyclo[2.2.2]oct-5-en-2-yl)-2-(5-methoxy-1H-indol-3-yl)ethan-1-one (13a)

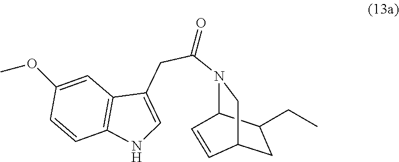 [SMILES: CCC1CC2C=CC1N(C(=O)Cc1c[nH]c3ccc(OC)cc13)C2]

The title compound, 1-(-7-ethyl-2-azabicyclo[2.2.2]oct-5-en-2-yl)-2-(5-methoxy-1H-indol-3-yl)ethan-1-one (13a), was prepared according to the protocol described in general procedure (IV) starting from (2,3,4,5,6-pentafluorophenyl) 2-(5-methoxy-1H-indol-3-yl)acetate to afford the desired product (175 mg, 90% yield). ¹H NMR (400 MHz, CDCl₃) δ 7.99 (br s, 1H), 7.27-7.21 (m, 1H), 7.13-6.98 (m, 2H), 6.90-6.82 (m, 1H), 6.54-6.44 (m, 0.6H), 6.40-6.30 (m, 1H), 6.20-6.11 (m, 0.4H), 5.24-5.17 (m, 0.4H), 4.34-4.27 (m, 0.6H), 3.92-3.83 (m, 3.5H), 3.81-3.65 (m, 1.5H), 3.43-3.29 (m, 1H), 3.22-3.13 (m, 1H), 2.79-2.67 (m, 1H), 1.72-1.61 (m, 1H), 1.54-1.43 (m, 1H), 1.42-1.27 (m, 3H), 1.01-0.92 (m, 3H). ESI-MS: measured m/z 325.20 [M+1]⁺.

### Example 59: Synthesis of racemic exo-7-ethyl-2-azabicyclo[2.2.2]oct-5-en-2-yl)-2-(5-methoxy-1H-pyrrolo[3,2-b]pyridin-3-yl)ethan-1-one (13b)

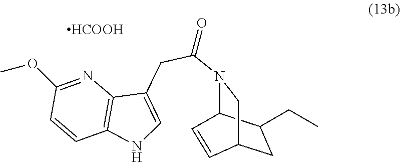 [SMILES: CCC1CC2C=CC1N(C(=O)Cc1c[nH]c3ccc(OC)nc13)C2 ; "·HCOOH"]

The title compound, exo-7-ethyl-2-azabicyclo[2.2.2]oct-5-en-2-yl)-2-(5-methoxy-1H-pyrrolo[3,2-b]pyridin-3-yl)ethan-1-one (13b), was prepared (350 mg, 47% yield) according to the protocol described in general procedure (XII); ¹H NMR (400 MHz, CDCl₃) δ 8.76-8.41 (m, 1H), 8.12 (s, 1H), 7.59-7.42 (m, 1H), 6.64-6.50 (m, 1H), 6.48-6.31 (m, 1H), 6.27-6.19 (m, 0.5H), 5.99-5.90 (m, 0.5H), 5.18-5.08 (m, 0.5H), 5.89-5.81 (m, 0.5H), 4.16 4.03 (m, 0.5H), 4.02 3.78 (m, 3.5H), 3.84 3.60 (m, 2.5H), 3.47 2.99 (m, 1.5H), 2.70-2.64 (m, 1H), 1.72-1.56 (m, 2H), 1.55-1.22 (m, 3H), 1.06-0.85 (m, 3H). ESI-MS: measured m/z 326.27 [M+H]⁺.

### Example 60: Synthesis of racemic exo-2-(2-(5-methoxy-1H-indol-3-yl)acetyl)-2-azabicyclo[2.2.2]oct-7-en-6-yl)methyl acetate (13c)

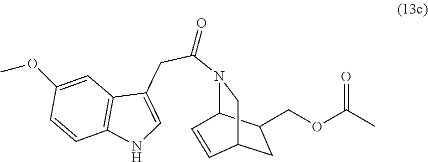 [SMILES: COc1ccc2[nH]cc(CC(=O)N3CC4C=CC3C(COC(C)=O)C4)c2c1]

The title compound, exo-2-(2-(5-methoxy-1H-indol-3-yl)acetyl)-2-azabicyclo[2.2.2]oct-7-en-6-yl)methyl acetate (13c), was prepared according to the protocol described in general procedure (IV) starting from (2,3,4,5,6-pentafluorophenyl) 2-(5-methoxy-1H-indol-3-yl)acetate to afford the desired product (300 mg, 86% yield). ¹H NMR (400 MHz, CDCl₃) δ 8.38-8.24 (m, 1H), 7.25-7.16 (m, 1H), 7.09-6.93 (m, 2H), 6.88-6.78 (m, 1H), 6.52-6.42 (m, 0.5H), 6.41-6.32 (m, 1H), 6.16-6.02 (m, 0.5H), 5.33-5.25 (m, 0.5H), 4.52-4.45 (m, 0.5H), 4.31-4.23 (m, 0.5H), 4.02-3.94 (m, 0.5H), 3.89-3.78 (m, 3H), 3.75-3.58 (m, 2H), 3.35-3.24 (m, 1H), 3.22-3.09 (m, 1H), 2.83-2.79 (m, 3H), 2.09-1.91 (m, 3H), 1.69-1.54 (m, 1H), 1.07-0.89 (m, 1H). ESI-MS: measured m/z 369.13 [M+H]⁺. Purity by HPLC: 97% at 254 nm.

### Example 61: Synthesis of racemic exo-2-(2-(5-methoxy-1H-pyrrolo[3,2-b]pyridin-3-yl)acetyl)-2-azabicyclo[2.2.2]oct-7-en-6-yl)methyl acetate (13d)

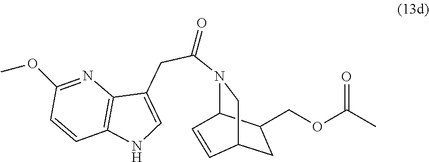 [SMILES: COc1ccc2[nH]cc(CC(=O)N3CC4C=CC3C(COC(C)=O)C4)c2n1]

The title compound, exo-2-(2-(5-methoxy-1H-pyrrolo[3,2-b]pyridin-3-yl)acetyl)-2-azabicyclo[2.2.2]oct-7-en-6-yl)methyl acetate (13d), was prepared according to the protocol described in general procedure (XII) (350 mg, 93% yield). ¹H NMR (400 MHz, CDCl₃) δ 8.79-8.67 (m, 1H), 7.54-7.42 (m, 1H), 7.24-7.17 (m, 1H), 6.61-6.39 (m, 2H), 6.36-6.29 (m, 0.5H), 6.03-5.95 (m, 0.5H), 5.34-5.28 (m, 0.5H), 5.18-5.08 (m, 0.5H), 4.33-4.24 (m, 0.5H), 4.16-4.08 (m, 0.5H), 4.03-3.93 (m, 3H), 3.88-3.65 (m, 3H), 3.64-3.57 (m, 0.5H), 3.47-3.40 (m, 0.5H), 3.30-3.23 (m, 0.5H), 3.14-3.07 (m, 0.5H), 2.77 (br s, 1H), 2.13-1.95 (m, 4H), 1.69-1.59 (m, 1H), 1.08-0.97 (m, 1H). ESI-MS: measured m/z 370.2 [M+H]⁺. Purity by HPLC: 78% at 254 nm.

### Example 62: Synthesis of racemic7-ethyl-2-methoxy-5,6,6a,7,8,9,10,13-octahydro-12H-6,9-methanopyrido[1′,2′:1,2]azepino[4,5-b]indol-12-one (14a)

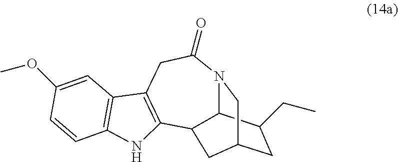 [SMILES: CCC1CC2CC3c4[nH]c5ccc(OC)cc5c4CC(=O)N(C2)C13]

The title compound, 7-ethyl-2-methoxy-5,6,6a,7,8,9,10,13-octahydro-12H-6,9-methanopyrido[1′,2′:1,2]azepino[4,5-b]indol-12-one (14a), was prepared according to the protocol described in general procedure (XIII) starting from compound (13a) to afford the desired product (67 mg, 39% yield). ¹H NMR (400 MHz, CDCl₃) δ 7.92 (s, 1H), 7.16 (d, J=8.7 Hz, 1H), 6.93 (d, J=2.4 Hz, 1H), 6.80 (dd, J=8.7, 2.4 Hz, 1H), 4.17 (s, 1H), 4.04-3.94 (m, 1H), 3.89-3.81 (m, 4H), 3.76-3.66 (m, 1H), 3.20 (d, J=11.9 Hz, 1H), 3.06-2.97 (m, 1H), 2.17-2.04 (m, 1H), 1.99-1.86 (m, 1H), 1.77-1.07 (m, 6H), 1.02 (t, J=7.3 Hz, 3H). ESI-MS: measured m/z 325.20 [M+H]⁺. Purity by HPLC: 98% at 254 nm.

### Example 63: Synthesis of racemic 7-ethyl-2-methoxy-5,6,6a,7,8,9,10,13-octahydro-12H-6,9-methanopyrido[1,2-a]pyrido[2′,3′:4,5]pyrrolo[2,3-d]azepin-12-one (14b)

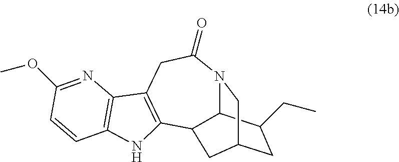 [SMILES: CCC1CC2CC3c4[nH]c5ccc(OC)nc5c4CC(=O)N(C2)C13]

The title compound, 7-ethyl-2-methoxy-5,6,6a,7,8,9,10,13-octahydro-12H-6,9-methanopyrido[1,2-a]pyrido[2′,3′:4,5]pyrrolo[2,3-d]azepin-12-one (14b), was prepared according to the protocol described in general procedure (XIII) starting from compound (13b) and isolated as white solid (56 mg, 28% yield). ¹H NMR (400 MHz, CDCl₃) δ 7.76 (br s, 1H), 7.42 (d, J=8.6 Hz, 1H), 6.52 (d, J=8.6 Hz, 1H), 4.17 (s, 1H), 4.11 (m, 1H), 3.97 (s, 3H), 3.91-3.79 (m, 2H), 3.17 (d, J=11.9 Hz, 1H), 3.03 (m, 1H), 2.06 (s, 1H), 1.89 (m, 1H), 1.62 (m, 6H), 0.99 (t, J=7.4 Hz, 3H). ESI-MS: measured m/z 326.33 [M+H]⁺. Purity by HPLC: 95% at 254 nm.

### Example 64: Synthesis of racemic (2-methoxy-12-oxo-6,6a,7,8,9,10,12,13-octahydro-5H-6,9-methanopyrido[1′,2′:1,2]azepino[4,5-b]indol-7-yl)methyl acetate (14c)

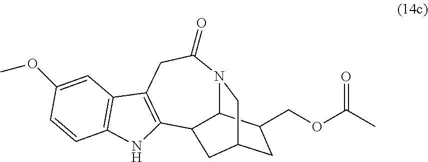 [SMILES: COc1ccc2[nH]c3c(c2c1)CC(=O)N1CC2CC(COC(C)=O)C1C3C2]

The title compound, (2-methoxy-12-oxo-6,6a,7,8,9,10,12,13-octahydro-5H-6,9-methanopyrido[1′,2′:1,2]azepino[4,5-b]indol-7-yl)methyl acetate (14c), was prepared according to the protocol described in general procedure (XIII) starting from compound (13c) and isolated as white solid (56 mg, 55% yield). ¹H NMR (400 MHz, CDCl₃) δ 8.03 (br s, 1H), 7.15 (d, J=8.7 Hz, 1H), 6.90 (d, J=2.4 Hz, 1H), 6.78 (dd, J=8.7, 2.4 Hz, 1H), 4.22 (m, 2H), 4.03 (m, 1H), 3.93 (m, 1H), 3.88-3.82 (m, 1H), 3.80 (s, 3H), 3.69 (m, 1H), 3.12 (m, 1H), 2.97 (m, 1H), 2.36 (m, 2H), 2.13 (m, 1H), 2.08 (s, 3H), 1.91-1.82 (m, 2H), 1.49-1.40 (m, 2H). ¹³C NMR DEPT-135 (101 MHz, CDCl₃) δ 112.05 (CH), 111.23 (CH), 100.09 (CH), 66.25 (CH₂), 55.85 (CH₃), 49.56 (CH₂), 49.29 (CH), 36.10 (CH), 35.47 (CH), 32.92 (CH₂), 32.12 (CH₂), 27.05 (CH₂), 26.87 (CH), 20.93 (CH₃). ESI-MS: measured m/z 369.2 [M+H]⁺. Purity by HPLC: 94.0% at 254 nm.

### Example 65: Synthesis of racemic (2-methoxy-5-methyl-12-oxo-6,6a,7,8,9,10,12,13-octahydro-5H-6,9-methanopyrido[1′,2′:1,2]azepino[4,5-b]indol-7-yl)methyl acetate (26)

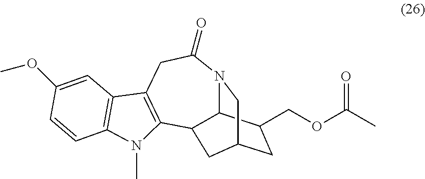 [SMILES: COc1ccc2c(c1)c1c(n2C)C2CC3CC(COC(C)=O)C2N(C3)C(=O)C1]

The title compound, (2-methoxy-5-methyl-12-oxo-6,6a,7,8,9,10,12,13-octahydro-5H-6,9-methanopyrido[1′,2′:1,2]azepino[4,5-b]indol-7-yl)methyl acetate (26), was prepared according to the protocol described in general procedure (XIV) starting from compound (14c) without deprotection of acetate group to afford the desired product (35 mg, 33% yield). ¹H NMR (400 MHz, CDCl₃) δ 7.16 (d, J=8.8 Hz, 1H), 7.00 (d, J=2.4 Hz, 1H), 6.86 (dd, J=8.8, 2.4 Hz, 1H), 4.34 (m, 1H), 4.29 (m, 1H), 4.05 (m, 1H), 3.99 (m, 1H), 3.90 (m, 1H), 3.87 (s, 3H), 3.75 (m, 1H), 3.63 (s, 3H), 3.19-3.14 (m, 1H), 3.10 (m, 1H), 2.47 (m, 1H), 2.30-2.19 (m, 1H), 2.19-2.13 (m, 1H), 2.09 (s, 3H), 1.98 (m, 1H), 1.54-1.44 (m, 2H). ¹³C NMR DEPT-135 (101 MHz, CDCl₃) δ 111.75 (CH), 109.45 (CH), 100.08 (CH), 66.18 (CH₂), 55.94 (CH₃), 49.43 (CH₂), 49.31 (CH), 36.37 (CH), 34.68 (CH), 33.08 (CH₂), 32.01 (CH₂), 29.91 (CH₃), 27.11 (CH₂), 27.00 (CH), 20.91 (CH₃). ESI-MS: measured m/z 383.27 [M+H]⁺. Purity by HPLC: 98% at 254 nm.

### Example 66: Synthesis of racemic (2-methoxy-5-(methyl-d3)-12-oxo-6,6a,7,8,9,10,12,13-octahydro-5H-6,9-methanopyrido[1′,2′:1,2]azepino[4,5-b]indol-7-yl)methyl acetate (27)

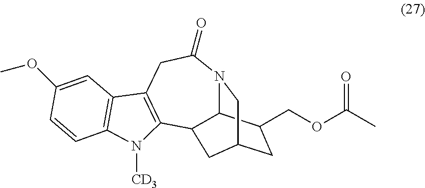 [SMILES: [2H]C([2H])([2H])n1c2c(c3cc(OC)ccc31)CC(=O)N1CC3CC(COC(C)=O)C1C2C3]

The title compound, (2-methoxy-5-(methyl-d3)-12-oxo-6,6a,7,8,9,10,12,13-octahydro-5H-6,9-methanopyrido[1′,2′:1,2]azepino[4,5-b]indol-7-yl)methyl acetate (27), was prepared according to the protocol described in general procedure (XIV) starting from compound (14c) to afford the desired product (45 mg, 43% yield). ¹H NMR (400 MHz, CDCl₃) δ 7.15 (d, J=8.8 Hz, 1H), 7.00 (d, J=2.4 Hz, 1H), 6.86 (dd, J=8.8, 2.4 Hz, 1H), 4.34 (m, 1H), 4.29 (m, 1H), 4.05 (m, 1H), 3.98 (m, 1H), 3.90 (m, 1H), 3.87 (s, 3H), 3.75 (m, 1H), 3.15 (m, 1H), 3.09 (m, 1H), 2.47 (m, 1H), 2.24 (m, 1H), 2.15 (m, 1H), 2.09 (s, 3H), 1.98 (m, 1H), 1.48 (m, 2H). ¹³C NMR DEPT-135 (101 MHz, CDCl₃) δ 111.73 (CH), 109.44 (CH), 100.08 (CH), 66.18 (CH₂), 55.94 (CH₃), 49.43 (CH₂), 49.31 (CH), 36.36 (CH), 34.68 (CH), 33.08 (CH₂), 32.00 (CH₂), 27.11 (CH₂), 26.99 (CH), 20.91 (CH₃). ESI-MS: measured m/z 386.27 [M+H]⁺. Purity by HPLC: 98% at 254 nm.

### Example 67: Synthesis of racemic (2-methoxy-12-oxo-6,6a,7,8,9,10,12,13-octahydro-5H-6,9-methanopyrido[1,2-a]pyrido[2′,3′:4,5]pyrrolo[2,3-d]azepin-7-yl)methyl acetate (14d)

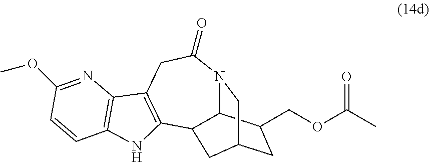 [SMILES: COc1ccc2[nH]c3c(c2n1)CC(=O)N1CC2CC(COC(C)=O)C1C3C2]

The title compound, (2-methoxy-12-oxo-6,6a,7,8,9,10,12,13-octahydro-5H-6,9-methanopyrido[1,2-a]pyrido[2′,3′:4,5]pyrrolo[2,3-d]azepin-7-yl)methyl acetate (14d), was prepared according to the protocol described in general procedure (XIII) starting from compound (13d) to afford the desired product (45 mg, 50% yield). ESI-MS: measured m/z 370.2 [M+H]⁺. Purity by HPLC: 90% at 254 nm.

### Example 68: Synthesis of racemic 7-(hydroxymethyl)-2-methoxy-5,6,6a,7,8,9,10,13-octahydro-12H-6,9-methanopyrido[1′,2′:1,2]azepino[4,5-b]indol-12-one (15c)

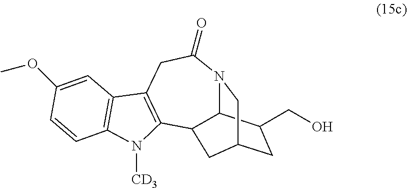 [SMILES: [2H]C([2H])([2H])n1c2c(c3cc(OC)ccc31)CC(=O)N1CC3CC(CO)C1C2C3]

The title compound, 7-(hydroxymethyl)-2-methoxy-5,6,6a,7,8,9,10,13-octahydro-12H-6,9-methanopyrido[1′,2′:1,2]azepino[4,5-b]indol-12-one (15c), was prepared according to the protocol described in general procedure (XVII) starting from compound (14c) to afford the desired product (15 mg, 85% yield). ¹H NMR (400 MHz, MeOD) δ 7.16 (d, J=8.7 Hz, 1H), 6.97 (d, J=2.4 Hz, 1H), 6.72 (dd, J=8.7, 2.4 Hz, 1H), 4.48 (m, 1H), 4.11 (m, 1H), 3.83 (s, 3H), 3.76 (m, 1H), 3.67-3.56 (m, 3H), 3.12 (m, 2H), 2.30 (m, 2H), 2.07 (m, 1H), 2.00-1.87 (m, 1H), 1.33 (m, 2H). ¹³C NMR; DEPT-135 (101 MHz, MeOD) δ 112.00 (CH), 111.80 (CH), 100.48 (CH), 65.24 (CH₂), 56.00 (CH₃), 50.77 (CH₂), 50.31 (CH), 40.37 (CH), 36.40 (CH), 33.39 (CH₂), 33.02 (CH₂), 28.36 (CH), 27.60 (CH₂). ESI-MS: measured m/z 327.2 [M+H]⁺. Purity by HPLC: 98.6% at 254 nm.

### Example 69: Synthesis of racemic 7-ethyl-2-methoxy-6,6a,7,8,9,10,12,13-octahydro-5H-6,9-methanopyrido[1,2-a]pyrido[2′,3′:4,5]pyrrolo[2,3-d]azepine formate (18b)

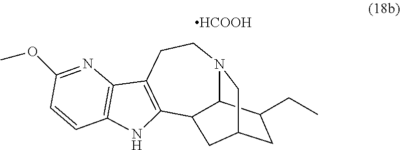 [SMILES: "·HCOOH" ; CCC1CC2CC3c4[nH]c5ccc(OC)nc5c4CCN(C2)C13]

The title compound, 7-ethyl-2-methoxy-6,6a,7,8,9,10,12,13-octahydro-5H-6,9-methanopyrido[1,2-a]pyrido[2′,3′:4,5]pyrrolo[2,3-d]azepine formate (18b), was prepared according to the protocol described in general procedure (XV) starting from compound (14b) and isolated as white solid (6.0 mg, 32% yield). ¹H NMR (400 MHz, MeOD) δ 7.58 (d, J=8.7 Hz, 1H), 6.55 (d, J=8.6 Hz, 1H), 3.93 (s, 3H), 3.71-3.64 (m, 1H), 3.62-3.51 (m, 2H), 3.49-3.34 (m, 4H), 2.34 (m, 1H), 2.20-2.10 (m, 2H), 2.05-1.98 (m, 1H), 1.82-1.73 (m, 2H), 1.69-1.59 (m, 2H), 1.43-1.38 (m, 1H), 1.04 (t, J=7.2 Hz, 3H). ¹³C NMR; DEPT-135 (101 MHz, MeOD) δ 121.50 (CH), 103.71 (CH), 59.63 (CH₃), 56.11 (CH₂), 52.44 (CH), 50.66 (CH₂), 39.17 (CH), 35.73 (CH), 31.13 (CH₂), 28.88 (CH₂), 26.05 (CH₂), 23.99 (CH), 16.77 (CH₂), 10.54 (CH₃). ESI-MS: measured m/z 312.27 [M+H]⁺. Purity by HPLC: 98.2% at 254 nm.

### Example 70: Synthesis of racemic 7-ethyl-6,6a,7,8,9,10,12,13-octahydro-5H-6,9-methanopyrido[1,2-a]pyrido[2′,3′:4,5]pyrrolo[2,3-d]azepin-2-ol (19b)

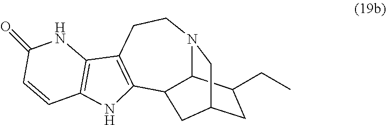 [SMILES: CCC1CC2CC3c4[nH]c5ccc(=O)[nH]c5c4CCN(C2)C13]

The title compound, 7-ethyl-6,6a,7,8,9,10,12,13-octahydro-5H-6,9-methanopyrido[1,2-a]pyrido[2′,3′:4,5]pyrrolo[2,3-d]azepin-2-ol (19b), was prepared according to the protocol described in general procedure (XVI) starting from compound (32b) and isolated as white solid (2.4 mg, 23% yield). ¹H NMR (400 MHz, MeOD) δ 7.64 (d, J=9.1 Hz, 1H), 6.23 (d, J=9.1 Hz, 1H), 3.69-3.61 (m, 1H), 3.57-3.48 (m, 1H), 3.35-3.10 (m, 5H), 3.05-2.98 (m, 1H), 2.28 (m, 1H), 2.11-2.03 (m, 2H), 1.92-1.87 (m, 1H), 1.78-1.72 (m, 2H), 1.67-1.59 (m, 2H), 1.02 (t, J=7.3 Hz, 3H). ESI-MS: measured m/z 298.27 [M+H]⁺. Purity by HPLC: 97.2% at 254 nm.

### Example 71: Synthesis of racemic (2-methoxy-6,6a,7,8,9,10,12,13-octahydro-5H-6,9-methanopyrido[1′,2′:1,2]azepino[4,5-b]indol-7-yl)methanol hydrochloride (18c)

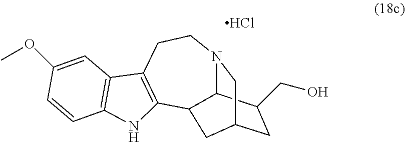 [SMILES: Cl ; COc1ccc2[nH]c3c(c2c1)CCN1CC2CC(CO)C1C3C2]

The title compound, (2-methoxy-6,6a,7,8,9,10,12,13-octahydro-5H-6,9-methanopyrido[1′,2′:1,2]azepino[4,5-b]indol-7-yl)methanol hydrochloride (18c), was prepared according to the protocol described in general procedure (XV) starting from compound (14c). The compound was purified by silica chromatography and the desired fractions were concentrated and acidified with excess HCl in diethyl ether to give a light yellow oil (15.2 mg, 70% yield). ¹H NMR (400 MHz, MeOD) δ 7.19 (d, J=8.8 Hz, 1H), 6.97 (d, J=2.4 Hz, 1H), 6.76 (m, 1H), 4.03 (m, 1H), 3.85 (m, 1H), 3.83 (s, 3H), 3.75 (m, 1H), 3.68 (m, 1H), 3.60 (m, 1H), 3.51 (m, 1H), 3.47-3.38 (m, 2H), 3.29-3.17 (m, 2H), 2.33 (m, 1H), 2.23 (m, 2H), 2.09 (m, 1H), 1.84 (m, 1H), 1.79-1.68 (m, 1H). ¹³C NMR; DEPT-135 (101 MHz, MeOD) δ 111.26 (CH), 111.09 (CH), 99.60 (CH), 63.95 (CH₂), 60.79 (CH), 54.91 (CH₃), 54.30 (CH₂), 50.07 (CH₂), 36.51 (CH), 34.77 (CH), 31.35 (CH₂), 24.80 (CH₂), 24.04 (CH), 17.90 (CH₂). ESI-MS: measured m/z 313.3 [M+H]⁺. Purity by HPLC: 98.2% at 254 nm.

### Example 72: Synthesis of racemic (2-methoxy-5-methyl-6,6a,7,8,9,10,12,13-octahydro-5H-6,9-methanopyrido [1′,2′:1,2]azepino[4,5-b]indol-7-yl)methanol formate (16a)

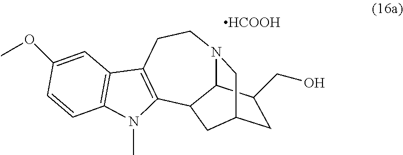 [SMILES: "·HCOOH" ; COc1ccc2c(c1)c1c(n2C)C2CC3CC(CO)C2N(CC1)C3]

The title compound, (2-methoxy-5-methyl-6,6a,7,8,9,10,12,13-octahydro-5H-6,9-methanopyrido[1′,2′:1,2]azepino[4,5-b]indol-7-yl)methanol formate (16a), was prepared by following the general procedure (XVII) then followed the procedure described in (XV) to obtain the desired compound as colourless oil (13.6 mg, 40% yield). ¹H NMR (400 MHz, MeOD) δ 7.21 (d, J=8.9 Hz, 1H), 6.98 (d, J=2.4 Hz, 1H), 6.82 (dd, J=8.9, 2.4 Hz, 1H), 4.05-3.95 (m, 1H), 3.82 (m, 5H), 3.67 (m, 4H), 3.63-3.54 (m, 2H), 3.54-3.47 (m, 1H), 3.40 (m, 1H), 3.24 (m, 2H), 2.38 (m, 1H), 2.33-2.26 (m, 1H), 2.24 (m, 1H), 2.09 (m, 1H), 1.81 (m, 1H), 1.66 (m, 1H). ¹³C NMR; DEPT-135 (101 MHz, MeOD) δ 111.26 (CH), 109.55 (CH), 99.72 (CH), 63.99 (CH₂), 59.97 (CH), 54.91 (CH₃), 54.66 (CH₂), 50.76 (CH₂), 37.01 (CH), 32.88 (CH), 30.86 (CH₂), 28.58 (CH₃), 24.92 (CH₂), 24.17 (CH), 18.19 (CH₂). ESI-MS: measured m/z 327.27 [M+H]⁺. Purity by HPLC: 97.8% at 254 nm.

### Example 73: Synthesis of racemic 7-(hydroxymethyl)-5-methyl-6,6a,7,8,9,10,12,13-octahydro-5H-6,9-methanopyrido[1′,2′:1,2]azepino[4,5-b]indol-2-ol formate (17a)

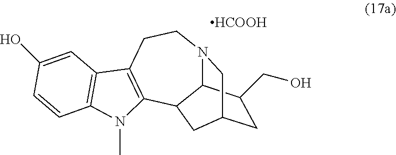 [SMILES: "·HCOOH" ; Cn1c2c(c3cc(O)ccc31)CCN1CC3CC(CO)C1C2C3]

The title compound, 7-(hydroxymethyl)-5-methyl-6,6a,7,8,9,10,12,13-octahydro-5H-6,9-methanopyrido[1′,2′:1,2]azepino[4,5-b]indol-2-ol formate (17a), was prepared according to the protocol described in general procedure (XVI) starting with (16a) and isolated as colourless oil (17 mg, 64.5% yield). ¹H NMR (400 MHz, MeOD) δ 7.14 (d, J=8.8 Hz, 1H), 6.84 (t, J=1.8 Hz, 1H), 6.73 (dd, J=8.7, 1.8 Hz, 1H), 4.03 (m, 1H), 3.89-3.78 (m, 2H), 3.71 (m, 1H), 3.65 (s, 3H), 3.64-3.56 (m, 2H), 3.53 (m, 1H), 3.43-3.37 (m, 1H), 3.28-3.09 (m, 2H), 2.44-2.27 (m, 2H), 2.25 (m, 1H), 2.11 (m, 1H), 1.88-1.77 (m, 1H), 1.63 (m, 1H). ¹³C NMR; DEPT-135 (101 MHz, MeOD) δ 111.24 (CH), 109.37 (CH), 101.82 (CH), 63.98 (CH2), 60.39 (CH), 54.65 (CH₂), 50.79 (CH₂), 36.66 (CH), 32.56 (CH), 30.71 (CH₂), 28.62 (CH₃), 24.77 (CH₂), 24.05 (CH), 18.09 (CH₂). ESI-MS: measured m/z 313.27 [M+H]⁺. Purity by HPLC: 98.5% at 254 nm.

### Example 74: Synthesis of racemic (2-methoxy-5-(methyl-d3)-6,6a,7,8,9,10,12,13-octahydro-5H-6,9-methanopyrido[1′,2′:1,2]azepino[4,5-b]indol-7-yl)methanol formate (16c)

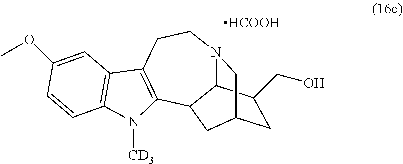 [SMILES: "·HCOOH" ; [2H]C([2H])([2H])n1c2c(c3cc(OC)ccc31)CCN1CC3CC(CO)C1C2C3]

The title compound, (2-methoxy-5-(methyl-d3)-6,6a,7,8,9,10,12,13-octahydro-5H-6,9-methanopyrido[1′,2′:1,2]azepino[4,5-b]indol-7-yl)methanol formate (16c), was prepared by removing the acetate group according to the protocol described in general procedure (XVII) then followed the procedure described in (XV) to obtain the desired compound as colourless oil (12.0 mg, 73% yield). ¹H NMR (400 MHz, MeOD) δ 7.21 (d, J=8.9 Hz, 1H), 6.98 (d, J=2.5 Hz, 1H), 6.82 (dd, J=8.9, 2.5 Hz, 1H), 4.00 (m, 1H), 3.83 (m, 1H), 3.82 (s, 3H), 3.70 (m, 1H), 3.66-3.55 (m, 2H), 3.52 (m, 1H), 3.40 (m, 1H), 3.24 (m, 2H), 2.44-2.37 (m, 1H), 2.37-2.27 (m, 2H), 2.24 (s, 1H), 2.15-2.05 (m, 1H), 1.85-1.77 (m, 1H), 1.65 (m, 1H). ¹³C NMR; DEPT-135 (101 MHz, MeOD) δ 111.27 (CH), 109.55 (CH), 99.71 (CH), 63.96 (CH₂), 60.06 (CH), 54.91 (CH₃), 54.69 (CH₂), 50.78 (CH₂), 36.94 (CH), 32.79 (CH), 30.82 (CH₂), 24.87 (CH₂), 24.13 (CH), 18.16 (CH₂). ESI-MS: measured m/z 330.3 [M+H]⁺. Purity by HPLC: 99.4% at 254 nm.

### Example 75: Synthesis of racemic (2-methoxy-6,6a,7,8,9,10,12,13-octahydro-5H-6,9-methanopyrido[1,2-a]pyrido[2′,3′:4,5]pyrrolo[2,3-d]azepin-7-yl)methanol formate (18d)

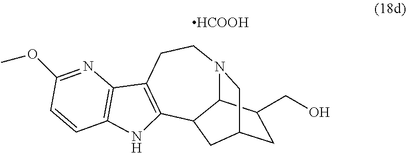 [SMILES: "·HCOOH" ; COc1ccc2[nH]c3c(c2n1)CCN1CC2CC(CO)C1C3C2]

The title compound, (2-methoxy-6,6a,7,8,9,10,12,13-octahydro-5H-6,9-methanopyrido[1,2-a]pyrido[2′,3′:4,5]pyrrolo[2,3-d]azepin-7-yl)methanol formate (18d), was prepared by removing the acetate group according to the protocol described in general procedure (XVII) then followed the procedure described in (XV) to obtain the desired compound as a colourless oil (11.0 mg, 32% yield). ¹H NMR (400 MHz, MeOD) δ 7.58 (d, J=8.5, 1H), 6.56 (d, J=8.7, 1H), 4.01 (m, 1H), 3.93 (m, 2H), 3.87 (m, 1H), 3.82 (m, 1H), 3.78 (m, 1H), 3.74 (m, 1H), 3.59 (m, 1H), 3.55-3.45 (m, 2H), 3.41 (m, 2H), 3.29-3.22 (m, 1H), 2.36 (m, 1H), 2.25 (m, 2H), 2.09 (m, 1H), 1.81 (m, 2H). ¹³C NMR (101 MHz, MeOD) δ 121.50 (CH), 103.88 (CH), 64.00 (CH₂), 60.47 (CH), 54.39 (CH₂), 52.42 (CH₃), 50.26 (CH₂), 36.59 (CH), 35.27 (CH), 31.22 (CH₂), 24.85 (CH₂), 24.06 (CH), 16.59 (CH₂). ESI-MS: measured m/z 314.27 [M+H]⁺. Purity by HPLC: 95.3% at 254 nm.

### Example 76: Synthesis of racemic 7-(hydroxymethyl)-6,6a,7,8,9,10,12,13-octahydro-5H-6,9-methanopyrido[1,2-a]pyrido[2′,3′:4,5]pyrrolo[2,3-d]azepin-2-ol formate (19d)

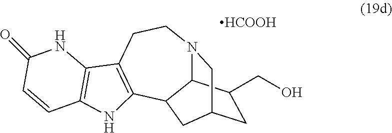 [SMILES: O=c1ccc2[nH]c3c(c2[nH]1)CCN1CC2CC(CO)C1C3C2 ; "·HCOOH"]

The title compound, 7-(hydroxymethyl)-6,6a,7,8,9,10,12,13-octahydro-5H-6,9-methanopyrido[1,2-a]pyrido[2′,3′:4,5]pyrrolo[2,3-d]azepin-2-ol (19d), was prepared according to the protocol described in general procedure (XVI) starting from compound (18d) to afford the desired product as a colourless oil (5.6 mg, 34% yield). ¹H NMR (400 MHz, MeOD) δ 7.64 (d, J=9.1 Hz, 1H), 6.23 (d, J=9.1 Hz, 1H), 3.94 (m, 1H), 3.76 (m, 2H), 3.65 (m, 1H), 3.56-3.45 (m, 1H), 3.43 (s, 2H), 3.34 (s, 1H), 3.22 (m, 1H), 3.14 (m, 1H), 2.31 (m, 1H), 2.20 (m, 2H), 2.03 (m, 1H), 1.74 (m, 2H). ¹³C NMR; DEPT-135 (101 MHz, MeOD) δ 129.35 (CH), 111.35 (CH), 65.25 (CH₂), 60.54 (CH), 55.14 (CH₂), 51.56 (CH₂), 38.28 (CH), 36.90 (CH), 32.41 (CH₂), 26.25 (CH₂), 25.35 (CH), 17.86 (CH₂). ESI-MS: measured m/z 300.33 [M+H]⁺. Purity by HPLC: 98.2% at 254 nm.

### Example 77: Synthesis of 7-ethyl-2-methoxy-5,6,6a,7,8,9,10,13-octahydro-12H-6,9-methanopyrido[1′,2′:1,2]azepino[4,5-b]indole-12-thione (28)

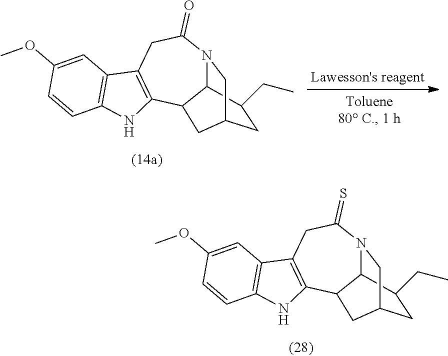

Under an inert atmosphere of argon gas, a solution of 7-ethyl-2-methoxy-5,6,6a,7,8,9,10,13-octahydro-12H-6,9-methanopyrido[1′,2′:1,2]azepino[4,5-b]indol-12-one (14a) (1 eq) in dry toluene (0.05 M) at 25° C. was added Lawesson's reagent (0.55 eq). The reaction was heated at 80° C. and stirred for 1 hour. The reaction completion was confirmed by LCMS and TLC. The organic solvent was removed under reduced pressure and dried. The crude was purified by normal phase silica gel column chromatography, running a mobile phase of 20% Ethyl acetate in 80% Hexane, and the product containing fractions were dried under reduced pressure to afford the desired product of 7-ethyl-2-methoxy-5,6,6a,7,8,9,10,13-octahydro-12H-6,9-methanopyrido[1′,2′:1,2]azepino[4,5-b]indole-12-thione (28) (20 mg, 63% yield). ¹H NMR (400 MHz, CDCl₃) δ 7.68 (s, 1H), 7.15 (d, J=8.7 Hz, 1H), 6.99 (d, J=2.4 Hz, 1H), 6.79 (dd, J=8.7, 2.4 Hz, 1H), 4.61 (s, 1H), 4.52-4.29 (m, 3H), 3.85 (s, 3H), 3.52 (d, J=12.9 Hz, 1H), 3.15-3.06 (m, 1H), 2.26-2.07 (m, 2H), 1.94 (d, J=8.5 Hz, 2H), 1.68-1.04 (m, 4H), 1.00 (t, J=7.4 Hz, 3H). DEPT-135 (101 MHz, CDCl₃) δ 112.22 (CH), 111.17 (CH), 100.16 (CH), 57.73 (CH₂), 56.20 (CH₃), 55.91 (CH), 42.51 (CH₂), 38.53 (CH), 35.86 (CH), 31.72 (CH₂), 30.06 (CH₂), 28.71 (CH₂), 28.55 (CH), 12.07 (CH₃). ESI-MS: measured m/z 341.20 [M+H]⁺. Purity by HPLC: 96% at 254 nm.

### Example 78: Synthesis of 7-ethyl-2-methoxy-5,6,6a,7,8,9,10,13-octahydro-12H-6,9-methanopyrido[1′,2′:1,2]azepino[4,5-b]indol-12-imine (30)

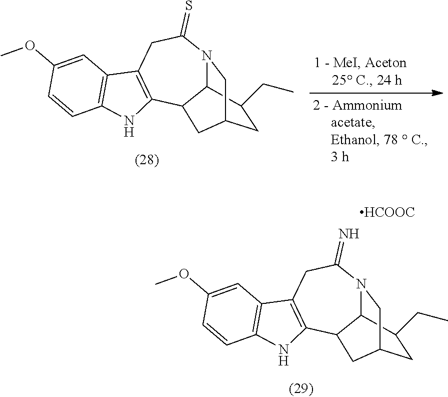

Under an inert atmosphere of argon gas, a solution of 7-ethyl-2-methoxy-5,6,6a,7,8,9,10,13-octahydro-12H-6,9-methanopyrido[1′,2′:1,2]azepino[4,5-b]indole-12-thione (28) (1 eq) in acetone (0.02 M) at 25° C. was added iodomethane (2.5 eq). The reaction was stirred for 24 hours. The reaction completion was confirmed by LCMS. The organic solvent was removed under reduced pressure and dried. The obtained crude product was dissolved in ethanol (0.04 M) and added ammonium acetate (10 eq) at 25° C. The reaction was heated at 78° C. and stirred for 3 hours. LCMS confirmed the formation of product. The organic solvent was removed under reduced pressure and dried. The crude was purified by reverse phase (C18) column chromatography, running a mobile phase of acetonitrile in 80% water, and 0.1% formic acid. The product containing fractions were dried under reduced pressure to afford the desired product of 7-ethyl-2-methoxy-5,6,6a,7,8,9,10,13-octahydro-12H-6,9-methanopyrido[1′,2′:1,2]azepino[4,5-b]indol-12-imine (29) (5 mg, 32% yield). ¹H NMR (400 MHz, MeOD) δ 8.44 (br s, 1H), 7.21 (d, J=8.7 Hz, 1H), 7.00 (d, J=2.4 Hz, 1H), 6.78 (dd, J=8.7, 2.4 Hz, 1H), 4.52 (s, 1H), 4.32 (d, J=16.7 Hz, 1H), 3.96 (d, J=16.7 Hz, 1H), 3.85 (s, 3H), 3.62 (d, J=11.6 Hz, 1H), 3.44 (d, J=11.8 Hz, 1H), 3.31-3.27 (m, 1H), 2.40-2.31 (m, 1H), 2.27-2.21 (m, 1H), 2.10-2.00 (m, 2H), 1.70-1.41 (m, 4H), 1.07 (t, J=7.3 Hz, 3H). DEPT-135 (101 MHz, CDCl₃) δ 111.30 (CH), 111.31 (CH), 99.18 (CH), 54.86 (CH₃), 53.86 (CH), 53.87 (CH₂), 37.51 (CH), 35.12 (CH), 35.86 (CH), 31.70 (CH₂), 28.90 (CH₂), 28.85 (CH₂), 28.18 (CH₂), 10.70 (CH₃). ESI-MS: measured m/z 324.33 [M+H]⁺. Purity by HPLC: 95% at 254 nm.

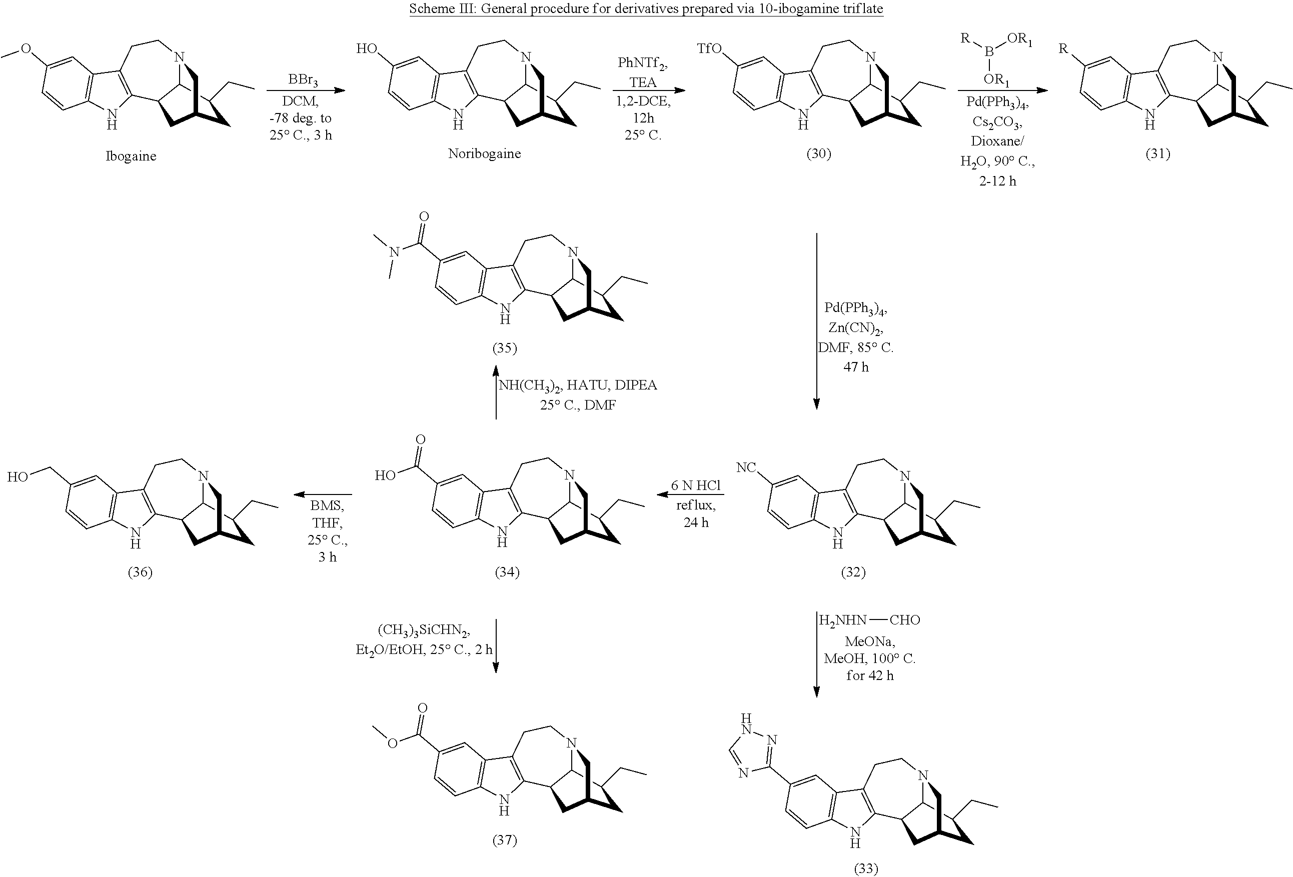

### General Procedure (XVIII): Synthesis of 10-Heterocyclic Ibogamine Derivatives (31)

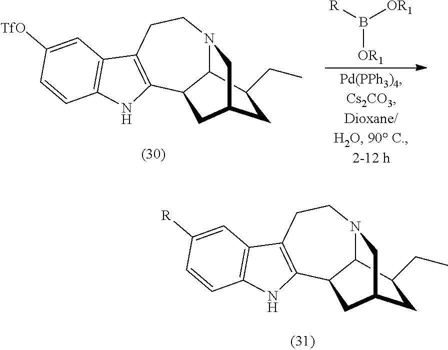

A microwave vial was charged with 16-ethyl-20,21-diazapentacyclononadeca-1,3(10),11,13-tetraen-10-yl) trifluoromethanesulfonate⁴ (30) (1 eq.), Boronic acid/ester (1.5 eq.), Cesium carbonate (2 eq.) then added Dioxane:Water (5:1; 0.1 M). The reaction mixture was degassed with argon balloon for 2 times, added Tetrakis(triphenylphosphine)palladium(0) (0.1 eq.) then the reaction vail was sealed and heated to 90° C. for 2-12 hours. After the starting material was consumed as judged by LCMS, reaction was cooled to room temp and diluted with 5 mL of EtOAC and 1 mL of water. Separate the layers and aqueous layer was extracted with EtOAc (20 mL), washed with brine, and dried on Na₂SO₄. The dried mixture was filed, the filtrate was concentrated, and the residue was purified by normal phase silica gel column chromatography, running a mobile phase of 0-100% EtOAc in Hexane, and the product containing fractions were dried under reduced pressure to afford the desired product.

### Example 79: Synthesis of 10-(1H-Pyrazol-4-Yl) Ibogamine (38)

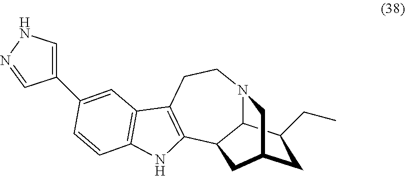

The title compound, 10-(1H-pyrazol-4-yl) ibogamine (38), was prepared according to the protocol described in general procedure (XVIII) and isolated as a white solid (10.0 mg, 25% yield). ¹H NMR (400 MHz, MeOD) δ 7.91 (s, 2H), 7.76 (br s, 1H), 7.62 (s, 1H), 7.34-7.23 (m, 2H), 3.68-3.45 (m, 2H), 3.29-2.91 (m, 6H), 2.22 (t, J=12.6 Hz, 1H), 2.06-1.95 (m, 2H), 1.86-1.74 (m, 1H), 1.72-1.49 (m, 4H), 1.00 (t, J=7.3 Hz, 3H). ESI-MS: measured m/z 347.27 [M+H]⁺. Purity by HPLC: 97.9% at 254 nm.

### Example 80: Synthesis of 10-(2-Hydroxy Pyrimidin-5-Yl) Ibogamine (39)

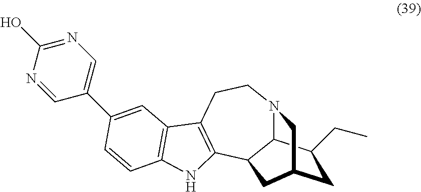

The title compound, 10-(2-hydroxy pyrimidin-5-yl) Ibogamine (39), was prepared according to the protocol described in general procedure (XVIII) and isolated as a white solid (10 mg, 23% yield). ¹H NMR (400 MHz, MeOD) δ 8.59 (s, 2H), 7.63 (d, J=1.8 Hz, 1H), 7.38 (d, J=8.4 Hz, 1H), 7.25 (dd, J=8.4, 1.7 Hz, 1H), 3.73-3.34 (m, 7H), 3.30-3.05 (m, 1H), 2.30 (t, J=12.6 Hz, 1H), 2.13-2.03 (m, 2H), 1.94-1.88 (m, 1H), 1.75-1.63 (m, 3H), 1.40-1.37 (m, 1H), 1.04 (t, J=7.3 Hz, 3H). ESI-MS: measured m/z 375.33 [M+H]⁺. Purity by HPLC: 98.3% at 254 nm.

### Example 81: Synthesis of 10-(Thiophen-2-Yl) Ibogamine (40)

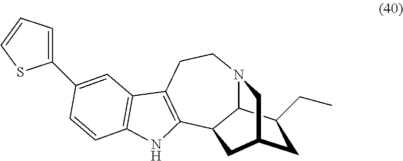

The title compound, 10-(thiophen-2-yl) Ibogamine (40), was prepared according to the protocol described in general procedure (XVIII) and isolated as a pale-yellow solid (23 mg, 41% yield). ¹H NMR (400 MHz, CDCl₃) δ 7.72 (d, J=1.7 Hz, 1H), 7.66 (s, 1H), 7.45-7.39 (m, 1H), 7.31-7.21 (m, 3H), 7.13-7.06 (m, 1H), 3.48-3.35 (m, 2H), 3.26-3.00 (m, 3H), 2.98-2.87 (m, 2H), 2.79-2.69 (m, 1H), 2.12-2.02 (m, 1H), 1.92-1.80 (m, 2H), 1.72-1.48 (m, 4H), 1.30-1.23 (m, 1H), 0.94 (t, J=7.0 Hz, 3H). ESI-MS: measured m/z 363.27[M+H]⁺. Purity by HPLC: 98.1% at 254 nm.

### Example 82: Synthesis of 10-(furan-2-yl) Ibogamine (41)

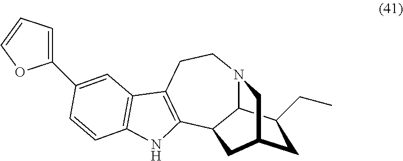

The title compound, 10-(furan-2-yl) Ibogamine (41), was prepared according to the protocol described in general procedure (XVIII) and isolated as a pale-yellow solid (12 mg, 40% yield). ¹H NMR (400 MHz, MeOD) δ 7.81 (d, J=1.6 Hz, 1H), 7.54-7.45 (m, 2H), 7.32 (d, J=8.5 Hz, 1H), 6.64 (d, J=3.3 Hz, 1H), 6.52-6.46 (m, 1H), 3.75-3.57 (m, 3H), 3.47-3.36 (m, 3H), 3.32-3.23 (m, 2H), 2.34 (t, J=12.8 Hz, 1H), 2.24-2.12 (m, 2H), 2.08-1.96 (m, 1H), 1.78-1.62 (m, 3H), 1.44-1.38 (m, 1H), 1.06 (t, J=7.3 Hz, 3H). ESI-MS: measured m/z 347.33[M+H]⁺. Purity by HPLC: 96.4% at 254 nm.

### Example 83: Synthesis of ibogamine-10-carbonitrile (32)

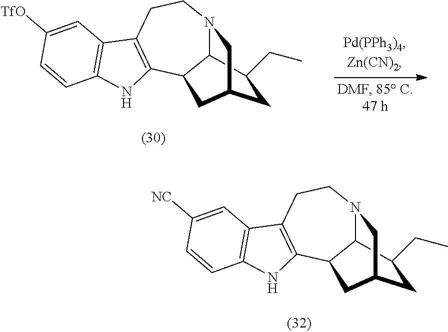

The titled compound, ibogamine-10-carbonitrile (32), was prepared according to literature procedure⁴ to afford the desired product (98 mg, 35% yield) as a white solid. ¹H NMR (400 MHz, CDCl₃) δ 8.15 (s, 1H), 7.81 (s, 1H), 7.40-7.26 (m, 2H), 3.49-3.29 (m, 2H), 3.24-2.94 (m, 4H), 2.92-2.84 (m, 1H), 2.69-2.59 (m, 1H), 2.15-2.04 (m, 1H), 1.93-1.79 (m, 2H), 1.71-1.43 (m, 4H), 1.33-1.20 (m, 1H), 0.92 (t, J=6.9 Hz, 3H). ESI-MS: measured m/z 306.27 [M+H]⁺. Purity by HPLC: 97.7% at 254 nm.

### Example 84: Synthesis of 10-(1H-1,2,4-triazol-3-yl) ibogamine (33)

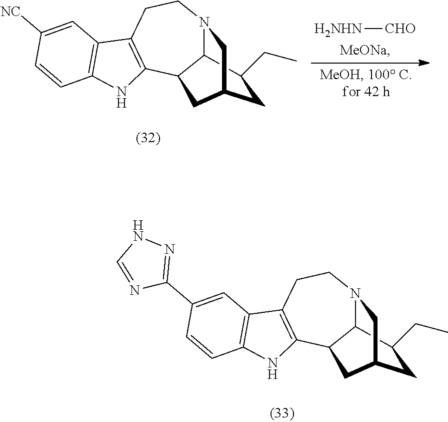

In a microwave vial, ibogamine-10-carbonitrile (32) (35.00 mg, 114.60 μmol, 1.0 eq.) was dissolved in Methanol (0.6 M). Sodium methoxide (10.0 eq.) was added to the reaction mixture at RT under an inert atmosphere and stirred for an hour. Then formic acid hydrazide (6.0 eq.) was added to the reaction mixture, the vial was sealed and heated to 100° C. for 42 hours. The reaction mixture was cooled to rt and transferred it to a 25 mL RBF, removed all the volatiles from the reaction mixture under reduced pressure and the residue was purified by normal phase silica gel column chromatography, running a mobile phase of 0-30% MeOH in DCM, and the product containing fractions were dried under reduced pressure to afford the desired product (33) (7.0 mg, 17% yield). ¹H NMR (400 MHz, MeOD) δ 8.50 (br s, 1H), 8.25 (s, 1H), 8.16 (s, 1H), 7.76 (dd, J=8.5, 1.7 Hz, 1H), 7.42 (d, J=8.5 Hz, 1H), 3.80-3.71 (m, 1H), 3.71-3.60 (m, 2H), 3.49-3.34 (m, 5H), 2.37 (t, J=12.8 Hz, 1H), 2.23-2.12 (m, 2H), 2.08-2.01 (m, 1H), 1.83-1.62 (m, 3H), 1.46-1.38 (m, 1H), 1.07 (t, J=7.3 Hz, 3H). ESI-MS: measured m/z 348.27 [M+H]⁺. Purity by HPLC: 90.7% at 254 nm.

### Example 85: Synthesis of ibogamine-10-carboxylic acid (34)

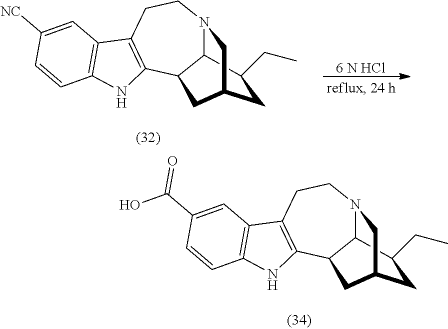

25 mL RBF was charged with ibogamine-10-carbonitrile (32) (22 mg, 72.03 μmol, 1.0 eq.) and added 6N HCl (1 mL/18 μmol). Heated to 100° C. for 22 hours, after completion of the starting material reaction mixture was concentrated. The residue was purified on reverse phase C18 silica gel column chromatography, running mobile phase of 0-100% Water/Acetonitrile (0.1% Formic acid), the product containing fractions were concentrated and lyophilized, obtained off-white solid (15.0 mg, 63% yield). ¹H NMR (400 MHz, MeOD) δ 8.68 (s, 1H), 8.33 (s, 1H), 7.93 (d, J=8.4 Hz, 1H), 7.41 (d, J=8.4 Hz, 1H), 3.81-3.70 (m, 1H), 3.70-3.55 (m, 2H), 3.53-3.41 (m, 4H), 3.34-3.21 (m, 1H), 2.42 (t, J=12.8 Hz, 1H), 2.22 (t, J=11.0 Hz, 2H), 2.06 (s, 1H), 1.90-1.68 (m, 3H), 1.53-1.45 (m, 1H), 1.15 (t, J=7.3 Hz, 3H). ESI-MS: measured m/z 325.27 [M+H]⁺. Purity by HPLC: 97.0% at 254 nm.

### Example 86: Synthesis of N,N-dimethyl ibogamine-10-carboxamide (35)

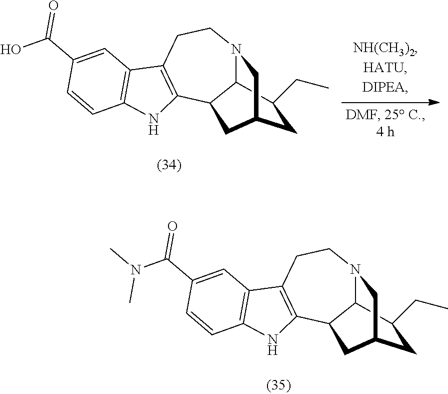

To a stirred solution of ibogamine-10-carboxylic acid (34) (20 mg, 61.65 μmol, 1.0 eq.) in DMF (0.2M), HATU (1.07 eq.), N,N-Diisopropylethylamine (2.0 eq.), and Dimethylamine in 2.0 M THF (15 eq.) were added at RT under inert atmosphere. After stirring for 4 h, 10 mL of EtOAc was added to the reaction mixture and followed by 2 mL of water. The reaction mixture was transferred to a separatory funnel, and the separated aqueous layer was extracted with EtOAc (2×10 mL). The combined the organic layers, washed with brine, and dried on Na₂SO₄. The organic phase was filtered, and the filtrate was concentrated under vacuum. The residue was purified by normal phase silica gel column chromatography, running a mobile phase of 0-20% MeOH in DCM, and the product-containing fractions were dried under reduced pressure to afford the desired product (17.0 mg, 78% yield). ¹H NMR (400 MHz, CD₃CN) δ 9.40 (s, 1H), 7.56 (d, J=1.5 Hz, 1H), 7.35 (d, J=8.3 Hz, 1H), 7.19 (dd, J=8.3, 1.6 Hz, 1H), 3.66-3.54 (m, 1H), 3.51-3.36 (m, 2H), 3.36-3.19 (m, 4H), 3.18-2.93 (m, 7H), 2.30-2.18 (m, 1H), 2.15-1.99 (m, 2H), 1.94-1.86 (m, 1H), 1.70-1.49 (m, 3H), 1.33-1.25 (m, 1H), 0.98 (t, J=7.3 Hz, 3H). ESI-MS: measured m/z 352.33 [M+H]⁺. Purity by HPLC: 96.4% at 254 nm.

### Example 87: Synthesis of ibogamine-10-methanol (36)

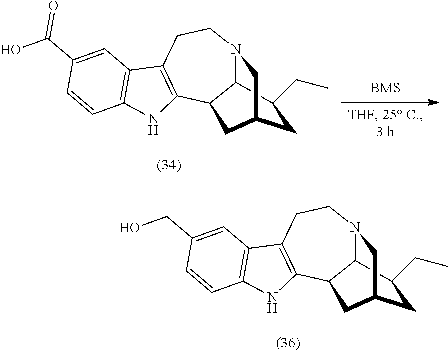

In a microwave vail ibogamine-10-carboxylic acid (34) (22.0 mg, 67.81 μmol, 1.0 eq.) was dissolved in THF (0.4 M) at RT under inert atmosphere. The reaction mixture was consequently cooled to 0° C. and Borane dimethyl sulfide complex (6.0 eq.) (diluted in 0.5 mL THF) was added in a dropwise manner. Next, the reaction was left at room temperature and continued to stir for 3 hours. The reaction was quenched with 0.3 mL MeOH and removed all the volatiles on the rotavapor. The reaction mixture was diluted with 15 mL of EtOAc, and 2 mL of 1 M Na₂CO₃ solution and transferred to a separating funnel. The aqueous layer was extracted with EtOAc (2×5 mL). Combined organic layers were dried over Na₂SO₄ and filtered. Filtrate was concentrated and the residue was purified by normal phase silica gel column chromatography, running a mobile phase of 0-100% MeOH in DCM, and the product containing fractions were dried under reduced pressure to afford the desired product (3.0 mg, 14% yield). ¹H NMR (400 MHz, MeOD) δ 7.44 (s, 1H), 7.25 (d, J=8.2 Hz, 1H), 7.11 (dd, J=8.3, 1.6 Hz, 1H), 4.68 (s, 2H), 3.59 (d, J=12.7 Hz, 1H), 3.49-3.35 (m, 3H), 3.32-3.20 (m, 3H), 3.10-3.01 (m, 1H), 2.27 (t, J=12.7 Hz, 1H), 2.11-2.02 (m, 2H), 1.93-1.85 (m, 1H), 1.75-1.57 (m, 4H), 1.02 (t, J=7.3 Hz, 3H). ESI-MS: measured m/z 311.27 [M+H]⁺. Purity by HPLC: 97.0% at 254 nm.

### Example 88: Synthesis of methyl ibogamine-10-carboxylate (37)

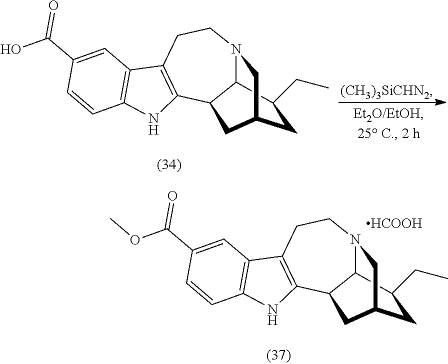

Ibogamine-10-carboxylic acid (34) (35.0 mg, 107.9 μmol, 1.0 eq.) was dissolved in methanol/diethylether (2:3; 0.9 M) at rt and treated with trimethylsilyldiazomethane, 2M in diethyl ether (37.0 eq.) for 2 hours. The volatiles were removed under reduced pressure from the reaction mixture and the residue was purified by reverse phase C18 silica gel column chromatography, running mobile phase of 0-100% Water/Acetonitrile (0.1% Formic acid). Product containing fractions were concentrated and lyophilized, obtained white solid (21.0 mg, 50% yield). ¹H NMR (400 MHz, MeOD) δ 8.51 (br s, 1H), 8.26-8.21 (m, 1H), 7.80 (dd, J=8.5, 1.5 Hz, 1H), 7.35 (d, J=8.5 Hz, 1H), 3.91 (s, 3H), 3.77-3.69 (m, 1H), 3.64 (d, J=8.4 Hz, 2H), 3.52-3.39 (m, 3H), 3.37 (s, 1H), 3.31-3.28 (m, 1H), 2.35 (t, J=12.8 Hz, 1H), 2.21-2.09 (m, 2H), 2.02 (t, J=7.8 Hz, 1H), 1.80-1.62 (m, 3H), 1.42 (dd, J=13.2, 5.9 Hz, 1H), 1.05 (t, J=7.1 Hz, 3H). ESI-MS: measured m/z 339.27 [M+H]⁺. Purity by HPLC: 98.4% at 254 nm.

1. A compound of Formula (I): 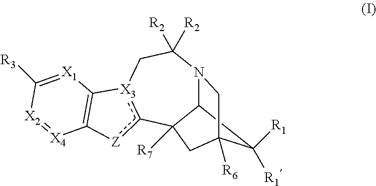
or a pharmaceutically acceptable salt or stereoisomer thereof, wherein:
X₁, X₂ and X₄ are independently C(R₃) or N;
X₃ is C or N;
R₁ and R₁′ are independently hydrogen, alkyl, —CF₃, —CH(Rₐ)(R_{b}), —CH₂ORₐ, —CH₂CH₂ORₐ, —CH₂NH₂, —CH₂NRₐRₐ, —CH₂SRₐ, —C(═O)ORₐ, —C(═O)NHRₐ, —ORₐ, or —C(═O)N(Rₐ)(R_{b});
R₂ and R₂′ are independently hydrogen, deuterium, —CH₂OH, —CH₂O-alkyl, —C(═O)OH, —C(═O)N(Rₐ)(R_{b}), or R₂ and R₂′ taken together with the atom to which they are attached form ═O, ═S, ═NH, or ═N-alkyl;
each R₃ is independently hydrogen, deuterium, halogen, alkyl, —ORₐ, —NO₂, —CN, —CF₃, cycloalkyl, aryl, heteroaryl, —OAc, —SRₐ, —NH₂, —NH(alkyl), —NH(alkenyl), —NH(alkynyl), —NH(aryl), —NH(heteroaryl), —N(cycloalkyl), —C(═O)R_{b}, —S(═O)R_{b}, —S(═NH)(═O)R_{b}, —S(═O)₂R_{b}, —NHS(═O)₂R_{b}, —OC(═O)R_{b}, SC(═O)R_{b}, —NHC(═O)R_{c}, or —NHC(═S)R_{c};
R₆ is hydrogen, alkyl, —CH₂ORₐ, —CH₂NH₂, —CH₂N(Rₐ)(R_{b}), —CH₂SRₐ, —C(═O)ORₐ, —C(═O)NHRₐ, or —C(═O)N(Rₐ)(R_{b});
R₇ is hydrogen, halogen, deuterium, alkyl, alkoxy, alkylene-OH, alkylene-O-alkyl, alkylene-NH₂, alkylene-NH(alkyl), alkylene-N(alkyl)₂, —C(═O)ORₐ, or —C(═O)NRₐ;
each Rₐ is independently hydrogen, alkyl, deuterated alkyl, alkenyl, alkylene-aryl, alkylene-cycloalkyl, aryl, or heteroaryl;
each R_{b} is independently alkyl, aryl, heteroaryl, —OH, —O-alkyl, —NH₂, —NH(alkyl), or —N(alkyl)₂;
each R_{c} is independently alkyl, aryl, —O-alkyl, —S-alkyl, —S-aryl, —NH₂, —NH(alkyl), or —N(alkyl)₂; and
Z is O, S, or N(R₄), wherein R₄ is absent, hydrogen, alkyl, deuterated alkyl, heteroaryl, alkenyl, alkylene-aryl, alkylene-cycloalkyl, aryl, or —SO₂Rₐ.

2-5. (canceled)

6. The compound of claim 1, wherein the compound is a compound of Formula (I′): 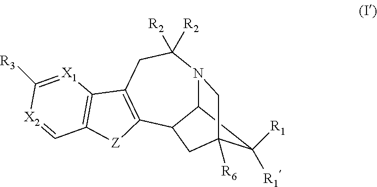
or a pharmaceutically acceptable salt, or stereoisomer thereof, wherein:
X₁ and X₂ are independently C(R₃), or N;
R₁ and R₁′ are independently hydrogen, alkyl, —CF₃, —CH(Rₐ)(R_{b}), —CH₂ORₐ, —CH₂CH₂ORₐ, —CH₂NH₂, —CH₂N(Rₐ)(Rₐ), —CH₂SRₐ, —C(═O)ORₐ, —CONHRₐ, or —CON(Rₐ)(Rₐ);
R₂ and R₂′ are independently hydrogen, deuterium, —CH₂OH, —CH₂O-alkyl, —COOH, —CON(Rₐ)(Rₐ), or R₂ and R₂′ taken together with the atom to which they are attached form ═O, ═S, ═NH, or ═N-alkyl;
each R₃ is independently hydrogen, deuterium, halogen, alkyl, —ORₐ, —NO₂, —CN, —CF₃, cycloalkyl, aryl, heteroaryl, —OAc, —SRₐ, —NH₂, —NH(alkyl), —NH(alkenyl), —NH(alkynyl), —NH(aryl), —NH(heteroaryl), —N(cycloalkyl), —C(═O)R_{b}, —S(═O)R_{b}, —S(═NH)(═O)R_{b}, —SO₂R_{b}, —NHSO₂R_{b}, —OC(═O)R_{b}, SC(═O)R_{b}, —NHC(═O)R_{c} or —NHC(═S)R_{c};
R₆ is hydrogen, alkyl, —CH₂ORₐ, —CH₂NH₂, —CH₂NRₐRₐ, —CH₂SRₐ, —C(═O)ORₐ, —CONHRₐ, or —CON(Rₐ)(Rₐ);
each Rₐ is independently hydrogen, alkyl, deuterated alkyl, alkenyl, alkylene-aryl, alkylene-cycloalkyl, aryl, or heteroaryl;
each R_{b} is independently alkyl, aryl, heteroaryl, —OH, —O-alkyl, —NH₂, —NH(alkyl), or —N(alkyl)₂;
each R_{c} is independently alkyl, aryl, —O-alkyl, —S-alkyl, —S-aryl, —NH₂, —NH(alkyl), or —N(alkyl)₂; and
Z is O, S, or NR₄, wherein R₄ is hydrogen, alkyl, deuterated alkyl, heteroaryl, alkenyl, alkylene-aryl, alkylene-cycloalkyl, aryl, or SO₂Rₐ.

7. (canceled)

8. The compound of claim 1, wherein X₁ is C(R₃).

9. (canceled)

10. (canceled)

11. The compound of claim 1, wherein X₂ is C(R₃).

12. The compound of claim 1, wherein R₃ is hydrogen, —CH₃, —F, —OH, or —OCH₃.

13-16. (canceled)

17. The compound of claim 1, wherein R₁ and R₁′ are hydrogen.

18-21. (canceled)

22. The compound of claim 1, wherein R₂ and R₂′ are hydrogen.

23-25. (canceled)

26. The compound of claim 6, wherein R₃ is —OH, hydrogen, —CH₃, —F, or —OCH₃.

27-30. (canceled)

31. The compound of claim 1, wherein R₆ is hydrogen or —CH₃.

32. (canceled)

33. (canceled)

34. The compound of claim 1, wherein Z is O or N(R₄).

35. (canceled)

36. The compound of claim 34, wherein R₄ is hydrogen.

37. (canceled)

38. (canceled)

39. The compound of claim 1 is selected from: 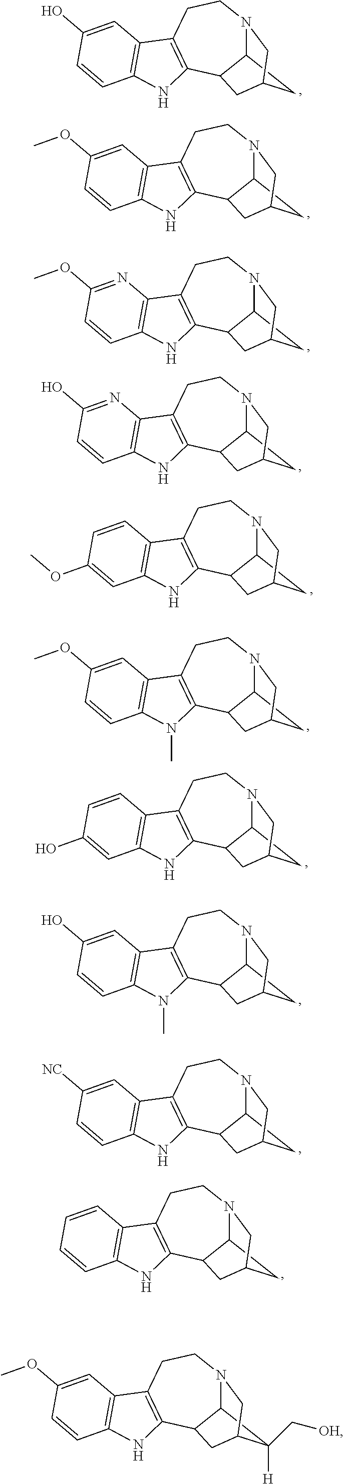 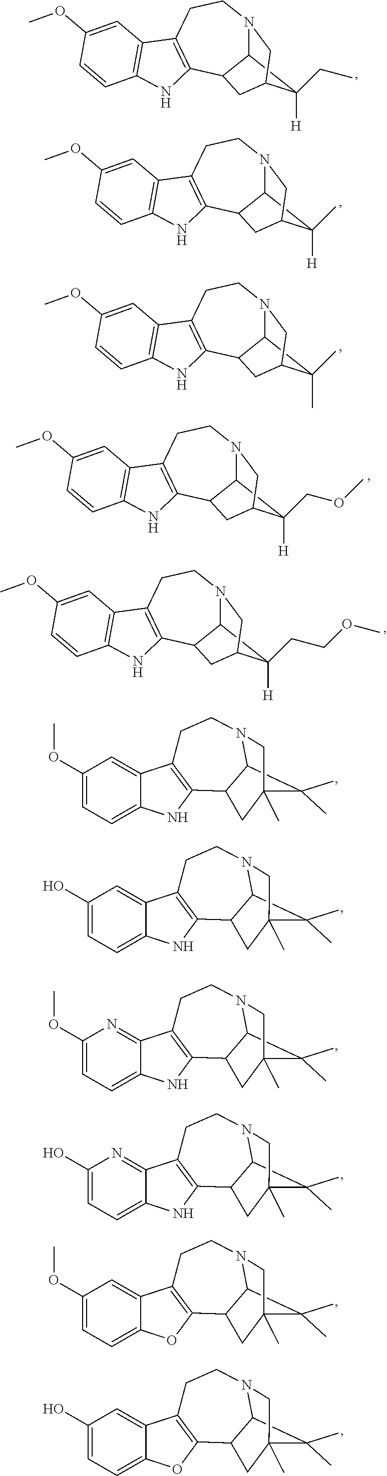 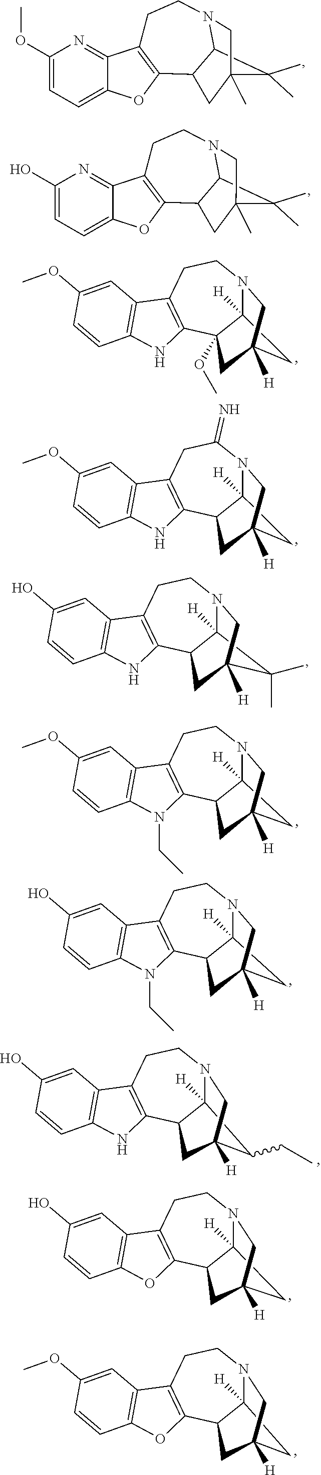 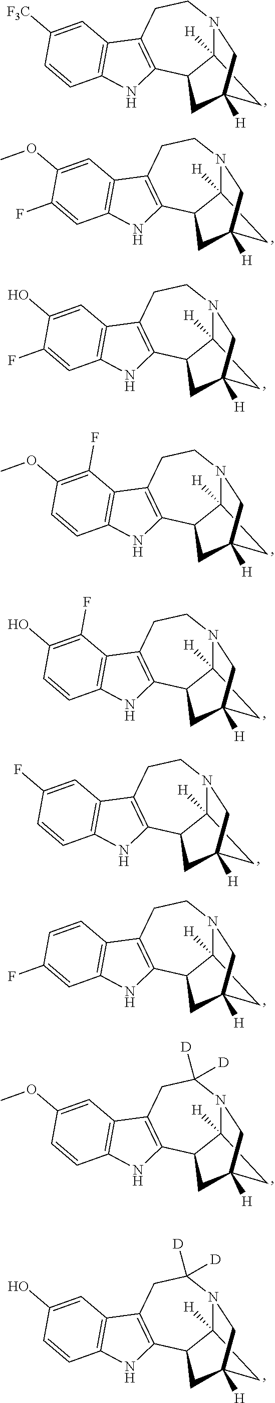 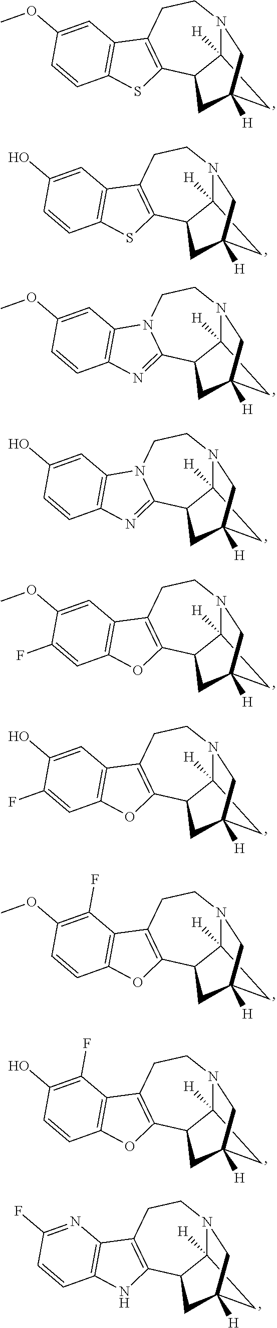 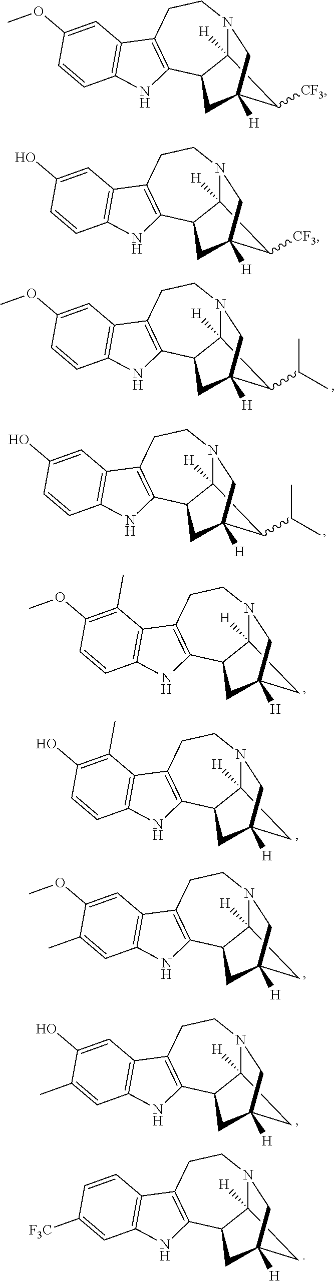
or a pharmaceutically acceptable salt or stereoisomer thereof.

40. A compound of Formula (II): 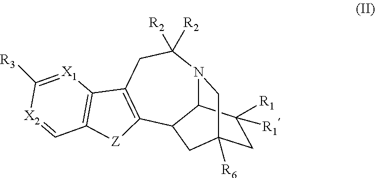
or a pharmaceutically acceptable salt or stereoisomer thereof, wherein:
X₁ and X₂ are independently C(R₃) or N, wherein at least one of X₁ and X₂ is N;
R₁ and R₁′ are independently hydrogen, alkyl, —CH₂ORₐ, —CH₂NH₂, —CH₂N(Rₐ)(Rₐ), —CH₂SRₐ, —C(═O)ORₐ, —CONHRₐ, or —CON(Rₐ)(Rₐ);
R₂ and R₂′ are independently hydrogen, deuterium, —CH₂OH, —CH₂O-alkyl, —COOH, —CON(Rₐ)Rₐ, or R₂ and R₂′ taken together with the atom to which they are attached form ═O, ═S, ═NH, or ═N-alkyl;
each R₃ is independently hydrogen, deuterium, halogen, alkyl, —ORₐ, —NO₂, —CN, cycloalkyl, aryl, heteroaryl, —OAc, —SRₐ, —NH₂, —NH(alkyl), —NH(alkenyl), —NH(alkynyl), —NH(aryl), —NH(heteroaryl), —N(cycloalkyl), —C(═O)R_{b}, —S(═O)R_{b}, —S(═NH)(═O)R_{b}, —SO₂R_{b}, —NHSO₂R_{b}, —OC(═O)R_{b}, —SC(═O)R_{b}, —NHC(═O)R_{c}, or —NHC(═S)R_{c};
each Rₐ is independently hydrogen, alkyl, deuterated alkyl, alkenyl, alkylene-aryl, alkylene-cycloalkyl, aryl, or heteroaryl;
R₆ is hydrogen, alkyl, —CH₂ORₐ, —CH₂NH₂, —CH₂N(Rₐ)(Rₐ), —CH₂SRₐ, —C(═O)ORₐ, —CONHRₐ, or —CON(Rₐ)(Rₐ);
each R_{b} is independently alkyl, aryl, heteroaryl, —OH, —O-alkyl, —NH₂, —NH(alkyl), or —N(alkyl)₂;
each R_{c} is independently alkyl, aryl, —O-alkyl, —S-alkyl, —S-aryl, —NH₂, —NH(alkyl), or —N(alkyl)₂; and
Z is O, S, or NR₄, wherein R₄ is hydrogen, alkyl, deuterated alkyl, heteroaryl, alkenyl, alkylene-aryl, alkylene-cycloalkyl, aryl, or —SO₂Rₐ.

41-50. (canceled)

51. The compound of claim 40, is selected from: 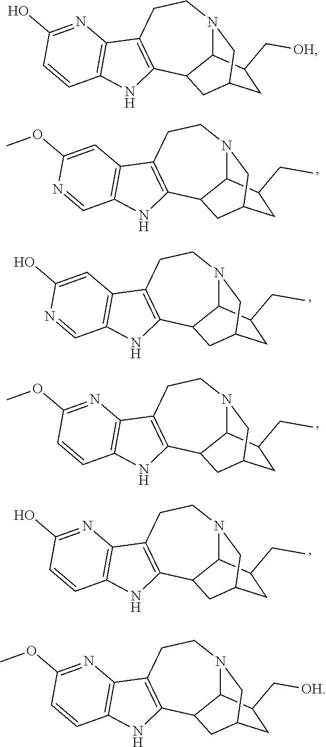
or a pharmaceutically acceptable salt or stereoisomer thereof.

52. A compound of Formula (II): 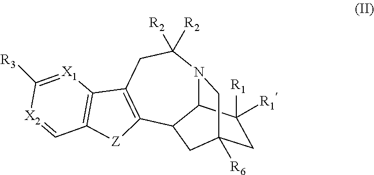
or a pharmaceutically acceptable salt or stereoisomer thereof, wherein:
X₁ and X₂ are independently C(R₃), or N;
R₁ and R₁′ are independently hydrogen, alkyl, —CH₂ORₐ, —CH₂NH₂, —CH₂N(Rₐ)(Rₐ), —CH₂SRₐ, —C(═O)ORₐ, —CONHRₐ, or —CON(Rₐ)Rₐ;
R₂ and R₂′ are independently hydrogen, —CH₂OH, —CH₂O-alkyl, —COOH, —CON(Rₐ)(Rₐ), or R₂ and R₂′ taken together with the atom to which they are attached form ═O, ═S, ═NH, or ═N— alkyl;
each R₃ is independently deuterium, halogen, alkyl, —ORₐ, —NO₂, —CN, cycloalkyl, aryl, heteroaryl, -OAc, —SRₐ, —NH₂, —NH(alkyl), —NH(alkenyl), —NH(alkynyl), —NH(aryl), —NH(heteroaryl), —N(cycloalkyl), —C(═O)R_{b}, —S(═O)R_{b}, —S(═NH)(═O)R_{b}, —SO₂R_{b}, —NHSO₂R_{b}, —SC(═O)R_{b}, —NHC(═O)R_{c} or —NHC(═S)R_{c};
R₆ is hydrogen, alkyl, —CH₂ORₐ, —CH₂NH₂, —CH₂N(Rₐ)(Rₐ), —CH₂SRₐ, —C(═O)ORₐ, —CONHRₐ, or —CON(Rₐ)(Rₐ);
each Rₐ is independently hydrogen, alkyl, deuterated alkyl, alkenyl, alkylene-aryl, alkylene-cycloalkyl, aryl, or heteroaryl;
each R_{b} is independently alkyl, aryl, heteroaryl, —OH, —O-alkyl, —NH₂, —NH(alkyl), or —N(alkyl)₂;
each R_{c} is independently alkyl, aryl, —O-alkyl, —S-alkyl, —S-aryl, —NH₂, —NH(alkyl), or —N(alkyl)₂; and
Z is N(R₄), wherein R₄ is hydrogen, alkyl, deuterated alkyl, heteroaryl, alkenyl, alkylene-aryl, alkylene-cycloalkyl, aryl, or —SO₂Rₐ;
wherein when R₂ and R₂′ are hydrogen, R₃ is —OH, and X₁ and X₂ are CH, then R₁ or R₁′ is not —CH₂CH₃, —(CH₂)₂CN, —(CH₂)₂OCH₂C₆H₅, —CD₂CD₃, —CH₂CD₃, —(CH₂)₂OH, —(CH₂)₂OCH₂CH₃, or —(CH₂)₂CF₃;
and wherein when R₁ or R₁′ is —CH₂CH₃ or —CH₂OH, and X₁ and X₂ are CH, then R₃ is not —OCH₂CH₃, —OC(CH₃)₃, or —CH═CH₂, or R₄ is not —(CH₂)₃N(CH₃)₂;
and the compound is not

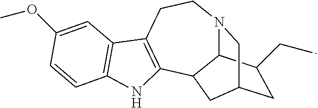 [SMILES: CCC1CC2CC3c4[nH]c5ccc(OC)cc5c4CCN(C2)C13]

53-81. (canceled)

82. The compound of claim 52, is selected from: 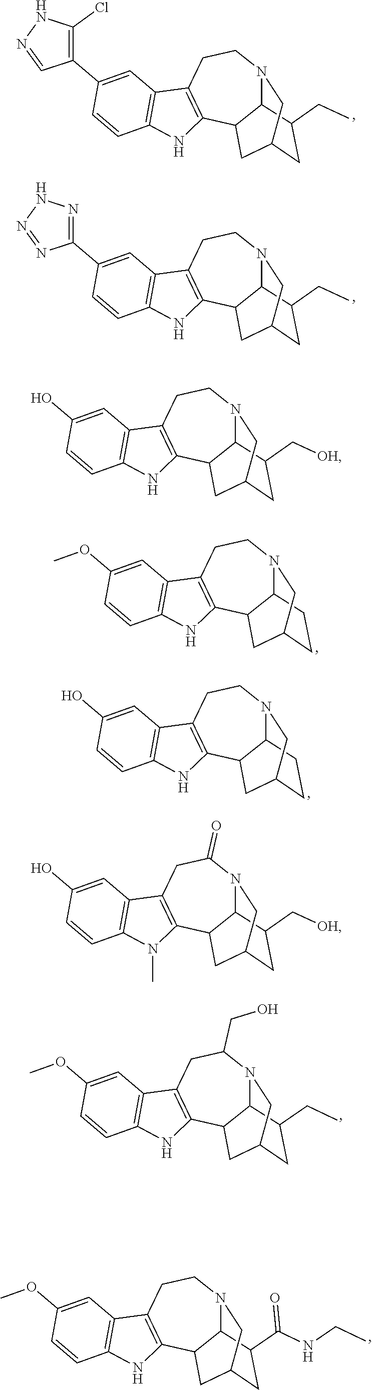 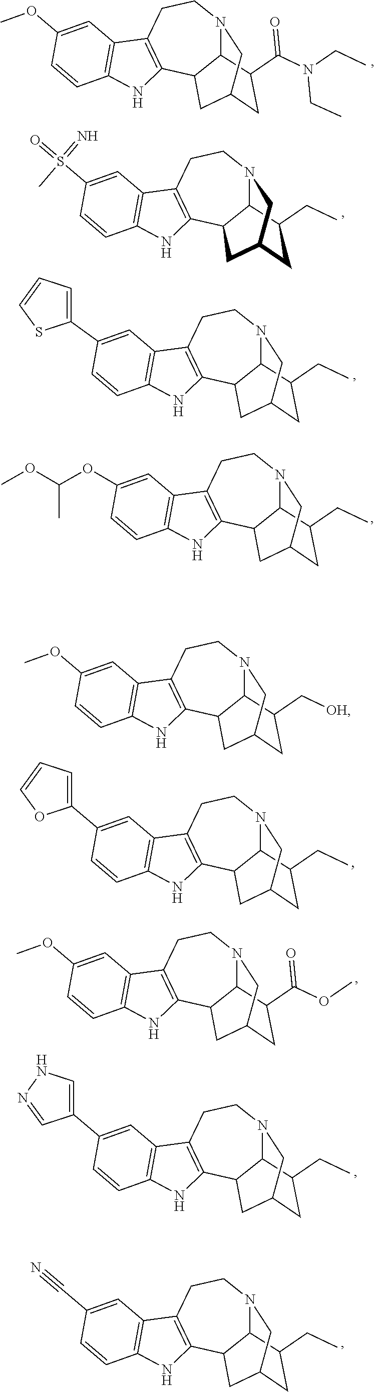 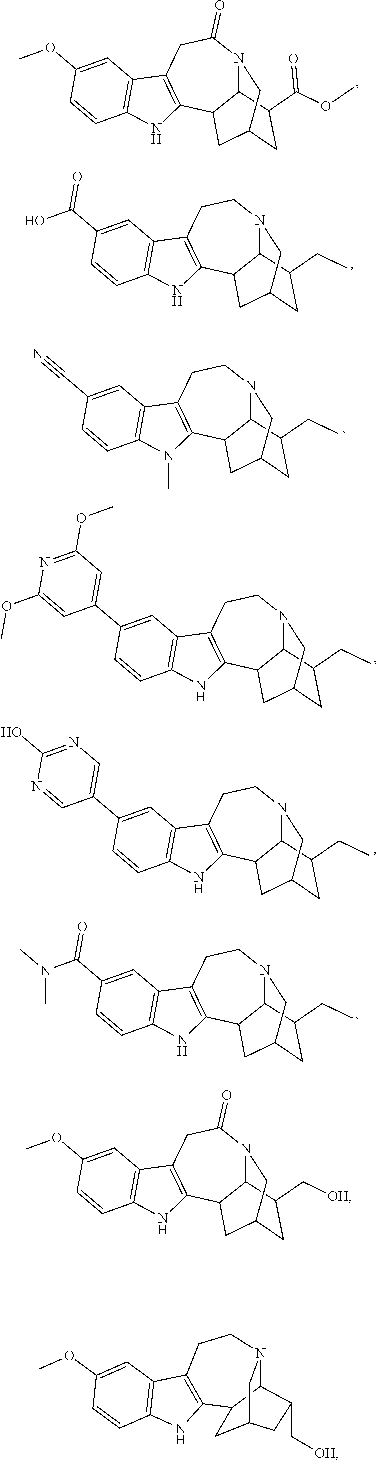 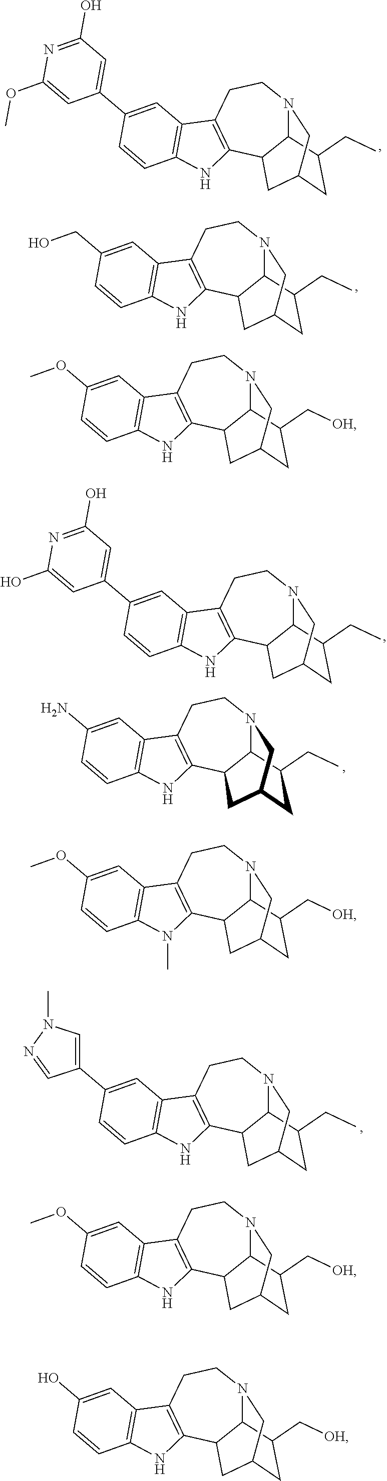 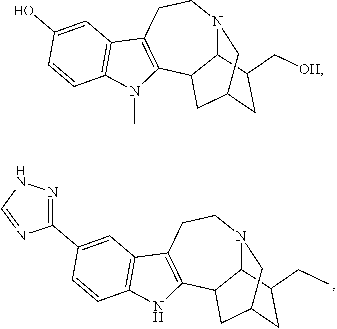 [SMILES: Cn1c2c(c3cc(O)ccc31)CCN1CC3CC(CO)C1C2C3 ; CCC1CC2CC3c4[nH]c5ccc(-c6nc[nH]n6)cc5c4CCN(C2)C13] 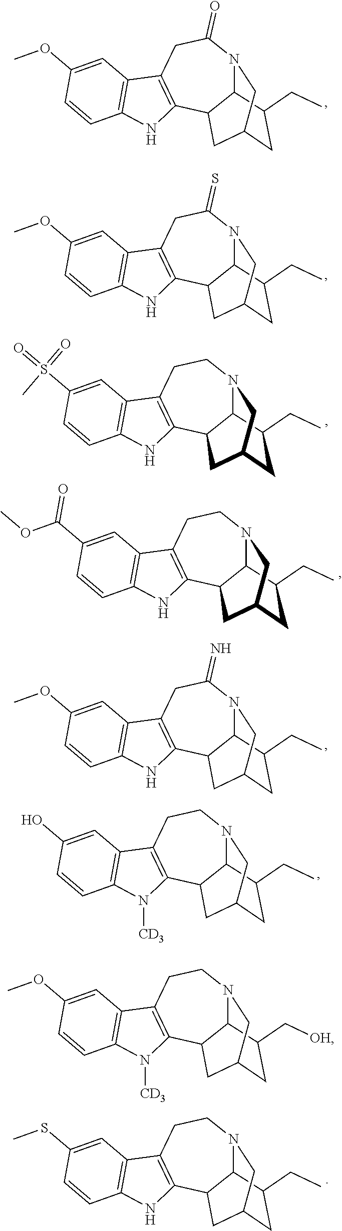
or a pharmaceutically acceptable salt or stereoisomer thereof.

83. A compound of Formula (III): 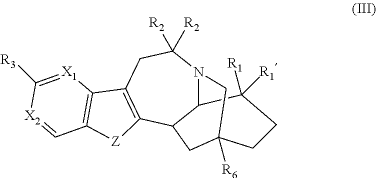
or a pharmaceutically acceptable salt or stereoisomer thereof, wherein:
X₁ and X₂ are independently C(R₃), or N;
R₁ and R₁′ are independently hydrogen, alkyl, —CH₂ORₐ, —CH₂CH₂ORₐ, —CH₂NH₂, —CH₂N(Rₐ)(Rₐ), —CH₂SRₐ, —C(═O)ORₐ, —CONRₐ, or —CON(Rₐ)(Rₐ);
R₂ and R₂′ are independently hydrogen, deuterium, —CH₂OH, —CH₂O-alkyl, —COOH, —CON(Rₐ)Rₐ, or R₂ and R₂′ taken together with the atom to which they are attached form ═O, ═S, ═NH, or ═N-alkyl;
each R₃ is independently hydrogen, deuterium, halogen, alkyl, —ORₐ, —NO₂, —CN, cycloalkyl, aryl, heteroaryl, -OAc, —SRₐ, —NH₂, —NH(alkyl), —NH(alkenyl), —NH(alkynyl), —NH(aryl), —NH(heteroaryl), —N(cycloalkyl), —C(═O)R_{b}, —S(═O)R_{b}, —S(═NH)(═O)R_{b}, —SO₂R_{b}, —NHSO₂R_{b}, —OC(═O)R_{b}, SC(═O)R_{b}, —NHC(═O)R_{c} or —NHC(═S)R_{c};
R₆ is hydrogen, alkyl, —CH₂ORₐ, —CH₂NH₂, —CH₂NRₐRₐ, —CH₂SRₐ, —C(═O)ORₐ, —CONHRₐ, or —CON(Rₐ)Rₐ;
each Rₐ is independently hydrogen, alkyl, deuterated alkyl, alkenyl, alkylene-aryl, alkylene-cycloalkyl, aryl, or heteroaryl;
each R_{b} is independently alkyl, aryl, heteroaryl, —OH, —O-alkyl, —NH₂, —NH(alkyl), or —N(alkyl)₂;
each R_{c} is independently alkyl, aryl, —O-alkyl, —S-alkyl, —S-aryl, —NH₂, —NH(alkyl), or —N(alkyl)₂; and
Z is N(R₄), wherein R₄ is hydrogen, alkyl, deuterated alkyl, heteroaryl, alkenyl, alkylene-aryl, alkylene-cycloalkyl, aryl, or, SO₂Rₐ.

84-102. (canceled)

103. A pharmaceutical composition comprising the compound of claim 1 or pharmaceutically acceptable salt, or stereoisomer thereof.

104. (canceled)

105. A method of treating a disease or disorder in a subject in need thereof comprising administering a therapeutically effective amount of the compound of claim 1 to the subject.

106. The method of claim 105, wherein the disease or disorder is alcoholism, substance abuse disorder, opioid use disorder, depression, major depression, chronic pain, acute pain, eating disorder, anxiety disorder, obsessive-compulsive disorder (OCD), stress disorder, post-traumatic stress disorder (PTSD), acute stress disorder, panic disorder, social anxiety disorder, generalized anxiety disorder, specific phobia, opioid use disorder (OUD), alcohol use disorder (AUD), polydrug use disorder, headache, migraine, traumatic brain injury (TBI), Parkinson's disease, substance use disorder (SUD), or nicotine/tobacco use disorder.

107. (canceled)
